(19) United States
(12) Patent Application Publication
(10) Pub. No.: US 2024/0000971 A1
(43) Pub. Date: Jan. 4, 2024

(54) COMPOSITIONS AND METHODS FOR THE TREATMENT OF TAUOPATHY

(71) Applicant: Voyager Therapeutics, Inc., Cambridge, MA (US)

(72) Inventors: Todd Carter, Cambridge, MA (US); Wencheng Liu, Cambridge, MA (US); Jerrah Holth, Cambridge, MA (US); Jinzhao Hou, Cambridge, MA (US); Yanqun Shu, Cambridge, MA (US); Giridhar Murlidharan, Cambridge, MA (US); Xiao-Qin Ren, Cambridge, MA (US)

(21) Appl. No.: 18/035,236

(22) PCT Filed: Nov. 3, 2021
(86) PCT No.: PCT/US2021/057823

§ 371 Date: May 3, 2023

Related U.S. Application Data
(60) Provisional application No. 63/109,706, filed on Nov. 4, 2020.

(51) Int. Cl. A61K 48/00; C12N 15/86; A61P 25/28; C07K 16/18

(52) U.S. Cl. CPC A61K 48/0058; C12N 15/86; A61K 48/0066; A61P 25/28; C07K 16/18

(57) ABSTRACT

The disclosure provides compositions and methods for the preparation, manufacture and therapeutic use of viral vectors, such as adeno-associated virus (AAV) particles having viral genomes encoding one or more antibodies or antibody fragments or antibody-like polypeptides, for the prevention and/or treatment of diseases and/or disorders.

### REFERENCE TO RELATED APPLICATION

The present application claims priority to U.S. Provisional Patent Application No. 63/109,706, filed on Nov. 4, 2020, the entire contents of which, including all drawings and sequence listing, are incorporated herein by reference.

### REFERENCE TO THE SEQUENCE LISTING

The present application is being filed along with a Sequence Listing in electronic format. The Sequence Listing file, entitled SEQ_LST_135333_02020, was created on Nov. 2, 2021, and is 18,388,346 bytes in size. The information in electronic format of the Sequence Listing is incorporated herein by reference in its entirety.

### FIELD OF THE DISCLOSURE

The disclosure relates to compositions and methods for vectored antibody delivery (VAD), wherein the antibody may be an anti-tau antibody.

### BACKGROUND OF THE INVENTION

Tauopathies are a group of neurodegenerative diseases characterized by the dysfunction and/or aggregation of the microtubule associated protein tau. Tau is normally a very soluble protein known to associate with microtubules based on the extent of its phosphorylation. Tau is considered a critical component of intracellular trafficking processes, particularly in neuronal cells, given their unique and extended structure. Hyperphosphorylation of tau depresses its binding to microtubules and microtubule assembly activity. Further, hyperphosphorylation of tau renders it prone to misfolding and aggregation. In tauopathies, the tau becomes hyperphosphorylated, misfolds and aggregates as neurofibrillary tangles (NFT) of paired helical filaments (PHF), twisted ribbons or straight filaments. These NFT are largely considered indicative of impending neuronal cell death and thought to contribute to widespread neuronal cell loss, leading to a variety of behavioral and cognitive deficits.

The first genetically defined tauopathy was described when mutations in the tau gene were shown to lead to an autosomal dominantly inherited tauopathy known as frontotemporal dementia and parkinsonism linked to chromosome 17 (FTDP-17). This was the first causal evidence that changes in tau could lead to neurodegenerative changes in the brain. These molecules are considered to be more amyloidogenic, meaning they are more likely to become hyperphosphorylated and more likely to aggregate into NFT (Hutton, M. et al., 1998, Nature 393(6686):702-5).

Other known tauopathies include, but are not limited to, Alzheimer's disease (AD), Frontotemporal lobar degeneration (FTLD), Frontotemporal dementia, chronic traumatic encephalopathy (CTE), Progressive Supranuclear Palsy (PSP), Down's syndrome, Pick's disease, Corticobasal degeneration (CBD), Corticobasal syndrome, Amyotrophic lateral sclerosis (ALS), Prion diseases, Creutzfeldt-Jakob disease (CJD), Multiple system atrophy, Tangle-only dementia, and Progressive subcortical gliosis.

Several approaches have been proposed for therapeutically interfering with progression of tau pathology and preventing the subsequent molecular and cellular consequences. Given that NFT are composed of hyperphosphorylated, misfolded and aggregated forms of tau, interference at each of these stages has yielded a set of avidly pursued targets. Introducing agents that limit phosphorylation, block misfolding or prevent aggregation have all generated promising results. Passive and active immunization with late stage anti-phospho-tau antibodies in mouse models have led to dramatic decreases in tau aggregation and improvements in cognitive parameters. It has also been suggested that introduction of anti-tau antibodies can prevent the trans-neuronal spread of tau pathology.

Antibodies have relatively short half-lives, and this presents an ongoing and long-felt challenge for antibody-based therapies. In order to achieve a sufficiently high concentration of an antibody for long lasting therapeutic effects, antibody therapies are traditionally delivered by repeated administration, e.g. by multiple injections. This dosing regimen results in an inconsistent level of antibody throughout the treatment period, limited efficiency per administration, high cost of administration and consumption of the antibody. Hence, there remains a need in the art for delivery of antibodies and antibody-based therapeutics through alternative routes or modalities of administration.

One such alternative route of administration is by expression vectors (e.g. plasmid or viral vector), including but not limited to, adeno-associated viral vectors (AAVs). Adeno-associated viral vectors are widely used in gene therapy approaches due to a number of advantageous features. As dependoparvoviruses, AAV are non-replicating in infected cells and therefore not associated with any known disease. Further, AAVs may be introduced to a wide variety of host cells, do not integrate into the genome of the host cell, and are capable of infecting both quiescent and dividing cells. AAVs transduce non-replicating and long-lived cells in vivo, resulting in long term expression of the protein of interest. Further, AAVs can be manipulated with cellular and molecular biology techniques to produce non-toxic particles carrying a payload encoded in the AAV viral genome that can be delivered to a target tissue or set of cells with limited or no side-effects. Given the foregoing, the use of AAVs for vectored antibody delivery (VAD) of anti-tau antibodies would allow for longer lasting efficacy, fewer dose treatments, and more consistent levels of the antibody throughout the treatment period.

In vectored antibody delivery (VAD) of anti-tau antibodies, an AAV is used as the delivery modality for a nucleic acid sequence encoding the anti-tau antibody, or a fragment thereof, which results in in vivo expression of the encoded payload, e.g., functional anti-tau antibody, or a fragment thereof.

The mechanism underlying VAD is thought to proceed through the following steps. First, the AAV vector enters the cell via endocytosis, then escapes from the endosomal compartment and is transported to the nucleus wherein the viral genome is released and converted into a double-stranded episomal molecule of DNA by the host. The transcriptionally active episome results in the expression of encoded anti-tau antibodies that may then be secreted from the cell into the circulation. VAD may therefore enable continuous, sustained and long-term delivery of anti-tau antibodies administered by a single injection of AAV particles.

Previous studies of an AAV-mediated antibody technique known as vectored immunoprophylaxis (VIP) have focused on neutralization of human immunodeficiency virus (HIV) (see, e.g. Johnson et al., 2009, Nature Med., 15, 901-906, Saunders et al., 2015, J. Virol., 89(16), 8334-8345, Balasz et al., 2012, Nature 481, 81-84, the contents of which are incorporated herein by reference in their entirety). Balasz et al. reported a long-term, even lifelong, expression of monoclonal antibody at high concentration from a single intramuscular administration in mice that resulted in full protection against HIV infection. AAV-mediated VIP has also been demonstrated against influenza strains (see, e.g. Balasz, et al. Nat. Biotechnol., 2013, 31(7):647-52) and Plasmodium Falciparum, a sporozoite causing malaria infection (see, e.g. Deal at al., 2014, PNAS, 111 (34), 12528-12532), as well as cancer, RSV and drug addiction (see, e.g. review by Schnepp and Johnson, Microbiol. Spectrum 2(4), 2014). Though promising, these studies emphasize efforts to prevent disease. There still remains a need for improved methods of prevention, and new antibody-mediated therapies for research, diagnosis, and treatment of disease.

The present disclosure addresses this need by providing novel AAV particles having viral genomes engineered to encode anti-tau antibodies and antibody-based compositions and methods of using these constructs (e.g., VAD) for the treatment, prevention, diagnosis and research of diseases, disorders and/or conditions associated with tau pathology. The present disclosure further embraces optimized AAV particles for delivery of nucleic acids (e.g., viral genomes) encoding anti-tau antibodies and antibody-based compositions to a subject in need thereof.

### SUMMARY OF THE INVENTION

The present disclosure describes AAV particles for delivery of anti-tau antibodies to a target tissue. AAV particles of the present disclosure may comprise an AAV capsid and a viral genome.

In one aspect, the disclosure provides an isolated, e.g., recombinant, nucleic acid comprising a transgene encoding an antibody, e.g., an antibody fragment, that binds to tau comprising a heavy chain variable region (VH) and a light chain variable region (VL), wherein: (i) the nucleotide sequence encoding the VH, comprises the nucleotide sequence of SEQ ID NO: 1839, 1841, or 2170, or a nucleotide sequence having at least 90%, 92%, 95%, 96%, 97%, 98%, or 99% sequence identity thereto; and/or (ii) the nucleotide sequence encoding the VL comprises the nucleotide sequence of SEQ ID NO: 1957 or 4565, or a nucleotide sequence having at least 90%, 92%, 95%, 96%, 97%, 98%, or 99% sequence identity thereto.

Another aspect of the disclosure provides an isolated, e.g., recombinant, antibody, e.g., antibody fragment, encoded by the nucleic acid of the disclosure.

Another aspect of the disclosure provides a viral genome, e.g., an adeno-associated (AAV) viral genome, which comprises a first promoter operably linked to the transgene encoding an antibody, e.g., antibody fragment, described herein.

Another aspect of the disclosure provides an AAV viral genome comprising: (i) the nucleotide sequence of any one of SEQ ID NOs: 4547, 4548, 4551, 4552, 4555, 4556, 4559, or 4560, or a nucleotide sequence having at least 90%, 92%, 95%, 96%, 97%, 98%, or 99% sequence identity thereto; (ii) the nucleotide sequence of any one of SEQ ID NOs: 4549, 4550, 4553, 4554, 4557, 4558, 4561, or 4562, or a nucleotide sequence having at least 90%, 92%, 95%, 96%, 97%, 98%, or 99% sequence identity thereto, optionally wherein the nucleotide sequence comprises or does not comprise a tag, e.g., comprising the nucleotide sequence of SEQ ID NO: 2118.

Another aspect of the disclosure provides a viral particle, e.g., an AAV viral particle, comprising the AAV viral genome of the disclosure, and a capsid protein.

Another aspect of the disclosure provides a vector comprising the nucleic acid of the disclosure, the AAV viral genome of the disclosure, or the viral particle of the disclosure.

Another aspect of the disclosure provides a host cell comprising the nucleic acid of the disclosure, the AAV viral genome of the disclosure, the AAV viral particle of the disclosure, or the vector of the disclosure, optionally wherein the host cell is an insect cell, a bacterial cell or a mammalian cell.

Another aspect of the disclosure provides a method of making an isolated, e.g., recombinant, AAV particle, the method comprising: (i) providing a cell comprising the AAV viral genome of the disclosure, or an AAV viral genome comprising the nucleic acid of the disclosure; and (ii) incubating the cell under conditions suitable to enclose the viral genome in a capsid protein, e.g., a VOY101 capsid protein; thereby making the isolated AAV particle.

Another aspect of the disclosure provides a pharmaceutical composition comprising the nucleic acid of the disclosure, the AAV viral genome of the disclosure, or the AAV viral particle of the disclosure, and a pharmaceutically acceptable carrier or excipient; optionally, wherein the pharmaceutically acceptable carrier or excipient comprises 10 mM disodium phosphate, 2 mM monopotassium phosphate, 2.7 mM potassium chloride, 192 mM sodium chloride, and 0.001% Pluronic Acid (F-68).

Another aspect of the disclosure provides a method for treating tauopathy in a subject in need thereof, the method comprising administering to said subject a therapeutically effective amount of a composition comprising the pharmaceutical composition of the disclosure, an AAV viral particle comprising nucleic acid of the disclosure, an AAV particle comprising the AAV viral genome of the disclosure, or the AAV viral particle of the disclosure, thereby treating the tauopathy in the subject.

Another aspect of the disclosure provides a use of the nucleic acid of the disclosure, the AAV viral genome of the disclosure, the AAV viral particle of the disclosure, or the pharmaceutical composition of the disclosure, in the manufacture of a medicament for treating tauopathy in a subject in need thereof.

Another aspect of the disclosure provides the nucleic acid of the disclosure, the AAV viral genome of the disclosure, the AAV viral particle of the disclosure, or the pharmaceutical composition of the disclosure, for use in treating tauopathy in a subject in need thereof.

### BRIEF DESCRIPTIONS OF THE DRAWINGS

FIG. 1 is a schematic of the Tau protein, depicting the various protein domains, phosphorylation sites, and target domains/epitopes of certain anti-tau antibodies, such as IPN002, AT08, PT3, UCB, PT7, PHF1, and C10.2. Other anti-tau antibodies such as 6C5 are not included.

FIGS. 2A-2B depict reduction of positive staining by anti-tau antibody AT100 in olfactory bulb (OB), entorhinal cortex (EC), hippocampus (Hipp) and thalamus (Thal) in mice receiving intravenous injection of AAV particles comprising vectorized antibody constructs compared to vehicle control in the hTau olfactory bulb model. FIG. 2A shows results of VOY101.CBA.PT3 construct injected at 1.4E13 vg/kg. FIG. 2B shows results of VOY101.GFAP.PHF1 construct injected at 1.4E13 (low), 4.4E13 (medium), and 1.4E14 (high) vg/kg.

FIG. 3 depicts efficacy of AAV particles comprising a VOY101.CBA.PT3 construct in P301S hippocampal seeding model. Briefly, AAV particles comprising VOY101.CBA.PT3 were intravenously injected into P301S mice of 6 weeks old at 5E+12 (5×10¹²) or 1.4E+13 (1.4×10¹³) Vg/Kg. PHF-containing lysates from patients was seeded into mouse hippocampus at 8 weeks old. Samples were collected at seeding or at termination of the experiment, when mice are 14 weeks old. The graph shows immunoreactivity (IR) in immunohistochemical (IHC) staining by anti-tau antibody AT100 in ipsilateral and contralateral hippocampus of mice injected with the vectorized PT3 antibody.

FIGS. 4A-4B depict efficacy of AAV particles comprising a VOY101.CBA.PHF1 construct in P301S hippocampal seeding model. Briefly, AAV particles comprising VOY101.CBA.PHF1 were intravenously injected into P301S mice of 6 weeks old at 1.4E+13 or 1.4E+14 Vg/Kg. PHF-containing lysates from patients was seeded into mouse hippocampus at 8 weeks old. Samples were collected at seeding or at termination of the experiment, when mice are 14 weeks old. FIG. 4A shows expression level of vectorized antibody PHF1 (or “antibody B”) in the cortex, hippocampus and serum at seeding. FIG. 4B shows immunoreactivity (IR) by anti-tau antibody AT100 in IHC in ipsilateral and contralateral hippocampus of mice injected with the vectorized PHF1 antibody in FIG. 4A.

FIGS. 5A-5B depict efficacy of AAV particles comprising a VOY101.CBA.PHF1 construct in P301S intrinsic model. Briefly, AAV particles comprising VOY101.CBA.PHF1 were intravenously injected into P301S mice of 8 weeks old at 1.4E+13, 4.4E+13 or 1.4E+14 Vg/Kg on day 1, and samples were collected on day 91. FIG. 5A shows expression levels of vectorized PHF1 antibody (“antibody B”) in the cortex and cerebrospinal fluid (CSF) at termination of the experiment. FIG. 5B shows anti-tau antibody AT8 immunoreactivity (IR) in the cortex, measured by ELISA in mice injected with the vectorized PHF1 antibody in FIG. 5A.

FIGS. 6A-6B show kinetics of AAV distribution in the wild-type mouse hippocampus (viral genome/cell, FIG. 6A), and kinetics of antibody expression (ng/g in cortex and hippocampus, ng/ml in CSF and serum. FIG. 6B) after intravenous injection of the respective AAV particles comprising vectorized PT3 antibody (“antibody A”) under CBA, GFAP or SYN promoters. AAV particles were injected at 1.4E+13 vg/kg dose. Samples were collected on day 1, 2, 3, 7, 14 and 28 post-injection for each data point.

### DETAILED DESCRIPTION OF THE INVENTION

### I. Compositions

According to the present disclosure, compositions for delivering functional anti-tau antibodies and/or antibody-based compositions by adeno-associated viruses (AAVs) are provided. AAV particles may be provided via any of several routes of administration, to a cell, tissue, organ, or organism, in vivo, ex vivo, or in vitro.

As used herein, an “AAV particle” is a virus which comprises a viral genome with at least one payload region and at least one inverted terminal repeat (ITR) region.

As used herein, “viral genome” or “vector genome” refers to the nucleic acid sequence(s) encapsulated in an AAV particle. Viral genomes comprise at least one payload region encoding polypeptides, e.g., antibodies, antibody-based compositions or fragments thereof.

As used herein, a “payload” or “payload region” is any nucleic acid molecule which encodes one or more polypeptides. At a minimum, a payload region comprises nucleic acid sequences that encode an antibody, an antibody-based composition, or a fragment thereof, but may also optionally comprise one or more functional or regulatory elements to facilitate transcriptional expression and/or polypeptide translation.

As used herein, “VL” and “VH” refer to components of a light chain or heavy chain of an antibody, respectively, or a fragment thereof. In some embodiments, “VL” and “VH” refer to the variable regions of the light or heavy chain of an antibody, respectively, or a fragment thereof. In another embodiment, “VL” and “VH” may also embrace a constant region of a light or heavy chain of an antibody, or a fragment thereof. In another embodiment, “VL” and “VH” may embrace the entirety of an antibody light chain or heavy chain, respectively.

In some embodiments, AAV particles, viral genomes and/or payloads, and the methods of their use may be as described in WO2017189963, the contents of which are herein incorporated by reference in their entirety.

The nucleic acid sequences and polypeptides disclosed herein may be engineered to contain modular elements and/or sequence motifs assembled to enable expression of the antibodies or antibody-based compositions. In some embodiments, the nucleic acid sequence comprising the payload region may comprise one or more of a promoter region, an intron, a Kozak sequence, an enhancer, or a polyadenylation sequence. Payload regions typically encode antibodies or antibody-based compositions, which may include an antibody heavy chain domain, an antibody light chain domain, both antibody heavy and light chain domains, or fragments of the foregoing in combination with each other or in combination with other polypeptide moieties. In some cases, payload regions may also encode one or more linkers or joining regions between antibody heavy and light chain domains or fragments. The order of expression, structural position, or concatemer count (heavy chain, light chain, or linker) may be different within or among different payload regions. The identity, position and number of linkers expressed by payload regions may also vary.

The payload regions may be delivered to one or more target cells, tissues, organs, or organisms within the viral genome of an AAV particle.

Viral genomes of the present disclosure may comprise a 5′ ITR with a sequence selected from SEQ ID NO: 2076 or 2077, one or more promoter regions with a sequence selected from SEQ ID NO: 2080-2089 and 2238-2239, an antibody polynucleotide with a sequence selected from SEQ ID NO: 1740-1989, 2241-2243, 2169-2170, and 4564-4565, or encoding a sequence selected from SEQ ID NO: 1740-1989, 2241-2243, 2169-2170, and 4564-4565, a polyadenylation signal sequence with a sequence selected from SEQ ID NO: 2122-2124, and a 3′ ITR with a sequence selected from SEQ ID NO: 2078-2079. In some embodiments, viral genomes described herein may comprise one or more exon sequences with a sequence selected from SEQ ID NO: 2090-2094. In some embodiments, viral genomes described herein may comprise one or more intron sequences with a sequence selected from SEQ ID NO: 2095-2105, 2240 and 2256-2258. In some embodiments, viral genomes described herein may comprise a BSA sequence comprising SEQ ID NO: 4563. In some embodiments, viral genomes described herein may comprise one or signal sequence regions with a sequence selected from SEQ ID NO: 1740, 1741, 1861, 2106-2117, 2241 and 4564. In some embodiments, the signal sequence region is derived from an antibody sequence. In some embodiments, viral genomes described herein may comprise one or more tag sequence regions with a sequence selected from SEQ ID NO: 2118-2121 and 2255. In some embodiments, the viral genomes described herein may comprise a filler sequence region with a sequence selected from SEQ ID NO: 2125-2126. Viral genomes described herein may comprise a sequence selected from SEQ ID NO: 1990-2075, 2137-2168, 2171-2237, 2260-2321, and 4547-4562.

Viral genomes described herein may comprise more than one antibody polynucleotide. When more than one antibody polynucleotide exists in a viral genome, these antibody polynucleotides may be separated by a linker sequence, with a sequence selected from SEQ ID NO: 1724-1739, 2244-2254 and 2259. In some embodiments, viral genomes described herein comprise a first antibody polynucleotide sequence and a second antibody polynucleotide sequence, wherein each may encode an antibody heavy or light chain or a fragment thereof. In some embodiments, the viral genome comprises more than two antibody polynucleotides.

In some embodiments, a viral genome, when read 5′ to 3′, may encode an antibody heavy chain, at least one linker, and an antibody light chain. This viral genome may be described as comprising a heavy-linker-light configuration.

In some embodiments, a viral genome, when read 5′ to 3′, may encode an antibody light chain, at least one linker, and an antibody heavy chain. This viral genome may be described as comprising a light-linker-heavy configuration.

The viral genomes described herein may be packaged into an AAV particle comprising any AAV serotype known in the art, or selected from VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2 variants, AAV2G9, AAV3, AAV2/3 variants AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9 K449R, or variants thereof.

In some embodiments, the capsid of the AAV particle is one of VOY101, PHP.B, AAV9, AAV9K449, AAV1, AAV2, VOY201, AAV2 variant or AAV2/3 variant.

AAV particles described herein may be prepared as a pharmaceutical composition. In some embodiments, the pharmaceutical composition may be administered to a subject. In some embodiments, a method of producing a functional antibody in a subject may comprise administration of a pharmaceutical composition described herein to the subject. In some embodiments, the functional antibody may be encoded by one or more antibody polynucleotides of a viral genome described herein, packaged into an AAV particle. In some embodiments, the functional antibody may be encoded by two different viral genomes, packaged into separate AAV particles. The functional antibody may be expressed in a target cell or tissue in a range from 0.001 μg/mL to 100 mg/mL.

Pharmaceutical compositions described herein may be used in a method of treating tauopathy, wherein a therapeutically effective amount of a pharmaceutical composition described herein is administered to a subject in need. The tauopathy may be any one of Alzheimer's disease (AD), frontotemporal dementia and parkinsonism linked to chromosome 17 (FTDP-17), Frontotemporal lobar degeneration (FTLD), Frontotemporal dementia, chronic traumatic encephalopathy (CTE), Progressive Supranuclear Palsy (PSP), Down's syndrome, Pick's disease, Corticobasal degeneration (CBD), Corticobasal syndrome, Amyotrophic lateral sclerosis (ALS), Prion diseases, Creutzfeldt-Jakob disease (CJD), Multiple system atrophy, Tangle-only dementia, and Progressive subcortical gliosis or other tau associated disease.

Pharmaceutical compositions described herein may be used in a method of preventing tauopathy, wherein a therapeutically effective amount of a pharmaceutical composition described herein is administered to a subject in need. The tauopathy may be any one of Alzheimer's disease (AD), frontotemporal dementia and parkinsonism linked to chromosome 17 (FTDP-17), Frontotemporal lobar degeneration (FTLD), Frontotemporal dementia, chronic traumatic encephalopathy (CTE), Progressive Supranuclear Palsy (PSP), Down's syndrome, Pick's disease, Corticobasal degeneration (CBD), Corticobasal syndrome, Amyotrophic lateral sclerosis (ALS), Prion diseases, Creutzfeldt-Jakob disease (CJD), Multiple system atrophy, Tangle-only dementia, and Progressive subcortical gliosis or other tau associated disease.

### Adeno-Associated Viruses (AAVs) and AAV Particles

Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family comprises the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in FIELDS VIROLOGY (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

The wild-type AAV vector genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145nt in wild-type AAV) at the 5′ and 3′ ends of the ssDNA which form an energetically stable double stranded region. The double stranded hairpin structures comprise multiple functions including, but not limited to, acting as an origin for DNA replication by functioning as primers for the endogenous DNA polymerase complex of the host viral replication cell.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the contents of which are herein incorporated by reference in their entirety) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

For use as a biological tool, the wild-type AAV viral genome can be modified to replace the rep/cap sequences with a nucleic acid sequence comprising a payload region with at least one ITR region. Typically, in recombinant AAV viral genomes there are two ITR regions. The rep/cap sequences can be provided in trans during production to generate AAV particles.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entirety.

In some embodiments, AAV particles of the present disclosure are recombinant AAV viral vectors which are replication defective and lacking sequences encoding functional Rep and Cap proteins within their viral genome. These defective AAV vectors may lack most or all parental coding sequences and essentially carry only one or two AAV ITR sequences and the nucleic acid of interest for delivery to a cell, a tissue, an organ, or an organism.

In some embodiments, the viral genome of the AAV particles of the present disclosure comprise at least one control element which provides for the replication, transcription, and translation of a coding sequence encoded therein. Not all of the control elements need always be present as long as the coding sequence is capable of being replicated, transcribed, and/or translated in an appropriate host cell. Non-limiting examples of expression control elements include sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV vectors of the present disclosure may be produced recombinantly and may be based on adeno-associated virus (AAV) parent or reference sequences. As used herein, a “vector” is any molecule or moiety which transports, transduces, or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV vector genomes contain DNA strands which anneal together to form double stranded DNA. By skipping second strand synthesis, scAAVs allow for rapid expression in the transduced cell.

In some embodiments, the AAV particle of the present disclosure is an scAAV.

In some embodiments, the AAV particle of the present disclosure is an ssAAV.

Methods for producing and/or modifying AAV particles are disclosed in the art such as pseudotyped AAV vectors (PCT Patent Publication Nos. WO200028004; WO200123001; WO2004112727; WO2005005610; and WO2005072364, the content of each of which is incorporated herein by reference in its entirety).

AAV particles may be modified to enhance the efficiency of delivery. Such modified AAV particles can be packaged efficiently and be used to successfully infect the target cells at high frequency and with minimal toxicity. In some embodiments, the capsids of the AAV particles are engineered according to the methods described in US Publication Number US20130195801, the contents of which are incorporated herein by reference in their entirety.

In some embodiments, the AAV particles comprising a payload region encoding the polypeptides may be introduced into mammalian cells.

### AAV Serotypes

AAV particles of the present disclosure may comprise or be derived from any natural or recombinant AAV serotype. According to the present disclosure, the AAV particles may utilize or be based on a serotype or include a peptide selected from any of the following VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9 K449R, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9 and variants thereof.

In some embodiments, the AAV serotype may be, or have, a sequence as described in US20030138772, U.S. Pat. Nos. 7,198,951, 6,156,303, US20140359799, WO2015168666, U.S. Pat. No. 9,233,131, US20150376607, US20150376240, US20160017295, US20150238550, US20150315612, WO2015121501, WO2016049230, U.S. Pat. No. 8,734,809, WO2016065001, U.S. Pat. No. 9,624,274, the contents of which are herein incorporated by reference in their entirety, such as, but not limited to, the AAV serotypes listed in Table 1, or a variant or a derivative thereof.

In some embodiments, the AAV serotype may be, or have, a sequence as described in United States Publication No. US20150159173, the contents of which are herein incorporated by reference in their entirety, such as, but not limited to, the AAV serotypes listed in Table 1, or a variant thereof including, but not limited to Cy5R1, Cy5R2, Cy5R3, Cy5R4, rh.13R, rh.37R2, rh.2R, rh.8R, rh.48.1, rh.48.2, rh.48.1.2, hu.44R1, hu.44R2, hu.44R3, hu.29R, ch.5R1, rh64R1, rh64R2, AAV6.2, AAV6.1, AAV6.12, hu.48R1, hu.48R2, and hu.48R3.

In some embodiments, the AAV serotype may be, or have, a mutation in the AAV9 sequence as described by N Pulicherla et al. (Molecular Therapy 19(6):1070-1078 (2011), herein incorporated by reference in its entirety), such as but not limited to, AAV9.9, AAV9.11, AAV9.13, AAV9.16, AAV9.24, AAV9.45, AAV9.47, AAV9.61, AAV9.68, AAV9.84.

In some embodiments, the serotype may be AAVDJ or a variant thereof, such as AAVDJ8 (or AAV-DJ8), as described by Grimm et al. (Journal of Virology 82(12): 5887-5911 (2008), herein incorporated by reference in its entirety). The amino acid sequence of AAVDJ8 may comprise two or more mutations in order to remove the heparin binding domain (HBD). As a non-limiting example, the AAV-DJ sequence described as SEQ ID NO: 1 in U.S. Pat. No. 7,588,772, the contents of which are herein incorporated by reference in their entirety, may comprise two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may comprise three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In some embodiments, the AAV serotype may be, or have, a sequence of AAV4 as described in International Publication No. WO1998011244, the contents of which are herein incorporated by reference in their entirety, such as, but not limited to AAV4 (SEQ ID NO: 1-20 of WO1998011244).

In some embodiments, the AAV serotype may be, or have, a mutation in the AAV2 sequence to generate AAV2G9 as described in International Publication No. WO2014144229 and herein incorporated by reference in its entirety.

In some embodiments, the AAV serotype may be, or have, a sequence as described in International Publication No. WO2005033321, the contents of which are herein incorporated by reference in their entirety, such as, but not limited to AAV3-3 (SEQ ID NO: 217 of WO2005033321), AAV1 (SEQ ID NO: 219 and 202 of WO2005033321), AAV106.1/hu.37 (SEQ ID No: 10 of WO2005033321), AAV114.3/hu.40 (SEQ ID No: 11 of WO2005033321), AAV127.2/hu.41 (SEQ ID NO:6 and 8 of WO2005033321), AAV128.3/hu.44 (SEQ ID No: 81 of WO2005033321), AAV130.4/hu.48 (SEQ ID NO: 78 of WO2005033321), AAV145.1/hu.53 (SEQ ID No: 176 and 177 of WO2005033321), AAV145.6/hu.56 (SEQ ID NO: 168 and 192 of WO2005033321), AAV16.12/hu.11 (SEQ ID NO: 153 and 57 of WO2005033321), AAV16.8/hu.10 (SEQ ID NO: 156 and 56 of WO2005033321), AAV161.10/hu.60 (SEQ ID No: 170 of WO2005033321), AAV161.6/hu.61 (SEQ ID No: 174 of WO2005033321), AAV1-7/rh.48 (SEQ ID NO: 32 of WO2005033321), AAV1-8/rh.49 (SEQ ID NOs: 103 and 25 of WO2005033321), AAV2 (SEQ ID NO: 211 and 221 of WO2005033321), AAV2-15/rh.62 (SEQ ID No: 33 and 114 of WO2005033321), AAV2-3/rh.61 (SEQ ID NO: 21 of WO2005033321), AAV2-4/rh.50 (SEQ ID No: 23 and 108 of WO2005033321), AAV2-5/rh.51 (SEQ ID NO: 104 and 22 of WO2005033321), AAV3.1/hu.6 (SEQ ID NO: 5 and 84 of WO2005033321), AAV3.1/hu.9 (SEQ ID NO: 155 and 58 of WO2005033321), AAV3-11/rh.53 (SEQ ID NO: 186 and 176 of WO2005033321), AAV3-3 (SEQ ID NO: 200 of WO2005033321), AAV33.12/hu.17 (SEQ ID NO:4 of WO2005033321), AAV33.4/hu.15 (SEQ ID No: 50 of WO2005033321), AAV33.8/hu.16 (SEQ ID No: 51 of WO2005033321), AAV3-9/rh.52 (SEQ ID NO: 96 and 18 of WO2005033321), AAV4-19/rh.55 (SEQ ID NO: 117 of WO2005033321), AAV4-4 (SEQ ID NO: 201 and 218 of WO2005033321), AAV4-9/rh.54 (SEQ ID NO: 116 of WO2005033321), AAV5 (SEQ ID NO: 199 and 216 of WO2005033321), AAV52.1/hu.20 (SEQ ID NO: 63 of WO2005033321), AAV52/hu.19 (SEQ ID NO: 133 of WO2005033321), AAV5-22/rh.58 (SEQ ID No: 27 of WO2005033321), AAV5-3/rh.57 (SEQ ID NO: 105 of WO2005033321), AAV5-3/rh.57 (SEQ ID No: 26 of WO2005033321), AAV58.2/hu.25 (SEQ ID No: 49 of WO2005033321), AAV6 (SEQ ID NO: 203 and 220 of WO2005033321), AAV7 (SEQ ID NO: 222 and 213 of WO2005033321), AAV7.3/hu.7 (SEQ ID No: 55 of WO2005033321), AAV8 (SEQ ID NO: 223 and 214 of WO2005033321), AAVH-1/hu.1 (SEQ ID No: 46 of WO2005033321), AAVH-5/hu.3 (SEQ ID No: 44 of WO2005033321), AAVhu.1 (SEQ ID NO: 144 of WO2005033321), AAVhu.10 (SEQ ID NO: 156 of WO2005033321), AAVhu.11 (SEQ ID NO: 153 of WO2005033321), AAVhu.12 (WO2005033321 SEQ ID NO: 59), AAVhu.13 (SEQ ID NO: 129 of WO2005033321), AAVhu.14/AAV9 (SEQ ID NO: 123 and 3 of WO2005033321), AAVhu.15 (SEQ ID NO: 147 of WO2005033321), AAVhu.16 (SEQ ID NO: 148 of WO2005033321), AAVhu.17 (SEQ ID NO: 83 of WO2005033321), AAVhu.18 (SEQ ID NO: 149 of WO2005033321), AAVhu.19 (SEQ ID NO: 133 of WO2005033321), AAVhu.2 (SEQ ID NO: 143 of WO2005033321), AAVhu.20 (SEQ ID NO: 134 of WO2005033321), AAVhu.21 (SEQ ID NO: 135 of WO2005033321), AAVhu.22 (SEQ ID NO: 138 of WO2005033321), AAVhu.23.2 (SEQ ID NO: 137 of WO2005033321), AAVhu.24 (SEQ ID NO: 136 of WO2005033321), AAVhu.25 (SEQ ID NO: 146 of WO2005033321), AAVhu.27 (SEQ ID NO: 140 of WO2005033321), AAVhu.29 (SEQ ID NO: 132 of WO2005033321), AAVhu.3 (SEQ ID NO: 145 of WO2005033321), AAVhu.31 (SEQ ID NO: 121 of WO2005033321), AAVhu.32 (SEQ ID NO: 122 of WO2005033321), AAVhu.34 (SEQ ID NO: 125 of WO2005033321), AAVhu.35 (SEQ ID NO: 164 of WO2005033321), AAVhu.37 (SEQ ID NO: 88 of WO2005033321), AAVhu.39 (SEQ ID NO: 102 of WO2005033321), AAVhu.4 (SEQ ID NO: 141 of WO2005033321), AAVhu.40 (SEQ ID NO: 87 of WO2005033321), AAVhu.41 (SEQ ID NO: 91 of WO2005033321), AAVhu.42 (SEQ ID NO: 85 of WO2005033321), AAVhu.43 (SEQ ID NO: 160 of WO2005033321), AAVhu.44 (SEQ ID NO: 144 of WO2005033321), AAVhu.45 (SEQ ID NO: 127 of WO2005033321), AAVhu.46 (SEQ ID NO: 159 of WO2005033321), AAVhu.47 (SEQ ID NO: 128 of WO2005033321), AAVhu.48 (SEQ ID NO: 157 of WO2005033321), AAVhu.49 (SEQ ID NO: 189 of WO2005033321), AAVhu.51 (SEQ ID NO: 190 of WO2005033321), AAVhu.52 (SEQ ID NO: 191 of WO2005033321), AAVhu.53 (SEQ ID NO: 186 of WO2005033321), AAVhu.54 (SEQ ID NO: 188 of WO2005033321), AAVhu.55 (SEQ ID NO: 187 of WO2005033321), AAVhu.56 (SEQ ID NO: 192 of WO2005033321), AAVhu.57 (SEQ ID NO: 193 of WO2005033321), AAVhu.58 (SEQ ID NO: 194 of WO2005033321), AAVhu.6 (SEQ ID NO: 84 of WO2005033321), AAVhu.60 (SEQ ID NO: 184 of WO2005033321), AAVhu.61 (SEQ ID NO: 185 of WO2005033321), AAVhu.63 (SEQ ID NO: 195 of WO2005033321), AAVhu.64 (SEQ ID NO: 196 of WO2005033321), AAVhu.66 (SEQ ID NO: 197 of WO2005033321), AAVhu.67 (SEQ ID NO: 198 of WO2005033321), AAVhu.7 (SEQ ID NO: 150 of WO2005033321), AAVhu.8 (WO2005033321 SEQ ID NO: 12), AAVhu.9 (SEQ ID NO: 155 of WO2005033321), AAVLG-10/rh.40 (SEQ ID No: 14 of WO2005033321), AAVLG-4/rh.38 (SEQ ID NO: 86 of WO2005033321), AAVLG-4/rh.38 (SEQ ID No: 7 of WO2005033321), AAVN721-8/rh.43 (SEQ ID NO: 163 of WO2005033321), AAVN721-8/rh.43 (SEQ ID No: 43 of WO2005033321), AAVpi.1 (WO2005033321 SEQ ID NO: 28), AAVpi.2 (WO2005033321 SEQ ID NO: 30), AAVpi.3 (WO2005033321 SEQ ID NO: 29), AAVrh.38 (SEQ ID NO: 86 of WO2005033321), AAVrh.40 (SEQ ID NO: 92 of WO2005033321), AAVrh.43 (SEQ ID NO: 163 of WO2005033321), AAVrh.44 (WO2005033321 SEQ ID NO: 34), AAVrh.45 (WO2005033321 SEQ ID NO: 41), AAVrh.47 (WO2005033321 SEQ ID NO: 38), AAVrh.48 (SEQ ID NO: 115 of WO2005033321), AAVrh.49 (SEQ ID NO: 103 of WO2005033321), AAVrh.50 (SEQ ID NO: 108 of WO2005033321), AAVrh.51 (SEQ ID NO: 104 of WO2005033321), AAVrh.52 (SEQ ID NO: 96 of WO2005033321), AAVrh.53 (SEQ ID NO: 97 of WO2005033321), AAVrh.55 (WO2005033321 SEQ ID NO: 37), AAVrh.56 (SEQ ID NO: 152 of WO2005033321), AAVrh.57 (SEQ ID NO: 105 of WO2005033321), AAVrh.58 (SEQ ID NO: 106 of WO2005033321), AAVrh.59 (WO2005033321 SEQ ID NO: 42), AAVrh.60 (WO2005033321 SEQ ID NO: 31), AAVrh.61 (SEQ ID NO: 107 of WO2005033321), AAVrh.62 (SEQ ID NO: 114 of WO2005033321), AAVrh.64 (SEQ ID NO: 99 of WO2005033321), AAVrh.65 (WO2005033321 SEQ ID NO: 35), AAVrh.68 (WO2005033321 SEQ ID NO: 16), AAVrh.69 (WO2005033321 SEQ ID NO: 39), AAVrh.70 (WO2005033321 SEQ ID NO: 20), AAVrh.72 (WO2005033321 SEQ ID NO: 9), or variants thereof including, but not limited to, AAVcy.2, AAVcy.3, AAVcy.4, AAVcy.5, AAVcy.6, AAVrh.12, AAVrh.17, AAVrh.18, AAVrh.19, AAVrh.21, AAVrh.22, AAVrh.23, AAVrh.24, AAVrh.25, AAVrh.25/42 15, AAVrh.31, AAVrh.32, AAVrh.33, AAVrh.34, AAVrh.35, AAVrh.36, AAVrh.37, AAVrh14. Non limiting examples of variants include SEQ ID NO: 13, 15, 17, 19, 24, 36, 40, 45, 47, 48, 51-54, 60-62, 64-77, 79, 80, 82, 89, 90, 93-95, 98, 100, 101, 109-113, 118-120, 124, 126, 131, 139, 142, 151,154, 158, 161, 162, 165-183, 202, 204-212, 215, 219, 224-236, of WO2005033321, the contents of which are herein incorporated by reference in their entirety.

In some embodiments, the AAV serotype may be, or have, a sequence as described in U.S. Pat. No. 9,163,261, the contents of which are herein incorporated by reference in their entirety, such as, but not limited to, AAV-2-pre-miRNA-101 (SEQ ID NO: 1 U.S. Pat. No. 9,163,261), or variants thereof.

According to the present disclosure, AAV capsid serotype selection or use may be from a variety of species. In some embodiments, the AAV may be an avian AAV (AAAV). The AAV serotype may be, or have, a sequence as described in U.S. Pat. No. 9,238,800, the contents of which are herein incorporated by reference in their entirety, such as, but not limited to, AAAV (SEQ ID NO: 1, 2, 4, 6, 8, 10, 12, and 14 of U.S. Pat. No. 9,238,800), or variants thereof.

In some embodiments, the AAV may be a bovine AAV (BAAV). The BAAV serotype may be, or have, a sequence as described in U.S. Pat. No. 9,193,769, the contents of which are herein incorporated by reference in their entirety, such as, but not limited to, BAAV (SEQ ID NO: 1 and 6 of U.S. Pat. No. 9,193,769), or variants thereof. The BAAV serotype may be or have a sequence as described in U.S. Pat. No. 7,427,396, the contents of which are herein incorporated by reference in their entirety, such as, but not limited to, BAAV (SEQ ID NO: 5 and 6 of U.S. Pat. No. 7,427,396), or variants thereof.

In some embodiments, the AAV may be a caprine AAV. The caprine AAV serotype may be, or have, a sequence as described in U.S. Pat. No. 7,427,396, the contents of which are herein incorporated by reference in their entirety, such as, but not limited to, caprine AAV (SEQ ID NO: 3 of U.S. Pat. No. 7,427,396), or variants thereof.

In other embodiments the AAV may be engineered as a hybrid AAV from two or more parental serotypes. In some embodiments, the AAV may be AAV2G9 which comprises sequences from AAV2 and AAV9. The AAV2G9 AAV serotype may be, or have, a sequence as described in United States Patent Publication No. US20160017005, the contents of which are herein incorporated by reference in its entirety.

In some embodiments, the AAV may be a serotype generated by the AAV9 capsid library with mutations in amino acids 390-627 (VP1 numbering) as described by Pulicherla et al. (Molecular Therapy 19(6):1070-1078 (2011), the contents of which are herein incorporated by reference in their entirety. The serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V606I), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A, G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In some embodiments, the AAV particle may have, or may be a serotype selected from any of those found in Table 1.

In some embodiments, the AAV capsid may comprise a sequence, fragment or variant thereof, of any of the sequences in Table 1.

In some embodiments, the AAV capsid may be encoded by a sequence, fragment or variant as described in Table 1.

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ilie) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine; U (See) for Selenocysteine; X (Xaa) for any amino acid; and Z (Glx) for Glutamine or Glutamic acid.

TABLE 1
AAV Serotypes

| | SEQ | | SEQ | | SEQ | | SEQ |
|---|---|---|---|---|---|---|---|
| | ID | | ID | | ID | | ID |
| Serotype | NO | Serotype | NO | Serotype | NO | Serotype | NO |
| VOY101 | 1 | AAV CLv-K6 | 848 | AAV2/3 | 2789 | AAV variant | 3665 |
| | | | | variant | | | |
| VOY101 | 2 | AAV CLv-M1 | 849 | AAV2/3 | 2790 | AAV variant | 3666 |
| | | | | variant | | | |
| VOY201 | 3 | AAV CLv-M11 | 850 | AAV2/3 | 2791 | AAV variant | 3667 |
| | | | | variant | | | |
| VOY201 | 4534 | AAV CLv-M2 | 851 | AAV2/3 | 2792 | AAV variant | 3668 |
| | | | | variant | | | |
| PHP.N/PHP.B- | 4 | AAV CLv-M5 | 852 | AAV2/3 | 2793 | AAV variant | 3669 |
| DGT | | | | variant | | | |
| AAVPHP.B or | 5 | AAV CLv-M6 | 853 | AAV2/3 | 2794 | AAV variant | 3670 |
| G2B-26 | | | | variant | | | |
| AAVPHP.B | 6 | AAV CLv-M7 | 854 | AAV2/3 | 2795 | AAV variant | 3671 |
| | | | | variant | | | |
| AAVG2B-13 | 7 | AAV CLv-M8 | 855 | AAV2/3 | 2796 | AAV variant | 3672 |
| | | | | variant | | | |
| AAVTH1.1-32 | 8 | AAV CLv-M9 | 856 | AAV2/3 | 2797 | AAV variant | 3673 |
| | | | | variant | | | |
| AAVTH1.1-35 | 9 | AAV CHt-P1 | 857 | AAV2/3 | 2798 | AAV variant | 3674 |
| | | | | variant | | | |
| PHP.S/G2A12 | 10 | AAV CHt-P6 | 858 | AAV2/3 | 2799 | AAV variant | 3675 |
| | | | | variant | | | |
| AAV9/hu.14 | 11 | AAV CHt-P8 | 859 | AAV2/3 | 2800 | AAV variant | 3676 |
| K449R | | | | variant | | | |
| AAV1 | 12 | AAV CHt-6.1 | 860 | AAV2/3 | 2801 | AAV variant | 3677 |
| | | | | variant | | | |
| AAV1 | 13 | AAV CHt-6.10 | 861 | AAV2/3 | 2802 | AAV variant | 3678 |
| | | | | variant | | | |
| AAV1 | 14 | AAV CHt-6.5 | 862 | AAV2/3 | 2803 | AAV variant | 3679 |
| | | | | variant | | | |
| AAV1.3 | 15 | AAV CHt-6.6 | 863 | AAV2/3 | 2804 | AAV variant | 3680 |
| | | | | variant | | | |
| AAV10 | 16 | AAV CHt-6.7 | 864 | AAV2/3 | 2805 | AAV variant | 3681 |
| | | | | variant | | | |
| AAV10 | 17 | AAV CHt-6.8 | 865 | AAV2/3 | 2806 | AAV variant | 3682 |
| | | | | variant | | | |
| AAV10 | 18 | AAV CSp-8.10 | 866 | AAV2/3 | 2807 | AAV variant | 3683 |
| | | | | variant | | | |
| AAV11 | 19 | AAV CSp-8.2 | 867 | AAV2/3 | 2808 | AAV variant | 3684 |
| | | | | variant | | | |
| AAV12 | 20 | AAV CSp-8.4 | 868 | AAV2/3 | 2809 | AAV variant | 3685 |
| | | | | variant | | | |
| AAV2 | 21 | AAV CSp-8.5 | 869 | AAV2/3 | 2810 | AAV variant | 3686 |
| | | | | variant | | | |
| AAV2 | 22 | AAV CSp-8.6 | 870 | AAV2/3 | 2811 | AAV variant | 3687 |
| | | | | variant | | | |
| AAV2 | 23 | AAV CSp-8.7 | 871 | AAV2/3 | 2812 | AAV variant | 3688 |
| | | | | variant | | | |
| AAV2 | 24 | AAV CSp-8.8 | 872 | AAV2/3 | 2813 | AAV variant | 3689 |
| | | | | variant | | | |
| AAV2 | 25 | AAV CSp-8.9 | 873 | AAV2/3 | 2814 | AAV variant | 3690 |
| | | | | variant | | | |
| AAV5 | 102 | AAV CBr-B7.3 | 874 | AAV2/3 | 2815 | AAV variant | 3691 |
| | | | | variant | | | |
| AAV5 | 103 | AAV CBr-B7.4 | 875 | AAV2/3 | 2816 | AAV variant | 3692 |
| | | | | variant | | | |
| AAV5 | 104 | AAV3B | 876 | AAV2/3 | 2817 | AAV variant | 3693 |
| | | | | variant | | | |
| AAV5 | 105 | AAV4 | 877 | AAV2/3 | 2818 | AAV variant | 3694 |
| | | | | variant | | | |
| AAV6 | 106 | AAV5 | 878 | AAV2/3 | 2819 | AAV variant | 3695 |
| | | | | variant | | | |
| AAV6 | 107 | GPV | 879 | AAV2/3 | 2820 | AAV variant | 3696 |
| | | | | variant | | | |
| AAV6 | 108 | B19 | 880 | AAV2/3 | 2821 | AAV variant | 3697 |
| | | | | variant | | | |
| AAV6 | 109 | MVM | 881 | AAV2/3 | 2822 | AAV variant | 3698 |
| | | | | variant | | | |
| AAV6 | 110 | FPV | 882 | AAV2/3 | 2823 | AAV variant | 3699 |
| | | | | variant | | | |
| AAV6 | 111 | CPV | 883 | AAV2/3 | 2824 | AAV variant | 3700 |
| | | | | variant | | | |
| AAV7 | 115 | AAV6 | 884 | AAV2/3 | 2825 | AAV variant | 3701 |
| | | | | variant | | | |
| AAV7 | 116 | AAV6 | 885 | AAV2/3 | 2826 | AAV variant | 3702 |
| | | | | variant | | | |
| AAV7 | 117 | AAV2 | 886 | AAV2/3 | 2827 | AAV variant | 3703 |
| | | | | variant | | | |
| AAV7 | 118 | ShH10 | 887 | AAV2/3 | 2828 | AAV variant | 3704 |
| | | | | variant | | | |
| AAV7 | 119 | ShH13 | 888 | AAV2/3 | 2829 | AAV variant | 3705 |
| | | | | variant | | | |
| AAV7 | 120 | ShH10 | 889 | AAV2/3 | 2830 | AAV variant | 3706 |
| | | | | variant | | | |
| AAV7 | 121 | ShH10 | 890 | AAV2/3 | 2831 | AAV variant | 3707 |
| | | | | variant | | | |
| AAV8 | 122 | ShH10 | 891 | AAV2/3 | 2832 | AAV variant | 3708 |
| | | | | variant | | | |
| AAV8 | 123 | ShH10 | 892 | AAV2/3 | 2833 | AAV variant | 3709 |
| | | | | variant | | | |
| AAV8 | 124 | ShH10 | 893 | AAV2/3 | 2834 | AAV variant | 3710 |
| | | | | variant | | | |
| AAV8 | 125 | rh74 | 894 | AAV2/3 | 2835 | AAV variant | 3711 |
| | | | | variant | | | |
| AAV8 | 126 | rh74 | 895 | AAV2/3 | 2836 | AAV variant | 3712 |
| | | | | variant | | | |
| AAV8 | 127 | AAV8 | 896 | AAV2/3 | 2837 | AAV variant | 3713 |
| | | | | variant | | | |
| AAV9 | 132 | rh74 | 897 | AAV2/3 | 2838 | AAV variant | 3714 |
| | | | | variant | | | |
| AAV9 | 133 | rh74 (RHM4-1) | 898 | AAV2/3 | 2839 | AAV variant | 3715 |
| | | | | variant | | | |
| AAV9 | 134 | rh74 (RHM15-1) | 899 | AAV2/3 | 2840 | AAV variant | 3716 |
| | | | | variant | | | |
| AAV9 | 135 | rh74 (RHM15-2) | 900 | AAV2/3 | 2841 | AAV variant | 3717 |
| | | | | variant | | | |
| AAV9 | 136 | rh74 (RHM15- | 901 | AAV2/3 | 2842 | AAV variant | 3718 |
| | | 3/RHM15-5) | | variant | | | |
| AAV2.5T | 26 | rh74 (RHM15-4) | 902 | AAV2/3 | 2843 | AAV variant | 3719 |
| | | | | variant | | | |
| AAV223.10 | 27 | rh74 (RHM15-6) | 903 | AAV2/3 | 2844 | AAV variant | 3720 |
| | | | | variant | | | |
| AAV223.2 | 28 | rh74 (RHM4-1) | 904 | AAV2/3 | 2845 | AAV variant | 3721 |
| | | | | variant | | | |
| AAV223.2 | 29 | rh74 (RHM15-1) | 905 | AAV2/3 | 2846 | AAV variant | 3722 |
| | | | | variant | | | |
| AAV223.4 | 30 | rh74 (RHM15-2) | 906 | AAV2/3 | 2847 | AAV variant | 3723 |
| | | | | variant | | | |
| AAV223.4 | 31 | rh74 (RHM15- | 907 | AAV2/3 | 2848 | AAV variant | 3724 |
| | | 3/RHM15-5) | | variant | | | |
| AAV223.5 | 32 | rh74 (RHM15-4) | 908 | AAV2/3 | 2849 | AAV variant | 3725 |
| | | | | variant | | | |
| AAV223.5 | 33 | rh74 (RHM15-6) | 909 | AAV2/3 | 2850 | AAV variant | 3726 |
| | | | | variant | | | |
| AAV223.6 | 34 | Anc80 | 912 | AAV2/3 | 2851 | AAV variant | 3727 |
| | | | | variant | | | |
| AAV223.6 | 35 | Anc80 | 913 | AAV2/3 | 2852 | AAV variant | 3728 |
| | | | | variant | | | |
| AAV223.7 | 36 | Anc81 | 914 | AAV2/3 | 2853 | AAV variant | 3729 |
| | | | | variant | | | |
| AAV223.7 | 37 | Anc80 | 915 | AAV2/3 | 2854 | AAV variant | 3730 |
| | | | | variant | | | |
| AAV29.3 | 38 | Anc82 | 916 | AAV2/3 | 2855 | AAV variant | 3731 |
| | | | | variant | | | |
| AAV29.4 | 39 | Anc82 | 917 | AAV2/3 | 2856 | AAV variant | 3732 |
| | | | | variant | | | |
| AAV29.5 | 40 | Anc83 | 918 | AAV2/3 | 2857 | AAV variant | 3733 |
| | | | | variant | | | |
| AAV29.5 | 41 | Anc83 | 919 | AAV2/3 | 2858 | AAV variant | 3734 |
| (AAVbb.2) | | | | variant | | | |
| AAV3 | 42 | Anc84 | 920 | AAV2/3 | 2859 | AAV variant | 3735 |
| | | | | variant | | | |
| AAV3 | 43 | Anc84 | 921 | AAV2/3 | 2860 | AAV variant | 3736 |
| | | | | variant | | | |
| AAV3 | 44 | Anc94 | 922 | AAV2/3 | 2861 | AAV variant | 3737 |
| | | | | variant | | | |
| AAV3.3b | 45 | Anc94 | 923 | AAV2/3 | 2862 | AAV variant | 3738 |
| | | | | variant | | | |
| AAV3-3 | 46 | Anc113 | 924 | AAV2/3 | 2863 | AAV variant | 3739 |
| | | | | variant | | | |
| AAV3-3 | 47 | Anc113 | 925 | AAV2/3 | 2864 | AAV variant | 3740 |
| | | | | variant | | | |
| AAV3a | 48 | Anc126 | 926 | AAV2/3 | 2865 | AAV variant | 3741 |
| | | | | variant | | | |
| AAV3a | 49 | Anc126 | 927 | AAV2/3 | 2866 | AAV variant | 3742 |
| | | | | variant | | | |
| AAV3b | 50 | Anc127 | 928 | AAV2/3 | 2867 | AAV variant | 3743 |
| | | | | variant | | | |
| AAV3b | 51 | Anc127 | 929 | AAV2/3 | 2868 | AAV variant | 3744 |
| | | | | variant | | | |
| AAV3b | 52 | Anc80L27 | 930 | AAV2/3 | 2869 | AAV variant | 3745 |
| | | | | variant | | | |
| AAV4 | 53 | Anc80L59 | 931 | AAV2/3 | 2870 | AAV variant | 3746 |
| | | | | variant | | | |
| AAV4 | 54 | Anc80L60 | 932 | AAV2/3 | 2871 | AAV variant | 3747 |
| | | | | variant | | | |
| AAV4 | 55 | Anc80L62 | 933 | AAV2/3 | 2872 | AAV variant | 3748 |
| | | | | variant | | | |
| AAV4 | 56 | Anc80L65 | 934 | AAV2/3 | 2873 | AAV variant | 3749 |
| | | | | variant | | | |
| AAV4 | 57 | Anc80L33 | 935 | AAV2/3 | 2874 | AAV variant | 3750 |
| | | | | variant | | | |
| AAV4 | 58 | Anc80L36 | 936 | AAV2/3 | 2875 | AAV variant | 3751 |
| | | | | variant | | | |
| AAV4 | 59 | Anc80L44 | 937 | AAV2/3 | 2876 | AAV variant | 3752 |
| | | | | variant | | | |
| AAV4 | 60 | Anc80L1 | 938 | AAV2/3 | 2877 | AAV variant | 3753 |
| | | | | variant | | | |
| AAV4 | 61 | Anc80L1 | 939 | AAV2/3 | 2878 | AAV variant | 3754 |
| | | | | variant | | | |
| AAV4 | 62 | AAV-X1 | 940 | AAV2/3 | 2879 | AAV variant | 3755 |
| | | | | variant | | | |
| AAV4 | 63 | AAV-X1b | 941 | AAV2/3 | 2880 | AAV variant | 3756 |
| | | | | variant | | | |
| AAV4 | 64 | AAV-X5 | 942 | AAV2/3 | 2881 | AAV variant | 3757 |
| | | | | variant | | | |
| AAV4 | 65 | AAV-X19 | 943 | AAV2/3 | 2882 | AAV variant | 3758 |
| | | | | variant | | | |
| AAV4 | 66 | AAV-X21 | 944 | AAV2/3 | 2883 | AAV variant | 3759 |
| | | | | variant | | | |
| AAV4 | 67 | AAV-X22 | 945 | AAV2/3 | 2884 | AAV variant | 3760 |
| | | | | variant | | | |
| AAV4 | 68 | AAV-X23 | 946 | AAV2/3 | 2885 | AAV variant | 3761 |
| | | | | variant | | | |
| AAV4 | 69 | AAV-X24 | 947 | AAV2/3 | 2886 | AAV variant | 3762 |
| | | | | variant | | | |
| AAV4 | 70 | AAV-X25 | 948 | AAV2/3 | 2887 | AAV variant | 3763 |
| | | | | variant | | | |
| AAV4 | 71 | AAV-X26 | 949 | AAV2/3 | 2888 | AAV variant | 3764 |
| | | | | variant | | | |
| AAV4 | 72 | AAV-X1 | 950 | AAV2/3 | 2889 | AAV variant | 3765 |
| | | | | variant | | | |
| AAV42.2 | 73 | AAV-X1b | 951 | AAV2/3 | 2890 | AAV variant | 3766 |
| | | | | variant | | | |
| AAV42.2 | 74 | AAV-X5 | 952 | AAV2/3 | 2891 | AAV variant | 3767 |
| | | | | variant | | | |
| AAV42.3b | 75 | AAV-X19 | 953 | AAV2/3 | 2892 | AAV variant | 3768 |
| | | | | variant | | | |
| AAV42.3B | 76 | AAV-X21 | 954 | AAV2/3 | 2893 | AAV variant | 3769 |
| | | | | variant | | | |
| AAV42.4 | 77 | AAV-X22 | 955 | AAV2/3 | 2894 | AAV variant | 3770 |
| | | | | variant | | | |
| AAV42.4 | 78 | AAV-X23 | 956 | AAV2/3 | 2895 | AAV variant | 3771 |
| | | | | variant | | | |
| AAV42.8 | 79 | AAV-X24 | 957 | AAV2/3 | 2896 | AAV variant | 3772 |
| | | | | variant | | | |
| AAV42.8 | 80 | AAV-X25 | 958 | AAV2/3 | 2897 | AAV variant | 3773 |
| | | | | variant | | | |
| AAV43.1 | 81 | AAV-X26 | 959 | AAV2/3 | 2898 | AAV variant | 3774 |
| | | | | variant | | | |
| AAV43.1 | 82 | AAVrh8 | 960 | AAV2/3 | 2899 | AAV variant | 3775 |
| | | | | variant | | | |
| AAV43.12 | 83 | AAVrh8VP2FC5 | 961 | AAV2/3 | 2900 | AAV variant | 3776 |
| | | | | variant | | | |
| AAV43.12 | 84 | AAVrh8VP2FC44 | 962 | AAV2/3 | 2901 | AAV variant | 3777 |
| | | | | variant | | | |
| AAV43.20 | 85 | AAVrh8VP2ApoB100 | 963 | AAV2/3 | 2902 | AAV variant | 3778 |
| | | | | variant | | | |
| AAV43.20 | 86 | AAVrh8VP2RVG | 964 | AAV2/3 | 2903 | AAV variant | 3779 |
| | | | | variant | | | |
| AAV43.21 | 87 | AAVrh8VP2Angiopep-2 | 965 | AAV2/3 | 2904 | AAV variant | 3780 |
| | | VP2 | | variant | | | |
| AAV43.21 | 88 | AAV9.47VP1.3 | 966 | AAV2/3 | 2905 | AAV variant | 3781 |
| | | | | variant | | | |
| AAV43.23 | 89 | AAV9.47VP2ICAMg3 | 967 | AAV2/3 | 2906 | AAV variant | 3782 |
| | | | | variant | | | |
| AAV43.23 | 90 | AAV9.47VP2RVG | 968 | AAV2/3 | 2907 | AAV variant | 3783 |
| | | | | variant | | | |
| AAV43.25 | 91 | AAV9.47VP2Angiopep-2 | 969 | AAV2/3 | 2908 | AAV variant | 3784 |
| | | | | variant | | | |
| AAV43.25 | 92 | AAV9.47VP2A-string | 970 | AAV2/3 | 2909 | AAV variant | 3785 |
| | | | | variant | | | |
| AAV43.5 | 93 | AAVrh8VP2FC5 VP2 | 971 | AAV2/3 | 2910 | AAV variant | 3786 |
| | | | | variant | | | |
| AAV43.5 | 94 | AAVrh8VP2FC44 VP2 | 972 | AAV2/3 | 2911 | AAV variant | 3787 |
| | | | | variant | | | |
| AAV4-4 | 95 | AAVrh8VP2ApoB100 VP2 | 973 | AAV2/3 | 2912 | AAV variant | 3788 |
| | | | | variant | | | |
| AAV4-4 | 96 | AAVrh8VP2RVG | 74 | AAV2/3 | 2913 | AAV variant | 3789 |
| | | VP2 | | variant | | | |
| AAV44.1 | 97 | AAVrh8VP2Angiopep-2 | 975 | AAV2/3 | 2914 | AAV variant | 3790 |
| | | VP2 | | variant | | | |
| AAV44.1 | 98 | AAV9.47VP2ICAMg3 | 976 | AAV2/3 | 2915 | AAV variant | 3791 |
| | | VP2 | | variant | | | |
| AAV44.5 | 99 | AAV9.47VP2RVG | 977 | AAV2/3 | 2916 | AAV variant | 3792 |
| | | VP2 | | variant | | | |
| AAV44.5 | 100 | AAV9.47VP2Angiopep-2 | 978 | AAV2/3 | 2917 | AAV variant | 3793 |
| | | VP2 | | variant | | | |
| AAV4407 | 101 | AAV9.47VP2A- | 979 | AAV2/3 | 2918 | AAV variant | 3794 |
| | | string VP2 | | variant | | | |
| AAV5 | 102 | rAAV-B1 | 980 | AAV2/3 | 2919 | AAV variant | 3795 |
| | | | | variant | | | |
| AAV5 | 103 | rAAV-B2 | 981 | AAV2/3 | 2920 | AAV variant | 3796 |
| | | | | variant | | | |
| AAV5 | 104 | rAAV-B3 | 982 | AAV2/3 | 2921 | AAV variant | 3797 |
| | | | | variant | | | |
| AAV5 | 105 | rAAV-B4 | 983 | AAV2/3 | 2922 | AAV variant | 3798 |
| | | | | variant | | | |
| AAV6 | 106 | rAAV-B1 | 984 | AAV2/3 | 2923 | AAV variant | 3799 |
| | | | | variant | | | |
| AAV6 | 107 | rAAV-B2 | 985 | AAV2/3 | 2924 | AAV variant | 3800 |
| | | | | variant | | | |
| AAV6 | 108 | rAAV-B3 | 986 | AAV8 | 2925 | AAV variant | 3801 |
| | | | | Variant | | | |
| AAV6 | 109 | rAAV-B4 | 987 | AAV8 | 2926 | AAV variant | 3802 |
| | | | | Variant | | | |
| AAV6 | 110 | rAAV-L1 | 988 | AAV8 | 2927 | AAV variant | 3803 |
| | | | | Variant | | | |
| AAV6 | 111 | rAAV-L2 | 989 | AAV8 | 2928 | AAV variant | 3804 |
| | | | | Variant | | | |
| AAV6.1 | 112 | rAAV-L3 | 990 | AAV8 | 2929 | AAV variant | 3805 |
| | | | | Variant | | | |
| AAV6.12 | 113 | rAAV-L4 | 991 | AAV8 | 2930 | AAV variant | 3806 |
| | | | | Variant | | | |
| AAV6.2 | 114 | rAAV-L1 | 992 | AAV8 | 2931 | AAV variant | 3807 |
| | | | | Variant | | | |
| AAV7 | 115 | rAAV-L2 | 993 | AAV8 | 2932 | AAV variant | 3808 |
| | | | | Variant | | | |
| AAV7 | 116 | rAAV-L3 | 994 | AAV8 | 2933 | AAV variant | 3809 |
| | | | | Variant | | | |
| AAV7 | 117 | rAAV-L4 | 995 | AAV8 | 2934 | AAV variant | 3810 |
| | | | | Variant | | | |
| AAV7 | 118 | AAV9 | 996 | AAV8 | 2935 | AAV variant | 3811 |
| | | | | Variant | | | |
| AAV7 | 119 | rAAV | 997 | AAV8 | 2936 | AAV variant | 3812 |
| | | | | Variant | | | |
| AAV7 | 120 | rAAV | 998 | AAV8 | 2937 | AAV variant | 3813 |
| | | | | Variant | | | |
| AAV7 | 121 | rAAV | 999 | AAV8 | 2938 | AAV variant | 3814 |
| | | | | Variant | | | |
| AAV8 | 122 | rAAV | 1000 | AAV8 | 2939 | AAV variant | 3815 |
| | | | | Variant | | | |
| AAV8 | 123 | rAAV | 1001 | AAV8 | 2940 | AAV variant | 3816 |
| | | | | Variant | | | |
| AAV8 | 124 | rAAV | 1002 | AAV8 | 2941 | AAV variant | 3817 |
| | | | | Variant | | | |
| AAV8 | 125 | rAAV | 1003 | AAV8 | 2942 | AAV variant | 3818 |
| | | | | Variant | | | |
| AAV8 | 126 | rAAV | 1004 | AAV8 | 2943 | AAV variant | 3819 |
| | | | | Variant | | | |
| AAV8 | 127 | rAAV | 1005 | AAV8 | 2944 | AAV variant | 3820 |
| | | | | Variant | | | |
| AAV-8b | 128 | rAAV | 1006 | AAV8 | 2945 | AAV variant | 3821 |
| | | | | Variant | | | |
| AAV-8b | 129 | rAAV | 1007 | AAV8 | 2946 | AAV variant | 3822 |
| | | | | Variant | | | |
| AAV-8h | 130 | rAAV | 1008 | AAV8 | 2947 | AAV variant | 3823 |
| | | | | Variant | | | |
| AAV-8h | 131 | rAAV | 1009 | AAV8 | 2948 | AAV variant | 3824 |
| | | | | Variant | | | |
| AAV9 | 132 | rAAV | 1010 | AAV8 | 2949 | AAV variant | 3825 |
| | | | | Variant | | | |
| AAV9 | 133 | rAAV | 1011 | AAV8 | 2950 | AAV variant | 3826 |
| | | | | Variant | | | |
| AAV9 | 134 | rAAV | 1012 | AAV8 | 2951 | AAV variant | 3827 |
| | | | | Variant | | | |
| AAV9 | 135 | rAAV | 1013 | AAV8 | 2952 | AAV variant | 3828 |
| | | | | Variant | | | |
| AAV9 | 136 | rAAV | 1014 | AAV8 | 2953 | AAV variant | 3829 |
| | | | | Variant | | | |
| AAV9 | 137 | rAAV | 1015 | AAV8 | 2954 | AAV variant | 3830 |
| (AAVhu.14) | | | | Variant | | | |
| AAV9 | 138 | rAAV | 1016 | AAV8 | 2955 | AAV variant | 3831 |
| (AAVhu.14) | | | | Variant | | | |
| AAVA3.1 | 139 | rAAV | 1017 | AAV8 | 2956 | AAV variant | 3832 |
| | | | | Variant | | | |
| AAVA3.3 | 140 | rAAV | 1018 | AAV8 | 2957 | AAV variant | 3833 |
| | | | | Variant | | | |
| AAVA3.3 | 141 | rAAV | 1019 | AAV8 | 2958 | AAV variant | 3834 |
| | | | | Variant | | | |
| AAVA3.4 | 142 | rAAV | 1020 | AAV8 | 2959 | AAV variant | 3835 |
| | | | | Variant | | | |
| AAVA3.4 | 143 | rAAV | 1021 | AAV8 | 2960 | AAV variant | 3836 |
| | | | | Variant | | | |
| AAVA3.5 | 144 | rAAV | 1022 | AAV8 | 2961 | AAV variant | 3837 |
| | | | | Variant | | | |
| AAVA3.5 | 145 | rAAV | 1023 | AAV8 | 2962 | AAV variant | 3838 |
| | | | | Variant | | | |
| AAVA3.7 | 146 | rAAV | 1024 | AAV8 | 2963 | AAV variant | 3839 |
| | | | | Variant | | | |
| AAVA3.7 | 147 | rAAV | 1025 | AAV8 | 2964 | AAV variant | 3840 |
| | | | | Variant | | | |
| AAV29.3 | 148 | rAAV | 1026 | AAV8 | 2965 | AAV variant | 3841 |
| (AAVbb.1) | | | | Variant | | | |
| AAVC2 | 149 | rAAV | 1027 | AAV8 | 2966 | AAV variant | 3842 |
| | | | | Variant | | | |
| AAVCh.5 | 150 | rAAV | 1028 | AAV8 | 2967 | AAV variant | 3843 |
| | | | | Variant | | | |
| AAVcy.2 | 151 | rAAV | 1029 | AAV8 | 2968 | AAV variant | 3844 |
| (AAV13.3) | | | | Variant | | | |
| AAV24.1 | 152 | rAAV | 1030 | AAV8 | 2969 | AAV variant | 3845 |
| | | | | Variant | | | |
| AAVcy.3 | 153 | rAAV | 1031 | AAV8 | 2970 | AAV variant | 3846 |
| (AAV24.1) | | | | Variant | | | |
| AAV27.3 | 154 | rAAV | 1032 | AAV8 | 2971 | AAV variant | 3847 |
| | | | | Variant | | | |
| AAVcy.4 | 155 | rAAV | 1033 | AAV8 | 2972 | AAV variant | 3848 |
| (AAV27.3) | | | | Variant | | | |
| AAVcy.5 | 156 | rAAV | 1034 | AAV8 | 2973 | AAV variant | 3849 |
| | | | | Variant | | | |
| AAV7.2 | 157 | rAAV | 1035 | AAV8 | 2974 | AAV variant | 3850 |
| | | | | Variant | | | |
| AAVcy.5 | 158 | rAAV | 1036 | AAV8 | 2975 | AAV variant | 3851 |
| (AAV7.2) | | | | Variant | | | |
| AAV16.3 | 159 | rAAV | 1037 | AAV8 | 2976 | AAV variant | 3852 |
| | | | | Variant | | | |
| AAVcy.6 | 160 | rAAV | 1038 | AAV8 | 2977 | AAV variant | 3853 |
| (AAV16.3) | | | | Variant | | | |
| AAVcy.5 | 161 | rAAV | 1039 | AAV8 | 2978 | AAV variant | 3854 |
| | | | | Variant | | | |
| AAVcy.5 | 162 | rAAV | 1040 | AAV8 | 2979 | AAV variant | 3855 |
| | | | | Variant | | | |
| AAVCy.5R1 | 163 | rAAV | 1041 | AAV8 | 2980 | AAV variant | 3856 |
| | | | | Variant | | | |
| AAVCy.5R2 | 164 | rAAV | 1042 | AAV8 | 2981 | AAV variant | 3857 |
| | | | | Variant | | | |
| AAVCy.5R3 | 165 | rAAV | 1043 | AAV8 | 2982 | AAV variant | 3858 |
| | | | | Variant | | | |
| AAVCy.5R4 | 166 | rAAV | 1044 | AAV8 | 2983 | AAV variant | 3859 |
| | | | | Variant | | | |
| AAVDJ | 167 | rAAV | 1045 | AAV8 | 2984 | AAV variant | 3860 |
| | | | | Variant | | | |
| AAVDJ | 168 | rAAV | 1046 | AAV8 | 2985 | AAV variant | 3861 |
| | | | | Variant | | | |
| AAVDJ-8 | 169 | rAAV | 1047 | AAV8 | 2986 | AAV variant | 3862 |
| | | | | Variant | | | |
| AAVDJ-8 | 170 | rAAV | 1048 | AAV8 | 2987 | AAV variant | 3863 |
| | | | | Variant | | | |
| AAVF5 | 171 | rAAV | 1049 | AAV8 | 2988 | AAV variant | 3864 |
| | | | | Variant | | | |
| AAVH2 | 172 | rAAV | 1050 | AAV8 | 2989 | AAV variant | 3865 |
| | | | | Variant | | | |
| AAVH6 | 173 | rAAV | 1051 | AAV8 | 2990 | AAV variant | 3866 |
| | | | | Variant | | | |
| AAVhE1.1 | 174 | rAAV | 1052 | AAV8 | 2991 | AAV variant | 3867 |
| | | | | Variant | | | |
| AAVhEr1.14 | 175 | rAAV | 1053 | AAV8 | 2992 | AAV variant | 3868 |
| | | | | Variant | | | |
| AAVhEr1.16 | 176 | rAAV | 1054 | AAV8 | 2993 | AAV variant | 3869 |
| | | | | Variant | | | |
| AAVhEr1.18 | 177 | rAAV | 1055 | AAV8 | 2994 | AAV variant | 3870 |
| | | | | Variant | | | |
| AAVhEr1.23 | 178 | rAAV | 1056 | AAV8 | 2995 | AAV variant | 3871 |
| (AAVhEr2.29) | | | | Variant | | | |
| AAVhEr1.35 | 179 | rAAV | 1057 | AAV8 | 2996 | AAV variant | 3872 |
| | | | | Variant | | | |
| AAVhEr1.36 | 180 | rAAV | 1058 | AAV8 | 2997 | AAV variant | 3873 |
| | | | | Variant | | | |
| AAVhEr1.5 | 181 | rAAV | 1059 | AAV8 | 2998 | AAV variant | 3874 |
| | | | | Variant | | | |
| AAVhEr1.7 | 182 | rAAV | 1060 | AAV8 | 2999 | AAV variant | 3875 |
| | | | | Variant | | | |
| AAVhEr1.8 | 183 | rAAV | 1061 | AAV8 | 3000 | AAV variant | 3876 |
| | | | | Variant | | | |
| AAVhEr2.16 | 184 | rAAV | 1062 | AAV8 | 3001 | AAV variant | 3877 |
| | | | | Variant | | | |
| AAVhEr2.30 | 185 | rAAV | 1063 | AAV8 | 3002 | AAV variant | 3878 |
| | | | | Variant | | | |
| AAVhEr2.31 | 186 | rAAV | 1064 | AAV8 | 3003 | AAV variant | 3879 |
| | | | | Variant | | | |
| AAVhEr2.36 | 187 | rAAV | 1065 | AAV8 | 3004 | AAV variant | 3880 |
| | | | | Variant | | | |
| AAVhEr2.4 | 188 | rAAV | 1066 | AAV8 | 3005 | AAV variant | 3881 |
| | | | | Variant | | | |
| AAVhEr3.1 | 189 | rAAV | 1067 | AAV8 | 3006 | AAV variant | 3882 |
| | | | | Variant | | | |
| AAVhu.1 | 190 | rAAV | 1068 | AAV8 | 3007 | AAV variant | 3883 |
| | | | | Variant | | | |
| AAVhu.1 | 191 | rAAV | 1069 | AAV8 | 3008 | AAV variant | 3884 |
| | | | | Variant | | | |
| AAVhu.10 | 192 | rAAV | 1070 | AAV8 | 3009 | AAV variant | 3885 |
| (AAV16.8) | | | | Variant | | | |
| AAVhu.10 | 193 | rAAV | 1071 | AAV8 | 3010 | AAV variant | 3886 |
| (AAV16.8) | | | | Variant | | | |
| AAVhu.11 | 194 | rAAV | 1072 | AAV8 | 3011 | AAV variant | 3887 |
| (AAV16.12) | | | | Variant | | | |
| AAVhu.11 | 195 | rAAV | 1073 | AAV8 | 3012 | AAV variant | 3888 |
| (AAV16.12) | | | | Variant | | | |
| AAVhu.12 | 196 | rAAV | 1074 | AAV8 | 3013 | AAV variant | 3889 |
| | | | | Variant | | | |
| AAVhu.12 | 197 | rAAV | 1075 | AAV8 | 3014 | AAV variant | 3890 |
| | | | | Variant | | | |
| AAVhu.13 | 198 | rAAV | 1076 | AAV8 | 3015 | AAV variant | 3891 |
| | | | | Variant | | | |
| AAVhu.13 | 199 | rAAV | 1077 | AAV8 | 3016 | AAV variant | 3892 |
| | | | | Variant | | | |
| AAVhu.136.1 | 200 | rAAV | 1078 | AAV8 | 3017 | AAV variant | 3893 |
| | | | | Variant | | | |
| AAVhu.140.1 | 201 | rAAV | 1079 | AAV8 | 3018 | AAV variant | 3894 |
| | | | | Variant | | | |
| AAVhu.140.2 | 202 | rAAV | 1080 | AAV8 | 3019 | AAV variant | 3895 |
| | | | | Variant | | | |
| AAVhu.145.6 | 203 | rAAV | 1081 | AAV8 | 3020 | AAV variant | 3896 |
| | | | | Variant | | | |
| AAVhu.15 | 204 | rAAV | 1082 | AAV8 | 3021 | AAV variant | 3897 |
| | | | | Variant | | | |
| AAVhu.15 | 205 | rAAV | 1083 | AAV8 | 3022 | AAV variant | 3898 |
| (AAV33.4) | | | | Variant | | | |
| AAVhu.156.1 | 206 | rAAV | 1084 | AAV8 | 3023 | AAV variant | 3899 |
| | | | | Variant | | | |
| AAVhu.16 | 207 | rAAV | 1085 | AAV8 | 3024 | AAV variant | 3900 |
| | | | | Variant | | | |
| AAVhu.16 | 208 | rAAV | 1086 | AAV8 | 3025 | AAV variant | 3901 |
| (AAV33.8) | | | | Variant | | | |
| AAVhu.17 | 209 | rAAV | 1087 | AAV8 | 3026 | AAV variant | 3902 |
| | | | | Variant | | | |
| AAVhu.17 | 210 | rAAV | 1088 | AAV8 | 3027 | AAV variant | 3903 |
| (AAV33.12) | | | | Variant | | | |
| AAVhu.172.1 | 211 | rAAV | 1089 | AAV8 | 3028 | AAV variant | 3904 |
| | | | | Variant | | | |
| AAVhu.172.2 | 212 | rAAV | 1090 | AAV8 | 3029 | AAV variant | 3905 |
| | | | | Variant | | | |
| AAVhu.173.4 | 213 | rAAV | 1091 | AAV8 | 3030 | AAV variant | 3906 |
| | | | | Variant | | | |
| AAVhu.173.8 | 214 | rAAV | 1092 | AAV8 | 3031 | AAV variant | 3907 |
| | | | | Variant | | | |
| AAVhu.18 | 215 | rAAV | 1093 | AAV8 | 3032 | AAV variant | 3908 |
| | | | | Variant | | | |
| AAVhu.18 | 216 | rAAV | 1094 | AAV8 | 3033 | AAV variant | 3909 |
| | | | | Variant | | | |
| AAVhu.19 | 217 | rAAV | 1095 | AAV8 | 3034 | AAV variant | 3910 |
| | | | | Variant | | | |
| AAVhu.19 | 218 | rAAV | 1096 | AAV8 | 3035 | AAV variant | 3911 |
| | | | | Variant | | | |
| AAVhu.2 | 219 | rAAV | 1097 | AAV8 | 3036 | AAV variant | 3912 |
| | | | | Variant | | | |
| AAVhu.2 | 220 | rAAV | 1098 | AAV8 | 3037 | AAV variant | 3913 |
| | | | | Variant | | | |
| AAVhu.20 | 221 | rAAV | 1099 | AAV8 | 3038 | AAV variant | 3914 |
| | | | | Variant | | | |
| AAVhu.20 | 222 | rAAV | 1100 | AAV8 | 3039 | AAV variant | 3915 |
| | | | | Variant | | | |
| AAVhu.21 | 223 | rAAV | 1101 | AAV8 | 3040 | AAV variant | 3916 |
| | | | | Variant | | | |
| AAVhu.21 | 224 | rAAV | 1102 | AAV8 | 3041 | AAV variant | 3917 |
| | | | | Variant | | | |
| AAVhu.22 | 225 | rAAV | 1103 | AAV8 | 3042 | AAV variant | 3918 |
| | | | | Variant | | | |
| AAVhu.22 | 226 | rAAV | 1104 | AAV8 | 3043 | AAV variant | 3919 |
| | | | | Variant | | | |
| AAVhu.23 | 227 | rAAV | 1105 | AAV8 | 3044 | AAV variant | 3920 |
| | | | | Variant | | | |
| AAVhu.23.2 | 228 | rAAV | 1106 | AAV8 | 3045 | AAV variant | 3921 |
| | | | | Variant | | | |
| AAVhu.24 | 229 | rAAV | 1107 | AAV8 | 3046 | AAV variant | 3922 |
| | | | | Variant | | | |
| AAVhu.24 | 230 | rAAV | 1108 | AAV8 | 3047 | AAV variant | 3923 |
| | | | | Variant | | | |
| AAVhu.25 | 231 | rAAV | 1109 | AAV8 | 3048 | AAV variant | 3924 |
| | | | | Variant | | | |
| AAVhu.25 | 232 | rAAV | 1110 | AAV8 | 3049 | AAV variant | 3925 |
| | | | | Variant | | | |
| AAVhu.26 | 233 | rAAV | 1111 | AAV8 | 3050 | AAV variant | 3926 |
| | | | | Variant | | | |
| AAVhu.26 | 234 | rAAV | 1112 | AAV8 | 3051 | AAV variant | 3927 |
| | | | | Variant | | | |
| AAVhu.27 | 235 | rAAV | 1113 | AAV8 | 3052 | AAV variant | 3928 |
| | | | | Variant | | | |
| AAVhu.27 | 236 | rAAV | 1114 | AAV8 | 3053 | AAV variant | 3929 |
| | | | | Variant | | | |
| AAVhu.28 | 237 | rAAV | 1115 | AAV8 | 3054 | AAV variant | 3930 |
| | | | | Variant | | | |
| AAVhu.28 | 238 | rAAV | 1116 | AAV8 | 3055 | AAV variant | 3931 |
| | | | | Variant | | | |
| AAVhu.29 | 239 | rAAV | 1117 | AAV8 | 3056 | AAV variant | 3932 |
| | | | | Variant | | | |
| AAVhu.29 | 240 | rAAV | 1118 | AAV8 | 3057 | AAV variant | 3933 |
| | | | | Variant | | | |
| AAVhu.29 | 241 | rAAV | 1119 | AAV8 | 3058 | AAV variant | 3934 |
| | | | | Variant | | | |
| AAVhu.29R | 242 | rAAV | 1120 | AAV8 | 3059 | AAV variant | 3935 |
| | | | | Variant | | | |
| AAVhu.3 | 243 | rAAV | 1121 | AAV8 | 3060 | AAV variant | 3936 |
| | | | | Variant | | | |
| AAVhu.3 | 244 | rAAV | 1122 | AAV8 | 3061 | AAV variant | 3937 |
| | | | | Variant | | | |
| AAVhu.30 | 245 | rAAV | 1123 | AAV8 | 3062 | AAV variant | 3938 |
| | | | | Variant | | | |
| AAVhu.30 | 246 | rAAV | 1124 | AAV8 | 3063 | AAV variant | 3939 |
| | | | | Variant | | | |
| AAVhu.31 | 247 | AAV8 E532K | 1125 | AAV8 | 3064 | AAV variant | 3940 |
| | | | | Variant | | | |
| AAVhu.31 | 248 | AAV8 E532K | 1126 | AAV8 | 3065 | AAV variant | 3941 |
| | | | | Variant | | | |
| AAVhu.32 | 249 | rAAV4 | 1127 | AAV8 | 3066 | AAV variant | 3942 |
| | | | | Variant | | | |
| AAVhu.32 | 250 | rAAV4 | 1128 | AAV8 | 3067 | AAV variant | 3943 |
| | | | | Variant | | | |
| AAVhu.33 | 251 | rAAV4 | 1129 | AAV8 | 3068 | AAV variant | 3944 |
| | | | | Variant | | | |
| AAVhu.33 | 252 | rAAV4 | 1130 | AAV8 | 3069 | AAV variant | 3945 |
| | | | | Variant | | | |
| AAVhu.34 | 253 | rAAV4 | 1131 | AAV8 | 3070 | AAV variant | 3946 |
| | | | | Variant | | | |
| AAVhu.34 | 254 | rAAV4 | 1132 | AAV8 | 3071 | AAV variant | 3947 |
| | | | | Variant | | | |
| AAVhu.35 | 255 | rAAV4 | 1133 | AAV8 | 3072 | AAV variant | 3948 |
| | | | | Variant | | | |
| AAVhu.35 | 256 | rAAV4 | 1134 | AAV8 | 3073 | AAV variant | 3949 |
| | | | | Variant | | | |
| AAVhu.36 | 257 | rAAV4 | 1135 | AAV8 | 3074 | AAV variant | 3950 |
| | | | | Variant | | | |
| AAVhu.36 | 258 | rAAV4 | 1136 | AAV8 | 3075 | AAV variant | 3951 |
| | | | | Variant | | | |
| AAVhu.37 | 259 | rAAV4 | 1137 | AAV8 | 3076 | AAV variant | 3952 |
| | | | | Variant | | | |
| AAVhu.37 | 260 | rAAV4 | 1138 | AAV8 | 3077 | AAV variant | 3953 |
| (AAV106.1) | | | | Variant | | | |
| AAVhu.38 | 261 | rAAV4 | 1139 | AAV8 | 3078 | AAV variant | 3954 |
| | | | | Variant | | | |
| AAVhu.39 | 262 | rAAV4 | 1140 | AAV8 | 3079 | AAV variant | 3955 |
| | | | | Variant | | | |
| AAVhu.39 | 263 | rAAV4 | 1141 | AAV8 | 3080 | AAV variant | 3956 |
| (AAVLG-9) | | | | Variant | | | |
| AAVhu.4 | 264 | rAAV4 | 1142 | AAV8 | 3081 | AAV variant | 3957 |
| | | | | Variant | | | |
| AAVhu.4 | 265 | rAAV4 | 1143 | AAV8 | 3082 | AAV variant | 3958 |
| | | | | Variant | | | |
| AAVhu.40 | 266 | rAAV4 | 1144 | AAV8 | 3083 | AAV variant | 3959 |
| | | | | Variant | | | |
| AAVhu.40 | 267 | rAAV4 | 1145 | AAV8 | 3084 | AAV variant | 3960 |
| (AAV114.3) | | | | Variant | | | |
| AAVhu.41 | 268 | rAAV4 | 1146 | AAV8 | 3085 | AAV variant | 3961 |
| | | | | Variant | | | |
| AAVhu.41 | 269 | AAV11 | 1147 | AAV8 | 3086 | AAV variant | 3962 |
| (AAV127.2) | | | | Variant | | | |
| AAVhu.42 | 270 | AAV12 | 1148 | AAV8 | 3087 | AAV variant | 3963 |
| | | | | Variant | | | |
| AAVhu.42 | 271 | rh32 | 1149 | AAV8 | 3088 | AAV variant | 3964 |
| (AAV127.5) | | | | Variant | | | |
| AAVhu.43 | 272 | rh33 | 1150 | AAV8 | 3089 | AAV variant | 3965 |
| | | | | Variant | | | |
| AAVhu.43 | 273 | rh34 | 1151 | AAV8 | 3090 | AAV variant | 3966 |
| | | | | Variant | | | |
| AAVhu.43 | 274 | rAAV4 | 1152 | AAV8 | 3091 | AAV variant | 3967 |
| (AAV128.1) | | | | Variant | | | |
| AA Vhu.44 | 275 | rAAV4 | 1153 | AAV8 | 3092 | AAV variant | 3968 |
| | | | | Variant | | | |
| AA Vhu.44 | 276 | rAAV4 | 1154 | AAV8 | 3093 | AAV variant | 3969 |
| (AAV128.3) | | | | Variant | | | |
| AAVhu.44R1 | 277 | rAAV4 | 1155 | AAV8 | 3094 | AAV variant | 3970 |
| | | | | Variant | | | |
| AAVhu.44R2 | 278 | rAAV4 | 1156 | AAV8 | 3095 | AAV variant | 3971 |
| | | | | Variant | | | |
| AAVhu.44R3 | 279 | rAAV4 | 1157 | AAV8 | 3096 | AAV variant | 3972 |
| | | | | Variant | | | |
| AAVhu.45 | 280 | AAV2/8 | 1158 | AAV8 | 3097 | AAV variant | 3973 |
| | | | | Variant | | | |
| AAVhu.45 | 281 | AAV2/8 | 1159 | AAV8 | 3098 | AAV variant | 3974 |
| | | | | Variant | | | |
| AAVhu.46 | 282 | ancestral AAV | 1160 | AAV8 | 3099 | AAV variant | 3975 |
| | | | | Variant | | | |
| AAVhu.46 | 283 | ancestral AAV | 1161 | AAV8 | 3100 | AAV variant | 3976 |
| | | variant C4 | | Variant | | | |
| AAVhu.46 | 284 | ancestral AAV | 1162 | AAV8 | 3101 | AAV variant | 3977 |
| | | variant C7 | | Variant | | | |
| AAVhu.47 | 285 | ancestral AAV | 1163 | AAV8 | 3102 | AAV variant | 3978 |
| | | variant G4 | | Variant | | | |
| AAVhu.47 | 286 | AAV VR-942n | 1167 | AAV8 | 3103 | AAV variant | 3979 |
| | | | | Variant | | | |
| AAVhu.48 | 287 | AAV5-A (M569V) | 1168 | AAV8 | 3104 | AAV variant | 3980 |
| | | | | Variant | | | |
| AAVhu.48 | 288 | AAV5-A (M569V) | 1169 | AAV8 | 3105 | AAV variant | 3981 |
| | | | | Variant | | | |
| AAVhu.48 | 289 | AAV5-A (Y585V) | 1170 | AAV8 | 3106 | AAV variant | 3982 |
| (AAV130.4) | | | | Variant | | | |
| AAVhu.48R1 | 290 | AAV5-A (Y585V) | 1171 | AAV8 | 3107 | AAV variant | 3983 |
| | | | | Variant | | | |
| AAVhu.48R2 | 291 | AAV5-A (L587T) | 1172 | AAV8 | 3108 | AAV variant | 3984 |
| | | | | Variant | | | |
| AAVhu.48R3 | 292 | AAV5-A (L587T) | 1173 | AAV8 | 3109 | AAV variant | 3985 |
| | | | | Variant | | | |
| AAVhu.49 | 293 | AAV5-A | 1174 | AAV8 | 3110 | AAV variant | 3986 |
| | | (Y585V/L587T) | | Variant | | | |
| AAVhu.49 | 294 | AAV5-A | 1175 | AAV8 | 3111 | AAV variant | 3987 |
| | | (Y585V/L587T) | | Variant | | | |
| AAVhu.5 | 295 | AAV5-B (D652A) | 1176 | AAV8 | 3112 | AAV variant | 3988 |
| | | | | Variant | | | |
| AAVhu.5 | 296 | AAV5-B (D652A) | 1177 | AAV8 | 3113 | AAV variant | 3989 |
| | | | | Variant | | | |
| AAVhu.51 | 297 | AAV5-B (T362M) | 1178 | AAV8 | 3114 | AAV variant | 3990 |
| | | | | Variant | | | |
| AAVhu.51 | 298 | AAV5-B (T362M) | 1179 | AAV8 | 3115 | AAV variant | 3991 |
| | | | | Variant | | | |
| AAVhu.52 | 299 | AAV5-B (Q359D) | 1180 | AAV8 | 3116 | AAV variant | 3992 |
| | | | | Variant | | | |
| AAVhu.52 | 300 | AAV5-B (Q359D) | 1181 | AAV8 | 3117 | AAV variant | 3993 |
| | | | | Variant | | | |
| AAVhu.53 | 301 | AAV5-B (E350Q) | 1182 | AAV8 | 3118 | AAV variant | 3994 |
| | | | | Variant | | | |
| AAVhu.53 | 302 | AAV5-B (E350Q) | 1183 | AAV8 | 3119 | AAV variant | 3995 |
| | | | | Variant | | | |
| AAVhu.53 | 303 | AAV5-B (P533S) | 1184 | AAV8 | 3120 | AAV variant | 3996 |
| (AAV145.1) | | | | Variant | | | |
| AAVhu.54 | 304 | AAV5-B (P533S) | 1185 | AAV8 | 3121 | AAV variant | 3997 |
| | | | | Variant | | | |
| AAVhu.54 | 305 | AAV5-B (P533G) | 1186 | AAV8 | 3122 | AAV variant | 3998 |
| (AAV145.5) | | | | Variant | | | |
| AAVhu.55 | 306 | AAV5-B (P533G) | 1187 | AAV8 | 3123 | AAV variant | 3999 |
| | | | | Variant | | | |
| AAVhu.56 | 307 | AAV5-mutation in | 1188 | AAV8 | 3124 | AAV variant | 4000 |
| | | loop VII | | Variant | | | |
| AAVhu.56 | 308 | AAV5-mutation in | 1189 | AAV8 | 3125 | AAV variant | 4001 |
| (AAV145.6) | | loop VII | | Variant | | | |
| AAVhu.56 | 309 | AAV8 | 1190 | AAV8 | 3126 | AAV variant | 4002 |
| AAV145.6) | | | | Variant | | | |
| AAVhu.57 | 310 | Mut A (LK03/AAV8) | 1191 | AAV8 | 3127 | AAV variant | 4003 |
| | | | | Variant | | | |
| AAVhu.57 | 311 | Mut B (LK03/AAV5) | 1192 | AAV8 | 3128 | AAV variant | 4004 |
| | | | | Variant | | | |
| AAVhu.57 | 312 | Mut C | 1193 | AAV8 | 3129 | AAV variant | 4005 |
| | | (AAV8/AAV3B) | | Variant | | | |
| AAVhu.58 | 313 | Mut D | 1194 | AAV8 | 3130 | AAV variant | 4006 |
| | | (AAV5/AAV3B) | | Variant | | | |
| AAVhu.58 | 314 | Mut E | 1195 | AAV8 | 3131 | AAV variant | 4007 |
| | | (AAV8/AAV3B) | | Variant | | | |
| AAVhu.6 | 315 | Mut F | 1196 | AAV8 | 3132 | AAV2 variant | 4008 |
| (AAV3.1) | | (AAV3B/AAV8) | | Variant | | | |
| AAVhu.6 | 316 | AAV44.9 | 1197 | AAV2 | 3133 | AAV2 variant | 4009 |
| (AAV3.1) | | | | variant | | | |
| AAVhu.60 | 317 | AAV44.9 | 1198 | AAV2 | 3134 | AAV2 variant | 4010 |
| | | | | variant | | | |
| AAVhu.60 | 318 | AAVrh8 | 1199 | AAV2 | 3135 | AAV2 variant | 4011 |
| (AAV161.10) | | | | variant | | | |
| AAVhu.61 | 319 | AAV44.9 (S470N) | 1200 | AAV2 | 3136 | AAV2 variant | 4012 |
| | | | | variant | | | |
| AAVhu.61 | 320 | rh74 VP1 | 1201 | AAV2 | 3137 | AAV2 variant | 4013 |
| (AAV161.6) | | | | variant | | | |
| AAVhu.63 | 321 | AAV-LK03 (L125I) | 1202 | AAV2 | 3138 | AAV2 variant | 4014 |
| | | | | variant | | | |
| AAVhu.63 | 322 | AAV3B | 1203 | AAV2 | 3139 | AAV2 variant | 4015 |
| | | (S663V + T492V) | | variant | | | |
| AAVhu.64 | 323 | Anc80 | 1204 | AAV2 | 3140 | AAV2 variant | 4016 |
| | | | | variant | | | |
| AAVhu.64 | 324 | Anc80 | 1205 | AAV2 | 3141 | AAV2 variant | 4017 |
| | | | | variant | | | |
| AAVhu.66 | 325 | Anc81 | 1206 | AAV2 | 3142 | AAV2 variant | 4018 |
| | | | | variant | | | |
| AAVhu.67 | 326 | Anc81 | 1207 | AAV2 | 3143 | AAV2 variant | 4019 |
| | | | | variant | | | |
| AAVhu.67 | 327 | Anc82 | 1208 | AAV2 | 3144 | AAV2 variant | 4020 |
| | | | | variant | | | |
| AAVhu.7 | 328 | Anc82 | 1209 | AAV2 | 3145 | AAV2 variant | 4021 |
| | | | | variant | | | |
| AAVhu.7 | 329 | Anc83 | 1210 | AAV2 | 3146 | AAV2 variant | 4022 |
| | | | | variant | | | |
| AAVhu.7 | 330 | Anc83 | 1211 | AAV2 | 3147 | AAV2 variant | 4023 |
| (AAV7.3) | | | | variant | | | |
| AAVhu.71 | 331 | Anc84 | 1212 | AAV2 | 3148 | AAV2 variant | 4024 |
| | | | | variant | | | |
| AAVhu.8 | 332 | Anc84 | 1213 | AAV8 B1 | 3149 | AAV2 variant | 4025 |
| AAVhu.8 | 333 | Anc94 | 1214 | AAV8 B2 | 3150 | AAV2 variant | 4026 |
| AAVhu.8 | 334 | Anc94 | 1215 | AAV8 B3 | 3151 | AAV2 variant | 4027 |
| AAVhu.9 | 335 | Anc113 | 1216 | AAV8 B4 | 3152 | AAV2 variant | 4028 |
| (AAV3.1) | | | | | | | |
| AAVhu.9 | 336 | Anc113 | 1217 | AAV8 B12 | 3153 | AAV2 variant | 4029 |
| (AAV3.1) | | | | | | | |
| AAV-LK01 | 337 | Anc126 | 1218 | AAV8 B18 | 3154 | AAV2 variant | 4030 |
| AAV-LK01 | 338 | Anc126 | 1219 | AAV8 B24 | 3155 | AAV2 variant | 4031 |
| AAV-LK02 | 339 | Anc127 | 1220 | AAV8 B41 | 3156 | AAV2 variant | 4032 |
| AAV-LK02 | 340 | Anc127 | 1221 | AAV8 B44 | 3157 | AAV2 variant | 4033 |
| AAV-LK03 | 341 | Anc80L27 | 1222 | AAV8 B45 | 3158 | AAV2 variant | 4034 |
| AAV-LK03 | 342 | Anc80L59 | 1223 | AAV8 B46 | 3159 | AAV2 variant | 4035 |
| AAV-LK04 | 343 | Anc80L60 | 1224 | AAV8 B60 | 3160 | AAV2 variant | 4036 |
| AAV-LK04 | 344 | Anc80L62 | 1225 | AAV8 B61 | 3161 | AAV2 variant | 4037 |
| AAV-LK05 | 345 | Anc80L65 | 1226 | AAV8 B62 | 3162 | AAV2 variant | 4038 |
| AAV-LK05 | 346 | Anc80L33 | 1227 | AAV8 B63 | 3163 | AAV2 variant | 4039 |
| AAV-LK06 | 347 | Anc80L36 | 1228 | AAV8 B64 | 3164 | AAV2 variant | 4040 |
| AAV-LK06 | 348 | Anc80L44 | 1229 | AAV variant | 3165 | AAV2 variant | 4041 |
| AAV-LK07 | 349 | Anc80L1 | 1230 | AAV variant | 3166 | AAV2 variant | 4042 |
| AAV-LK07 | 350 | Anc80L1 | 1231 | AAV variant | 3167 | AAV2 variant | 4043 |
| AAV-LK08 | 351 | AAVrh10 | 1232 | AAV variant | 3168 | AAV2 variant | 4044 |
| AAV-LK08 | 352 | Anc110 | 1233 | AAV variant | 3169 | AAV2 variant | 4045 |
| AAV-LK09 | 353 | Anc110 | 1234 | AAV variant | 3170 | AAV2 variant | 4046 |
| AAV-LK09 | 354 | AAVrh32.33 | 1235 | AAV variant | 3171 | AAV2 variant | 4047 |
| AAV-LK10 | 355 | AAVrh74 | 1236 | AAV variant | 3172 | AAV2 variant | 4048 |
| AAV-LK10 | 356 | AAV2 | 1237 | AAV variant | 3173 | AAV2 variant | 4049 |
| AAV-LK11 | 357 | AAV2 | 1238 | AAV variant | 3174 | AAV2 variant | 4050 |
| AAV-LK11 | 358 | AAV2 | 1239 | AAV variant | 3175 | AAV2 variant | 4051 |
| AAV-LK12 | 359 | Parvo-like virus | 1240 | AAV variant | 3176 | AAV2 variant | 4052 |
| AAV-LK12 | 360 | Parvo-like virus | 1241 | AAV variant | 3177 | AAV2 variant | 4053 |
| AAV-LK13 | 361 | Parvo-like virus | 1242 | AAV variant | 3178 | AAV2 variant | 4054 |
| AAV-LK13 | 362 | Parvo-like virus | 1243 | AAV variant | 3179 | AAV2 variant | 4055 |
| AAV-LK14 | 363 | Parvo-like virus | 1244 | AAV variant | 3180 | AAV2 variant | 4056 |
| AAV-LK14 | 364 | Parvo-like virus | 1245 | AAV variant | 3181 | AAV2 variant | 4057 |
| AAV-LK15 | 365 | AAVrh.10 | 1246 | AAV variant | 3182 | AAV2 variant | 4058 |
| AAV-LK15 | 366 | AAVrh.10 | 1247 | AAV variant | 3183 | AAV2 variant | 4059 |
| AAV-LK16 | 367 | AAV2tYF | 1248 | AAV variant | 3184 | AAV2 variant | 4060 |
| AAV-LK16 | 368 | AAV-SPK | 1249 | AAV variant | 3185 | AAV2 variant | 4061 |
| AAV-LK17 | 369 | AAV2.5 | 1250 | AAV variant | 3186 | AAV2 variant | 4062 |
| AAV-LK17 | 370 | AAV1.1 | 1251 | AAV variant | 3187 | AAV2 variant | 4063 |
| AAV-LK18 | 371 | AAV6.1 | 1252 | AAV variant | 3188 | AAV2 variant | 4064 |
| AAV-LK18 | 372 | AAV6.3.1 | 1253 | AAV variant | 3189 | AAV2 variant | 4065 |
| AAV-LK19 | 373 | AAV218 | 1254 | AAV variant | 3190 | AAV2 variant | 4066 |
| AAV-LK19 | 374 | AAV218 | 1255 | AAV variant | 3191 | AAV2 variant | 4067 |
| AAV-PAEC | 375 | ttAAV | 1256 | AAV variant | 3192 | AAV2 variant | 4068 |
| AAV-PAEC | 376 | ttAAV-S312N | 1257 | AAV variant | 3193 | AAV2 variant | 4069 |
| AAV-PAEC11 | 377 | ttAAV-S312N | 1258 | AAV variant | 3194 | AAV2 variant | 4070 |
| AAV-PAEC11 | 378 | AAV6 (Y705, Y731, | 1259 | AAV variant | 3195 | AAV2 variant | 4071 |
| | | and T492) | | | | | |
| AAV-PAEC12 | 379 | AAV2 | 1260 | AAV variant | 3196 | AAV2 variant | 4072 |
| AAV-PAEC12 | 380 | AAV2 | 1261 | AAV variant | 3197 | AAV2 variant | 4073 |
| AAV-PAEC13 | 381 | AAV2 variant | 2322 | AAV variant | 3198 | AAV2 variant | 4074 |
| AAV-PAEC13 | 382 | AAV2 variant | 2323 | AAV variant | 3199 | AAV2 variant | 4075 |
| AAV-PAEC2 | 383 | AAV2 variant | 2324 | AAV variant | 3200 | AAV2 variant | 4076 |
| AAV-PAEC2 | 384 | AAV2 variant | 2325 | AAV variant | 3201 | AAV2 variant | 4077 |
| AAV-PAEC4 | 385 | AAV2 variant | 2326 | AAV variant | 3202 | AAV2 variant | 4078 |
| AAV-PAEC4 | 386 | AAV2 variant | 2327 | AAV variant | 3203 | AAV2 variant | 4079 |
| AAV-PAEC6 | 387 | AAV2 variant | 2328 | AAV variant | 3204 | AAV2 variant | 4080 |
| AAV-PAEC6 | 388 | AAV2 variant | 2329 | AAV variant | 3205 | AAV2 variant | 4081 |
| AAV-PAEC7 | 389 | AAV2 variant | 2330 | AAV variant | 3206 | AAV2 variant | 4082 |
| AAV-PAEC7 | 390 | AAV2 variant | 2331 | AAV variant | 3207 | AAV2 variant | 4083 |
| AAV-PAEC8 | 391 | AAV2 variant | 2332 | AAV variant | 3208 | AAV2 variant | 4084 |
| AAV-PAEC8 | 392 | AAV2 variant | 2333 | AAV variant | 3209 | AAV2 variant | 4085 |
| AAVpi.1 | 393 | AAV2 variant | 2334 | AAV variant | 3210 | AAV2 variant | 4086 |
| AAVpi.1 | 394 | AAV2 variant | 2335 | AAV variant | 3211 | AAV2 variant | 4087 |
| AAVpi.2 | 395 | AAV2 variant | 2336 | AAV variant | 3212 | AAV2 variant | 4088 |
| AAVpi.2 | 396 | AAV2 variant | 2337 | AAV variant | 3213 | AAV2 variant | 4089 |
| AAVpi.3 | 397 | AAV2 variant | 2338 | AAV variant | 3214 | AAV2 variant | 4090 |
| AAVpi.3 | 398 | AAV2 variant | 2339 | AAV variant | 3215 | AAV2 variant | 4091 |
| AAVrh.10 | 399 | AAV2 variant | 2340 | AAV variant | 3216 | AAV2 variant | 4092 |
| AAVrh.10 | 400 | AAV2 variant | 2341 | AAV variant | 3217 | AAV2 variant | 4093 |
| AAV44.2 | 401 | AAV2 variant | 2342 | AAV variant | 3218 | AAV2 variant | 4094 |
| AAVrh.10 | 402 | AAV2 variant | 2343 | AAV variant | 3219 | AAV2 variant | 4095 |
| (AAV44.2) | | | | | | | |
| AAV42.1B | 403 | AAV2 variant | 2344 | AAV variant | 3220 | AAV2 variant | 4096 |
| AAVrh.12 | 404 | AAV2 variant | 2345 | AAV variant | 3221 | AAV2/3 variant | 4097 |
| (AAV42.1b) | | | | | | | |
| AAVrh.13 | 405 | AAV2 variant | 2346 | AAV variant | 3222 | AAV2/3 variant | 4098 |
| AAVrh.13 | 406 | AAV2 variant | 2347 | AAV variant | 3223 | AAV2/3 variant | 4099 |
| AAVrh.13 | 407 | AAV2 variant | 2348 | AAV variant | 3224 | AAV2/3 variant | 4100 |
| AAVrh.13R | 408 | AAV2 variant | 2349 | AAV variant | 3225 | AAV2/3 variant | 4101 |
| AAV42.3A | 409 | AAV2 variant | 2350 | AAV variant | 3226 | AAV2/3 variant | 4102 |
| AAVrh.14 | 410 | AAV2 variant | 2351 | AAV variant | 3227 | AAV2/3 variant | 4103 |
| (AAV42.3a) | | | | | | | |
| AAV42.5A | 411 | AAV2 variant | 2352 | AAV variant | 3228 | AAV2/3 variant | 4104 |
| AAVrh.17 | 412 | AAV2 variant | 2353 | AAV variant | 3229 | AAV2/3 variant | 4105 |
| (AAV42.5a) | | | | | | | |
| AAV42.5B | 413 | AAV2 variant | 2354 | AAV variant | 3230 | AAV2/3 variant | 4106 |
| AAVrh.18 | 414 | AAV2 variant | 2355 | AAV variant | 3231 | AAV2/3 variant | 4107 |
| (AAV42.5b) | | | | | | | |
| AAV42.6B | 415 | AAV2 variant | 2356 | AAV variant | 3232 | AAV2/3 variant | 4108 |
| AAVrh.19 | 416 | AAV2 variant | 2357 | AAV variant | 3233 | AAV2/3 variant | 4109 |
| (AAV42.6b) | | | | | | | |
| AAVrh.2 | 417 | AAV2 variant | 2358 | AAV variant | 3234 | AAV2/3 variant | 4110 |
| AAVrh.2 | 418 | AAV2 variant | 2359 | AAV variant | 3235 | AAV2/3 variant | 4111 |
| AAVrh.20 | 419 | AAV2 variant | 2360 | AAV variant | 3236 | AAV2/3 variant | 4112 |
| AAV42.10 | 420 | AAV2 variant | 2361 | AAV variant | 3237 | AAV2/3 variant | 4113 |
| AAVrh.21 | 421 | AAV2 variant | 2362 | AAV variant | 3238 | AAV2/3 variant | 4114 |
| (AAV42.10) | | | | | | | |
| AAV42.11 | 422 | AAV2 variant | 2363 | AAV variant | 3239 | AAV2/3 variant | 4115 |
| AAVrh.22 | 423 | AAV2 variant | 2364 | AAV variant | 3240 | AAV2/3 variant | 4116 |
| (AAV42.11) | | | | | | | |
| AAV42.12 | 424 | AAV2 variant | 2365 | AAV variant | 3241 | AAV2/3 variant | 4117 |
| AAVrh.23 | 425 | AAV2 variant | 2366 | AAV variant | 3242 | AAV2/3 variant | 4118 |
| (AAV42.12) | | | | | | | |
| AAV42.13 | 426 | AAV2 variant | 2367 | AAV variant | 3243 | AAV2/3 variant | 4119 |
| AAVrh.24 | 427 | AAV2 variant | 2368 | AAV variant | 3244 | AAV2/3 variant | 4120 |
| (AAV42.13) | | | | | | | |
| AAV42.15 | 428 | AAV2 variant | 2369 | AAV variant | 3245 | AAV2/3 variant | 4121 |
| AAVrh.25 | 429 | AAV2 variant | 2370 | AAV variant | 3246 | AAV2/3 variant | 4122 |
| (AAV42.15) | | | | | | | |
| AAVrh.2R | 430 | AAV2 variant | 2371 | AAV variant | 3247 | AAV2/3 variant | 4123 |
| AAVrh.31 | 431 | AAV2 variant | 2372 | AAV variant | 3248 | AAV2/3 variant | 4124 |
| (AAV223.1) | | | | | | | |
| AAVC1 | 432 | AAV2 variant | 2373 | AAV variant | 3249 | AAV2/3 variant | 4125 |
| AAVrh.32 | 433 | AAV2 variant | 2374 | AAV variant | 3250 | AAV2/3 variant | 4126 |
| (AAVC1) | | | | | | | |
| AAVrh.32/33 | 434 | AAV2 variant | 2375 | AAV variant | 3251 | AAV2/3 variant | 4127 |
| AAVrh.33 | 435 | AAV2 variant | 2376 | AAV variant | 3252 | AAV2/3 variant | 4128 |
| (AAVC3) | | | | | | | |
| AAVC5 | 436 | AAV2 variant | 2377 | AAV variant | 3253 | AAV2/3 variant | 4129 |
| AAVrh.34 | 437 | AAV2 variant | 2378 | AAV variant | 3254 | AAV2/3 variant | 4130 |
| (AAVC5) | | | | | | | |
| AAVF1 | 438 | AAV2 variant | 2379 | AAV variant | 3255 | AAV2/3 variant | 4131 |
| AAVrh.35 | 439 | AAV2 variant | 2380 | AAV variant | 3256 | AAV2/3 variant | 4132 |
| (AAVF1) | | | | | | | |
| AAVF3 | 440 | AAV2 variant | 2381 | AAV variant | 3257 | AAV2/3 variant | 4133 |
| AAVrh.36 | 441 | AAV2 variant | 2382 | AAV variant | 3258 | AAV2/3 variant | 4134 |
| (AAVF3) | | | | | | | |
| AAVrh.37 | 442 | AAV2 variant | 2383 | AAV variant | 3259 | AAV2/3 variant | 4135 |
| AAVrh.37 | 443 | AAV2 variant | 2384 | AAV variant | 3260 | AAV2/3 variant | 4136 |
| AAVrh.37 | 444 | AAV2 variant | 2385 | AAV variant | 3261 | AAV2/3 variant | 4137 |
| AAVrh.37R2 | 445 | AAV2 variant | 2386 | AAV variant | 3262 | AAV2/3 variant | 4138 |
| AAVrh.38 | 446 | AAV2 variant | 2387 | AAV variant | 3263 | AAV2/3 variant | 4139 |
| (AAVLG-4) | | | | | | | |
| AAVrh.38 | 447 | AAV2 variant | 2388 | AAV variant | 3264 | AAV2/3 variant | 4140 |
| (AAVLG-4) | | | | | | | |
| AAVrh.39 | 448 | AAV2 variant | 2389 | AAV variant | 3265 | AAV2/3 variant | 4141 |
| AAVrh.39 | 449 | AAV2 variant | 2390 | AAV variant | 3266 | AAV2/3 variant | 4142 |
| AAVrh.40 | 450 | AAV2 variant | 2391 | AAV variant | 3267 | AAV2/3 variant | 4143 |
| AAVrh.40 | 451 | AAV2 variant | 2392 | AAV variant | 3268 | AAV2/3 variant | 4144 |
| (AAVLG-10) | | | | | | | |
| AAVrh.43 | 452 | AAV2 variant | 2393 | AAV variant | 3269 | AAV2/3 variant | 4145 |
| (AAVN721-8) | | | | | | | |
| AAVrh.43 | 453 | AAV2 variant | 2394 | AAV variant | 3270 | AAV2/3 variant | 4146 |
| (AAVN721-8) | | | | | | | |
| AAVrh.44 | 454 | AAV2 variant | 2395 | AAV variant | 3271 | AAV2/3 variant | 4147 |
| AAVrh.44 | 455 | AAV2 variant | 2396 | AAV variant | 3272 | AAV2/3 variant | 4148 |
| AAVrh.45 | 456 | AAV2 variant | 2397 | AAV variant | 3273 | AAV2/3 variant | 4149 |
| AAVrh.45 | 457 | AAV2 variant | 2398 | AAV variant | 3274 | AAV2/3 variant | 4150 |
| AAVrh.46 | 458 | AAV2 variant | 2399 | AAV variant | 3275 | AAV2/3 variant | 4151 |
| AAVrh.46 | 459 | AAV2 variant | 2400 | AAV variant | 3276 | AAV2/3 variant | 4152 |
| AAVrh.47 | 460 | AAV2 variant | 2401 | AAV variant | 3277 | AAV2/3 variant | 4153 |
| AAVrh.47 | 461 | AAV2 variant | 2402 | AAV variant | 3278 | AAV2/3 variant | 4154 |
| AAVrh.48 | 462 | AAV2 variant | 2403 | AAV variant | 3279 | AAV2/3 variant | 4155 |
| AAVrh.48.1 | 463 | AAV2 variant | 2404 | AAV variant | 3280 | AAV2/3 variant | 4156 |
| AAVrh.48.1.2 | 464 | AAV2 variant | 2405 | AAV variant | 3281 | AAV2/3 variant | 4157 |
| AAVrh.48.2 | 465 | AAV2 variant | 2406 | AAV variant | 3282 | AAV2/3 variant | 4158 |
| AAVrh.48 | 466 | AAV2 variant | 2407 | AAV variant | 3283 | AAV2/3 variant | 4159 |
| (AAV1-7) | | | | | | | |
| AAVrh.49 | 467 | AAV2 variant | 2408 | AAV variant | 3284 | AAV2/3 variant | 4160 |
| (AAV1-8) | | | | | | | |
| AAVrh.49 | 468 | AAV2 variant | 2409 | AAV variant | 3285 | AAV2/3 variant | 4161 |
| (AAV1-8) | | | | | | | |
| AAVrh.50 | 469 | AAV2 variant | 2410 | AAV variant | 3286 | AAV2/3 variant | 4162 |
| (AAV2-4) | | | | | | | |
| AAVrh.50 | 470 | AAV2 variant | 2411 | AAV variant | 3287 | AAV2/3 variant | 4163 |
| (AAV2-4) | | | | | | | |
| AAVrh.51 | 471 | AAV2 variant | 2412 | AAV variant | 3288 | AAV2/3 variant | 4164 |
| (AAV2-5) | | | | | | | |
| AAVrh.51 | 472 | AAV2 variant | 2413 | AAV variant | 3289 | AAV2/3 variant | 4165 |
| (AAV2-5) | | | | | | | |
| AAVrh.52 | 473 | AAV2 variant | 2414 | AAV variant | 3290 | AAV2/3 variant | 4166 |
| (AAV3-9) | | | | | | | |
| AAVrh.52 | 474 | AAV2 variant | 2415 | AAV variant | 3291 | AAV2/3 variant | 4167 |
| (AAV3-9) | | | | | | | |
| AAVrh.53 | 475 | AAV2 variant | 2416 | AAV variant | 3292 | AAV2/3 variant | 4168 |
| AAVrh.53 | 476 | AAV2 variant | 2417 | AAV variant | 3293 | AAV2/3 variant | 4169 |
| (AAV3-11) | | | | | | | |
| AAVrh.53 | 477 | AAV2 variant | 2418 | AAV variant | 3294 | AAV2/3 variant | 4170 |
| AAV3-11) | | | | | | | |
| AAVrh.54 | 478 | AAV2 variant | 2419 | AAV variant | 3295 | AAV2/3 variant | 4171 |
| AAVrh.54 | 479 | AAV2 variant | 2420 | AAV variant | 3296 | AAV2/3 variant | 4172 |
| AAVrh.55 | 480 | AAV2 variant | 2421 | AAV variant | 3297 | AAV2/3 variant | 4173 |
| AAVrh.55 | 481 | AAV2 variant | 2422 | AAV variant | 3298 | AAV2/3 variant | 4174 |
| (AAV4-19) | | | | | | | |
| AAVrh.56 | 482 | AAV2 variant | 2423 | AAV variant | 3299 | AAV2/3 variant | 4175 |
| AAVrh.56 | 483 | AAV2 variant | 2424 | AAV variant | 3300 | AAV2/3 variant | 4176 |
| AAVrh.57 | 484 | AAV2 variant | 2425 | AAV variant | 3301 | AAV2/3 variant | 4177 |
| AAVrh.57 | 485 | AAV2 variant | 2426 | AAV variant | 3302 | AAV2/3 variant | 4178 |
| AAVrh.58 | 486 | AAV2 variant | 2427 | AAV variant | 3303 | AAV2/3 variant | 4179 |
| AAVrh.58 | 487 | AAV2 variant | 2428 | AAV variant | 3304 | AAV2/3 variant | 4180 |
| AAVrh.58 | 488 | AAV2 variant | 2429 | AAV variant | 3305 | AAV2/3 variant | 4181 |
| AAVrh.59 | 489 | AAV2 variant | 2430 | AAV variant | 3306 | AAV2/3 variant | 4182 |
| AAVrh.59 | 490 | AAV2 variant | 2431 | AAV variant | 3307 | AAV2/3 variant | 4183 |
| AAVrh.60 | 491 | AAV2 variant | 2432 | AAV variant | 3308 | AAV2/3 variant | 4184 |
| AAVrh.60 | 492 | AAV2 variant | 2433 | AAV variant | 3309 | AAV2/3 variant | 4185 |
| AAVrh.61 | 493 | AAV2 variant | 2434 | AAV variant | 3310 | AAV2/3 variant | 4186 |
| AAVrh.61 | 494 | AAV2 variant | 2435 | AAV variant | 3311 | AAV2/3 variant | 4187 |
| (AAV2-3) | | | | | | | |
| AAVrh.62 | 495 | AAV2 variant | 2436 | AAV variant | 3312 | AAV2/3 variant | 4188 |
| (AAV2-15) | | | | | | | |
| AAVrh.62 | 496 | AAV2 variant | 2437 | AAV variant | 3313 | AAV2/3 variant | 4189 |
| (AAV2-15) | | | | | | | |
| AAVrh.64 | 497 | AAV2 variant | 2438 | AAV variant | 3314 | AAV2/3 variant | 4190 |
| AAVrh.64 | 498 | AAV2 variant | 2439 | AAV variant | 3315 | AAV2/3 variant | 4191 |
| AAVrh.64 | 499 | AAV2 variant | 2440 | AAV variant | 3316 | AAV2/3 variant | 4192 |
| AAVRh.64R1 | 500 | AAV2 variant | 2441 | AAV variant | 3317 | AAV2/3 variant | 4193 |
| AAVRh.64R2 | 501 | AAV2 variant | 2442 | AAV variant | 3318 | AAV2/3 variant | 4194 |
| AAVrh.65 | 502 | AAV2 variant | 2443 | AAV variant | 3319 | AAV2/3 variant | 4195 |
| AAVrh.65 | 503 | AAV2 variant | 2444 | AAV variant | 3320 | AAV2/3 variant | 4196 |
| AAVrh.67 | 504 | AAV2 variant | 2445 | AAV variant | 3321 | AAV2/3 variant | 4197 |
| AAVrh.67 | 505 | AAV2 variant | 2446 | AAV variant | 3322 | AAV2/3 variant | 4198 |
| AAVrh.67 | 506 | AAV2 variant | 2447 | AAV variant | 3323 | AAV2/3 variant | 4199 |
| AAVrh.68 | 507 | AAV2 variant | 2448 | AAV variant | 3324 | AAV2/3 variant | 4200 |
| AAVrh.68 | 508 | AAV2 variant | 2449 | AAV variant | 3325 | AAV2/3 variant | 4201 |
| AAVrh.69 | 509 | AAV2 variant | 2450 | AAV variant | 3326 | AAV2/3 variant | 4202 |
| AAVrh.69 | 510 | AAV2 variant | 2451 | AAV variant | 3327 | AAV2/3 variant | 4203 |
| AAVrh.70 | 511 | AAV2 variant | 2452 | AAV variant | 3328 | AAV2/3 variant | 4204 |
| AAVrh.70 | 512 | AAV2 variant | 2453 | AAV variant | 3329 | AAV2/3 variant | 4205 |
| AAVrh.71 | 513 | AAV2 variant | 2454 | AAV variant | 3330 | AAV2/3 variant | 4206 |
| AAVrh.72 | 514 | AAV2 variant | 2455 | AAV variant | 3331 | AAV2/3 variant | 4207 |
| AAVrh.73 | 515 | AAV2 variant | 2456 | AAV variant | 3332 | AAV2/3 variant | 4208 |
| AAVrh.74 | 516 | AAV2 variant | 2457 | AAV variant | 3333 | AAV2/3 variant | 4209 |
| AAVrh.8 | 517 | AAV2 variant | 2458 | AAV variant | 3334 | AAV2/3 variant | 4210 |
| AAVrh.8 | 518 | AAV2 variant | 2459 | AAV variant | 3335 | AAV2/3 variant | 4211 |
| AAVrh.8R | 519 | AAV2 variant | 2460 | AAV variant | 3336 | AAV2/3 variant | 4212 |
| AAVrh.8R A586R | 520 | AAV2 variant | 2461 | AAV variant | 3337 | AAV2/3 variant | 4213 |
| mutant | | | | | | | |
| AAVrh.8R R533A | 521 | AAV2 variant | 2462 | AAV variant | 3338 | AAV2/3 variant | 4214 |
| mutant | | | | | | | |
| BAAV (bovine | 522 | AAV2 variant | 2463 | AAV variant | 3339 | AAV2/3 variant | 4215 |
| AAV) | | | | | | | |
| BAAV (bovine | 523 | AAV2 variant | 2464 | AAV variant | 3340 | AAV2/3 variant | 4216 |
| AAV) | | | | | | | |
| BAAV (bovine | 524 | AAV2 variant | 2465 | AAV variant | 3341 | AAV2/3 variant | 4217 |
| AAV) | | | | | | | |
| BAAV (bovine | 525 | AAV2 variant | 2466 | AAV variant | 3342 | AAV2/3 variant | 4218 |
| AAV) | | | | | | | |
| BAAV (bovine | 526 | AAV2 variant | 2467 | AAV variant | 3343 | AAV2/3 variant | 4219 |
| AAV) | | | | | | | |
| BAAV (bovine | 527 | AAV2 variant | 2468 | AAV variant | 3344 | AAV2/3 variant | 4220 |
| AAV) | | | | | | | |
| BAAV (bovine | 528 | AAV2 variant | 2469 | AAV variant | 3345 | AAV2/3 variant | 4221 |
| AAV) | | | | | | | |
| BAAV (bovine | 529 | AAV2 variant | 2470 | AAV variant | 3346 | AAV2/3 variant | 4222 |
| AAV) | | | | | | | |
| BAAV (bovine | 530 | AAV2 variant | 2471 | AAV variant | 3347 | AAV2/3 variant | 4223 |
| AAV) | | | | | | | |
| BAAV (bovine | 531 | AAV2 variant | 2472 | AAV variant | 3348 | AAV2/3 variant | 4224 |
| AAV) | | | | | | | |
| BAAV (bovine | 532 | AAV2 variant | 2473 | AAV variant | 3349 | AAV2/3 variant | 4225 |
| AAV) | | | | | | | |
| BAAV (bovine | 533 | AAV2 variant | 2474 | AAV variant | 3350 | AAV2/3 variant | 4226 |
| AAV) | | | | | | | |
| BAAV (bovine | 534 | AAV2 variant | 2475 | AAV variant | 3351 | AAV2/3 variant | 4227 |
| AAV) | | | | | | | |
| BNP61 AAV | 535 | AAV2 variant | 2476 | AAV variant | 3352 | AAV2/3 variant | 4228 |
| BNP61 AAV | 536 | AAV2 variant | 2477 | AAV variant | 3353 | AAV2/3 variant | 4229 |
| BNP62 AAV | 537 | AAV2 variant | 2478 | AAV variant | 3354 | AAV2/3 variant | 4230 |
| BNP63 AAV | 538 | AAV2 variant | 2479 | AAV variant | 3355 | AAV2/3 variant | 4231 |
| caprine AAV | 539 | AAV2 variant | 2480 | AAV variant | 3356 | AAV2/3 variant | 4232 |
| caprine AAV | 540 | AAV2 variant | 2481 | AAV variant | 3357 | AAV2/3 variant | 4233 |
| true type AAV | 541 | AAV2 variant | 2482 | AAV variant | 3358 | AAV2/3 variant | 4234 |
| (ttAAV) | | | | | | | |
| AAAV (Avian | 542 | AAV2 variant | 2483 | AAV variant | 3359 | AAV2/3 variant | 4235 |
| AAV) | | | | | | | |
| AAAV (Avian | 543 | AAV2 variant | 2484 | AAV variant | 3360 | AAV2/3 variant | 4236 |
| AAV) | | | | | | | |
| AAAV (Avian | 544 | AAV2 variant | 2485 | AAV variant | 3361 | AAV2/3 variant | 4237 |
| AAV) | | | | | | | |
| AAAV (Avian | 545 | AAV2 variant | 2486 | AAV variant | 3362 | AAV2/3 variant | 4238 |
| AAV) | | | | | | | |
| AAAV (Avian | 546 | AAV2 variant | 2487 | AAV variant | 3363 | AAV2/3 variant | 4239 |
| AAV) | | | | | | | |
| AAAV (Avian | 547 | AAV2 variant | 2488 | AAV variant | 3364 | AAV2/3 variant | 4240 |
| AAV) | | | | | | | |
| AAAV (Avian | 548 | AAV2 variant | 2489 | AAV variant | 3365 | AAV2/3 variant | 4241 |
| AAV) | | | | | | | |
| AAAV (Avian | 549 | AAV2 variant | 2490 | AAV variant | 3366 | AAV2/3 variant | 4242 |
| AAV) | | | | | | | |
| AAAV (Avian | 550 | AAV2 variant | 2491 | AAV variant | 3367 | AAV2/3 variant | 4243 |
| AAV) | | | | | | | |
| AAAV (Avian | 551 | AAV2 variant | 2492 | AAV variant | 3368 | AAV2/3 variant | 4244 |
| AAV) | | | | | | | |
| AAAV (Avian | 552 | AAV2 variant | 2493 | AAV variant | 3369 | AAV2/3 variant | 4245 |
| AAV) | | | | | | | |
| AAAV (Avian | 553 | AAV2 variant | 2494 | AAV variant | 3370 | AAV2/3 variant | 4246 |
| AAV) | | | | | | | |
| AAAV (Avian | 554 | AAV2 variant | 2495 | AAV variant | 3371 | AAV2/3 variant | 4247 |
| AAV) | | | | | | | |
| AAAV (Avian | 555 | AAV2 variant | 2496 | AAV variant | 3372 | AAV2/3 variant | 4248 |
| AAV) | | | | | | | |
| AAAV (Avian | 556 | AAV2 variant | 2497 | AAV variant | 3373 | AAV2/3 variant | 4249 |
| AAV) | | | | | | | |
| AAV Shuffle 100- | 557 | AAV2 variant | 2498 | AAV variant | 3374 | AAV2/3 variant | 4250 |
| 1 | | | | | | | |
| AAV Shuffle 100- | 558 | AAV2 variant | 2499 | AAV variant | 3375 | AAV2/3 variant | 4251 |
| 1 | | | | | | | |
| AAV Shuffle 100- | 559 | AAV2 variant | 2500 | AAV variant | 3376 | AAV2/3 variant | 4252 |
| 2 | | | | | | | |
| AAV Shuffle 100- | 560 | AAV2 variant | 2501 | AAV variant | 3377 | AAV2/3 variant | 4253 |
| 2 | | | | | | | |
| AAV Shuffle 100- | 561 | AAV2 variant | 2502 | AAV variant | 3378 | AAV2/3 variant | 4254 |
| 3 | | | | | | | |
| AAV Shuffle 100- | 562 | AAV2 variant | 2503 | AAV variant | 3379 | AAV2/3 variant | 4255 |
| 3 | | | | | | | |
| AAV Shuffle 100- | 563 | AAV2 variant | 2504 | AAV variant | 3380 | AAV2/3 variant | 4256 |
| 7 | | | | | | | |
| AAV Shuffle 100- | 564 | AAV2 variant | 2505 | AAV variant | 3381 | AAV2/3 variant | 4257 |
| 7 | | | | | | | |
| AAV Shuffle 10-2 | 565 | AAV2 variant | 2506 | AAV variant | 3382 | AAV2/3 variant | 4258 |
| AAV Shuffle 10-2 | 566 | AAV2 variant | 2507 | AAV variant | 3383 | AAV2/3 variant | 4259 |
| AAV Shuffle 10-6 | 567 | AAV2 variant | 2508 | AAV variant | 3384 | AAV2/3 variant | 4260 |
| AAV Shuffle 10-6 | 568 | AAV2 variant | 2509 | AAV variant | 3385 | AAV2/3 variant | 4261 |
| AAV Shuffle 10-8 | 569 | AAV2 variant | 2510 | AAV variant | 3386 | AAV2/3 variant | 4262 |
| AAV Shuffle 10-8 | 570 | AAV2 variant | 2511 | AAV variant | 3387 | AAV2/3 variant | 4263 |
| AAV SM 100-10 | 571 | AAV2 variant | 2512 | AAV variant | 3388 | AAV2/3 variant | 4264 |
| AAV SM 100-10 | 572 | AAV2 variant | 2513 | AAV variant | 3389 | AAV2/3 variant | 4265 |
| AAV SM 100-3 | 573 | AAV2 variant | 2514 | AAV variant | 3390 | AAV2/3 variant | 4266 |
| AAV SM 100-3 | 574 | AAV2 variant | 2515 | AAV variant | 3391 | AAV2/3 variant | 4267 |
| AAV SM 10-1 | 575 | AAV2 variant | 2516 | AAV variant | 3392 | AAV2/3 variant | 4268 |
| AAV SM 10-1 | 576 | AAV2 variant | 2517 | AAV variant | 3393 | AAV2/3 variant | 4269 |
| AAV SM 10-2 | 577 | AAV2 variant | 2518 | AAV variant | 3394 | AAV2/3 variant | 4270 |
| AAV SM 10-2 | 578 | AAV2 variant | 2519 | AAV variant | 3395 | AAV2 variant | 4271 |
| AAV SM 10-8 | 579 | AAV2 variant | 2520 | AAV variant | 3396 | AAV2 variant | 4272 |
| AAV SM 10-8 | 580 | AAV2 variant | 2521 | AAV variant | 3397 | AAV2 variant | 4273 |
| AAVF1/HSC1 | 581 | AAV2 variant | 2522 | AAV variant | 3398 | AAV2 variant | 4274 |
| AAVF2/HSC2 | 582 | AAV2 variant | 2523 | AAV variant | 3399 | AAV2 variant | 4275 |
| AAVF3/HSC3 | 583 | AAV2 variant | 2524 | AAV variant | 3400 | AAV2 variant | 4276 |
| AAVF4/HSC4 | 584 | AAV2 variant | 2525 | AAV variant | 3401 | AAV2 variant | 4277 |
| AAVF5/HSC5 | 585 | AAV2 variant | 2526 | AAV variant | 3402 | AAV2 variant | 4278 |
| AAVF6/HSC6 | 586 | AAV2 variant | 2527 | AAV variant | 3403 | AAV2 variant | 4279 |
| AAVF7/HSC7 | 587 | AAV2 variant | 2528 | AAV variant | 3404 | AAV2 variant | 4280 |
| AAVF8/HSC8 | 588 | AAV2 variant | 2529 | AAV variant | 3405 | AAV2 variant | 4281 |
| AAVF9/HSC9 | 589 | AAV2 variant | 2530 | AAV variant | 3406 | AAV2 variant | 4282 |
| AAVF11/HSC11 | 590 | AAV2 variant | 2531 | AAV variant | 3407 | AAV2 variant | 4283 |
| AAVF12/HSC12 | 591 | AAV2 variant | 2532 | AAV variant | 3408 | AAV2 variant | 4284 |
| AAVF13/HSC13 | 592 | AAV2 variant | 2533 | AAV variant | 3409 | AAV2 variant | 4285 |
| AAVF14/HSC14 | 593 | AAV2 variant | 2534 | AAV variant | 3410 | AAV2 variant | 4286 |
| AAVF15/HSC15 | 594 | AAV2 variant | 2535 | AAV variant | 3411 | AAV2 variant | 4287 |
| AAVF16/HSC16 | 595 | AAV2 variant | 2536 | AAV variant | 3412 | AAV2 variant | 4288 |
| AAVF17/HSC17 | 596 | AAV2 variant | 2537 | AAV variant | 3413 | AAV2 variant | 4289 |
| AAVF1/HSC1 | 597 | AAV2 variant | 2538 | AAV variant | 3414 | AAV2 variant | 4290 |
| AAVF2/HSC2 | 598 | AAV2 variant | 2539 | AAV variant | 3415 | AAV2 variant | 4291 |
| AAVF3/HSC3 | 599 | AAV2 variant | 2540 | AAV variant | 3416 | AAV2 variant | 4292 |
| AAVF4/HSC4 | 600 | AAV2 variant | 2541 | AAV variant | 3417 | AAV2 variant | 4293 |
| AAVF5/HSC5 | 601 | AAV2 variant | 2542 | AAV variant | 3418 | AAV2 variant | 4294 |
| AAVF6/HSC6 | 602 | AAV2 variant | 2543 | AAV variant | 3419 | AAV2 variant | 4295 |
| AAVF7/HSC7 | 603 | AAV2 variant | 2544 | AAV variant | 3420 | AAV2 variant | 4296 |
| AAVF8/HSC8 | 604 | AAV2 variant | 2545 | AAV variant | 3421 | AAV2 variant | 4297 |
| AAVF9/HSC9 | 605 | AAV2 variant | 2546 | AAV variant | 3422 | AAV2 variant | 4298 |
| AAVF11/HSC11 | 606 | AAV2 variant | 2547 | AAV variant | 3423 | AAV2 variant | 4299 |
| AAVF12/HSC12 | 607 | AAV2 variant | 2548 | AAV variant | 3424 | AAV2 variant | 4300 |
| AAVF13/HSC13 | 608 | AAV2 variant | 2549 | AAV variant | 3425 | AAV2 variant | 4301 |
| AAVF14/HSC14 | 609 | AAV2 variant | 2550 | AAV variant | 3426 | AAV2 variant | 4302 |
| AAVF15/HSC15 | 610 | AAV2 variant | 2551 | AAV variant | 3427 | AAV2 variant | 4303 |
| AAVF16/HSC16 | 611 | AAV2 variant | 2552 | AAV variant | 3428 | AAV2 variant | 4304 |
| AAVF17/HSC17 | 612 | AAV2 variant | 2553 | AAV variant | 3429 | AAV2 variant | 4305 |
| AAV CBr-E1 | 613 | AAV2 variant | 2554 | AAV variant | 3430 | AAV2 variant | 4306 |
| AAV CBr-E2 | 614 | AAV2 variant | 2555 | AAV variant | 3431 | AAV2 variant | 4307 |
| AAV CBr-E3 | 615 | AAV2 variant | 2556 | AAV variant | 3432 | AAV2 variant | 4308 |
| AAV CBr-E4 | 616 | AAV2 variant | 2557 | AAV variant | 3433 | AAV2 variant | 4309 |
| AAV CBr-E5 | 617 | AAV2 variant | 2558 | AAV variant | 3434 | AAV2 variant | 4310 |
| AAV CBr-e5 | 618 | AAV2 variant | 2559 | AAV variant | 3435 | AAV2 variant | 4311 |
| AAV CBr-E6 | 619 | AAV2 variant | 2560 | AAV variant | 3436 | AAV2 variant | 4312 |
| AAV CBr-E7 | 620 | AAV2 variant | 2561 | AAV variant | 3437 | AAV2 variant | 4313 |
| AAV CBr-E8 | 621 | AAV2 variant | 2562 | AAV variant | 3438 | AAV2 variant | 4314 |
| AAV CLv-D1 | 622 | AAV2 variant | 2563 | AAV variant | 3439 | AAV2 variant | 4315 |
| AAV CLv-D2 | 623 | AAV2 variant | 2564 | AAV variant | 3440 | AAV2 variant | 4316 |
| AAV CLv-D3 | 624 | AAV2 variant | 2565 | AAV variant | 3441 | AAV2 variant | 4317 |
| AAV CLv-D4 | 625 | AAV2 variant | 2566 | AAV variant | 3442 | AAV2 variant | 4318 |
| AAV CLv-D5 | 626 | AAV2 variant | 2567 | AAV variant | 3443 | AAV2 variant | 4319 |
| AAV CLv-D6 | 627 | AAV2 variant | 2568 | AAV variant | 3444 | AAV2 variant | 4320 |
| AAV CLv-D7 | 628 | AAV2 variant | 2569 | AAV variant | 3445 | AAV2 variant | 4321 |
| AAV CLv-D8 | 629 | AAV2 variant | 2570 | AAV variant | 3446 | AAV2 variant | 4322 |
| AAV CLv-E1 | 630 | AAV2 variant | 2571 | AAV variant | 3447 | AAV2 variant | 4323 |
| AAV CLv-R1 | 631 | AAV2 variant | 2572 | AAV variant | 3448 | AAV2 variant | 4324 |
| AAV CLv-R2 | 632 | AAV2 variant | 2573 | AAV variant | 3449 | AAV2 variant | 4325 |
| AAV CLv-R3 | 633 | AAV2 variant | 2574 | AAV variant | 3450 | AAV2 variant | 4326 |
| AAV CLv-R4 | 634 | AAV2 variant | 2575 | AAV variant | 3451 | AAV2 variant | 4327 |
| AAV CLv-R5 | 635 | AAV2 variant | 2576 | AAV variant | 3452 | AAV2 variant | 4328 |
| AAV CLv-R6 | 636 | AAV2 variant | 2577 | AAV variant | 3453 | AAV2 variant | 4329 |
| AAV CLv-R7 | 637 | AAV2 variant | 2578 | AAV variant | 3454 | AAV2 variant | 4330 |
| AAV CLv-R8 | 638 | AAV2 variant | 2579 | AAV variant | 3455 | AAV2 variant | 4331 |
| AAV CLv-R9 | 639 | AAV2 variant | 2580 | AAV variant | 3456 | AAV2 variant | 4332 |
| AAV CLg-F1 | 640 | AAV2 variant | 2581 | AAV variant | 3457 | AAV2 variant | 4333 |
| AAV CLg-F2 | 641 | AAV2 variant | 2582 | AAV variant | 3458 | AAV2 variant | 4334 |
| AAV CLg-F3 | 642 | AAV2 variant | 2583 | AAV variant | 3459 | AAV2 variant | 4335 |
| AAV CLg-F4 | 643 | AAV2 variant | 2584 | AAV variant | 3460 | AAV2 variant | 4336 |
| AAV CLg-F5 | 644 | AAV2 variant | 2585 | AAV variant | 3461 | AAV2 variant | 4337 |
| AAV CLg-F6 | 645 | AAV2 variant | 2586 | AAV variant | 3462 | AAV2 variant | 4338 |
| AAV CLg-F7 | 646 | AAV2 variant | 2587 | AAV variant | 3463 | AAV2 variant | 4339 |
| AAV CLg-F8 | 647 | AAV2 variant | 2588 | AAV variant | 3464 | AAV2 variant | 4340 |
| AAV CSp-1 | 648 | AAV2 variant | 2589 | AAV variant | 3465 | AAV2 variant | 4341 |
| AAV CSp-10 | 649 | AAV2 variant | 2590 | AAV variant | 3466 | AAV2 variant | 4342 |
| AAV CSp-11 | 650 | AAV2 variant | 2591 | AAV variant | 3467 | AAV2 variant | 4343 |
| AAV CSp-2 | 651 | AAV2 variant | 2592 | AAV variant | 3468 | AAV2 variant | 4344 |
| AAV CSp-3 | 652 | AAV2 variant | 2593 | AAV variant | 3469 | AAV2 variant | 4345 |
| AAV CSp-4 | 653 | AAV2 variant | 2594 | AAV variant | 3470 | AAV2 variant | 4346 |
| AAV CSp-6 | 654 | AAV2 variant | 2595 | AAV variant | 3471 | AAV2 variant | 4347 |
| AAV CSp-7 | 655 | AAV2 variant | 2596 | AAV variant | 3472 | AAV2 variant | 4348 |
| AAV CSp-8 | 656 | AAV2 variant | 2597 | AAV variant | 3473 | AAV2 variant | 4349 |
| AAV CSp-9 | 657 | AAV2 variant | 2598 | AAV variant | 3474 | AAV2 variant | 4350 |
| AAV CHt-2 | 658 | AAV2 variant | 2599 | AAV variant | 3475 | AAV2 variant | 4351 |
| AAV CHt-3 | 659 | AAV2 variant | 2600 | AAV variant | 3476 | AAV2 variant | 4352 |
| AAV CKd-1 | 660 | AAV2 variant | 2601 | AAV variant | 3477 | AAV2 variant | 4353 |
| AAV CKd-10 | 661 | AAV2 variant | 2602 | AAV variant | 3478 | AAV2 variant | 4354 |
| AAV CKd-2 | 662 | AAV2 variant | 2603 | AAV variant | 3479 | AAV2 variant | 4355 |
| AAV CKd-3 | 663 | AAV2 variant | 2604 | AAV variant | 3480 | AAV2 variant | 4356 |
| AAV CKd-4 | 664 | AAV2 variant | 2605 | AAV variant | 3481 | AAV2 variant | 4357 |
| AAV CKd-6 | 665 | AAV2 variant | 2606 | AAV variant | 3482 | AAV2 variant | 4358 |
| AAV CKd-7 | 666 | AAV2 variant | 2607 | AAV variant | 3483 | AAV2 variant | 4359 |
| AAV CKd-8 | 667 | AAV2 variant | 2608 | AAV variant | 3484 | AAV2/3 variant | 4360 |
| AAV CLv-1 | 668 | AAV2 variant | 2609 | AAV variant | 3485 | AAV2/3 variant | 4361 |
| AAV CLv-12 | 669 | AAV2 variant | 2610 | AAV variant | 3486 | AAV2/3 variant | 4362 |
| AAV CLv-13 | 670 | AAV2 variant | 2611 | AAV variant | 3487 | AAV2/3 variant | 4363 |
| AAV CLv-2 | 671 | AAV2 variant | 2612 | AAV variant | 3488 | AAV2/3 variant | 4364 |
| AAV CLv-3 | 672 | AAV2 variant | 2613 | AAV variant | 3489 | AAV2/3 variant | 4365 |
| AAV CLv-4 | 673 | AAV2 variant | 2614 | AAV variant | 3490 | AAV2/3 variant | 4366 |
| AAV CLv-6 | 674 | AAV2 variant | 2615 | AAV variant | 3491 | AAV2/3 variant | 4367 |
| AAV CLv-8 | 675 | AAV2 variant | 2616 | AAV variant | 3492 | AAV2/3 variant | 4368 |
| AAV CKd-B1 | 676 | AAV2 variant | 2617 | AAV variant | 3493 | AAV2/3 variant | 4369 |
| AAV CKd-B2 | 677 | AAV2 variant | 2618 | AAV variant | 3494 | AAV2/3 variant | 4370 |
| AAV CKd-B3 | 678 | AAV2 variant | 2619 | AAV variant | 3495 | AAV2/3 variant | 4371 |
| AAV CKd-B4 | 679 | AAV2 variant | 2620 | AAV variant | 3496 | AAV2/3 variant | 4372 |
| AAV CKd-B5 | 680 | AAV2 variant | 2621 | AAV variant | 3497 | AAV2/3 variant | 4373 |
| AAV CKd-B6 | 681 | AAV2 variant | 2622 | AAV variant | 3498 | AAV2/3 variant | 4374 |
| AAV CKd-B7 | 682 | AAV2 variant | 2623 | AAV variant | 3499 | AAV2/3 variant | 4375 |
| AAV CKd-B8 | 683 | AAV2 variant | 2624 | AAV variant | 3500 | AAV2/3 variant | 4376 |
| AAV CKd-H1 | 684 | AAV2 variant | 2625 | AAV variant | 3501 | AAV2/3 variant | 4377 |
| AAV CKd-H2 | 685 | AAV2 variant | 2626 | AAV variant | 3502 | AAV2/3 variant | 4378 |
| AAV CKd-H3 | 686 | AAV2 variant | 2627 | AAV variant | 3503 | AAV2/3 variant | 4379 |
| AAV CKd-H4 | 687 | AAV2 variant | 2628 | AAV variant | 3504 | AAV2/3 variant | 4380 |
| AAV CKd-H5 | 688 | AAV2 variant | 2629 | AAV variant | 3505 | AAV2/3 variant | 4381 |
| AAV CKd-H6 | 689 | AAV2 variant | 2630 | AAV variant | 3506 | AAV2/3 variant | 4382 |
| AAV CHt-1 | 690 | AAV2 variant | 2631 | AAV variant | 3507 | AAV2/3 variant | 4383 |
| AAV CLv1-1 | 691 | AAV2 variant | 2632 | AAV variant | 3508 | AAV2/3 variant | 4384 |
| AAV CLv1-2 | 692 | AAV2 variant | 2633 | AAV variant | 3509 | AAV2/3 variant | 4385 |
| AAV CLv1-3 | 693 | AAV2 variant | 2634 | AAV variant | 3510 | AAV2/3 variant | 4386 |
| AAV CLv1-4 | 694 | AAV2 variant | 2635 | AAV variant | 3511 | AAV2/3 variant | 4387 |
| AAV Clv1-7 | 695 | AAV2 variant | 2636 | AAV variant | 3512 | AAV2/3 variant | 4388 |
| AAV Clv1-8 | 696 | AAV2 variant | 2637 | AAV variant | 3513 | AAV2/3 variant | 4389 |
| AAV Clv1-9 | 697 | AAV2 variant | 2638 | AAV variant | 3514 | AAV2/3 variant | 4390 |
| AAV Clv1-10 | 698 | AAV2 variant | 2639 | AAV variant | 3515 | AAV2/3 variant | 4391 |
| AAV.VR-355 | 699 | AAV2 variant | 2640 | AAV variant | 3516 | AAV2/3 variant | 4392 |
| AAV.hu.48R3 | 700 | AAV2 variant | 2641 | AAV variant | 3517 | AAV2/3 variant | 4393 |
| AAV CBr-E1 | 701 | AAV2 variant | 2642 | AAV variant | 3518 | AAV2/3 variant | 4394 |
| AAV CBr-E2 | 702 | AAV2 variant | 2643 | AAV variant | 3519 | AAV2/3 variant | 4395 |
| AAV CBr-E3 | 703 | AAV2 variant | 2644 | AAV variant | 3520 | AAV2/3 variant | 4396 |
| AAV CBr-E4 | 704 | AAV2 variant | 2645 | AAV variant | 3521 | AAV2/3 variant | 4397 |
| AAV CBr-E5 | 705 | AAV2 variant | 2646 | AAV variant | 3522 | AAV2/3 variant | 4398 |
| AAV CBr-e5 | 706 | AAV2 variant | 2647 | AAV variant | 3523 | AAV2/3 variant | 4399 |
| AAV CBr-E6 | 707 | AAV2 variant | 2648 | AAV variant | 3524 | AAV2/3 variant | 4400 |
| AAV CBr-E7 | 708 | AAV2 variant | 2649 | AAV variant | 3525 | AAV2/3 variant | 4401 |
| AAV CBr-E8 | 709 | AAV2 variant | 2650 | AAV variant | 3526 | AAV2/3 variant | 4402 |
| AAV CLv-D1 | 710 | AAV2 variant | 2651 | AAV variant | 3527 | AAV2/3 variant | 4403 |
| AAV CLv-D2 | 711 | AAV2 variant | 2652 | AAV variant | 3528 | AAV2/3 variant | 4404 |
| AAV CLv-D3 | 712 | AAV2 variant | 2653 | AAV variant | 3529 | AAV2/3 variant | 4405 |
| AAV CLv-D4 | 713 | AAV2 variant | 2654 | AAV variant | 3530 | AAV2/3 variant | 4406 |
| AAV CLv-D5 | 714 | AAV2 variant | 2655 | AAV variant | 3531 | AAV2/3 variant | 4407 |
| AAV CLv-D6 | 715 | AAV2 variant | 2656 | AAV variant | 3532 | AAV2/3 variant | 4408 |
| AAV CLv-D7 | 716 | AAV2 variant | 2657 | AAV variant | 3533 | AAV2/3 variant | 4409 |
| AAV CLv-D8 | 717 | AAV2 variant | 2658 | AAV variant | 3534 | AAV2/3 variant | 4410 |
| AAV CLv-E1 | 718 | AAV2 variant | 2659 | AAV variant | 3535 | AAV2/3 variant | 4411 |
| AAV CLv-R1 | 719 | AAV2 variant | 2660 | AAV variant | 3536 | AAV2/3 variant | 4412 |
| AAV CLv-R2 | 720 | AAV2 variant | 2661 | AAV variant | 3537 | AAV2/3 variant | 4413 |
| AAV CLv-R3 | 721 | AAV2 variant | 2662 | AAV variant | 3538 | AAV2/3 variant | 4414 |
| AAV CLv-R4 | 722 | AAV2 variant | 2663 | AAV variant | 3539 | AAV2/3 variant | 4415 |
| AAV CLv-R5 | 723 | AAV2 variant | 2664 | AAV variant | 3540 | AAV2/3 variant | 4416 |
| AAV CLv-R6 | 724 | AAV2 variant | 2665 | AAV variant | 3541 | AAV2/3 variant | 4417 |
| AAV CLv-R7 | 725 | AAV2 variant | 2666 | AAV variant | 3542 | AAV2/3 variant | 4418 |
| AAV CLv-R8 | 726 | AAV2 variant | 2667 | AAV variant | 3543 | AAV2/3 variant | 4419 |
| AAV CLv-R9 | 727 | AAV2 variant | 2668 | AAV variant | 3544 | AAV2/3 variant | 4420 |
| AAV CLg-F1 | 728 | AAV2 variant | 2669 | AAV variant | 3545 | AAV2/3 variant | 4421 |
| AAV CLg-F2 | 729 | AAV2 variant | 2670 | AAV variant | 3546 | AAV2/3 variant | 4422 |
| AAV CLg-F3 | 730 | AAV2 variant | 2671 | AAV variant | 3547 | AAV2/3 variant | 4423 |
| AAV CLg-F4 | 731 | AAV2 variant | 2672 | AAV variant | 3548 | AAV2/3 variant | 4424 |
| AAV CLg-F5 | 732 | AAV2 variant | 2673 | AAV variant | 3549 | AAV2/3 variant | 4425 |
| AAV CLg-F6 | 733 | AAV2 variant | 2674 | AAV variant | 3550 | AAV2/3 variant | 4426 |
| AAV CLg-F7 | 734 | AAV2 variant | 2675 | AAV variant | 3551 | AAV2/3 variant | 4427 |
| AAV CLg-F8 | 735 | AAV2 variant | 2676 | AAV variant | 3552 | AAV2/3 variant | 4428 |
| AAV CSp-1 | 736 | AAV2 variant | 2677 | AAV variant | 3553 | AAV2/3 variant | 4429 |
| AAV CSp-10 | 737 | AAV2 variant | 2678 | AAV variant | 3554 | AAV2/3 variant | 4430 |
| AAV CSp-11 | 738 | AAV2 variant | 2679 | AAV variant | 3555 | AAV2/3 variant | 4431 |
| AAV CSp-2 | 739 | AAV2 variant | 2680 | AAV variant | 3556 | AAV2/3 variant | 4432 |
| AAV CSp-3 | 740 | AAV2 variant | 2681 | AAV variant | 3557 | AAV2/3 variant | 4433 |
| AAV CSp-4 | 741 | AAV2 variant | 2682 | AAV variant | 3558 | AAV2/3 variant | 4434 |
| AAV CSp-6 | 742 | AAV2 variant | 2683 | AAV variant | 3559 | AAV2/3 variant | 4435 |
| AAV CSp-7 | 743 | AAV2 variant | 2684 | AAV variant | 3560 | AAV2/3 variant | 4436 |
| AAV CSp-8 | 744 | AAV2 variant | 2685 | AAV variant | 3561 | AAV2/3 variant | 4437 |
| AAV CSp-9 | 745 | AAV2 variant | 2686 | AAV variant | 3562 | AAV2/3 variant | 4438 |
| AAV CHt-2 | 746 | AAV2 variant | 2687 | AAV variant | 3563 | AAV2/3 variant | 4439 |
| AAV CHt-3 | 747 | AAV2 variant | 2688 | AAV variant | 3564 | AAV2/3 variant | 4440 |
| AAV CKd-1 | 748 | AAV2 variant | 2689 | AAV variant | 3565 | AAV2/3 variant | 4441 |
| AAV CKd-10 | 749 | AAV2 variant | 2690 | AAV variant | 3566 | AAV2/3 variant | 4442 |
| AAV CKd-2 | 750 | AAV2 variant | 2691 | AAV variant | 3567 | AAV2/3 variant | 4443 |
| AAV CKd-3 | 751 | AAV2 variant | 2692 | AAV variant | 3568 | AAV2/3 variant | 4444 |
| AAV CKd-4 | 752 | AAV2 variant | 2693 | AAV variant | 3569 | AAV2/3 variant | 4445 |
| AAV CKd-6 | 753 | AAV2 variant | 2694 | AAV variant | 3570 | AAV2/3 variant | 4446 |
| AAV CKd-7 | 754 | AAV2 variant | 2695 | AAV variant | 3571 | AAV2/3 variant | 4447 |
| AAV CKd-8 | 755 | AAV2 variant | 2696 | AAV variant | 3572 | AAV2/3 variant | 4448 |
| AAV CLv-1 | 756 | AAV2 variant | 2697 | AAV variant | 3573 | AAV2/3 variant | 4449 |
| AAV CLv-12 | 757 | AAV2 variant | 2698 | AAV variant | 3574 | AAV2/3 variant | 4450 |
| AAV CLv-13 | 758 | AAV2 variant | 2699 | AAV variant | 3575 | AAV2/3 variant | 4451 |
| AAV CLv-2 | 759 | AAV2 variant | 2700 | AAV variant | 3576 | AAV2/3 variant | 4452 |
| AAV CLv-3 | 760 | AAV2 variant | 2701 | AAV variant | 3577 | AAV2/3 variant | 4453 |
| AAV CLv-4 | 761 | AAV2 variant | 2702 | AAV variant | 3578 | AAV2/3 variant | 4454 |
| AAV CLv-6 | 762 | AAV2 variant | 2703 | AAV variant | 3579 | AAV2/3 variant | 4455 |
| AAV CLv-8 | 763 | AAV2 variant | 2704 | AAV variant | 3580 | AAV2/3 variant | 4456 |
| AAV CKd-B1 | 764 | AAV2 variant | 2705 | AAV variant | 3581 | AAV2/3 variant | 4457 |
| AAV CKd-B2 | 765 | AAV2 variant | 2706 | AAV variant | 3582 | AAV2/3 variant | 4458 |
| AAV CKd-B3 | 766 | AAV2 variant | 2707 | AAV variant | 3583 | AAV2/3 variant | 4459 |
| AAV CKd-B4 | 767 | AAV2 variant | 2708 | AAV variant | 3584 | AAV2/3 variant | 4460 |
| AAV CKd-B5 | 768 | AAV2 variant | 2709 | AAV variant | 3585 | AAV2/3 variant | 4461 |
| AAV CKd-B6 | 769 | AAV2 variant | 2710 | AAV variant | 3586 | AAV2/3 variant | 4462 |
| AAV CKd-B7 | 770 | AAV2 variant | 2711 | AAV variant | 3587 | AAV2/3 variant | 4463 |
| AAV CKd-B8 | 771 | AAV2 variant | 2712 | AAV variant | 3588 | AAV2/3 variant | 4464 |
| AAV CKd-H1 | 772 | AAV2 variant | 2713 | AAV variant | 3589 | AAV2/3 variant | 4465 |
| AAV CKd-H2 | 773 | AAV2 variant | 2714 | AAV variant | 3590 | AAV2/3 variant | 4466 |
| AAV CKd-H3 | 774 | AAV2 variant | 2715 | AAV variant | 3591 | AAV2/3 variant | 4467 |
| AAV CKd-H4 | 775 | AAV2 variant | 2716 | AAV variant | 3592 | AAV2/3 variant | 4468 |
| AAV CKd-H5 | 776 | AAV2 variant | 2717 | AAV variant | 3593 | AAV2/3 variant | 4469 |
| AAV CKd-H6 | 777 | AAV2 variant | 2718 | AAV variant | 3594 | AAV2/3 variant | 4470 |
| AAV CHt-1 | 778 | AAV2 variant | 2719 | AAV variant | 3595 | AAV2/3 variant | 4471 |
| AAV CHt-P2 | 779 | AAV2 variant | 2720 | AAV variant | 3596 | AAV2/3 variant | 4472 |
| AAV CHt-P5 | 780 | AAV2 variant | 2721 | AAV variant | 3597 | AAV2/3 variant | 4473 |
| AAV CHt-P9 | 781 | AAV2 variant | 2722 | AAV variant | 3598 | AAV2/3 variant | 4474 |
| AAV CBr-7.1 | 782 | AAV2 variant | 2723 | AAV variant | 3599 | AAV2/3 variant | 4475 |
| AAV CBr-7.2 | 783 | AAV2 variant | 2724 | AAV variant | 3600 | AAV2/3 variant | 4476 |
| AAV CBr-7.3 | 784 | AAV2 variant | 2725 | AAV variant | 3601 | AAV2/3 variant | 4477 |
| AAV CBr-7.4 | 785 | AAV2 variant | 2726 | AAV variant | 3602 | AAV2/3 variant | 4478 |
| AAV CBr-7.5 | 786 | AAV2 variant | 2727 | AAV variant | 3603 | AAV2/3 variant | 4479 |
| AAV CBr-7.7 | 787 | AAV2 variant | 2728 | AAV variant | 3604 | AAV2/3 variant | 4480 |
| AAV CBr-7.8 | 788 | AAV2 variant | 2729 | AAV variant | 3605 | AAV2/3 variant | 4481 |
| AAV CBr-7.10 | 789 | AAV2 variant | 2730 | AAV variant | 3606 | AAV2/3 variant | 4482 |
| AAV CKd-N3 | 790 | AAV2/3 variant | 2731 | AAV variant | 3607 | AAV2/3 variant | 4483 |
| AAV CKd-N4 | 791 | AAV2/3 variant | 2732 | AAV variant | 3608 | AAV2/3 variant | 4484 |
| AAV CKd-N9 | 792 | AAV2/3 variant | 2733 | AAV variant | 3609 | AAV2/3 variant | 4485 |
| AAV CLv-L4 | 793 | AAV2/3 variant | 2734 | AAV variant | 3610 | AAV2/3 variant | 4486 |
| AAV CLv-L5 | 794 | AAV2/3 variant | 2735 | AAV variant | 3611 | AAV2/3 variant | 4487 |
| AAV CLv-L6 | 795 | AAV2/3 variant | 2736 | AAV variant | 3612 | AAV2/3 variant | 4488 |
| AAV CLv-K1 | 796 | AAV2/3 variant | 2737 | AAV variant | 3613 | AAV2/3 variant | 4489 |
| AAV CLv-K3 | 797 | AAV2/3 variant | 2738 | AAV variant | 3614 | AAV2/3 variant | 4490 |
| AAV CLv-K6 | 798 | AAV2/3 variant | 2739 | AAV variant | 3615 | AAV2/3 variant | 4491 |
| AAV CLv-M1 | 799 | AAV2/3 variant | 2740 | AAV variant | 3616 | AAV2/3 variant | 4492 |
| AAV CLv-M11 | 800 | AAV2/3 variant | 2741 | AAV variant | 3617 | AAV2/3 variant | 4493 |
| AAV CLv-M2 | 801 | AAV2/3 variant | 2742 | AAV variant | 3618 | AAV2/3 variant | 4494 |
| AAV CLv-M5 | 802 | AAV2/3 variant | 2743 | AAV variant | 3619 | AAV2/3 variant | 4495 |
| AAV CLv-M6 | 803 | AAV2/3 variant | 2744 | AAV variant | 3620 | AAV2/3 variant | 4496 |
| AAV CLv-M7 | 804 | AAV2/3 variant | 2745 | AAV variant | 3621 | AAV2/3 variant | 4497 |
| AAV CLv-M8 | 805 | AAV2/3 variant | 2746 | AAV variant | 3622 | AAV2/3 variant | 4498 |
| AAV CLv-M9 | 806 | AAV2/3 variant | 2747 | AAV variant | 3623 | AAV2/3 variant | 4499 |
| AAV CHt-P1 | 807 | AAV2/3 variant | 2748 | AAV variant | 3624 | AAV2/3 variant | 4500 |
| AAV CHt-P6 | 808 | AAV2/3 variant | 2749 | AAV variant | 3625 | AAV2/3 variant | 4501 |
| AAV CHt-P8 | 809 | AAV2/3 variant | 2750 | AAV variant | 3626 | AAV2/3 variant | 4502 |
| AAV CHt-6.1 | 810 | AAV2/3 variant | 2751 | AAV variant | 3627 | AAV2/3 variant | 4503 |
| AAV CHt-6.10 | 811 | AAV2/3 variant | 2752 | AAV variant | 3628 | AAV2/3 variant | 4504 |
| AAV CHt-6.5 | 812 | AAV2/3 variant | 2753 | AAV variant | 3629 | AAV2/3 variant | 4505 |
| AAV CHt-6.6 | 813 | AAV2/3 variant | 2754 | AAV variant | 3630 | AAV2/3 variant | 4506 |
| AAV CHt-6.7 | 814 | AAV2/3 variant | 2755 | AAV variant | 3631 | AAV2/3 variant | 4507 |
| AAV CHt-6.8 | 815 | AAV2/3 variant | 2756 | AAV variant | 3632 | AAV2/3 variant | 4508 |
| AAV CSp-8.10 | 816 | AAV2/3 variant | 2757 | AAV variant | 3633 | AAV2/3 variant | 4509 |
| AAV CSp-8.2 | 817 | AAV2/3 variant | 2758 | AAV variant | 3634 | AAV2/3 variant | 4510 |
| AAV CSp-8.4 | 818 | AAV2/3 variant | 2759 | AAV variant | 3635 | AAV2/3 variant | 4511 |
| AAV CSp-8.5 | 819 | AAV2/3 variant | 2760 | AAV variant | 3636 | AAV2/3 variant | 4512 |
| AAV CSp-8.6 | 820 | AAV2/3 variant | 2761 | AAV variant | 3637 | AAV2/3 variant | 4513 |
| AAV CSp-8.7 | 821 | AAV2/3 variant | 2762 | AAV variant | 3638 | AAV2/3 variant | 4514 |
| AAV CSp-8.8 | 822 | AAV2/3 variant | 2763 | AAV variant | 3639 | AAV2/3 variant | 4515 |
| AAV CSp-8.9 | 823 | AAV2/3 variant | 2764 | AAV variant | 3640 | AAV2/3 variant | 4516 |
| AAV CBr-B7.3 | 824 | AAV2/3 variant | 2765 | AAV variant | 3641 | AAV2/3 variant | 4517 |
| AAV CBr-B7.4 | 825 | AAV2/3 variant | 2766 | AAV variant | 3642 | AAV2/3 variant | 4518 |
| AAV3B | 826 | AAV2/3 variant | 2767 | AAV variant | 3643 | AAV2/3 variant | 4519 |
| AAV4 | 827 | AAV2/3 variant | 2768 | AAV variant | 3644 | AAV2/3 variant | 4520 |
| AAV5 | 828 | AAV2/3 variant | 2769 | AAV variant | 3645 | AAV2/3 variant | 4521 |
| AAV CHt-P2 | 829 | AAV2/3 variant | 2770 | AAV variant | 3646 | AAV2/3 variant | 4522 |
| AAV CHt-P5 | 830 | AAV2/3 variant | 2771 | AAV variant | 3647 | AAV2/3 variant | 4523 |
| AAV CHt-P9 | 831 | AAV2/3 variant | 2772 | AAV variant | 3648 | AAV2/3 variant | 4524 |
| AAV CBr-7.1 | 832 | AAV2/3 variant | 2773 | AAV variant | 3649 | AAV2/3 variant | 4525 |
| AAV CBr-7.2 | 833 | AAV2/3 variant | 2774 | AAV variant | 3650 | AAV2/3 variant | 4526 |
| AAV CBr-7.3 | 834 | AAV2/3 variant | 2775 | AAV variant | 3651 | AAV2/3 variant | 4527 |
| AAV CBr-7.4 | 835 | AAV2/3 variant | 2776 | AAV variant | 3652 | AAV2/3 variant | 4528 |
| AAV CBr-7.5 | 836 | AAV2/3 variant | 2777 | AAV variant | 3653 | AAV2/3 variant | 4529 |
| AAV CBr-7.7 | 837 | AAV2/3 variant | 2778 | AAV variant | 3654 | AAV2/3 variant | 4530 |
| AAV CBr-7.8 | 838 | AAV2/3 variant | 2779 | AAV variant | 3655 | AAV2/3 variant | 4531 |
| AAV CBr-7.10 | 839 | AAV2/3 variant | 2780 | AAV variant | 3656 | AAV2/3 variant | 4532 |
| AAV CKd-N3 | 840 | AAV2/3 variant | 2781 | AAV variant | 3657 | AAV2/3 variant | 4533 |
| AAV CKd-N4 | 841 | AAV2/3 variant | 2782 | AAV variant | 3658 | AAV2 | 910 |
| | | | | | | (comprising lung | |
| | | | | | | specific | |
| | | | | | | polypeptide) | |
| AAV CKd-N9 | 842 | AAV2/3 variant | 2783 | AAV variant | 3659 | AAV2 | 911 |
| | | | | | | (comprising lung | |
| | | | | | | specific | |
| | | | | | | polypeptide) | |
| AAV CLv-L4 | 843 | AAV2/3 variant | 2784 | AAV variant | 3660 | consensus amino | 1164 |
| | | | | | | acid sequence of | |
| | | | | | | ancestral AAV | |
| | | | | | | variants, C4, C7 | |
| | | | | | | and G4 | |
| AAV CLv-L5 | 844 | AAV2/3 variant | 2785 | AAV variant | 3661 | consensus amino | 1165 |
| | | | | | | acid sequence of | |
| | | | | | | ancestral AAV | |
| | | | | | | variants, C4 and | |
| | | | | | | C7 | |
| AAV CLv-L6 | 845 | AAV2/3 variant | 2786 | AAV variant | 3662 | AAV8 (with a | 1166 |
| | | | | | | AAV2 | |
| | | | | | | phospholipase | |
| | | | | | | domain) | |
| AAV CLv-K1 | 846 | AAV2/3 variant | 2787 | AAV variant | 3663 | | |
| AAV CLv-K3 | 847 | AAV2/3 variant | 2788 | AAV variant | 3664 | | |

In some embodiments, the AAV serotype may be, or may have a sequence as described in International Patent Publication WO2015038958, the contents of which are herein incorporated by reference in their entirety, such as, but not limited to, AAV9 (SEQ ID NO: 137 and 138 herein), PHP.B (SEQ ID NO: 5 and 6), G2B-13 (SEQ ID NO: 7), G2B-26 (SEQ ID NO: 5), TH1.1-32 (SEQ ID NO: 8), TH1.1-35 (SEQ ID NO: 9) or variants thereof. Further, any of the targeting peptides or amino acid inserts described in WO2015038958, may be inserted into any parent AAV serotype, such as, but not limited to, AAV9 (SEQ ID NO: 137 for the DNA sequence and SEQ ID NO: 138 for the amino acid sequence). In some embodiments, the amino acid insert is inserted between amino acids 586-592 of the parent AAV (e.g., AAV9). In another embodiment, the amino acid insert is inserted between amino acids 588-589 of the parent AAV sequence. The amino acid insert may be, but is not limited to, any of the following amino acid sequences: SEQ ID NO: 1262, 1263, 1264, 1265, 1266, 1267, 1268, 1269, 1270, 1271, 1271, 1273, 1274, 1275, 1276, and 1277. Non-limiting examples of nucleotide sequences that may encode the amino acid inserts include the following, SEQ ID NO: 1278, SEQ ID NO: 1279, SEQ ID NO: 1280, SEQ ID NO: 1281, SEQ ID NO: 1282, SEQ ID NO: 1283, SEQ ID NO: 1284, SEQ ID NO: 1285, SEQ ID NO: 1286, or SEQ ID NO: 1287.

In some embodiments, the AAV serotype may be, or may have a sequence as described in International Patent Publication WO2017100671, the contents of which are herein incorporated by reference in their entirety, such as, but not limited to, AAV9 (SEQ ID NO: 45 of WO2017100671, herein SEQ ID NO: 11), PHP.N (SEQ ID NO: 46 of WO2017100671, herein SEQ ID NO: 4), PHP.S (SEQ ID NO: 47 of WO2017100671, herein SEQ ID NO: 10), or variants thereof. Further, any of the targeting peptides or amino acid inserts described in WO2017100671 may be inserted into any parent AAV serotype, such as, but not limited to, AAV9. In some embodiments, the amino acid insert is inserted between amino acids 586-592 of the parent AAV (e.g., AAV9). In another embodiment, the amino acid insert is inserted between amino acids 588-589 of the parent AAV sequence. The amino acid insert may be, but is not limited to, any of the following amino acid sequences: SEQ ID NO: 1262, 1270, 1271, 1277, 1288, 1289, 1290, 1291, 1292, 1293, 1294, 1295, 1296, 1297, 1298, 1299, 1300, 1301, 1302, 1303, 1304, 1305, 1306, 1307, 1308, 1309, 1310, 1311, 1312, 1313, 1314, 1315, 1316, 1317, 1318, 1319, 1320, 1321, 1322, 1323, 1324, 1325, 1326, 1327, 1328, 1329, 1330, 1331, 1332, 1333, 1334, 1335, 1336, 1337, 1338, 1339, 1340, 1341, 1342, 1343, 1344, 1345, 1346, 1347, and 1348.

Non-limiting examples of nucleotide sequences that may encode the amino acid inserts include the following, SEQ ID NO: 1349, SEQ ID NO: 1350, SEQ ID NO: 1351, SEQ ID NO: 1352, SEQ ID NO: 1353, SEQ ID NO: 1354, SEQ ID NO: 1355, SEQ ID NO: 1356, SEQ ID NO: 1357, SEQ ID NO: 1358 (wherein N may be A, C, T, or G), SEQ ID NO: 1359 (wherein N may be A, C, T, or G), SEQ ID NO: 1360 (wherein N may be A, C, T, or G), SEQ ID NO: 1361 (wherein N may be A, C, T, or G), herein SEQ ID NO: 1362 (wherein N may be A, C, T, or G), SEQ ID NO: 1279, SEQ ID NO: 1280, SEQ ID NO: 1281, SEQ ID NO: 1287, or SEQ ID NO: 1363.

In some embodiments, the AAV serotype may be, or may have a sequence as described in U.S. Pat. No. 9,624,274, the contents of which are herein incorporated by reference in their entirety, such as, but not limited to, the AAV serotypes in table 1, or variants or derivative thereof. Further, any of the structural protein inserts described in U.S. Pat. No. 9,624,274, may be inserted into, but not limited to, I-453 and I-587 of any parent AAV serotype, such as, but not limited to, AAV2 (SEQ ID NO: 183 of U.S. Pat. No. 9,624,274).

The amino acid insert may be, but is not limited to, any of the following amino acid sequences: SEQ ID NO: 1364, 1365, 1366, 1367, 1368, 1369, 1370, 1371, 1372, 1373, 1374, 1375, 1376, 1377, 1378, 1379, 1380, 1381, 1382, 1383, 1384, 1385, 1386, 1387, 1388, 1389, 1390, 1391, 1392, 1393, 1394, 1395, 1396, 1397, 1398, 1399, 1400, 1401, 1402, 1403, 1404, 1405, and 1406.

In some embodiments, the AAV serotype may be, or may have a sequence as described in U.S. Pat. No. 9,475,845, the contents of which are herein incorporated by reference in their entirety, such as, but not limited to, AAV capsid proteins comprising modification of one or more amino acids at amino acid positions 585 to 590 of the native AAV2 capsid protein. Further the modification may result in, but not be limited to, the amino acid sequence of any one of SEQ ID NOs: 1407-1429.

In some embodiments, the amino acid modification is a substitution at amino acid positions 262 through 265 in the native AAV2 capsid protein or the corresponding position in the capsid protein of another AAV with a targeting sequence. The targeting sequence may be, but is not limited to, any of the amino acid sequences of SEQ ID NO: 1430, 1431, 1432, 1433, 1434, 1435, 1436, 1437, 1438, 1439, 1440, 1441, 1442, 1443, 1444, 1445, 1446, 1447, 1448, 1449, 1450, 1451, 1452, 1453, 1454, 1455, 1456, 1457, 1458, 1459, 1460, 1461, 1462, 1463, 1464, 1465, 1466, 1467, 1468, 1469, 1470, 1471, 1472, 1473, 1474, 1475, 1476, 1477, 1478, 1479, 1480, 1481, 1482, 1483, 1484, 1485, 1486, 1487, 1488, 1489, 1490, 1491, 1492, 1493, 1494, 1495, 1496, 1497, 1498, 1499, 1500, 1501, 1502, 1503, 1504, 1505, 1506, 1507, 1508, 1509, 1510, 1511, 1512, 1513, 1514, 1515, 1516, 1517, 1518, 1519, 1520, 1521, 1522, 1523, 1524, 1525, 1526, 1527, 1528, 1529, 1530, 1531, 1532, 1533, 1534, 1535, 1536, 1537, 1538, 1539, 1540, 1541, 1542, 1543, 1544, 1545, 1546, 1547, 1548, GGG, GFS, LWS, EGG, LLV, LSP, LBS, AGG, GRR, GGH and GTV.

In some embodiments, the AAV serotype may be, or may have a sequence as described in United States Publication No. US 20160369298, the contents of which are herein incorporated by reference in their entirety, such as, but not limited to, site-specific mutated capsid protein of AAV2 (SEQ ID NO: 97 of US 20160369298; herein SEQ ID NO: 1549) or variants thereof, wherein the specific site is at least one site selected from sites R447, G453, S578, N587, N587+1, S662 of VP1 or fragment thereof.

Further, any of the mutated sequences described in US 20160369298, may be or may have, but not limited to, any one of SEQ ID NO: 1550-1694. Non-limiting examples of nucleotide sequences that may encode the amino acid mutated sites include any of SEQ ID NO: 1695-1717.

In some embodiments, the AAV serotype may comprise an ocular cell targeting peptide as described in International Patent Publication WO2016134375, the contents of which are herein incorporated by reference in their entirety, such as, but not limited to SEQ ID NO: 9, and SEQ ID NO:10 of WO2016134375. Further, any of the ocular cell targeting peptides or amino acids described in WO2016134375, may be inserted into any parent AAV serotype, such as, but not limited to, SEQ ID NOs: 1718 and 1719. In some embodiments, modifications, such as insertions are made in AAV2 proteins at P34-A35, T138-A139, A139-P140, G453-T454, N587-R588, and/or R588-Q589. In certain embodiments, insertions are made at D384, G385, 1560, T561, N562, E563, E564, E565, N704, and/or Y705 of AAV9. The ocular cell targeting peptide may be, but is not limited to, any of SEQ ID NO: 1720 and 1721.

In some embodiments, the AAV serotype may be modified as described in the United States Publication US 20170145405 the contents of which are herein incorporated by reference in their entirety. AAV serotypes may include, modified AAV2 (e.g., modifications at Y444F, Y500F, Y730F and/or S662V), modified AAV3 (e.g., modifications at Y705F, Y731F and/or T492V), and modified AAV6 (e.g., modifications at S663V and/or T492V).

In some embodiments, the AAV serotype may be modified as described in the International Publication WO2017083722 the contents of which are herein incorporated by reference in their entirety. AAV serotypes may include, AAV1 (Y705+731F+T492V), AAV2 (Y444+500+730F+T491V), AAV3 (Y705+731F), AAV5, AAV 5(Y436+693+719F), AAV6 (VP3 variant Y705F/Y731F/T492V), AAV8 (Y733F), AAV9, AAV9 (VP3 variant Y731F), and AAV10 (Y733F).

In some embodiments, the AAV serotype may comprise, as described in International Patent Publication WO2017015102, the contents of which are herein incorporated by reference in their entirety, an engineered epitope comprising the amino acids of SEQ ID NO: 1722 or 1723. The epitope may be inserted in the region of amino acids 665 to 670 based on the numbering of the VP1 capsid of AAV8 (SEQ ID NO: 3 of WO2017015102) and/or residues 664 to 668 of AAV3B (SEQ ID NO: 3 of WO2017015102).

In some embodiments, the AAV serotype may be, or may have a sequence as described in International Patent Publication WO2017058892, the contents of which are herein incorporated by reference in their entirety, such as, but not limited to, AAV variants with capsid proteins that may comprise a substitution at one or more (e.g., 2, 3, 4, 5, 6, or 7) of amino acid residues 262-268, 370-379, 451-459, 472-473, 493-500, 528-534, 547-552, 588-597, 709-710, 716-722 of AAV1, in any combination, or the equivalent amino acid residues in AAV2, AAV3, AAV4, AAV5, AAV6, AAV7, AAV8, AAV9, AAV10, AAV11, AAV12, AAVrh8, AAVrh10, AAVrh32.33, bovine AAV or avian AAV. The amino acid substitution may be, but is not limited to, any of the amino acid sequences described in WO2017058892. In some embodiments, the AAV may comprise an amino acid substitution at residues 256L, 258K, 259Q, 261S, 263A, 264S, 265T, 266G, 272H, 385S, 386Q, S472R, V473D, N500E 547S, 709A, 710N, 716D, 717N, 718N, 720L, A456T, Q457T, N458Q, K459S, T492S, K493A, S586R, S587G, S588N, T589R and/or 722T of AAV1 (SEQ ID NO: 1 of WO2017058892) in any combination, 244N, 246Q, 248R, 249E, 250I, 251K, 252S, 253G, 254S, 255V, 256D, 263Y, 377E, 378N, 453L, 456R, 532Q, 533P, 535N, 536P, 537G, 538T, 539T, 540A, 541T, 542Y, 543L, 546N, 653V, 654P, 656S, 697Q, 698F, 704D, 705S, 706T, 707G, 708E, 709Y and/or 710R of AAV5 (SEQ ID NO:5 of WO2017058892) in any combination, 248R, 316V, 317Q, 318D, 319S, 443N, 530N, 531S, 532Q 533P, 534A, 535N, 540A, 541 T, 542Y, 543L, 545G, 546N, 697Q, 704D, 706T, 708E, 709Y and/or 710R of AAV5 (SEQ ID NO: 5 of WO2017058892) in any combination, 264S, 266G, 269N, 272H, 457Q, 588S and/or 5891 of AAV6 (SEQ ID NO:6 WO2017058892) in any combination, 457T, 459N, 496G, 499N, 500N, 589Q, 590N and/or 592A of AAV8 (SEQ ID NO: 8 of WO2017058892) in any combination, 451I, 452N, 453G, 454S, 455G, 456Q, 457N and/or 458Q of AAV9 (SEQ ID NO: 9 of WO2017058892) in any combination.

In some embodiments, the AAV may include a sequence of amino acids at positions 155, 156 and 157 of VP1 or at positions 17, 18, 19 and 20 of VP2, as described in International Publication No. WO 2017066764, the contents of which are herein incorporated by reference in their entirety. The sequences of amino acid may be, but not limited to, N-S-S, S-X-S, S-S-Y, N-X-S, N-S-Y, S-X-Y and N-X-Y, where N, X and Y are, but not limited to, independently non-serine, or non-threonine amino acids, wherein the AAV may be, but not limited to AAV1, AAV2, AAV3, AAV4, AAV5, AAV6, AAV7, AAV8, AAV9, AAV10, AAV11 and AAV12. In some embodiments, the AAV may include a deletion of at least one amino acid at positions 156, 157 or 158 of VP1 or at positions 19, 20 or 21 of VP2, wherein the AAV may be, but not limited to AAV1, AAV2, AAV3, AAV4, AAV5, AAV6, AAV7, AAV8, AAV9, AAV10, AAV11 and AAV12.

In some embodiments, the AAV may be a serotype generated by Cre-recombination-based AAV targeted evolution (CREATE) as described by Deverman et al., (Nature Biotechnology 34(2):204-209 (2016)), the contents of which are herein incorporated by reference in their entirety. In some embodiments, AAV serotypes generated in this manner have improved CNS transduction and/or neuronal and astrocytic tropism, as compared to other AAV serotypes. As non-limiting examples, the AAV serotype may include a peptide such as, but not limited to, PHP.B, PHP.B2, PHP.B3, PHP.A, PHP.S, G2A12, G2A15, G2A3, G2B4, and G2B5. In some embodiments, these AAV serotypes may be AAV9 (SEQ ID NO: 11 or 138) derivatives with a 7-amino acid insert between amino acids 588-589. Non-limiting examples of these 7-amino acid inserts include TLAVPFK (PHP.B; SEQ ID NO: 1262), SVSKPFL (PHP.B2; SEQ ID NO: 1270), FTLTTPK (PHP.B3; SEQ ID NO: 1271), YTLSQGW (PHP.A; SEQ ID NO: 1277), QAVRTSL (PHP.S; SEQ ID NO: 1321), LAKERLS (G2A3; SEQ ID NO: 1322), MNSTKNV (G2B4; SEQ ID NO: 1323), and/or VSGGHHS (G2B5; SEQ ID NO: 1324).

In some embodiments, the AAV serotype may be as described in Jackson et al (Frontiers in Molecular Neuroscience 9:154 (2016)), the contents of which are herein incorporated by reference in their entirety. In some embodiments, the AAV serotype is PHP.B or AAV9. In some embodiments, the AAV serotype is paired with a synapsin promoter to enhance neuronal transduction, as compared to when more ubiquitous promoters are used (i.e., CBA or CMV).

In some embodiments, the AAV serotype is a serotype comprising the AAVPHP.N (PHP.N) peptide, or a variant thereof. In some embodiments the AAV serotype is a serotype comprising the AAVPHP.B (PHP.B) peptide, or a variant thereof. In some embodiments, the AAV serotype is a serotype comprising the AAVPHP.A (PHP.A) peptide, or a variant thereof. In some embodiments, the AAV serotype is a serotype comprising the PHP.S peptide, or a variant thereof. In some embodiments, the AAV serotype is a serotype comprising the PHP.B2 peptide, or a variant thereof. In some embodiments, the AAV serotype is a serotype comprising the PHP.B3 peptide, or a variant thereof. In some embodiments, the AAV serotype is a serotype comprising the G2B4 peptide, or a variant thereof. In some embodiments, the AAV serotype is a serotype comprising the G2B5 peptide, or a variant thereof.

In some embodiments the AAV serotype is VOY101, or a variant thereof. In some embodiments, the VOY101 capsid comprises the amino acid sequence SEQ ID NO: 1. In some embodiments, the VOY101 amino acid sequence is encoded by a nucleotide sequence comprising SEQ ID NO: 2. In some embodiments, the VOY101 capsid comprises an amino acid sequence at least 70% identical to SEQ ID NO: 1, such as, 70%, 75%, 80%, 85%, 90%, 91%, 92%, 93%, 94%, 95%, 96%, 97%, 98%, 99%, or greater than 99%. In some embodiments, the VOY101 capsid comprises a nucleotide sequence at least 70% identical to SEQ ID NO: 2, such as, 70%, 75%, 80%, 85%, 90%, 91%, 92%, 93%, 94%, 95%, 96%, 97%, 98%, 99%, or greater than 99%.

In some embodiments, the AAV serotype is VOY201, or a variant thereof. In some embodiments, the VOY201 capsid comprises the amino acid sequence SEQ ID NO: 4534. In some embodiments, the VOY201 amino acid sequence is encoded by a nucleotide sequence comprising SEQ ID NO: 3. In some embodiments, the VOY201 capsid comprises an amino acid sequence at least 70% identical to SEQ ID NO: 4534, such as, 70%, 75%, 80%, 85%, 90%, 91%, 92%, 93%, 94%, 95%, 96%, 97%, 98%, 99%, or greater than 99%. In some embodiments, the VOY201 capsid comprises a nucleotide sequence at least 70% identical to SEQ ID NO: 3, such as, 70%, 75%, 80%, 85%, 90%, 91%, 92%, 93%, 94%, 95%, 96%, 97%, 98%, 99%, or greater than 99%.

In some embodiments, the AAV serotype is PHP.B, or a variant thereof. In some embodiments, the PHP.B capsid comprises the amino acid sequence SEQ ID NO: 5. In some embodiments, the PHP.B amino acid sequence is encoded by a nucleotide sequence comprising SEQ ID NO: 6. In some embodiments, the PHP.B capsid comprises an amino acid sequence at least 70% identical to SEQ ID NO: 5, such as, 70%, 75%, 80%, 85%, 90%, 91%, 92%, 93%, 94%, 95%, 96%, 97%, 98%, 99%, or greater than 99%. In some embodiments, the PHP.B capsid comprises a nucleotide sequence at least 70% identical to SEQ ID NO: 6, such as, 70%, 75%, 80%, 85%, 90%, 91%, 92%, 93%, 94%, 95%, 96%, 97%, 98%, 99%, or greater than 99%.

In some embodiments, the AAV serotype is PHP.N, or a variant thereof. In some embodiments, the PHP.N capsid comprises the amino acid sequence SEQ ID NO: 4. In some embodiments, the PHP.N capsid comprises an amino acid sequence at least 70% identical to SEQ ID NO: 4, such as, 70%, 75%, 80%, 85%, 90%, 91%, 92%, 93%, 94%, 95%, 96%, 97%, 98%, 99%, or greater than 99%.

In some embodiments the AAV serotype is AAV9, or a variant thereof. In some embodiments, the AAV9 capsid comprises the amino acid sequence SEQ ID NO: 138. In some embodiments, the AAV9 amino acid sequence is encoded by a nucleotide sequence comprising SEQ ID NO: 137. In some embodiments, the AAV9 capsid comprises an amino acid sequence at least 70% identical to SEQ ID NO: 138, such as, 70%, 75%, 80%, 85%, 90%, 91%, 92%, 93%, 94%, 95%, 96%, 97%, 98%, 99%, or greater than 99%. In some embodiments, the AAV9 capsid comprises a nucleotide sequence at least 70% identical to SEQ ID NO: 137, such as, 70%, 75%, 80%, 85%, 90%, 91%, 92%, 93%, 94%, 95%, 96%, 97%, 98%, 99%, or greater than 99%.

In some embodiments, the AAV serotype is AAV9 K449R, or a variant thereof. In some embodiments, the AAV9 K449R capsid comprises the amino acid sequence SEQ ID NO: 11. In some embodiments, the AAV9 K449R capsid comprises an amino acid sequence at least 70% identical to SEQ ID NO: 11, such as, 70%, 75%, 80%, 85%, 90%, 91%, 92%, 93%, 94%, 95%, 96%, 97%, 98%, 99%, or greater than 99%.

In some embodiments, the AAV capsid allows for blood brain barrier penetration following intravenous administration. Non-limiting examples of such AAV capsids include AAV9, AAV9 K449R, VOY101, VOY201, or AAV capsids comprising a peptide insert such as, but not limited to, AAVPHP.N (PHP.N), AAVPHP.B (PHP.B), PHP.S, G2A3, G2B4, G2B5, G2A12, G2A15, PHP.B2, PHP.B3, or AAVPHP.A (PHP.A).

In some embodiments, the AAV capsid is suitable for intramuscular administration and/or transduction of muscle fibers. Non-limiting examples of such AAV capsids include AAV2, AAV3, AAV8 and variants thereof such as, but not limited to, AAV2 variants, AAV2/3 variants, AAV8 variants, and/or AAV2/3/8 variants.

In some embodiments, the AAV serotype is an AAV2 variant. As a non-limiting example, the AAV serotype is an AAV2 variant comprising SEQ ID NO: 2679 or a fragment or variant thereof. As a non-limiting example, the AAV serotype is at least 70% identical to SEQ ID NO: 2679, such as, 70%, 75%, 80%, 85%, 90%, 91%, 92%, 93%, 94%, 95%, 96%, 97%, 98%, 99%, or greater than 99%.

In some embodiments, the AAV serotype is an AAV2/3 variant. As a non-limiting example, the AAV serotype is an AAV2/3 variant comprising SEQ ID NO: 2809 or a fragment or variant thereof. As a non-limiting example, the AAV serotype is an AAV2/3 variant which is at least 70% identical to SEQ ID NO: 2809, such as, 70%, 75%, 80%, 85%, 90%, 91%, 92%, 93%, 94%, 95%, 96%, 97%, 98%, 99%, or greater than 99%. As a non-limiting example, the AAV serotype is an AAV2/3 variant comprising SEQ ID NO: 2871 or a fragment or variant thereof. As a non-limiting example, the AAV serotype is an AAV2/3 variant which is at least 70% identical to SEQ ID NO: 2871, such as, 70%, 75%, 80%, 85%, 90%, 91%, 92%, 93%, 94%, 95%, 96%, 97%, 98%, 99%, or greater than 99%.

In some embodiments, the AAV serotype may comprise a capsid amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of those described herein.

In some embodiments, the AAV serotype may be encoded by a capsid nucleic acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of those described herein.

In some embodiments, the AAV serotype is selected for use due to its tropism for cells of the central nervous system. In some embodiments, the cells of the central nervous system are neurons. In another embodiment, the cells of the central nervous system are astrocytes.

In some embodiments, the AAV serotype is selected for use due to its tropism for cells of the muscle(s).

In some embodiments, the initiation codon for translation of the AAV VP1 capsid protein may be CTG, TTG, or GTG as described in U.S. Pat. No. 8,163,543, the contents of which are herein incorporated by reference in its entirety.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in its entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a “capsid protein” or “capsid polypeptide” (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### Viral Genome Component: Inverted Terminal Repeats (ITRs)

The AAV particles of the present disclosure comprise a viral genome with at least one ITR region and a payload region. In some embodiments, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes may be comprised of naturally occurring polynucleotide sequences or recombinantly derived polynucleotide sequences.

The ITRs may be derived from the same serotype as the capsid, selected from any of the serotypes listed in Table 1, or a derivative or functional variant thereof. The ITR may be of a different serotype than the capsid. In some embodiments, the AAV particle has more than one ITR. In a non-limiting example, the AAV particle has a viral genome comprising two ITRs. In some embodiments, the ITRs are of the same serotype as one another. In another embodiment, the ITRs are of different serotypes. Non-limiting examples include zero, one or both of the ITRs having the same serotype as the capsid. In some embodiments both ITRs of the viral genome of the AAV particle are AAV2 ITRs or a functional variant thereof.

Independently, each ITR may be about 100 to about 150 nucleotides in length. An ITR may be about 100-180 nucleotides in length, e.g., 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In some embodiments, the ITRs are 140-142 nucleotides in length. Non-limiting examples of ITR length are 102, 130, 140, 141, 142, 145 nucleotides in length, and those having at least 95% identity thereto.

In some embodiments, each ITR may be 141 nucleotides in length.

In some embodiments, each ITR may be 130 nucleotides in length.

In some embodiments, the AAV particles comprise two ITRs and one ITR is 141 nucleotides in length and the other ITR is 130 nucleotides in length.

In some embodiments, the viral genome comprises an ITR region comprising the nucleotide sequence of any of the sequences provided in Table 7 or a nucleotide sequence with at least 70%, 75%, 80%, 85%, 90%, 95% or 99% sequence identity thereto. In some embodiments, the viral genome comprises two ITR regions comprising the nucleotide sequence of any of the sequences provided in Table 7 or a nucleotide sequence with at least 70%, 75%, 80%, 85%, 90%, 95% or 99% sequence identity thereto, wherein the first and second ITR comprise the same sequence or wherein the first and second ITR comprise different sequences.

TABLE 7
Inverted Terminal Repeat (ITR) Sequence Regions

| Sequence | | |
|---|---|---|
| Region Name | Sequence Length | SEQ ID NO |
| ITR1 | 130 | 2076 |
| ITR2 | 141 | 2077 |
| ITR3 | 130 | 2078 |
| ITR4 | 141 | 2079 |

In some embodiment, the viral genome comprises an ITR provided in Table 7. In some embodiments, the viral genome comprises an ITR chosen from any one of ITR1-ITR4 or a functional variant thereof. In some embodiments, the viral genome comprises ITR1. In some embodiments, the viral genome comprises ITR1. In some embodiments, the ITR comprises the nucleotide sequence of any one of SEQ ID NOs: 2076-2079, or a sequence with at least 70%, 75%, 80%, 85%, 90%, 95% or 99% sequence identity thereto. In some embodiments, the ITR comprises the nucleotide sequence of SEQ ID NO: 2076 or a nucleotide sequence with at least 70%, 75%, 80%, 85%, 90%, 95% or 99% sequence identity thereto. In some embodiments, the ITR comprises the nucleotide sequence of SEQ ID NO: 2078 or a nucleotide sequence with at least 70%, 75%, 80%, 85%, 90%, 95% or 99% sequence identity thereto.

In some embodiments, the viral genome comprises ITR1 and ITR2. In some embodiments, the viral genome comprises ITR1 at the 5′ end and ITR2 at the 3′ end. In some embodiments, the viral genome comprises an ITR, e.g., a 5′ ITR, comprising the nucleotide sequence of SEQ ID NO: 2076 or a sequence with at least with at least 70%, 75%, 80%, 85%, 90%, 95% or 99% sequence identity thereto; and an ITR, e.g., a 3′ ITR, comprising the nucleotide sequence of SEQ ID NO: 2078 or a nucleotide sequence at least 70%, 75%, 80%, 85%, 90%, 95% or 99% sequence identity thereto.

### Viral Genome Component: Promoters

In some embodiments, the viral genome comprises at least one element to enhance the transgene target specificity and/or expression (See e.g., Powell et al. Viral Expression Cassette Elements to Enhance Transgene Target Specificity and Expression in Gene Therapy, 2015; the contents of which are herein incorporated by reference in its entirety). Non-limiting examples of elements to enhance the transgene target specificity and expression include promoters, endogenous miRNAs, post-transcriptional regulatory elements (PREs), polyadenylation (PolyA) signal sequences and upstream enhancers (USEs), CMV enhancers and introns.

In some embodiments, an element to enhance the transgene target specificity and/or expression comprise a promoter, an enhancer, e.g., a CMV enhancer (such as CMV ie enhancer), or both. In some embodiments, the viral genome comprises a promoter operably linked to a transgene encoded by a nucleic acid molecule encoding a payload, e.g., antibody molecule (e.g., an anti-tau antibody molecule, such as any one of those described herein). In some embodiments, the viral genome comprises an enhancer, e.g., a CMV (such as CMVIe) enhancer. In some embodiment, the viral genome comprises at least two promoters, such as an EF1α promoter and a CMV promoter.

In some embodiments, the viral genome comprises a promoter that is species specific, inducible, tissue-specific, and/or cell cycle-specific (e.g., as described in Parr et al., Nat. Med. 3:1145-9 (1997); the contents of which are herein incorporated by reference in their entirety). In some embodiments, the viral genome comprises a promoter that is sufficient for expression, e.g., in a target cell, of a payload (e.g., an antibody, e.g., an anti-tau antibody) encoded by a transgene.

In some embodiments, the promoter results in expression of the polypeptide(s) encoded in the payload region of the viral genome of the AAV particle, in the cell being targeted for expression of the payload (e.g., a functional anti-tau antibody) for a sufficient period of time in targeted cells, tissues, or organs.

In some embodiments, the promoter results in expression of the payload for at least 1 hour to 24 hours, e.g., 1-5 hours, 1-10 hours, 1-15 hours, 1-20 hours, 2-5 hours, 2-10 hours, 2-15 hours, 2-20 hours, or 2-24 hours, 3-5 hours, 3-15 hours, 3-20 hours, 3-24 hours, 4-5 hours, 4-15 hours, 4-20 hours, 4-24 hours, 5-15 hours, 5-20 hours, 5-23 hours, 6-15 hours, 6-20 hours 6-24 hours, 7-15 hours, 7-20 hours, 7-24 hours, 8-10 hours, 8-15 hours, 8-20 hours, 8-24 hours, 9-10 hours, 9-15 hours, 9-20 hours, 9-24 hours, 10-15 hours, 10-20 hours, 10-23 hours, 11-15 hours, 11-20 hours 11-24 hours, 12-15 hours, 12-20 hours, 12-24 hours, 13-15 hours, 13-20 hours, 13-24 hours, 14-15 hours, 14-20 hours, 14-23 hours, 15-20 hours, 15-24 hours, 16-20 hours, 16-24 hours, 17-20 hours, 17-24 hours, 18-20 hours, 18-24 hours, 19-20 hours, 19-24 hours, 20-24 hours, 21-24 hours, 22-24 hours, or 23-24 hours, e.g., 1 hour, 5 hours, 10 hours, 12 hours, 14 hours, 18 hours, 20 hours, or 24 hours. In some embodiments, the promoter results in expression of the payload for at least 1-7 days, e.g., 1-6 days, 1-5 days, 1-4 days, 1-3 days, 1-2 days, 2-7 days, 2-6 days, 2-5 days, 2-4 days, 2-3 days, 3-7 days, 3-6 days, 3-5 days, 3-4 days, 4-7 days, 4-6 days, 4-5 days, 5-7 days, 5-6 days, or 6-7 days, e.g., 1 day, 5 days, or 7 days. In some embodiments, the promoter results in expression of the payload for 1 week to 4 weeks, e.g., 1-3 weeks, 1-2 weeks, 2-4 weeks, 2-3 weeks, or 3-4 weeks. In some embodiments, the promoter results in expression of the payload for at least 1-12 months, at least 10-24 months, or at least 1-10 years, e.g., at least 1 year, at least 5 years, at least 10 years, or more than 10 years.

In some embodiments, the promoter may be a naturally occurring promoter, or a non-naturally occurring promoter. In some embodiments, the promoter is from a naturally expressed protein. In some embodiments, the promoter is an engineered promoter. In some embodiments, the promoter comprises a viral promoter, plant promoter, and/or a mammalian promoter. In some embodiments, the promoter may be a human promoter. In some embodiments, the promoter may be truncated. In some embodiments, the promoter is not a cell specific promoter. In some embodiments, the promoter is a ubiquitous promoter. In some embodiments, the promoter is a cell- or tissue-specific promoter, such as neuronal or glial cell specific promoter.

In some embodiments, the viral genome comprises a promoter that results in expression in one or more, e.g., multiple, cells and/or tissues, e.g., a ubiquitous promoter. In some embodiments, a promoter that results in expression in one or more tissues includes but is not limited to a human elongation factor 1α-subunit (EF1α) promoter, a cytomegalovirus (CMV) immediate-early enhancer and/or promoter, a chicken β-actin (CBA) promoter (including a minimal CBA promoter) and its derivative CAG, a β glucuronidase (GUSB) promoter (such as one with a size of about 350-400 nts, or 378 nts), or ubiquitin C (UBC) promoter (such as one with a size of about 300-350 nts, or 332 nts).

Other promoters include is a neurofilament light (NFL) promoter (such as one with a size of about 600-700 nucleotides, or about 650 nucleotides); a neurofilament heavy (NFH) promoter (such as one with a size of about 900-950 nucleotides, or about 920 nucleotides); a scn8a promoter. (such as one with a size of about 450-500 nucleotides or about 470 nucleotides); a phosphoglycerate kinase 1 (PGK) promoter; a CB6 promoter.

In some embodiments, the viral genome comprises a ubiquitous promoter as described in Yu et al. (Molecular Pain 2011, 7:63), Soderblom et al. (E. Neuro 2015), Gill et al., (Gene Therapy 2001, Vol. 8, 1539-1546), and Husain et al. (Gene Therapy 2009), each of which are incorporated by reference in their entirety. In some embodiments, the viral genome comprises a ubiquitous promoter chosen from CMV, CBA (including derivatives CAG, CB6, CBh, etc.), EF-1α, PGK, UBC, GUSB (hGBp), or UCOE (promoter of HNRPA2B1-CBX3).

In some embodiments, a tissue-specific expression elements can be used to restrict expression to certain cell types such as, but not limited to, muscle specific promoters, B cell promoters, monocyte promoters, leukocyte promoters, macrophage promoters, pancreatic acinar cell promoters, endothelial cell promoters, lung tissue promoters, astrocyte promoters, or nervous system promoters which can be used to restrict expression to neurons, astrocytes, or oligodendrocytes.

In some embodiments, the viral genome comprises a muscle-specific promoter, e.g., a promoter that results in expression in a muscle cell. In some embodiments, a muscle-specific promoter includes but is not limited to a mammalian muscle creatine kinase (MCK) promoter, a mammalian desmin (DES) promoter, a mammalian troponin I (TNNI2) promoter, a synthetic C5-12 promoter, and a mammalian skeletal alpha-actin (ASKA) promoter (see, e.g., U.S. Pat. Publication US20110212529, the contents of which are herein incorporated by reference in their entirety).

In some embodiments, the viral genome comprises a nervous system specific promoter, e.g., a promoter that results in expression of a payload in a neuron, an astrocyte, and/or an oligodendrocyte. In some embodiments, a nervous system specific promoter that results in expression in neurons includes but is not limited to a neuron-specific enolase (NSE) promoter, a platelet-derived growth factor (PDGF) promoter, a platelet-derived growth factor B-chain (PDGF-β) promoter, a synapsin (Syn) promoter, a methyl-CpG binding protein 2 (MeCP2) promoter, a Ca²⁺/calmodulin-dependent protein kinase II (CaMKII) promoter, a metabotropic glutamate receptor 2 (mGluR2) promoter, a neurofilament light (NFL) or heavy (NFH) promoter, a β-globin minigene nβ2 promoter, a preproenkephalin (PPE) promoter, a enkephalin (Enk) promoter, and an excitatory amino acid transporter 2 (EAAT2) promoter.

In some embodiments, a nervous system specific promoter that results in expression in astrocytes includes but is not limited to a glial fibrillary acidic protein (GFAP) promoter and a EAAT2 promoter. In some embodiments, a nervous system specific promoter that results in expression in oligodendrocytes includes but is not limited to a myelin basic protein (MBP) promoter. In some embodiments, the viral genome comprises a nervous system specific promoter as described in Husain et al. (Gene Therapy 2009), Passini and Wolfe (J. Virol. 2001, 12382-12392), Xu et al. (Gene Therapy 2001, 8, 1323-1332), Drews et al. (Mamm Genome (2007) 18:723-731), and Raymond et al. (Journal of Biological Chemistry (2004) 279(44) 46234-46241), each of which are incorporated by reference in their entirety.

In some embodiments, the viral genome comprises a liver promoter, e.g. a promoter that results in expression a liver cell. In some embodiments, the liver promoter is chosen from human α-1-antitrypsin (hAAT) or thyroxine binding globulin (TBG). In some embodiments, the viral genome comprises an RNA pol III promoter. In some embodiments, the RNA pol III promoter is chosen from U6 or H1. In some embodiments, the viral genome comprises an endothelial cell promoter, e.g., a promoter that results in expression in an endothelial cell. In some embodiments, the endothelial cell promoter is an intercellular adhesion molecule 2 (ICAM2) promoter.

In some embodiments, the viral genome comprises a promoter chosen from a CAG promoter, a CBA promoter (e.g., a minimal CBA promoter), a CB promoter, a CMV(IE) promoter and/or enhancer, a GFAP promoter, a synapsin promoter, an ICAM2 promoter, or a functional variant thereof. In some embodiments, the viral genome comprises a CAG promoter, a CMVie enhancer, and a minimal CBA promoter. In some embodiments, the viral genome comprises a CMV(IE) promoter and a CB promoter.

In some embodiments, the CAG promoter comprises the nucleotide sequence of SEQ ID NO: 2080 or nucleotide sequence with at least 70%, 75%, 80%, 85%, 90%, 95%, or 99% sequence identity thereto. In some embodiments, the CBA promoter (e.g., a minimal CBA promoter) comprises the nucleotide sequence of SEQ ID NO: 2082 or nucleotide sequence with at least 70%, 75%, 80%, 85%, 90%, 95%, or 99% sequence identity thereto. In some embodiments, the CB promoter comprises the nucleotide sequence of SEQ ID NO: 2083 or nucleotide sequence with at least 70%, 75%, 80%, 85%, 90%, 95%, or 99% sequence identity thereto. In some embodiments, the GFAP promoter comprises the nucleotide sequence of SEQ ID NO: 2084 or nucleotide sequence with at least 70%, 75%, 80%, 85%, 90%, 95%, or 99% sequence identity thereto. In some embodiments, the GFAP promoter comprises the nucleotide sequence of SEQ ID NO: 2085 or nucleotide sequence with at least 70%, 75%, 80%, 85%, 90%, 95%, or 99% sequence identity thereto. In some embodiments, the synapsin promoter comprises the nucleotide sequence of SEQ ID NO: 2086 or nucleotide sequence with at least 70%, 75%, 80%, 85%, 90%, 95%, or 99% sequence identity thereto. In some embodiments, the CMV(IE) promoter comprises the nucleotide sequence of SEQ ID NO: 2239 or nucleotide sequence with at least 70%, 75%, 80%, 85%, 90%, 95%, or 99% sequence identity thereto. In some embodiments, the CMV(ie) enhancer comprises e nucleotide sequence of SEQ ID NO: 2081 or nucleotide sequence with at least 70%, 75%, 80%, 85%, 90%, 95%, or 99% sequence identity thereto.

In some embodiments, the viral genome comprises an enhancer element, a promoter and/or a 5′UTR intron. The enhancer element, also referred to herein as an “enhancer,” may be, but is not limited to, a CMV enhancer, the promoter may be, but is not limited to, a CMV, CBA, UBC, GUSB, NSE, Synapsin, MeCP2, and GFAP promoter and the 5′UTR/intron may be, but is not limited to, SV40, and CBA-MVM. As a non-limiting example, the enhancer, promoter and/or intron used in combination may be: (1) CMV enhancer, CMV promoter, SV40 5′UTR intron; (2) CMV enhancer, CBA promoter, SV 40 5′UTR intron; (3) CMV enhancer, CBA promoter, CBA-MVM 5′UTR intron; (4) UBC promoter; (5) GUSB promoter; (6) NSE promoter; (7) Synapsin promoter; (8) MeCP2 promoter; and (9) GFAP promoter.

In some embodiments, the viral genome comprises a promoter provided in Table 8. In some embodiments, the promoter is chosen from any one of Promoter 1-Promoter 12, or a functional variant thereof. In some embodiments, the promoter comprises the nucleotide sequence of any one of SEQ ID NOs: 2080-2089, 2238 and 2239, or a sequence with at least 70%, 75%, 80%, 85%, 90%, 95% or 99% sequence identity thereto. In some embodiments, the promoter comprises the nucleotide sequence of SEQ ID NO: 2080 or a sequence with at least 70%, 75%, 80%, 85%, 90%, 95% or 99% sequence identity thereto. In some embodiments, the promoter comprises the nucleotide sequence of SEQ ID NO: 2081, or a sequence with at least 70%, 75%, 80%, 85%, 90%, 95% or 99% sequence identity thereto. In some embodiments, the promoter comprises the nucleotide sequence of SEQ ID NO: 2082, or a sequence with at least 70%, 75%, 80%, 85%, 90%, 95% or 99% sequence identity thereto. In some embodiments, the promoter comprises the nucleotide sequence of SEQ ID NO: 2083, or a sequence with at least 70%, 75%, 80%, 85%, 90%, 95% or 99% sequence identity thereto. In some embodiments, the promoter comprises the nucleotide sequence of SEQ ID NO: 2084, or a sequence with at least 70%, 75%, 80%, 85%, 90%, 95% or 99% sequence identity thereto. In some embodiments, the promoter comprises the nucleotide sequence of SEQ ID NO: 2085, or a sequence with at least 70%, 75%, 80%, 85%, 90%, 95% or 99% sequence identity thereto. In some embodiments, the promoter comprises the nucleotide sequence of SEQ ID NO: 2086, or a sequence with at least 70%, 75%, 80%, 85%, 90%, 95% or 99% sequence identity thereto. In some embodiments, the promoter comprises the nucleotide sequence of SEQ ID NO: 2239, or a sequence with at least 70%, 75%, 80%, 85%, 90%, 95% or 99% sequence identity thereto.

In some embodiments, the viral genome comprises Promoter 1. In some embodiments, the viral genome comprises Promoter 2. In some embodiments, the viral genome comprises Promoter 3. In some embodiments, the viral genome comprises Promoter 4. In some embodiments, the viral genome comprises Promoter 5. In some embodiments, the viral genome comprises Promoter 6. In some embodiments, the viral genome comprises Promoter 7. In some embodiments, the viral genome comprises Promoter 8. In some embodiments, the viral genome comprises Promoter 9. In some embodiments, the viral genome comprises Promoter 10. In some embodiments, the viral genome comprises Promoter 11. In some embodiments, the viral genome comprises Promoter 12.

TABLE 8
Promoter Sequence Regions

| Sequence | | |
|---|---|---|
| Region Name | Sequence Length | SEQ ID NO |
| Promoter 1 | 1715 | 2080 |
| Promoter 2 | 299 | 2081 |
| Promoter 3 | 283 | 2082 |
| Promoter 4 | 260 | 2083 |
| Promoter 5 | 654 | 2084 |
| Promoter 6 | 699 | 2085 |
| Promoter 7 | 557 | 2086 |
| Promoter 8 | 382 | 2087 |
| Promoter 9 | 1736 | 2088 |
| Promoter 10 | 365 | 2089 |
| Promoter 11 | 1714 | 2238 |
| Promoter 12 | 380 | 2239 |

In some embodiments, the promoter of the viral genome further comprises at least one promoter sub-region. In some embodiments, the promoter comprises Promoter 1, further comprising Promoter 2 and Promoter 3 sub-regions.

In some embodiments, the viral genome comprises more than one promoter sequence region. In some embodiments, the viral genome comprises at least 2 or more promoters. In some embodiments, the viral genome comprises two promoter sequence regions. In some embodiments, the viral genome comprises three promoter sequence regions. In some embodiments, the viral genome comprises Promoter 4 and Promoter 8. In some embodiments, the viral genome comprises Promoter 12 and Promoter 4. In some embodiments, the promoter is a combination of a 382 nucleotide CMV-enhancer sequence (such as SEQ ID NO: 2087) and a 260 nucleotide CBA-promoter sequence (such as SEQ ID NO: 2083).

In some embodiments, the viral genome comprises a promoter that has a length between about 100-2000 nucleotides. In some embodiments, the promoter has a length between about 100-700 nucleotides, e.g., between about 100-600 nucleotides, 100-500 nucleotides, 100-400 nucleotides, 100-300 nucleotides, 100-200 nucleotides, 200-700 nucleotides, 200-600 nucleotides, 200-500 nucleotides, 200-400 nucleotides, 200-300 nucleotides, 300-700 nucleotides, 300-600 nucleotides, 300-500 nucleotides, 300-400 nucleotides, 400-700 nucleotides, 400-600 nucleotides, 400-500 nucleotides, 500-700 nucleotides, 500-600 nucleotides, or 600-700 nucleotides. In some embodiments, the promoter has a length between about 900-2000 nucleotides, e.g., between about 900-1000 nucleotides, 9000-1500 nucleotides, 1000-1500 nucleotides, 1000-2000 nucleotides, or 1500-2000 nucleotides. In some embodiments, the promoter has a length between about 1500 to about 1800 nucleotides, e.g., about 1715 nucleotides. In some embodiments, the promoter has a length of about 500 to about 750 nucleotides, e.g., about 557 nucleotides or about 699 nucleotides. In some embodiments, the promoter has a length of about 200 to about 450 nucleotides, e.g., about 260 nucleotides, about 283 nucleotides, about 299 nucleotides, about 365 nucleotides, about 380 nucleotides, about 382 nucleotides, about 399 nucleotides, about 557 nucleotides, about 654 nucleotides, or about 699 nucleotides. As a non-limiting example, the viral genome comprises a promoter region that is about 1714 nucleotides in length. As a non-limiting example, the viral genome comprises a promoter region that is about 1715 nucleotides in length. As a non-limiting example, the viral genome comprises a promoter region that is about 1736 nucleotides in length.

### Genetic Element Component: Exons

In some embodiments, the viral genome comprises an exon sequence region. In some embodiments, the viral genome comprises at least 2, at least 3, at least 4, or at least 5 exon regions. In some embodiments, the viral genome comprises 2 exon regions.

In some embodiments, the viral genome comprises an ie exon 1 region. In some embodiments, the ie exon 1 region comprises the nucleotide sequence of SEQ ID NO: 2090, or a nucleotide sequence with at least 70%, 75%, 80%, 85%, 90%, 95%, or 99% sequence identity thereto. In some embodiments, the viral genome comprises a human beta-globin exon region. In some embodiments, the human beta-globin exon region comprises a nucleotide sequence of SEQ ID NO: 2093 or a sequence with at least 70%, 75%, 80%, 85%, 90%, 95%, or 99% sequence identity thereto. In some embodiments, the viral genome comprises an ie exon 1 region and a human beta-globin exon region.

In some embodiments, the exon region is provided in Table 9. In some embodiments, the viral genome comprises an exon region chosen from Exon1, Exon2, Exon3, Exon4, or a function variant thereof. In some embodiments, the viral genome comprises Exon1. In some embodiments, the viral genome comprises Exon4. In some embodiments, the viral genome comprises Exon1 and Exon4. In some embodiments, the exon region comprises the nucleotide sequence of any one of SEQ ID NOs: 2090-2094, or a nucleotide sequence with at least 70%, 75%, 80%, 85%, 90%, 95%, or 99% sequence identity thereto. In some embodiments, the exon region comprises the nucleotide sequence of SEQ ID NO: 2090, or a nucleotide sequence with at least 70%, 75%, 80%, 85%, 90%, 95%, or 99% sequence identity thereto. In some embodiments, the exon region comprises the nucleotide sequence of SEQ ID NO: 2093, or a nucleotide sequence with at least 70%, 75%, 80%, 85%, 90%, 95%, or 99% sequence identity thereto.

TABLE 9
Exon Sequence Regions

| Sequence | | |
|---|---|---|
| Region Name | Sequence Length | SEQ ID NO |
| Exon1 | 134 | 2090 |
| Exon2 | 102 | 2091 |
| Exon3 | 59 | 2092 |
| Exon4 | 53 | 2093 |
| Exon5 | 54 | 2094 |

In some embodiments, the exon comprises about 50-nucleotides in length, e.g., about 50-140 nucleotides, about 50-130 nucleotides, about 50-120 nucleotides, about 50-110 nucleotides, about 50-100 nucleotides, about 50-90 nucleotides, about 50-80 nucleotides, about 50-80 nucleotides, about 50-70 nucleotides, about 50-60 nucleotides, about 60-150 nucleotides, about 60-140 nucleotides, about 60-130 nucleotides, about 60-120 nucleotides, about 60-110 nucleotides, about 60-100 nucleotides, about 60-90 nucleotides, about 60-80 nucleotides, about 60-80 nucleotides, about 60-70 nucleotides, about 70-150 nucleotides, about 70-140 nucleotides, about 70-130 nucleotides, about 70-120 nucleotides, about 70-110 nucleotides, about 70-100 nucleotides, about 70-90 nucleotides, about 70-80 nucleotides, about 80-150 nucleotides, about 80-140 nucleotides, about 80-130 nucleotides, about 80-120 nucleotides, about 80-110 nucleotides, about 80-100 nucleotides, about 80-90 nucleotides, about 90-150 nucleotides, about 90-140 nucleotides, about 90-130 nucleotides, about 90-120 nucleotides, about 90-110 nucleotides, about 90-100 nucleotides, about 100-150 nucleotides, about 100-140 nucleotides, about 100-130 nucleotides, about 100-120 nucleotides, about 100-110 nucleotides, about 110-150 nucleotides, about 110-140 nucleotides, about 110-130 nucleotides, about 110-120 nucleotides, about 120-150 nucleotides, about 120-140 nucleotides, about 120-130 nucleotides, about 130-150 nucleotides, about 130-140 nucleotides, or about 140-150 nucleotides. In some embodiments, the exon region comprises about 120 nucleotides to about 140 nucleotides in length, e.g., about 134 nucleotides. In some embodiments, the exon region comprises about 40 nucleotides to about 60 nucleotides in length, e.g., about 53 nucleotides.

In some embodiments, the exon region(s) may, independently, have a length between 20-80, 30-70, 40-60, about 50, 100-150, 110-140, 120-140, 120-150, 125-140, 130-145, 130-135, or about 135 nucleotides. In certain embodiments, the viral genome comprises an exon region that is about 53 nucleotides in length. In certain embodiments, the viral genome comprises an exon region that is about 54 nucleotides in length. In certain embodiments, the viral genome comprises an exon region that is about 59 nucleotides in length. In certain embodiments, the viral genome comprises an exon region that is about 102 nucleotides in length. In certain embodiments, the viral genome comprises an exon region that is about 134 nucleotides in length.

### Viral Genome Component: Introns

In some embodiments, the viral genome comprises at least one element to enhance the expression of a transgene encoding a payload. In some embodiments, an element that enhances expression of a transgene comprises an introns or functional variant thereof. In some embodiments, the viral genome comprises an intron or functional variant thereof. In some embodiments, the viral genome comprises at least two intron regions, e.g., at least 2 intron regions, at least 3 intron regions, at least 4 intron regions, or 5 or more intron regions.

In some embodiments, the viral genome comprises an intron chosen from a MVM intron (67-97 bps), an F.IX truncated intron 1 (300 bps), an β-globin SD/immunoglobulin heavy chain splice acceptor intron (250 bps), an adenovirus splice donor/immunoglobin splice acceptor intron (500 bps), SV40 late splice donor/splice acceptor intron (19S/16S) (180 bps), or a hybrid adenovirus splice donor/IgG splice acceptor intron (230 bps).

In some embodiments, the viral genome comprises a human beta-globin intron region. In some embodiments, the human beta-globin intron region comprises the nucleotide sequence of SEQ ID NO: 2097 or 2240, or a nucleotide sequence with at least 70%, 75%, 80%, 85%, 90%, 95% or 99% sequence identity to SEQ ID NO: 2097 or 2240. In some embodiments, the viral genome comprises an ie intron 1 region. In some embodiments, the ie intron 1 region comprises the nucleotide sequence of SEQ ID NO: 2095, or a nucleotide sequence with at least 70%, 75%, 80%, 85%, 90%, 95% or 99% sequence identity thereto. In some embodiments, the viral genome comprises a first human beta-globin intron region, e.g., a first human beta-globin intron region comprising SEQ ID NO: 2240, and a second human beta-globin intron region, e.g., a second human beta-globin intron region comprising SEQ ID NO: 2097. In some embodiments, an ie intron 1 region, e.g., an ie intron 1 region comprising SEQ ID NO: 2095, and a human beta-globin intron region, e.g., a human beta-globin intron region comprising SEQ ID NO: 2240.

In some embodiments, the viral genome comprises an intron region provided in Table 10. In some embodiments, the viral genome comprises an intron region chosen from any one of Intron1 to Intron15, or functional variants thereof. In some embodiments, the viral genome comprises Intron1, or a functional variant thereof. In some embodiments, the viral genome comprises Intron3, or a functional variant thereof. In some embodiments, the viral genome comprises Intron12, or a functional variant thereof. In some embodiments, the viral genome comprises Intron 12 and Intron3, or functional variants thereof. In some embodiments, the viral genome comprises Intron1 and Intron12, or functional variants thereof. In some embodiments, the viral genome comprises Intron1 and Intron3, or functional variants thereof. As used herein, functional variants refer to sequences with at least 70%, 75%, 80%, 85%, 90%, 95%, or 99% sequence identity to the reference sequence.

In some embodiments, the viral genome comprises an intron region of any one of SEQ ID NOs: 2095-2105, 2240, or 2256-2258, or a sequence with at least 70%, 75%, 80%, 85%, 90%, 95%, or 99% sequence identity thereto. In some embodiments, the intron region comprises the nucleotide sequence of SEQ ID NO: 2095, or a nucleotide sequence with at least 70%, 75%, 80%, 85%, 90%, 95%, or 99% sequence identity thereto. In some embodiments, the intron region comprises the nucleotide sequence of SEQ ID NO: 2097, or a nucleotide sequence with at least 70%, 75%, 80%, 85%, 90%, 95%, or 99% sequence identity thereto. In some embodiments, the intron region comprises the nucleotide sequence of SEQ ID NO: 2040, or a nucleotide sequence with at least 70%, 75%, 80%, 85%, 90%, 95%, or 99% sequence identity thereto. In some embodiments, the intron region comprises the nucleotide sequence of SEQ ID NO: 2095 or a nucleotide sequence with at least 70%, 75%, 80%, 85%, 90%, 95%, or 99% sequence identity thereto; and the nucleotide sequence of SEQ ID NO: 2097 or a nucleotide sequence with at least 70%, 75%, 80%, 85%, 90%, 95%, or 99% sequence identity thereto.

TABLE 10
Intron Sequence Regions

| Sequence | | |
|---|---|---|
| Region Name | Sequence Length | SEQ ID NO |
| Intron1 | 32 | 2095 |
| Intron2 | 15 | 2096 |
| Intron3 | 347 | 2097 |
| Intron4 | 168 | 2098 |
| Intron5 | 73 | 2099 |
| Intron6 | 73 | 2100 |
| Intron7 | 73 | 2101 |
| Intron8 | 53 | 2102 |
| Intron9 | 172 | 2103 |
| Intron10 | 1074 | 2104 |
| Intron11 | 41 | 2105 |
| Intron12 | 566 | 2240 |
| Intron13 | 491 | 2256 |
| Intron14 | 387 | 2257 |
| Intron15 | 292 | 2258 |

In some embodiments, the viral genome comprises an intron region comprising about 10 nucleotides to about 1200 nucleotides in length. In some embodiments, the intron region comprises about 10-100 nucleotides in length, e.g., about 10-90 nucleotides, about 10-80 nucleotides, about 10-70 nucleotides, about 10-60 nucleotides, about 10-50 nucleotides, about 10-40 nucleotides, about 10-30 nucleotides, about 10-20 nucleotides, about 20-100 nucleotides, about 20-90 nucleotides, about 20-80 nucleotides, about 20-70 nucleotides, about 20-60 nucleotides, about 20-50 nucleotides, about 20-40 nucleotides, about 20-30 nucleotides, about 30-100 nucleotides, about 30-90 nucleotides, about 30-80 nucleotides, about 30-70 nucleotides, about 30-60 nucleotides, about 30-50 nucleotides, about 30-40 nucleotides, about 40-100 nucleotides, about 40-90 nucleotides, about 40-80 nucleotides, about 40-70 nucleotides, about 40-60 nucleotides, about 40-50 nucleotides, about 50-100, about 50-90 nucleotides, about 50-80 nucleotides, about 50-70 nucleotides, about 50-60 nucleotides, about 60-100 nucleotides, about 60-90 nucleotides, about 60-80 nucleotides, about 60-70 nucleotides, about 70-100 nucleotides, about 70-90 nucleotides, about 70-80 nucleotides, about 80-100 nucleotides, about 80-90 nucleotides, or about 90-100 nucleotides in length. In some embodiments, the intron region comprises about 100-600 nucleotides in length, e.g., about 100-500 nucleotides, about 100-400 nucleotides, about 100-300 nucleotides, about 100-200 nucleotides, about 200-600 nucleotides, about 200-500 nucleotides, about 200-400 nucleotides, about 200-300 nucleotides, about 300-600 nucleotides, about 300-500 nucleotides, about 300-400 nucleotides, about 400-600 nucleotides, about 400-500 nucleotides, or about 500-600 nucleotides in length. In some embodiments, the intron region comprises about 900-1200 nucleotides in length, e.g., about 900-1100 nucleotides, about 900-1000 nucleotides, about 1000-1200 nucleotides, about 1000-1100 nucleotides, or about 1100-1200 nucleotides.

In some embodiments, the intron region comprises about 20 to about 40 nucleotides in length, e.g., about 32 nucleotides. In some embodiments, the intron region comprises about 340 to about 360 nucleotides in length, e.g., about 347 nucleotides. In some embodiments, the intron region comprises about 550 to about 570 nucleotides in length, e.g., about 566 nucleotides.

In some embodiments, the intron may be 15 nucleotides in length, 32 nucleotides in length, 41 nucleotides in length, 53 nucleotides in length, 54 nucleotides in length, 59 nucleotides in length, 73 nucleotides in length, 102 nucleotides in length, 134 nucleotides in length, 168 nucleotides in length, 172 nucleotides in length, 292 nucleotides in length, 347 nucleotides in length, 387 nucleotides in length, 491 nucleotides in length, 566 nucleotides in length, or 1074 nucleotides in length.

### Viral Genome Component: Tags

In some embodiments, the viral genome comprises a nucleic acid sequence encoding a tag polypeptide (e.g., a tag sequence or tag sequence region herein). As used herein, the term “tag” indicates a polynucleotide sequence appended to the payload, that once expressed may be used to identify the expressed payload. Alternatively, the term “tag” may indicate a polynucleotide sequence appended to the payload that signals for retention of the expressed payload in a particular region of the cell (e.g., endoplasmic reticulum).

In some embodiments, the AAV particle viral genome comprises more than one tag sequence region. In some embodiments, the AAV particle viral genome comprises two tag sequence regions. In some embodiments, the AAV particle viral genome comprises three tag sequence regions. In some embodiments, the AAV particle viral genome comprises more than three tag sequence regions.

In some embodiments, the viral genome comprises two or more tag sequences. In some embodiments, the tag comprises a nucleotide sequence appended to the transgene encoding the payload (e.g., an antibody molecule described herein), wherein the tag polypeptide encoded by the tag nucleic acid sequence is used to identify the encoded payload, e.g., antibody molecule. In other embodiments, a tag is a nucleotide sequence appended to the payload that signals for retention of the expressed payload in a region of the cell (e.g., endoplasmic reticulum or nucleus).

In some embodiments, the viral genome comprises a tag sequence provided in Table 12. In some embodiments, the viral genome comprises any one of Tag1-Tag5 or a functional variant thereof. In some embodiments, the tag sequence comprises the nucleotide sequence of any one of SEQ ID NOs: 2118-2121 or 2255, or a nucleotide sequence with at least 70%, 75%, 80%, 85%, 90%, 95% or 99% sequence identity thereto. In some embodiments, the encoded tag polypeptide comprises an amino acid sequence encoded by of any one of SEQ ID NOs: 2118-2121 or 2255, or an amino acid sequence with at least 70%, 75%, 80%, 85%, 90%, 95% or 99% sequence identity thereto.

TABLE 12
Tag Sequence Regions

| Sequence | | |
|---|---|---|
| Region Name | Sequence Length | SEQ ID NO |
| Tag1 | 27 | 2118 |
| Tag2 | 21 | 2119 |
| Tag3 | 18 | 2120 |
| Tag4 | 18 | 2121 |
| Tag5 | 18 | 2255 |

In some embodiments, the nucleotide sequence encoding the tag polypeptide comprises about 10-50 nucleotides in length, e.g., about 10-40 nucleotides, about 10-30 nucleotides, about 10-20 nucleotides, about 20-50 nucleotides, about 20-40 nucleotides, about 20-30 nucleotides, about 30-50 nucleotides, about 30-40 nucleotides, or about 40 to 50 nucleotides. In some embodiments, the nucleotide sequence encoding the tag polypeptide comprises about 10 nucleotides to about 30 nucleotides, e.g., about 18 nucleotides or about 21 nucleotides. In some embodiments, the nucleotide sequence encoding the tag polypeptide comprises about 20 nucleotides to about 40 nucleotides, e.g., about 27 nucleotides.

In some embodiments, the AAV particle viral genome comprises no tag sequence region. For example, the AAV particle viral genome of any one of Tables 94 and 95 may comprise a tag sequence region (see TAU_ITR252, TAU_ITR253, TAU_ITR256, TAU_ITR257, TAU_ITR260, TAU_ITR261, TAU_ITR264, and TAU_ITR265). Otherwise identical AAV particle viral genomes, except for the lack of the tag sequence region, are expressly contemplated. For example, an AAV particle viral genome corresponding to TAU_ITR252 but lacks the tag sequence may comprise, from 5′ to 3′, sequence elements represented by SEQ ID NOs: 2076, 2080, 4563, 1741, 2170, 1730, 1957, 2122, and 2078 (i.e., the tag sequence SEQ ID NO: 2118 is lacking).

Viral Genome Component: Polyadenylation Sequence

In some embodiments, the viral genome of the AAV particles of the present disclosure comprise a polyadenylation sequence. In some embodiments, the viral genome comprises a polyadenylation (referred to herein as poly A, polyA, or poly-A) sequence between the 3′ end of the transgene encoding the payload and the 5′ end of the 3′ITR. In some embodiments, the viral genome comprises two or more polyA sequences. In some embodiments, the viral genome does not comprise a polyA sequence.

In some embodiments, the viral genome comprises a rabbit globin polyA signal region. In some embodiments, the rabbit globin polyA signal region comprises the nucleotide sequence of SEQ ID NO: 2122, or a nucleotide sequence with at least 70%, 75%, 80%, 85%, 90%, 95%, or 99% sequence identity thereto.

TABLE 13
PolyA Signal Sequence Regions

| Sequence | | |
|---|---|---|
| Region Name | Sequence Length | SEQ ID NO |
| PolyA1 | 127 | 2122 |
| PolyA2 | 477 | 2123 |
| PolyA3 | 552 | 2124 |

In some embodiments, the polyA signal region is provided in Table 13. In some embodiments, the viral genome comprises a polyA sequence region chosen from polyA1, polyA2, polyA3, or a functional variant thereof. In some embodiments, the viral genome comprises the polyA signal region of PolyA1 or a functional variant thereof. In some embodiments, the polyA signal region comprises the nucleotide sequence of any one of SEQ ID NOs: 2122-2124, or a sequence with at least 70%, 75%, 80%, 85%, 90%, 95%, or 99% sequence identity thereto. In some embodiments, the polyA signal region comprises the nucleotide sequence of SEQ ID NO: 2122, or a nucleotide sequence with at least 70%, 75%, 80%, 85%, 90%, 95%, or 99% sequence identity thereto.

In some embodiments, the polyA signal region comprises a length of about 100-600 nucleotides, e.g., about 100-500 nucleotides, about 100-400 nucleotides, about 100-300 nucleotides, about 100-200 nucleotides, about 200-600 nucleotides, about 200-500 nucleotides, about 200-400 nucleotides, about 200-300 nucleotides, about 300-600 nucleotides, about 300-500 nucleotides, about 300-400 nucleotides, about 400-600 nucleotides, about 400-500 nucleotides, or about 500-600 nucleotides. In some embodiments, the polyA signal region comprises a length of about 100 to 150 nucleotides, e.g., about 127 nucleotides. In some embodiments, the polyA signal region comprises a length of about 450 to 500 nucleotides, e.g., about 477 nucleotides. In some embodiments, the polyA signal region comprises a length of about 520 to about 560 nucleotides, e.g., about 552 nucleotides. In some embodiments, the polyA signal region comprises a length of about 127 nucleotides.

### Viral Genome Component: Filler Sequences

In some embodiments, the viral genome comprises a filler sequence regions. In some embodiments, the viral genome comprises two or more filler sequences. A filler sequence is provided in Table 14. In some embodiments, the viral genome comprises FILLER1, FILLER2, or a functional variant thereof. In some embodiments, the viral genome comprises FILLER 1 and FILLER2. In some embodiments, the filler sequence comprises the nucleotide sequence of SEQ ID NO: 2125, or a nucleotide sequence with at least 70%, 75%, 80%, 85%, 90%, 95%, 99% or 100% sequence identity thereto. In some embodiments, the filler sequence comprises the nucleotide sequence of SEQ ID NO: 2126, or a nucleotide sequence with at least 70%, 75%, 80%, 85%, 90%, 95%, 99% or 100% sequence identity thereto. In some embodiments, the filler sequence comprises the nucleotide sequence of SEQ ID NO: 2125 or a nucleotide sequence with at least 70%, 75%, 80%, 85%, 90%, 95%, 99% or 100% sequence identity thereto; and the nucleotide sequence of SEQ ID NO: 2126 or a nucleotide sequence with at least 70%, 75%, 80%, 85%, 90%, 95%, 99% or 100% sequence identity thereto.

TABLE 14
Filler Sequence Regions

| Sequence | | |
|---|---|---|
| Region Name | Sequence Length | SEQ ID NO |
| FILLER1 | 1153 | 2125 |
| FILLER2 | 1240 | 2126 |

In some embodiments, the filler sequence comprises about 100-2000, about 200 to 1900, about 300 to 1800, about 400 to 1700, about 500 to 1600, about 600 to 1500, about 700 to 1500, about 800 to 1500, about 900 to 1500, about 1000-1500 nucleotides in length, e.g., about 1000-1400 nucleotides, about 1000-1300 nucleotides, about 1000-1200 nucleotides, about 1200-1500 nucleotides, about 1200-1400 nucleotides, about 1200-1300 nucleotides, about 1300-1500 nucleotides, about 1300-1400 nucleotides, or about 1400-1500 nucleotides. In some embodiments, the filler sequence comprises about 1140 nucleotides to about 1160 nucleotides in length, e.g., about 1153 nucleotides. In some embodiments, the filler sequence comprises about 1230 nucleotides to about 1250 nucleotides in length, e.g., about 1240 nucleotides.

In some embodiments, the AAV particle viral genome does not comprise a filler sequence region.

### Viral Genome Component: Untranslated Regions (UTRs)

In some embodiments, the viral genome comprises an untranslated region (UTR). In some embodiments, a wild type UTR of a gene are transcribed but not translated. In some embodiments, the 5′ UTR starts at the transcription start site and ends at the start codon and the 3′ UTR starts immediately following the stop codon and continues until the termination signal for transcription.

In some embodiments, a UTR comprises a feature found in abundantly expressed genes of specific target organs to enhance the stability and protein production. As a non-limiting example, a 5′ UTR from mRNA normally expressed in the liver (e.g., albumin, serum amyloid A, Apolipoprotein A/B/E, transferrin, alpha fetoprotein, erythropoietin, or Factor VIII) may be used in the viral genome of an AAV particle described herein to enhance expression in hepatic cell lines or liver.

In some embodiments, a viral genome comprises a 5′UTR, e.g., a wild-type (e.g., naturally occurring) 5′UTR or a recombinant (e.g., non-naturally occurring) 5′UTR. In some embodiments, a 5′ UTR comprises a feature which plays a role in translation initiation. In some embodiments, a UTR, e.g., a 5′ UTR, comprises a Kozak sequence. In some embodiments, a Kozak sequence is involved in the process by which the ribosome initiates translation of many genes. In some embodiments, a Kozak sequence has the consensus sequence of CCR(A/G)CCAUGG, where R is a purine (adenine or guanine) three bases upstream of the start codon (ATG), which is followed by another ‘G’. In some embodiments, a Kozak sequence comprises the nucleotide sequence of GAGGAGCCACC (SEQ ID NO: 4543) or a nucleotide sequence with at least 95-99% sequence identity thereto. In some embodiments, a Kozak sequence comprises the nucleotide sequence of GCCGCCACCATG (SEQ ID NO: 2114), or a nucleotide sequence with at least 95-99% sequence identity thereto. In some embodiments, a viral genome comprises a 5′UTR comprising a Kozak sequence. In some embodiments, a viral genome comprises a 5′UTR that does not comprise a Kozak sequence.

In some embodiments, the 5′UTR in the viral genome does not include a Kozak sequence.

In some embodiments, the viral genome comprises a 3′UTR, e.g., a wild-type (e.g., naturally occurring) 3′UTR or a recombinant (e.g., non-naturally occurring) 3′UTR. In some embodiments, a 3′ UTR comprises an element that modulates, e.g., increases or decreases, stability of a nucleic acid. In some embodiments, a 3′ UTR comprises stretches of Adenosines and Uridines embedded therein, e.g., an AU rich signature. These AU rich signatures are generally prevalent in genes with high rates of turnover and are described, e.g., in Chen et al, 1995, the contents of which are herein incorporated by reference in its entirety. In some embodiments, an AR rich signature comprises an AU rich element (ARE). In some embodiments, a 3′UTR comprises an ARE chosen from a class I ARE (e.g., c-Myc and MyoD), a class II ARE (e.g., GM-CSF and TNF-α), a class III ARE (e.g., c-Jun and Myogenin), or combination thereto. In some embodiments, a class I ARE comprises several dispersed copies of an AUUUA motif within U-rich regions. In some embodiments, a class II ARE comprises two or more overlapping UUAUUUA(U/A)(U/A) nonamers. In some embodiments, a class III ARE comprises U rich regions and/or do not contain an AUUUA motif. In some embodiments, an ARE destabilizes the messenger.

In some embodiments, a 3′UTR comprises a binding site for a protein member of the ELAV family. In some embodiments, a 3′ UTR comprises a binding site for an HuR protein. In some embodiments, an HuR protein binds to an ARE of any one of classes I-III and/or increases the stability of mRNA. Without wishing to be bound by theory, it is believed in some embodiments, that a 3′UTR comprising an HuR specific binding sites will lead to HuR binding and, stabilization of a message in vivo.

In some embodiments, the 3′ UTR of the viral genome comprises an oligo(dT) sequence for templated addition of a poly-A tail.

In some embodiments, the viral genome comprises a miRNA seed, binding site and/or full sequence. Generally, microRNAs (or miRNA or miR) are 19-25 nucleotide noncoding RNAs that bind to the sites of nucleic acid targets and down-regulate gene expression either by reducing nucleic acid molecule stability or by inhibiting translation. In some embodiments, the microRNA sequence comprises a seed region, e.g., a sequence in the region of positions 2-8 of the mature microRNA, which sequence has perfect Watson-Crick complementarity to the miRNA target sequence of the nucleic acid. In some embodiments, the viral genome may be engineered to include, alter or remove at least one miRNA binding site, sequence, or seed region.

In some embodiments, a UTR from any gene known in the art may be incorporated into the viral genome of an AAV particle described herein. These UTRs, or portions thereof, may be placed in the same orientation as in the gene from which they were selected, or they may be altered in orientation or location. In some embodiments, the UTR used in the viral genome of the AAV particle may be inverted, shortened, lengthened, made with one or more other 5′ UTRs or 3′ UTRs known in the art. In some embodiments, an altered UTR, comprises a UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides. In some embodiments, the viral genome comprises an artificial UTR, e.g., a UTR that is not a variant of a wild-type, e.g., a naturally occurring, UTR. In some embodiments, the viral genome comprises a UTR selected from a family of transcripts whose proteins share a common function, structure, feature or property.

In some embodiments, the viral genome of the AAV particle comprises at least one artificial UTRs which is not a variant of a wild type UTR. In some embodiments, the viral genome of the AAV particle comprises UTRs which have been selected from a family of transcripts whose proteins share a common function, structure, feature or property.

### Viral Genome Component: miR Binding Site

Tissue- or cell-specific expression of the AAV viral particles of the disclosure can be enhanced by introducing tissue- or cell-specific regulatory sequences, e.g., promoters, enhancers, microRNA binding sites, e.g., a detargeting site. Without wishing to be bound by theory, it is believed that an encoded miR binding site can modulate, e.g., prevent, suppress, or otherwise inhibit, the expression of a gene of interest on the viral genome of the disclosure, based on the expression of the corresponding endogenous microRNA (miRNA) or a corresponding controlled exogenous miRNA in a tissue or cell, e.g., a non-targeting cell or tissue. In some embodiments, a miR binding site modulates, e.g., reduces, expression of the payload encoded by a viral genome of an AAV particle described herein in a cell or tissue where the corresponding mRNA is expressed.

In some embodiments, the viral genome of an AAV particle described herein comprises a nucleotide sequence encoding a microRNA binding site, e.g., a detargeting site. In some embodiments, the viral genome of an AAV particle described herein comprises a nucleotide sequence encoding a miR binding site, a microRNA binding site series (miR BSs), or a reverse complement thereof.

In some embodiments, the nucleotide sequence encoding the miR binding site series or the miR binding site is located in the 3′-UTR region of the viral genome (e.g., 3′ relative to the nucleic acid sequence encoding a payload), e.g., before the polyA sequence, 5′-UTR region of the viral genome (e.g., 5′ relative to the nucleic acid sequence encoding a payload), or both.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, all copies are identical, e.g., comprise the same miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is at least about 5 to 10 nucleotides, e.g., about 7-8 nucleotides, nucleotides in length. In some embodiments, the spacer coding sequence or reverse complement thereof comprises one or more of (i) GGAT; (ii) CACGTG; (iii) GCATGC, or a repeat of one or more of (i)-(iii).

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, at least 1, 2, 3, 4, 5, or all of the copies are different, e.g., comprise a different miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is at least about 5 to 10 nucleotides, e.g., about 7-8 nucleotides, in length. In some embodiments, the spacer coding sequence or reverse complement thereof comprises one or more of (i) GGAT; (ii) CACGTG; (iii) GCATGC, or a repeat of one or more of (i)-(iii).

In some embodiments, the encoded miR binding site is substantially identical (e.g., at least 70%, 75%, 80%, 85%, 90%, 95%, 99% or 100% identical), to the miR in the host cell. In some embodiments, the encoded miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% identical to the miR in the host cell.

In some embodiments, the nucleotide sequence encoding the miR binding site is substantially complimentary (e.g., at least 70%, 75%, 80%, 85%, 90%, 95%, 99% or 100% identical), to the miR in the host cell. In some embodiments, to complementary sequence of the nucleotide sequence encoding the miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% identical to the miR in the host cell.

In some embodiments, an encoded miR binding site or sequence region is at least about 10 to about 125 nucleotides in length, e.g., at least about 10 to 50 nucleotides, 10 to 100 nucleotides, 50 to 100 nucleotides, 50 to 125 nucleotides, or 100 to 125 nucleotides in length. In some embodiments, an encoded miR binding site or sequence region is at least about 7 to about 28 nucleotides in length, e.g., at least about 8-28 nucleotides, 7-28 nucleotides, 8-18 nucleotides, 12-28 nucleotides, 20-26 nucleotides, 22 nucleotides, 24 nucleotides, or 26 nucleotides in length, and optionally comprises at least one consecutive region (e.g., 7 or 8 nucleotides) complementary to the seed sequence of a miRNA (e.g., a miR122, a miR142, a miR183).

In some embodiments, the encoded miR binding site is complementary to a miR expressed in liver or hepatocytes, such as miR122. In some embodiments, the encoded miR binding site or encoded miR binding site series comprises a miR122 binding site sequence. In some embodiments, the encoded miR122 binding site comprises the nucleotide sequence of ACAAACACCATTGTCACACTCCA (SEQ ID NO: 4566), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 4566, e.g., wherein the modification can result in a mismatch between the encoded miR binding site and the corresponding miRNA. In some embodiments, the viral genome comprises at least 3, 4, or 5 copies of the encoded miR122 binding site, e.g., an encoded miR122 binding site series, optionally wherein the encoded miR122 binding site series comprises the nucleotide sequence of: ACAAACACCATTGTCACACTCCACACAAACACCATTGT CACACTCCACACAAACACCATTGTCACACTCCA (SEQ ID NO: 4567), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 4567, e.g., wherein the modification can result in a mismatch between the encoded miR binding site and the corresponding miRNA. In some embodiments, at least two of the encoded miR122 binding sites are connected directly, e.g., without a spacer. In other embodiments, at least two of the encoded miR122 binding sites are separated by a spacer, e.g., 1, 2, 3, 4, 5, 6, 7, 8, 9, or 10 nucleotides in length, which is located between two or more consecutive encoded miR122 binding site sequences. In embodiments, the spacer is at least about 5 to 10 nucleotides, e.g., about 7-8, in length. In some embodiments, the spacer coding sequence or reverse complement thereof comprises one or more of (i) GGAT; (ii) CACGTG; (iii) GCATGC, or a repeat of one or more of (i)-(iii). In some embodiments, an encoded miR binding site series comprises at least 3-5 copies (e.g., 4 copies) of a miR122 binding site, with or without a spacer, wherein the spacer is at least about 5 to 10 nucleotides, e.g., about 7-8 nucleotides, in length.

In some embodiments, the encoded miR binding site is complementary to a miR expressed in hematopoietic lineage, including immune cells (e.g., antigen presenting cells or APC, including dendritic cells (DCs), macrophages, and B-lymphocytes). In some embodiments, the encoded miR binding site complementary to a miR expressed in hematopoietic lineage comprises a nucleotide sequence disclosed, e.g., in US 2018/0066279, the contents of which are incorporated by reference herein in its entirety.

In some embodiments, the encoded miR binding site or encoded miR binding site series comprises a miR-142-3p binding site sequence. In some embodiments, the encoded miR-142-3p binding site comprises the nucleotide sequence of TCCATAAAGTAGGAAACACTACA (SEQ ID NO: 4568), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 4568, e.g., wherein the modification can result in a mismatch between the encoded miR binding site and the corresponding miRNA. In some embodiments, the viral genome comprises at least 3, 4, or 5 copies of the encoded miR-142-3p binding site, e.g., an encoded miR-142-3p binding site series. In some embodiments, an encoded miR binding site series comprises at least 3-5 copies (e.g., 4 copies) of a miR-142-3p binding site, with or without a spacer, wherein the spacer is at least about 5 to 10 nucleotides, e.g., about 7-8 nucleotides, in length.

In some embodiments, the encoded miR binding site is complementary to a miR expressed in a DRG (dorsal root ganglion) neuron, e.g., a miR183, a miR182, and/or miR96 binding site. In some embodiments, the encoded miR binding site complementary to a miR expressed in expressed in a DRG neuron comprises a nucleotide sequence disclosed, e.g., in WO2020/132455, the contents of which are incorporated by reference herein in its entirety.

In some embodiments, the encoded miR binding site or encoded miR binding site series comprises a miR183 binding site sequence. In some embodiments, the encoded miR183 binding site comprises the nucleotide sequence of AGTGAATTCTACCAGTGCCATA (SEQ ID NO: 4569), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 4569, e.g., wherein the modification can result in a mismatch between the encoded miR binding site and the corresponding miRNA. In some embodiments, the sequence complementary to the seed sequence corresponds to the double underlined of the encoded miR-183 binding site sequence. In some embodiments, the viral genome comprises at least comprises at least 3, 4, or 5 copies of the encoded miR183 binding site, e.g. an encoded miR183 binding site. In some embodiments, an encoded miR binding site series comprises at least 3-5 copies (e.g., 4 copies) of a miR183 binding site, with or without a spacer, wherein the spacer is at least about 5 to 10 nucleotides, e.g., about 7-8 nucleotides, in length.

In some embodiments, the encoded miR binding site or encoded miR binding site series comprises a miR182 binding site sequence. In some embodiments, the encoded miR182 binding site comprises, the nucleotide sequence of AGTGTGAGTTCTACCATTGCCAAA (SEQ ID NO: 4570), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 4570, e.g., wherein the modification can result in a mismatch between the encoded miR binding site and the corresponding miRNA. In some embodiments, the viral genome comprises at least 3, 4, or 5 copies of the encoded miR182 binding site, e.g., an encoded miR182 binding site series. In some embodiments, an encoded miR binding site series comprises at least 3-5 copies (e.g., 4 copies) of a miR182 binding site, with or without a spacer, wherein the spacer is at least about 5 to 10 nucleotides, e.g., about 7-8 nucleotides, in length.

In some embodiments, the encoded miR binding site or encoded miR binding site series comprises a miR96 binding site sequence. In some embodiments, the encoded miR96 binding site comprises the nucleotide sequence of AGCAAAAATGTGCTAGTGCCAAA (SEQ ID NO: 4571), a sequence having at least 50%, 55%, 60%, 65%, 70%, 75%, 80%, 85%, 90%, at least 95%, at least 99%, or 100% sequence identity, or having at least one, two, three, four, five, six, or seven modifications but no more than ten modifications to SEQ ID NO: 4571, e.g., wherein the modification can result in a mismatch between the encoded miR binding site and the corresponding miRNA. In some embodiments, the viral genome comprises at least 3, 4, or 5 copies of the encoded miR96 binding site, e.g., an encoded miR96 binding site series. In some embodiments, an encoded miR binding site series comprises at least 3-5 copies (e.g., 4 copies) of a miR96 binding site, with or without a spacer, wherein the spacer is at least about 5 to 10 nucleotides, e.g., about 7-8, in length.

In some embodiments, the encoded miR binding site series comprises a miR122 binding site, a miR142 binding site, a miR183 binding site, a miR182 binding site, a miR 96 binding site, or a combination thereof. In some embodiments, the encoded miR binding site series comprises at least 3, 4, or 5 copies of a miR122 binding site, a miR142 binding site, a miR183 binding site, a miR182 binding site, a miR 96 binding site, or a combination thereof. In some embodiments, at least two of the encoded miR binding sites are connected directly, e.g., without a spacer. In other embodiments, at least two of the encoded miR binding sites are separated by a spacer, e.g., 1, 2, 3, 4, 5, 6, 7, 8, 9, or 10 nucleotides in length, which is located between two or more consecutive encoded miR binding site sequences. In embodiments, the spacer is at least about 5 to 10 nucleotides, e.g., about 7-8 nucleotides, in length. In some embodiments, the spacer coding sequence or reverse complement thereof comprises one or more of (i) GGAT; (ii) CACGTG; (iii) GCATGC, or a repeat of one or more of (i)-(iii). In some embodiments, an encoded miR binding site series comprises at least 3-5 copies (e.g., 4 copies) of a combination of at least two, three, four, five, or all of a miR122 binding site, a miR142 binding site, a miR183 binding site, a miR182 binding site, a miR96 binding site, with or without a spacer, wherein the spacer is at least about 5 to 10 nucleotides, e.g., about 7-8 nucleotides, in length.

### Payloads

The AAV particles of the present disclosure comprise at least one payload region. As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid. Payloads of the present disclosure typically encode polypeptides (e.g., antibodies or antibody-based compositions) or fragments or variants thereof.

The payload region may be constructed in such a way as to reflect a region similar to or mirroring the natural organization of an mRNA.

The payload region may comprise a combination of coding and non-coding nucleic acid sequences.

In some embodiments, the AAV payload region may encode a coding or non-coding RNA.

In some embodiments, the AAV particle comprises a viral genome with a payload region comprising nucleic acid sequences encoding more than one polypeptide of interest (e.g., an antibody). In such an embodiment, a viral genome encoding more than one polypeptide may be replicated and packaged into a viral particle. A target cell transduced with a viral particle comprising more than one polypeptide may express each of the polypeptides in a single cell.

In some embodiments, an AAV particle comprises a viral genome with a payload region comprising a nucleic acid sequence encoding a heavy chain and a light chain of an antibody, or fragments thereof. The heavy chain and light chain are expressed and assembled to form the antibody which is secreted.

In some embodiments, the payload region may comprise at least one inverted terminal repeat (ITR), a promoter region, an intron region, and a coding region. In some embodiments, the coding region comprises a heavy chain region and/or a light chain region of an antibody, or a fragment thereof, and any two components may be separated by a linker region.

In some embodiments, the coding region may comprise a payload region with a heavy chain and light chain sequence separated by a linker and/or a cleavage site. In some embodiments, the heavy and light chain sequence is separated by an IRES sequence. In some embodiments, the heavy and light chain sequence is separated by a foot and mouth virus sequence. In some embodiments, the heavy and light chain sequence is separated by a foot and mouth virus sequence and a furin cleavage site. In some embodiments, the heavy and light chain sequence is separated by a porcine teschovirus-1 virus sequence. In some embodiments, the heavy and light chain sequence is separated by a porcine teschovirus-1 virus and a furin cleavage site. In some embodiments, the heavy and light chain sequence is separated by a 5xG4S sequence (“5xG4S” disclosed as SEQ ID NO: 4538).

Where the AAV particle payload region encodes a polypeptide, the polypeptide may be a peptide or protein. A protein encoded by the AAV particle payload region may comprise an antibody, an antibody related composition, a secreted protein, an intracellular protein, an extracellular protein, and/or a membrane protein. The encoded proteins may be structural or functional. In addition to the antibodies or antibody-based composition, proteins encoded by the payload region may include, in combination, certain mammalian proteins involved in immune system regulation. The AAV viral genomes encoding polypeptides described herein may be useful in the fields of human disease, viruses, infections, veterinary applications and a variety of in vivo and in vitro settings.

In some embodiments, the AAV particles are useful in the field of medicine for the treatment, prophylaxis, palliation, or amelioration of neurological diseases and/or disorders.

### Antibodies and Antibody-Based Compositions

Payload regions of the AAV particles may encode polypeptides that form one or more functional antibodies or antibody-based compositions. As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), and antibody fragments (e.g., diabodies) so long as they exhibit a desired biological activity (e.g., “functional”). Antibodies are primarily amino-acid based molecules but may also comprise one or more modifications (including, but not limited to the addition of sugar moieties, fluorescent moieties, chemical tags, etc.).

In some embodiments, the payload region of the AAV particles encodes two or more functional antibodies or antigen-binding fragments thereof, such as scFv (in either VH-linker-VL configuration or VL-linker-VH configuration), Fab, F(ab′)₂, etc. In certain embodiments, the two or more antibodies or fragments thereof comprise one specific for tau N-terminus (such as the N-terminal domain N-terminal to the 1N domain, or residues 15-25 or 1-about 50), one specific for tau mid-domain (such as the mid-domain comprising, consisting essentially, of consisting of residues between the 2N and R1 domains, or residues about 95-about 250 of the human tau protein), and one specific for tau C-terminal domain (such as the C-terminal domain comprising, consisting essentially, of consisting of residues C-terminal to the R4 domain, or residues about 370-441 of the human tau protein). The antibody or fragment thereof may be specific for one or more epitopes or phosphorylated Ser and/or Thr residues in the N-, mid- and/or C-terminal domains. Exemplary and non-limiting N-terminal epitopes may include residues 15-25 or 15-30. Exemplary and non-limiting mid-domain epitopes may include residues 125-131 or 234-246. Exemplary and non-limiting mid-domain epitopes may include pT181, pS199, pS202/pT205, pT212/pS214, and/or pT217. Exemplary and non-limiting C-terminal domain epitopes may include pS396, pS404, and/or pS409. In certain embodiments, the antibody or fragment thereof recognizes a 3D conformation epitope with residues not contiguous on the preminary sequence. Exemplary and non-limiting N-terminal-specific antibodies include IPN002, and antibodies that either binds to the same epitope thereof or competes binding therewith. Exemplary and non-limiting mid-domain-specific antibodies include AT08, PT3, UCB, PT76, and antibodies that either binds to the same epitope thereof or competes binding therewith. Exemplary and non-limiting C-terminal domain-specific antibodies include PHF1, C10.2, and antibodies that either binds to the same epitope thereof or competes binding therewith. Exemplary and non-limiting C-terminal domain-specific antibody fragments include PHF1 antibody fragments described herein, such as those in any one of SEQ ID NOs: 4547-4562, those with a VH sequence comprising any one of SEQ ID NOs: 1839, 1841, and 2170, or those with a VL sequence comprising SEQ ID NO: 4565.

As used herein, “antibody-based” or “antibody-derived” compositions are monomeric or multi-meric polypeptides which comprise at least one amino-acid region derived from a known or parental antibody sequence and at least one amino acid region derived from a non-antibody sequence, e.g., mammalian protein.

Payload regions may encode polypeptides that form or function as any antibody, including antibodies that are known in the art and/or antibodies that are commercially available. The encoded antibodies may be therapeutic, diagnostic, or for research purposes. Further, polypeptides may include fragments of such antibodies or antibodies that have been developed to comprise one or more of such fragments (e.g., variable domains or complementarity determining regions (CDRs)).

In some embodiments, the viral genome of the AAV particles may comprise nucleic acids which have been engineered to enable expression of antibodies, antibody fragments, or components of any of those described in U.S. Pat. No. 7,041,807 related to YYX epitope; US20090175884, US20110305630, US20130330275 related to misfolded proteins in cancer; US20040175775 related to PrP in eye fluid; US20030114360 related to copolymers and methods of treating prion-related diseases; WO2009121176 related to insulin-induced gene peptide compositions; US20030022243, WO2003000853 related to protein aggregation assays; WO200078344 related to prion protein peptides and uses thereof. Each of these publications are incorporated by reference in their entireties.

### Antibody Generation

In some embodiments, viral genomes of the AAV particles may encode antibodies or antibody-based compositions produced using methods known in the art. Such methods may include, but are not limited to, immunization and display technologies (e.g., phage display, yeast display, and ribosomal display). Antibodies may be developed, for example, using any naturally occurring or synthetic antigen. As used herein, an “antigen” is an entity which induces or evokes an immune response in an organism. An immune response is characterized by the reaction of the cells, tissues and/or organs of an organism to the presence of a foreign entity. Such an immune response typically leads to the production by the organism of one or more antibodies against the foreign entity, e.g., antigen or a portion of the antigen. As used herein, “antigens” also refer to binding partners for specific antibodies or binding agents in a display library.

In some embodiments, the sequences of the polypeptides to be encoded in the viral genomes may be derived from antibodies produced using hybridoma technology. Host animals (e.g. mice, rabbits, goats, and llamas) may be immunized by an injection with an antigenic protein to elicit lymphocytes that specifically bind to the antigen. Lymphocytes may be collected and fused with immortalized cell lines to generate hybridomas which can be cultured in a suitable culture medium to promote growth. The antibodies produced by the cultured hybridomas may be subjected to analysis to determine binding specificity of the antibodies for the target antigen. Once antibodies with desirable characteristics are identified, corresponding hybridomas may be subcloned through limiting dilution procedures and grown by standard methods. The antibodies produced by these cells may be isolated and purified using standard immunoglobulin purification procedures.

In some embodiments, the sequences of the polypeptides to be encoded in the viral genomes may be produced using heavy and light chain variable region cDNA sequences selected from hybridomas or from other sources. Sequences encoding antibody variable domains expressed by hybridomas may be determined by extracting RNA molecules from antibody-producing hybridoma cells and producing cDNA by reverse transcriptase polymerase chain reaction (PCR). PCR may be used to amplify cDNA using primers specific for heavy and light chain sequences. PCR products may then be subcloned into plasmids for sequence analysis. Antibodies may be produced by insertion of resulting variable domain sequences into expression vectors.

In some embodiments, the sequences of the polypeptides to be encoded in the viral genomes may be generated using display technologies. Display technologies used to generate polypeptides may include any of the display techniques (e.g. display library screening techniques) disclosed in International Patent Application No. WO2014074532, the contents of which are herein incorporated by reference in their entirety. In some embodiments, synthetic antibodies may be designed, selected, or optimized by screening target antigens using display technologies (e.g. phage display technologies). Phage display libraries may comprise millions to billions of phage particles, each expressing unique antibody fragments on their viral coats. Such libraries may provide richly diverse resources that may be used to select potentially hundreds of antibody fragments with diverse levels of affinity for one or more antigens of interest (McCafferty, et al., 1990. Nature. 348:552-4; Edwards, B. M. et al., 2003. JMB. 334: 103-18; Schofield, D. et al., 2007. Genome Biol. 8, R254 and Pershad, K. et al., 2010. Protein Engineering Design and Selection. 23:279-88; the contents of each of which are herein incorporated by reference in their entirety). Often, the antibody fragments present in such libraries comprise scFv antibody fragments, comprising a fusion protein of V_{H} and V_{L} antibody domains joined by a flexible linker. In some cases, scFvs may contain the same sequence with the exception of unique sequences encoding variable loops of the CDRs. In some cases, scFvs are expressed as fusion proteins, linked to viral coat proteins (e.g. the N-terminus of the viral pIII coat protein). V_{L} chains may be expressed separately for assembly with V_{H} chains in the periplasm prior to complex incorporation into viral coats. Precipitated library members may be sequenced from the bound phage to obtain cDNA encoding desired scFvs. Antibody variable domains or CDRs from such sequences may be directly incorporated into antibody sequences for recombinant antibody production or mutated and utilized for further optimization through in vitro affinity maturation.

In some embodiments, the sequences of the polypeptides to be encoded in the viral genomes may be produced using yeast surface display technology, wherein antibody variable domain sequences may be expressed on the cell surface of Saccharomyces cerevisiae. Recombinant antibodies may be developed by displaying the antibody fragment of interest as a fusion to e.g. Aga2p protein on the surface of the yeast, where the protein interacts with proteins and small molecules in a solution. scFvs with affinity toward desired receptors may be isolated from the yeast surface using magnetic separation and flow cytometry. Several cycles of yeast surface display and isolation may be done to attain scFvs with desired properties through directed evolution.

In some embodiments, the sequence of the polypeptides to be encoded in the viral genomes (e.g., antibodies) may be designed by VERSITOPE™ Antibody Generation and other methods used by BIOATLA® and described in United States Patent Publication No. US20130281303, the contents of which are herein incorporated by reference in their entirety. In brief, recombinant monoclonal antibodies are derived from B-cells of a host immuno-challenged with one or more target antigens. These methods of antibody generation do not rely on immortalized cell lines, such as hybridoma, thereby avoiding some of the associated challenges i.e., genetic instability and low production capacity, producing high affinity and high diversity recombinant monoclonal antibodies. In some embodiments, the method is a natural diversity approach. In another embodiment, the method is a high diversity approach.

In some embodiments, the sequences of the polypeptides to be encoded in the viral genomes may be generated using the BIOATLA® natural diversity approach. In the natural diversity approach of generating recombinant monoclonal antibodies described in United States Patent Publication No. US20130281303, the original pairings of variable heavy (V_{H}) and variable light (V_{L}) domains are retained from the host, yielding recombinant monoclonal antibodies that are naturally paired. These may be advantageous due to a higher likelihood of functionality as compared to non-natural pairings of V_{H} and V_{L}. To produce the recombinant monoclonal antibodies, first a non-human host (i.e., rabbit, mouse, hamster, guinea pig, camel or goat) is immuno-challenged with an antigen of interest. In some embodiments, the host may be a previously challenged human patient. In other embodiments, the host may not have been immuno-challenged. B-cells are harvested from the host and screened by fluorescence activated cell sorting (FACS), or other method, to create a library of B-cells enriched in B-cells capable of binding the target antigen. The cDNA obtained from the mRNA of a single B-cell is then amplified to generate an immunoglobulin library of V_{H} and V_{L} domains. This library of immunoglobulins is then cloned into expression vectors capable of expressing the V_{H} and V_{L}domains, wherein the V_{H} and V_{L} domains remain naturally paired. The library of expression vectors is then used in an expression system to express the V_{H} and V_{L} domains in order to create an antibody library. Screening of the antibody library yields antibodies able to bind the target antigen, and these antibodies can be further characterized. Characterization may include one or more of the following: isoelectric point, thermal stability, sedimentation rate, folding rate, neutralization or antigen activity, antagonist or agonistic activity, expression level, specific and non-specific binding, inhibition of enzymatic activity, rigidity/flexibility, shape, charge, stability across pH, in solvents, under UV radiation, in mechanical stress conditions, or in sonic conditions, half-life, and glycosylation.

In some embodiments, the sequences of the polypeptides to be encoded in the viral genomes may be generated using the BIOATLA® high diversity approach. In the high diversity approach of generating recombinant monoclonal antibodies described in United States Patent Publication No. US20130281303, additional pairings of variable heavy (V_{H}) and variable light (V_{L}) domains are attained. To produce the recombinant monoclonal antibodies, B-cells harvested from the host are screened by fluorescence activated cell sorting (FACS), panning, or other method, to create a library of B-cells enriched in B-cells capable of binding the target antigen. The cDNA obtained from the mRNA of the pooled B-cells is then amplified to generate an immunoglobulin library of V_{H} and V_{L} domains. This library of immunoglobulins is then used in a biological display system (mammalian, yeast or bacterial cell surface display systems) to generate a population of cells displaying antibodies, fragments or derivatives comprising the V_{H} and V_{L} domains wherein, the antibodies, fragments or derivatives comprise V_{H} and V_{L} domain combinations that were not present in the B-cells in vivo. Screening of the cell population by FACS, with the target antigen, yields a subset of cells capable of binding the target antigen and the antibodies displayed on these cells can be further characterized. In an alternate embodiment of the high diversity approach, the immunoglobulin library comprises only V_{H} domains obtained from the B-cells of the immuno-challenged host, while the V_{L} domain(s) are obtained from another source.

In some embodiments, the sequences of the polypeptides to be encoded in the viral genomes may be evolved using BIOATLA® comprehensive approaches. The methods of generating recombinant monoclonal antibodies as described in United States Patent Publication No. US20130281303, further comprises evolving the recombinant antibody by comprehensive positional evolution (CPE™), CPE™ followed by comprehensive protein synthesis (CPS™) PCR shuffling, or other method.

In some embodiments, the sequence of the polypeptides to be encoded in the viral genomes (e.g., antibodies) may be derived from any of the BIOATLA® protein evolution methods described in International Publication WO2012009026, the contents of which are herein incorporated by reference in their entirety. In this method, mutations are systematically performed throughout the polypeptide or molecule of interest, a map is created providing useful informatics to guide the subsequent evolutionary steps. Not wishing to be bound by theory, these evolutionary methods typically start with a template polypeptide and a mutant is derived therefrom, which has desirable properties or characteristics. Non-limiting examples of evolutionary techniques include polymerase chain reaction (PCR), error prone PCR, oligonucleotide-directed mutagenesis, cassette mutagenesis, shuffling, assembly PCR, sexual PCR mutagenesis, in vivo mutagenesis, site-specific mutagenesis, gene reassembly, gene site saturated mutagenesis, in vitro mutagenesis, ligase chain reaction, oligonucleotide synthesis or any combination thereof.

In some embodiments, the BIOATLA® evolution method is Comprehensive Positional Evolution (CPE™). In CPE, naturally occurring amino acid variants are generated for each of the codons of the template polypeptide, wherein 63 different codon options exist for each amino acid variant. A set of polypeptides with single amino acid mutations are generated and the mutations are then confirmed by sequencing or other method known in the art and each amino acid change screened for improved function, neutral mutations, inhibitory mutations, expression, and compatibility with the host system. An EvoMap™ is created that describes in detail the effects of each amino acid mutation on the properties and characteristics of that polypeptide. The data from the EvoMap™ may be utilized to produce polypeptides with more than one amino acid mutation, wherein the resultant multi-site mutant polypeptides can be screened for desirable characteristics.

In some embodiments, the BIOATLA® evolution method is Synergy Evolution, wherein an EvoMap™ is used to identify amino acid positions to introduce 2-20 mutations simultaneously to produce a combinatorial effect. The resulting multi-site mutant polypeptides may be screened on one or more pre-determined characteristics to identify “upmutants” wherein the function of the mutant is improved as compared to the parent polypeptide. In some embodiments, Synergy Evolution is used to enhance binding affinity of an antibody.

In some embodiments, the BIOATLA® evolution method is Flex Evolution, wherein an EvoMap™ is used to identify fully mutable sites within a polypeptide that may then be targeted for alteration, such as introduction of glycosylation sites or chemical conjugation.

In some embodiments, the BIOATLA® evolution method is Comprehensive Positional Insertion Evolution (CPI™), wherein an amino acid is inserted after each amino acid of a template polypeptide to generate a set of lengthened polypeptides. CPI may be used to insert 1, 2, 3, 4, or 5 amino acids at each new position. The resultant lengthened polypeptides are sequenced and assayed for one or more pre-determined properties and evaluated in comparison to its template or parent molecule. In some embodiments, the binding affinity and immunogenicity of the resultant polypeptides are assayed. In some embodiments, the lengthened polypeptides are further mutated and mapped to identify polypeptides with desirable characteristics.

In some embodiments, the BIOATLA® evolution approach is Comprehensive Positional Deletion Evolution (CPD™), wherein each amino acid of the template polypeptide is individually and systematically deleted one at a time. The resultant shortened polypeptides are then sequenced and evaluated by assay for at least one pre-determined feature. In some embodiments, the shortened polypeptides are further mutated and mapped to identify polypeptides with desirable characteristics.

In some embodiments, the BIOATLA® evolution approach is Combinatorial Protein Synthesis (CPS™), wherein mutants identified in CPE, CPI, CPD, or other evolutionary techniques are combined for polypeptide synthesis. These combined mutant polypeptides are then screened for enhanced properties and characteristics. In some embodiments CPS is combined with any of the aforementioned evolutionary or polypeptide synthesis methods.

In some embodiments, the sequence of the polypeptides to be encoded in the viral genomes (e.g., antibodies) may be derived from the BIOATLA® Comprehensive Integrated Antibody Optimization (CIAO!™) described in U.S. Pat. No. 8,859,467, the contents of which are herein incorporated by reference in their entirety. The CIAO!™ method allows for simultaneous evolution of polypeptide performance and expression optimization, within a eukaryotic cell host (i.e., mammalian or yeast cell host). First, an antibody library is generated in a mammalian cell production host by antibody cell surface display, wherein the generated antibody library targets a particular antigen of interest. The antibody library is then screened by any method known in the art, for one or more properties or characteristics. One or more antibodies of the library, with desirable properties or characteristics are chosen for further polypeptide evolution by any of the methods known in the art, to produce a library of mutant antibodies by antibody cell surface display in a mammalian cell production host. The generated mutant antibodies are screened for one or more predetermined properties or characteristics, whereby an upmutant is selected, wherein the upmutant has enhanced or improved characteristics as compared to the parent template polypeptide.

In some embodiments, the sequences of the polypeptides to be encoded in the viral genomes may be humanized by the methods of BIOATLA® as described in United States Patent Publication US20130303399, the contents of which are herein incorporated by reference in their entirety. In this method, for generating enhanced full-length humanized antibodies in mammalian cells, no back-mutations are required to retain affinity to the antigen and no CDR grafting or phage-display is necessary. The generated humanized antibody has reduced immunogenicity and equal or greater affinity for the target antigen as compared to the parent antibody. The variable regions or CDRs of the generated humanized antibody are derived from the parent or template, whereas the framework and constant regions are derived from one or more human antibodies. To start, the parent, or template antibody is selected, cloned and each CDR sequence identified and synthesized into a CDR fragment library. Double stranded DNA fragment libraries for V_{H} and V_{L} are synthesized from the CDR fragment encoding libraries, wherein at least one CDR fragment library is derived from the template antibody and framework (FW) fragment encoding libraries, wherein the FW fragment library is derived from a pool of human frameworks obtained from natively expressed and functional human antibodies. Stepwise liquid phase ligation of FW and CDR encoding fragments is then used to generate both V_{H} and V_{L} fragment libraries. The V_{H} and V_{L} fragment libraries are then cloned into expression vectors to create a humanization library, which is further transfected into cells for expression of full length humanized antibodies and used to create a humanized antibody library. The humanized antibody library is then screened to determine expression level of the humanized antibodies, affinity or binding ability for the antigen, and additional improved or enhanced characteristics, as compared to the template or parent antibody. Non-limiting examples of characteristics that may be screened include equilibrium dissociation constant (K_{D}), stability, melting temperature (Tₘ), pI, solubility, expression level, reduced immunogenicity, and improved effector function.

In some embodiments, the sequences of the polypeptides to be encoded in the viral genomes may be generated by the BIOATLA® method for preparing conditionally active antibodies as described in International Publications WO2016033331 and WO2016036916, the contents of which are herein incorporated by reference in their entirety. As used herein, the term “conditionally active” refers to a molecule that is active at an aberrant condition. Further, the conditionally active molecule may be virtually inactive at normal physiological conditions. Aberrant conditions may result from changes in pH, temperature, osmotic pressure, osmolality, oxidative stress, electrolyte concentration, and/or chemical or proteolytic resistance, as non-limiting examples.

The method of preparing a conditionally active antibody is described in International Publications WO2016033331 and WO2016036916 and summarized herein. Briefly, a wild-type polypeptide is selected and the DNA is evolved to create mutant DNAs. Non-limiting examples of evolutionary techniques that may be used to evolve the DNA include polymerase chain reaction (PCR), error prone PCR, shuffling, oligonucleotide-directed mutagenesis, assembly PCR, sexual PCR mutagenesis, in vivo mutagenesis, site-specific mutagenesis, gene reassembly, gene site saturated mutagenesis, in vitro mutagenesis, ligase chain reaction, oligonucleotide synthesis or any combination thereof. Once mutant DNAs are created, they are expressed in a eukaryotic cell production host (i.e., fungal, insect, mammalian, adenoviral, plant), wherein a mutant polypeptide is produced. The mutant polypeptide and the corresponding wild-type polypeptide are then subjected to assays under both normal physiological conditions and aberrant conditions in order to identify mutants that exhibit a decrease in activity in the assay at normal physiological conditions as compared to the wild-type polypeptide and/or an increase in activity in the assay under aberrant conditions, as compared to the corresponding wild-type polypeptide. The desired conditionally active mutant may then be produced in the aforementioned eukaryotic cell production host.

In some embodiments, the conditionally active antibody is a “mirac protein” as described by BIOATLA® in U.S. Pat. No. 8,709,755, the contents of which are herein incorporated by reference in their entirety. As used herein “mirac protein” refers to a conditionally active antibody that is virtually inactive at body temperature but active at lower temperatures.

In some embodiments, the sequence of the polypeptides to be encoded in the viral genomes (e.g., antibodies) may be derived based on any of the BIOATLA™ methods including, but not limited to, VERSITOPE™ Antibody Generation, natural diversity approaches, and high diversity approaches for generating monoclonal antibodies, methods for generation of conditionally active polypeptides, humanized antibodies, mirac proteins, multi-specific antibodies or cross-species active mutant polypeptides, Comprehensive Integrated Antibody Optimization (CIAO!™), Comprehensive Positional Evolution (CPE™), Synergy Evolution, Flex Evolution, Comprehensive Positional Insertion Evolution (CPI™), Comprehensive Positional Deletion Evolution (CPD™), Combinatorial Protein Synthesis (CPS™), or any combination thereof. These methods are described in U.S. Pat. Nos. 8,859,467 and 8,709,755 and United States Publication Nos. US20130281303, US20130303399, US20150065690, US20150252119, US20150086562 and US20100138945, and International Publication Nos. WO2015105888, WO2012009026, WO2011109726, WO2016036916, and WO2016033331, the contents of each of which are herein incorporated by reference in their entirety.

In some embodiments, antibodies of the present disclosure are generated by any of the aforementioned means to target one or more of the following epitopes of the tau protein; phosphorylated tau peptides, pS396, pS396-pS404, pS404, pS396-pS404-pS422, pS422, pS199, pS199-pS202, pS202, pT181, pT231, cis-pT231, any of the following acetylated sites acK174, acK274, acK280, acK281 and/or any combination thereof

### Antibody Fragments and Variants

In some embodiments, antibody fragments encoded by payloads comprise antigen binding regions from intact antibodies. Examples of antibody fragments may include, but are not limited to Fab, Fab′, F(ab′)₂, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)2 fragment that has two antigen-binding sites and is still capable of cross-linking antigen. Compounds and/or compositions of the present disclosure may comprise one or more of these fragments. For the purposes herein, an “antibody” may comprise a heavy and light variable domain as well as an Fc region.

In some embodiments, the Fc region may be a modified Fc region, as described in US Patent Publication US20150065690, wherein the Fc region may have a single amino acid substitution as compared to the corresponding sequence for the wild-type Fc region, wherein the single amino acid substitution yields an Fc region with preferred properties to those of the wild-type Fc region. Non-limiting examples of Fc properties that may be altered by the single amino acid substitution include bind properties or response to pH conditions

As used herein, the term “native antibody” refers to a usually heterotetrameric glycoprotein of about 150,000 Daltons, composed of two identical light (L) chains and two identical heavy (H) chains. Genes encoding antibody heavy and light chains are known and segments making up each have been well characterized and described (Matsuda, F. et al., 1998. The Journal of Experimental Medicine. 188(11); 2151-62 and Li, A. et al., 2004. Blood. 103(12: 4602-9, the content of each of which are herein incorporated by reference in their entirety). Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains found on both the antibody heavy and light chains that differ extensively in sequence among antibodies and are used in the binding and specificity of each particular antibody for its particular antigen. Variable domains comprise hypervariable regions. As used herein, the term “hypervariable region” refers to a region within a variable domain comprising amino acid residues responsible for antigen binding. The amino acids present within the hypervariable regions determine the structure of the complementarity determining regions (CDRs) that become part of the antigen-binding site of the antibody. As used herein, the term “CDR” refers to a region of an antibody comprising a structure that is complimentary to its target antigen or epitope. Other portions of the variable domain, not interacting with the antigen, are referred to as framework (FW) regions. The antigen-binding site (also known as the antigen combining site or paratope) comprises the amino acid residues necessary to interact with a particular antigen. The exact residues making up the antigen-binding site are typically elucidated by co-crystallography with bound antigen, however computational assessments can also be used based on comparisons with other antibodies (Strohl, W. R. Therapeutic Antibody Engineering. Woodhead Publishing, Philadelphia PA. 2012. Ch. 3, p 47-54, the contents of which are herein incorporated by reference in their entirety). Determining residues making up CDRs may include the use of numbering schemes including, but not limited to, those taught by Kabat [Wu, T. T. et al., 1970, JEM, 132(2):211-50 and Johnson, G. et al., 2000, Nucleic Acids Res. 28(1): 214-8, the contents of each of which are herein incorporated by reference in their entirety], Chothia [Chothia and Lesk, J. Mol. Biol. 196, 901 (1987), Chothia et al., Nature 342, 877 (1989) and Al-Lazikani, B. et al., 1997, J. Mol. Biol. 273(4):927-48, the contents of each of which are herein incorporated by reference in their entirety], Lefranc (Lefranc, M. P. et al., 2005, Immunome Res. 1:3) and Honegger (Honegger, A. and Pluckthun, A. 2001. J. Mol. Biol. 309(3):657-70, the contents of which are herein incorporated by reference in their entirety).

V_{H} and V_{L} domains have three CDRs each. V_{L} CDRs are referred to herein as CDR-L1, CDR-L2 and CDR-L3, in order of occurrence when moving from N- to C-terminus along the variable domain polypeptide. V_{H} CDRs are referred to herein as CDR-H1, CDR-H2, and CDR-H3, in order of occurrence when moving from N- to C-terminus along the variable domain polypeptide. Each of CDRs have favored canonical structures with the exception of the CDR-H3, which comprises amino acid sequences that may be highly variable in sequence and length between antibodies resulting in a variety of three-dimensional structures in antigen-binding domains (Nikoloudis, D. et al., 2014. Peer J. 2:e456; the contents of which are herein incorporated by reference in their entirety). In some cases, CDR-H3s may be analyzed among a panel of related antibodies to assess antibody diversity. Various methods of determining CDR sequences are known in the art and may be applied to known antibody sequences (Strohl, W. R. Therapeutic Antibody Engineering. Woodhead Publishing, Philadelphia PA. 2012. Ch. 3, p 47-54, the contents of which are herein incorporated by reference in their entirety).

As used herein, the term “Fv” refers to an antibody fragment comprising the minimum fragment on an antibody needed to form a complete antigen-binding site. These regions consist of a dimer of one heavy chain and one light chain variable domain in tight, non-covalent association. Fv fragments can be generated by proteolytic cleavage but are largely unstable. Recombinant methods are known in the art for generating stable Fv fragments, typically through insertion of a flexible linker between the light chain variable domain and the heavy chain variable domain [to form a single chain Fv (scFv)] or through the introduction of a disulfide bridge between heavy and light chain variable domains (Strohl, W. R. Therapeutic Antibody Engineering. Woodhead Publishing, Philadelphia PA. 2012. Ch. 3, p 46-47, the contents of which are herein incorporated by reference in their entirety).

As used herein, the term “light chain” refers to a component of an antibody from any vertebrate species assigned to one of two clearly distinct types, called kappa and lambda based on amino acid sequences of constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

As used herein, the term “single chain Fv” or “scFv” refers to a fusion protein of V_{H} and V_{L} antibody domains, wherein these domains are linked together into a single polypeptide chain by a flexible peptide linker. In some embodiments, the Fv polypeptide linker enables the scFv to form the desired structure for antigen binding. In some embodiments, scFvs are utilized in conjunction with phage display, yeast display or other display methods where they may be expressed in association with a surface member (e.g. phage coat protein) and used in the identification of high affinity peptides for a given antigen.

As used herein, the term “bispecific antibody” refers to an antibody capable of binding two different antigens. Such antibodies typically comprise regions from at least two different antibodies. Bispecific antibodies may include any of those described in Riethmuller, G. 2012. Cancer Immunity. 12:12-18, Marvin, J. S. et al., 2005. Acta Pharmacologica Sinica. 26(6):649-58 and Schaefer, W. et al., 2011. PNAS. 108(27):11187-92, the contents of each of which are herein incorporated by reference in their entirety.

As used herein, the term “diabody” refers to a small antibody fragment with two antigen-binding sites. Diabodies comprise a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites. Diabodies are described more fully in, for example, EP 404097; WO 9311161; and Hollinger et al. (Hollinger, P. et al., “Diabodies”: Small bivalent and bispecific antibody fragments. PNAS. 1993. 90:6444-8) the contents of each of which are incorporated herein by reference in their entirety.

The term “intrabody” refers to a form of antibody that is not secreted from a cell in which it is produced, but instead targets one or more intracellular proteins. Intrabodies may be used to affect a multitude of cellular processes including, but not limited to intracellular trafficking, transcription, translation, metabolic processes, proliferative signaling, and cell division. In some embodiments, methods of the present disclosure may include intrabody-based therapies. In some such embodiments, variable domain sequences and/or CDR sequences disclosed herein may be incorporated into one or more constructs for intrabody-based therapy.

As used herein, the term “monoclonal antibody” refers to an antibody obtained from a population of substantially homogeneous cells (or clones), i.e., the individual antibodies comprising the population are identical and/or bind the same epitope, except for possible variants that may arise during production of the monoclonal antibodies, such variants generally being present in minor amounts. In contrast to polyclonal antibody preparations that typically include different antibodies directed against different determinants (epitopes), each monoclonal antibody is directed against a single determinant on the antigen

The modifier “monoclonal” indicates the character of the antibody as being obtained from a substantially homogeneous population of antibodies and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

As used herein, the term “humanized antibody” refers to a chimeric antibody comprising a minimal portion from one or more non-human (e.g., murine) antibody source(s) with the remainder derived from one or more human immunoglobulin sources. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and/or capacity.

In some embodiments, viral genomes of the present disclosure may encode antibody mimetics. As used herein, the term “antibody mimetic” refers to any molecule which mimics the function or effect of an antibody and which binds specifically and with high affinity to their molecular targets. In some embodiments, antibody mimetics may be monobodies, designed to incorporate the fibronectin type III domain (Fn3) as a protein scaffold (U.S. Pat. Nos. 6,673,901; 6,348,584). In some embodiments, antibody mimetics may be those known in the art including, but are not limited to affibody molecules, affilins, affitins, anticalins, avimers, Centyrins, DARPINS™, fynomers, Kunitz domains, and domain peptides. In other embodiments, antibody mimetics may include one or more non-peptide regions.

As used herein, the term “antibody variant” refers to a modified antibody (in relation to a native or starting antibody) or a biomolecule resembling a native or starting antibody in structure and/or function (e.g., an antibody mimetic). Antibody variants may be altered in their amino acid sequence, composition, or structure as compared to a native antibody. Antibody variants may include, but are not limited to, antibodies with altered isotypes (e.g., IgA, IgD, IgE, IgG₁, IgG₂, IgG₃, IgG₄, or IgM), humanized variants, optimized variants, multispecific antibody variants (e.g., bispecific variants), and antibody fragments.

The preparation of antibodies, whether monoclonal or polyclonal, is known in the art. Techniques for the production of antibodies are well known in the art and described, e.g. in Harlow and Lane “Antibodies, A Laboratory Manual”, Cold Spring Harbor Laboratory Press, 1988; Harlow and Lane “Using Antibodies: A Laboratory Manual” Cold Spring Harbor Laboratory Press, 1999 and “Therapeutic Antibody Engineering: Current and Future Advances Driving the Strongest Growth Area in the Pharmaceutical Industry” Woodhead Publishing, 2012.

### Multispecific Antibodies

In some embodiments, payloads may encode antibodies that bind more than one epitope. As used herein, the terms “multibody” or “multispecific antibody” refer to an antibody wherein two or more variable regions bind to different epitopes. The epitopes may be on the same or different targets. In certain embodiments, a multi-specific antibody is a “bispecific antibody,” which recognizes two different epitopes on the same or different antigens.

In some embodiments, multi-specific antibodies may be prepared by the methods used by BIOATLA® and described in International Patent publication WO201109726, the contents of which are herein incorporated by reference in their entirety. First a library of homologous, naturally occurring antibodies is generated by any method known in the art (i.e., mammalian cell surface display), then screened by FACSAria or another screening method, for multi-specific antibodies that specifically bind to two or more target antigens. In some embodiments, the identified multi-specific antibodies are further evolved by any method known in the art, to produce a set of modified multi-specific antibodies. These modified multi-specific antibodies are screened for binding to the target antigens. In some embodiments, the multi-specific antibody may be further optimized by screening the evolved modified multi-specific antibodies for optimized or desired characteristics.

In some embodiments, multi-specific antibodies may be prepared by the methods used by BIOATLA® and described in Unites States Publication No. US20150252119, the contents of which are herein incorporated by reference in their entirety. In one approach, the variable domains of two parent antibodies, wherein the parent antibodies are monoclonal antibodies are evolved using any method known in the art in a manner that allows a single light chain to functionally complement heavy chains of two different parent antibodies. Another approach requires evolving the heavy chain of a single parent antibody to recognize a second target antigen. A third approach involves evolving the light chain of a parent antibody so as to recognize a second target antigen. Methods for polypeptide evolution are described in International Publication WO2012009026, the contents of which are herein incorporated by reference in their entirety, and include as non-limiting examples, Comprehensive Positional Evolution (CPE), Combinatorial Protein Synthesis (CPS), Comprehensive Positional Insertion (CPI), Comprehensive Positional Deletion (CPD), or any combination thereof. The Fc region of the multi-specific antibodies described in United States Publication No. US20150252119 may be created using a knob-in-hole approach, or any other method that allows the Fc domain to form heterodimers. The resultant multi-specific antibodies may be further evolved for improved characteristics or properties such as binding affinity for the target antigen.

### Bispecific Antibodies

In some embodiments, payloads may encode bispecific antibodies. Bispecific antibodies are capable of binding two different antigens. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen.

In some cases, payloads encode bispecific antibodies comprising antigen-binding regions from two different anti-tau antibodies. For example, such bispecific antibodies may comprise binding regions from two different antibodies selected from Table 3.

Bispecific antibody frameworks may include any of those described in Riethmuller, G., 2012. Cancer Immunity. 12:12-18; Marvin, J. S. et al., 2005. Acta Pharmacologica Sinica. 26(6):649-58; and Schaefer, W. et al., 2011. PNAS. 108(27):11187-92, the contents of each of which are herein incorporated by reference in their entirety.

New generations of BsMAb, called “trifunctional bispecific” antibodies, have been developed. These consist of two heavy and two light chains, one each from two different antibodies, where the two Fab regions (the arms) are directed against two antigens, and the Fc region (the foot) comprises the two heavy chains and forms the third binding site.

Of the two paratopes that form the tops of the variable domains of a bispecific antibody, one can be directed against a target antigen and the other against a T-lymphocyte antigen like CD3. In the case of trifunctional antibodies, the Fc region may additionally bind to a cell that expresses Fc receptors, like a macrophage, a natural killer (NK) cell or a dendritic cell. In sum, the targeted cell is connected to one or two cells of the immune system, which subsequently destroy it.

Other types of bispecific antibodies have been designed to overcome certain problems, such as short half-life, immunogenicity and side-effects caused by cytokine liberation. They include chemically linked Fabs, consisting only of the Fab regions, and various types of bivalent and trivalent single-chain variable fragments (scFvs), fusion proteins mimicking the variable domains of two antibodies. The furthest developed of these newer formats are the bispecific T-cell engagers (BiTEs) and mAb2's, antibodies engineered to contain an Fcab antigen-binding fragment instead of the Fc constant region.

Using molecular genetics, two scFvs can be engineered in tandem into a single polypeptide, separated by a linker domain, called a “tandem scFv” (tascFv). TascFvs have been found to be poorly soluble and require refolding when produced in bacteria, or they may be manufactured in mammalian cell culture systems, which avoids refolding requirements but may result in poor yields. Construction of a tascFv with genes for two different scFvs yields a “bispecific single-chain variable fragments” (bis-scFvs). Only two tascFvs have been developed clinically by commercial firms; both are bispecific agents in active early phase development by Micromet for oncologic indications and are described as “Bispecific T-cell Engagers (BiTE).” Blinatumomab is an anti-CD19/anti-CD3 bispecific tascFv that potentiates T-cell responses to B-cell non-Hodgkin lymphoma in Phase 2. MT110 is an anti-EP-CAM/anti-CD3 bispecific tascFv that potentiates T-cell responses to solid tumors in Phase 1. Bispecific, tetravalent “TandAbs” are also being researched by Affimed (Nelson, A. L., MAbs. 2010. January-February; 2(1):77-83).

In some embodiments, payloads may encode antibodies comprising a single antigen-binding domain. These molecules are extremely small, with molecular weights approximately one-tenth of those observed for full-sized mAbs. Further antibodies may include “nanobodies” derived from the antigen-binding variable heavy chain regions (V_{HH}S) of heavy chain antibodies found in camels and llamas, which lack light chains (Nelson, A. L., MAbs. 2010. January-February; 2(1):77-83).

Disclosed and claimed in PCT Publication WO2014144573 to Memorial Sloan-Kettering Cancer Center are multimerization technologies for making dimeric multispecific binding agents (e.g., fusion proteins comprising antibody components) with improved properties over multispecific binding agents without the capability of dimerization.

In some cases, payloads may encode tetravalent bispecific antibodies (TetBiAbs as disclosed and claimed in PCT Publication WO2014144357). TetBiAbs feature a second pair of Fab fragments with a second antigen specificity attached to the C-terminus of an antibody, thus providing a molecule that is bivalent for each of the two antigen specificities. The tetravalent antibody is produced by genetic engineering methods, by linking an antibody heavy chain covalently to a Fab light chain, which associates with its cognate, co-expressed Fab heavy chain.

In some aspects, payloads may encode biosynthetic antibodies as described in U.S. Pat. No. 5,091,513, the contents of which are herein incorporated by reference in their entirety. Such antibody may include one or more sequences of amino acids constituting a region which behaves as a biosynthetic antibody binding site (BABS). The sites comprise 1) non-covalently associated or disulfide bonded synthetic V_{H} and V_{L} dimers, 2) V_{H}-V_{L} or V_{L}-V_{H} single chains wherein the V_{H} and V_{L} are attached by a polypeptide linker, or 3) individuals V_{H} or V_{L} domains. The binding domains comprise linked CDR and FR regions, which may be derived from separate immunoglobulins. The biosynthetic antibodies may also include other polypeptide sequences which function, e.g., as an enzyme, toxin, binding site, or site of attachment to an immobilization media or radioactive atom. Methods are disclosed for producing the biosynthetic antibodies, for designing BABS having any specificity that can be elicited by in vivo generation of antibody, and for producing analogs thereof.

In some embodiments, payloads may encode antibodies with antibody acceptor frameworks taught in U.S. Pat. No. 8,399,625. Such antibody acceptor frameworks may be particularly well suited accepting CDRs from an antibody of interest. In some cases, CDRs from anti-tau antibodies known in the art or developed according to the methods presented herein may be used.

### Miniaturized Antibody

In some embodiments, the antibody encoded by the payloads may be a “miniaturized” antibody. Among the best examples of mAb miniaturization are the small modular immunopharmaceuticals (SMIPs) from Trubion Pharmaceuticals. These molecules, which can be monovalent or bivalent, are recombinant single-chain molecules containing one V_{L}, one V_{H} antigen-binding domain, and one or two constant “effector” domains, all connected by linker domains. Presumably, such a molecule might offer the advantages of increased tissue or tumor penetration claimed by fragments while retaining the immune effector functions conferred by constant domains. At least three “miniaturized” SMIPs have entered clinical development. TRU-015, an anti-CD20 SMIP developed in collaboration with Wyeth, is the most advanced project, having progressed to Phase 2 for rheumatoid arthritis (RA). Earlier attempts in systemic lupus erythrematosus (SLE) and B cell lymphomas were ultimately discontinued. Trubion and Facet Biotechnology are collaborating in the development of TRU-016, an anti-CD37 SMIP, for the treatment of CLL and other lymphoid neoplasias, a project that has reached Phase 2. Wyeth has licensed the anti-CD20 SMIP SBI-087 for the treatment of autoimmune diseases, including RA, SLE, and possibly multiple sclerosis, although these projects remain in the earliest stages of clinical testing. (Nelson, A. L., MAbs. 2010. January-February; 2(1):77-83).

### Diabodies

In some embodiments, payloads may encode diabodies. Diabodies are functional bispecific single-chain antibodies (bscAb). These bivalent antigen-binding molecules are composed of non-covalent dimers of scFvs, and can be produced in mammalian cells using recombinant methods. (See, e.g., Mack et al, Proc. Natl. Acad. Sci., 92: 7021-7025, 1995). Few diabodies have entered clinical development. An iodine-123-labeled diabody version of the anti-CEA chimeric antibody cT84.66 has been evaluated for pre-surgical immunoscintigraphic detection of colorectal cancer in a study sponsored by the Beckman Research Institute of the City of Hope (Clinicaltrials.gov NCT00647153) (Nelson, A. L., MAbs., 2010. January-February; 2(1):77-83).

### Unibody

In some embodiments, payloads may encode a “unibody,” in which the hinge region has been removed from IgG4 molecules. While IgG4 molecules are unstable and can exchange light-heavy chain heterodimers with one another, deletion of the hinge region prevents heavy chain-heavy chain pairing entirely, leaving highly specific monovalent light/heavy heterodimers, while retaining the Fc region to ensure stability and half-life in vivo. This configuration may minimize the risk of immune activation or oncogenic growth, as IgG4 interacts poorly with FcRs and monovalent unibodies fail to promote intracellular signaling complex formation. These contentions are, however, largely supported by laboratory, rather than clinical, evidence. Other antibodies may be “miniaturized” antibodies, which are compacted 100 kDa antibodies (see, e.g., Nelson, A. L., MAbs., 2010. January-February; 2(1):77-83).

### Intrabodies

In some embodiments, payloads may encode intrabodies. Intrabodies are a form of antibody that is not secreted from a cell in which it is produced, but instead targets one or more intracellular proteins. Intrabodies are expressed and function intracellularly, and may be used to affect a multitude of cellular processes including, but not limited to intracellular trafficking, transcription, translation, metabolic processes, proliferative signaling and cell division. In some embodiments, methods described herein include intrabody-based therapies. In some such embodiments, variable domain sequences and/or CDR sequences disclosed herein are incorporated into one or more constructs for intrabody-based therapy. For example, intrabodies may target one or more glycated intracellular proteins or may modulate the interaction between one or more glycated intracellular proteins and an alternative protein.

More than two decades ago, intracellular antibodies against intracellular targets were first described (Biocca, Neuberger and Cattaneo EMBO J. 9: 101-108, 1990). The intracellular expression of intrabodies in different compartments of mammalian cells allows blocking or modulation of the function of endogenous molecules (Biocca, et al., EMBO J. 9: 101-108, 1990; Colby et al., Proc. Natl. Acad. Sci. U.S.A. 101: 17616-21, 2004). Intrabodies can alter protein folding, protein-protein, protein-DNA, protein-RNA interactions and protein modification. They can induce a phenotypic knockout and work as neutralizing agents by direct binding to the target antigen, by diverting its intracellular trafficking or by inhibiting its association with binding partners. They have been largely employed as research tools and are emerging as therapeutic molecules for the treatment of human diseases such as viral pathologies, cancer and misfolding diseases. The fast-growing bio-market of recombinant antibodies provides intrabodies with enhanced binding specificity, stability, and solubility, together with lower immunogenicity, for their use in therapy (Biocca, abstract in Antibody Expression and Production Cell Engineering Volume 7, 2011, pp. 179-195).

In some embodiments, intrabodies have advantages over interfering RNA (iRNA); for example, iRNA has been shown to exert multiple non-specific effects, whereas intrabodies have been shown to have high specificity and affinity to target antigens. Furthermore, as proteins, intrabodies possess a much longer active half-life than iRNA. Thus, when the active half-life of the intracellular target molecule is long, gene silencing through iRNA may be slow to yield an effect, whereas the effects of intrabody expression can be almost instantaneous. Lastly, it is possible to design intrabodies to block certain binding interactions of a particular target molecule, while sparing others.

Intrabodies are often single chain variable fragments (scFvs) expressed from a recombinant nucleic acid molecule and engineered to be retained intracellularly (e.g., retained in the cytoplasm, endoplasmic reticulum, or periplasm). Intrabodies may be used, for example, to ablate the function of a protein to which the intrabody binds. The expression of intrabodies may also be regulated through the use of inducible promoters in the nucleic acid expression vector comprising the intrabody. Intrabodies may be produced for use in the viral genomes using methods known in the art, such as those disclosed and reviewed in: (Marasco et al., 1993 Proc. Natl. Acad. Sci. USA, 90: 7889-7893; Chen et al., 1994, Hum. Gene Ther. 5:595-601; Chen et al., 1994, Proc. Natl. Acad. Sci. USA, 91: 5932-5936; Maciejewski et al., 1995, Nature Med., 1: 667-673; Marasco, 1995, Immunotech, 1: 1-19; Mhashilkar, et al., 1995, EMBO J. 14: 1542-51; Chen et al., 1996, Hum. Gene Therap., 7: 1515-1525; Marasco, Gene Ther. 4:11-15, 1997; Rondon and Marasco, 1997, Annu. Rev. Microbiol. 51:257-283; Cohen, et al., 1998, Oncogene 17:2445-56; Proba et al., 1998, J. Mol. Biol. 275:245-253; Cohen et al., 1998, Oncogene 17:2445-2456; Hassanzadeh, et al., 1998, FEBS Lett. 437:81-6; Richardson et al., 1998, Gene Ther. 5:635-44; Ohage and Steipe, 1999, J. Mol. Biol. 291:1119-1128; Ohage et al., 1999, J. Mol. Biol. 291:1129-1134; Wirtz and Steipe, 1999, Protein Sci. 8:2245-2250; Zhu et al., 1999, J. Immunol. Methods 231:207-222; Arafat et al., 2000, Cancer Gene Ther. 7:1250-6; der Maur et al., 2002, J. Biol. Chem. 277:45075-85; Mhashilkar et al., 2002, Gene Ther. 9:307-19; and Wheeler et al., 2003, FASEB J. 17: 1733-5; and references cited therein). In particular, a CCR5 intrabody has been produced by Steinberger et al., 2000, Proc. Natl. Acad. Sci. USA 97:805-810). See generally Marasco, W A, 1998, “Intrabodies: Basic Research and Clinical Gene Therapy Applications” Springer: New York; and for a review of scFvs, see Pluckthun in “The Pharmacology of Monoclonal Antibodies,” 1994, vol. 113, Rosenburg and Moore eds. Springer-Verlag, New York, pp. 269-315.

Sequences from donor antibodies may be used to develop intrabodies. Intrabodies are often recombinantly expressed as single domain fragments such as isolated V_{H} and V_{L} domains or as a single chain variable fragment (scFv) antibody within the cell. For example, intrabodies are often expressed as a single polypeptide to form a single chain antibody comprising the variable domains of the heavy and light chains joined by a flexible linker polypeptide. Intrabodies typically lack disulfide bonds and are capable of modulating the expression or activity of target genes through their specific binding activity. Single chain antibodies can also be expressed as a single chain variable region fragment joined to the light chain constant region.

As is known in the art, an intrabody can be engineered into recombinant polynucleotide vectors to encode sub-cellular trafficking signals at its N or C terminus to allow expression at high concentrations in the sub-cellular compartments where a target protein is located. For example, intrabodies targeted to the endoplasmic reticulum (ER) are engineered to incorporate a leader peptide and, optionally, a C-terminal ER retention signal, such as the KDEL amino acid motif (SEQ ID NO: 4545). Intrabodies intended to exert activity in the nucleus are engineered to include a nuclear localization signal. Lipid moieties are joined to intrabodies in order to tether the intrabody to the cytosolic side of the plasma membrane. Intrabodies can also be targeted to exert function in the cytosol. For example, cytosolic intrabodies are used to sequester factors within the cytosol, thereby preventing them from being transported to their natural cellular destination.

There are certain technical challenges with intrabody expression. In particular, protein conformational folding and structural stability of the newly synthesized intrabody within the cell is affected by reducing conditions of the intracellular environment.

Intrabodies may be promising therapeutic agents for the treatment of misfolding diseases, including Tauopathies, prion diseases, Alzheimer's, Parkinson's, and Huntington's, because of their virtually infinite ability to specifically recognize the different conformations of a protein, including pathological isoforms, and because they can be targeted to the potential sites of aggregation (both intra- and extracellular sites). These molecules can work as neutralizing agents against amyloidogenic proteins by preventing their aggregation, and/or as molecular shunters of intracellular traffic by rerouting the protein from its potential aggregation site (Cardinale, and Biocca, Curr. Mol. Med. 2008, 8:2-11).

### Maxibodies

In some embodiments, the payloads encode a maxibody (bivalent scFv fused to the amino terminus of the Fc (CH2-CH3 domains) of IgG.

### Chimeric Antigen Receptors

In some embodiments, the polypeptides encoded by the viral genomes (e.g., antibodies) may be used to generate chimeric antigen receptors (CARs) as described by BIOATLA® in International Publications WO2016033331 and WO2016036916, the contents of which are herein incorporated by reference in their entirety. As used herein, a “chimeric antigen receptor (CAR)” refers to an artificial chimeric protein comprising at least one antigen specific targeting region (ASTR), wherein the antigen specific targeting region comprises a full-length antibody or a fragment thereof that specifically binds to a target antigen. The ASTR may comprise any of the following: a full length heavy or light chain, an Fab fragment, a single chain Fv fragment, a divalent single chain antibody, or a diabody. As a non-limiting example, the ASTR of a CAR may be any of the antibodies listed in Table 3, antibody-based compositions or fragments thereof. Any molecule that is capable of binding a target antigen with high affinity can be used in the ASTR of a CAR. In some embodiments, the CAR may have more than one ASTR. These ASTRs may target two or more antigens or two or more epitopes of the same antigen. In some embodiments, the CAR is conditionally active. In some embodiments, the CAR is used to produce a genetically engineered cytotoxic cell carrying the CAR and capable of targeting the antigen bound by the ASTR.

Chimeric antigen receptors (CARs) are particularly useful in the treatment of cancers, though also therapeutically effective in treatment of a wide variety of other diseases and disorders. Non-limiting examples of disease categories that may be treated with CARs or CAR-based therapeutics include autoimmune disorders, B-cell mediated diseases, inflammatory diseases, neuronal disorders, cardiovascular disease and circulatory disorders, or infectious diseases. Not wishing to be bound by theory, CARs traditionally work by targeting antigens presented on the surface of or on the inside of cells to be destroyed e.g., cancer tumor cells, by the cytotoxic cell of the CAR.

### Senescent Cell Surface Protein Antibodies

In some embodiments, the AAV particles may comprise nucleic acids which have been engineered to express of antibodies that selectively bind to surface marker proteins of senescent cells. For example, the antibodies may selectively bind to proteins that are in misfolded conformation. The binding antibodies may reduce the number of senescent cells and be used to treat age-related conditions, such as, but not limited to, Alzheimer's disease, cardiovascular disease, emphysema, sarcopenia, and tumorigenesis as well as conditions more cosmetic in nature such as signs of skin aging including wrinkling, sagging, discoloration, age-related tissue dysfunction, tumor formation, and other age-related conditions.

In some embodiments, the expressed antibodies binding to epitopes of senescent cell surface proteins may be, but are not limited to, such as prion epitopes presented by SEQ ID NO: 1-14 of International Publication No. WO2014186878; CD44 epitopes presented by SEQ ID NO: 47-51 of International Publication No. WO2014186878; TNFR epitopes presented by SEQ ID NO: 52-56 of International Publication No. WO2014186878; NOTCHI epitope presented by SEQ ID NO: 57-61 of International Publication No. WO2014186878; FasR epitopes presented by SEQ ID NO: 62-66 of International Publication No. WO2014186878; epidermal growth factor epitopes presented by SEQ ID NO: 67-81 of International Publication No. WO2014186878; CD38 epitopes presented by SEQ ID NO: 82-86 of International Publication No. WO2014186878, the contents of each of which are herein incorporated by reference in their entirety.

In some embodiments, the expressed antibodies may comprise peptides binding to senescent cell surface prion proteins, such as, but not limited to, those presented by SEQ ID NO: 15-36 of International Publication No. WO2014186878, the contents of which are herein incorporated by reference in their entirety.

In some embodiments, the expressed antibody may be AMF-3a-118 or AMF 3d-19 (SEQ ID NO: 89-92 and 103-106 of International publication WO2014186878, respectively, the contents of which are herein incorporated by reference in their entirety) targeting senescent cell surface protein FasR. In some embodiments, the expressed antibody may be Ab c-120 (SEQ ID NO: 37-40 of International publication WO2014186878, the contents of which are herein incorporated by reference in their entirety) targeting senescent cell surface protein PrP.

### Payload Antibodies

In some embodiments, the payload region of the AAV particle comprises one or more nucleic acid sequences encoding tau associated disease antibodies, variants or fragments thereof.

In some embodiments, the payload region of the AAV particle comprises one or more nucleic acid sequences encoding one or more of the payload antibody polypeptides listed in Table 3, or variants or fragments thereof. As used herein, “antibody polynucleotide” refers to a nucleic acid sequence encoding an antibody polypeptide.

In some embodiments, the payload region of the AAV particle comprises one or more nucleic acid sequences listed in Table 3, or variants or fragments thereof.

In some embodiments, the payload region of the AAV particle comprises a nucleic acid sequence encoding a payload antibody with at least 50% identity to one or more payload antibody polypeptides listed in Tables 3. The encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 3, or variants or fragments thereof.

In some embodiments, the full sequence of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 3, or variants or fragments thereof.

In some embodiments, the variable region sequence(s) of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload antibody polypeptides listed in Table 3, or variants or fragments thereof.

In some embodiments, the heavy chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload heavy chain antibody polypeptides listed in Table 3, or variants or fragments thereof.

In some embodiments, the light chain of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more of the payload light chain antibody polypeptides listed in Table 3, or variants or fragments thereof.

In some embodiments, the CDR region of the encoded antibody polypeptide may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to the CDRs of one or more of the payload antibody polypeptides listed in Table 3, or variants or fragments thereof.

In some embodiments, the payload antibody has 90% identity to one or more of the antibody polypeptides listed in Table 3, or variants or fragments thereof.

In some embodiments, the payload antibody has 91% identity to one or more of the antibody polypeptides listed in Table 3, or variants or fragments thereof.

In some embodiments, the payload antibody has 92% identity to one or more of the antibody polypeptides listed in Table 3, or variants or fragments thereof.

In some embodiments, the payload antibody has 93% identity to one or more of the antibody polypeptides listed in Table 3, or variants or fragments thereof.

In some embodiments, the payload antibody has 94% identity to one or more of the antibody polypeptides listed in Table 3, or variants or fragments thereof.

In some embodiments, the payload antibody has 95% identity to one or more of the antibody polypeptides listed in Table 3, or variants or fragments thereof.

In some embodiments, the payload antibody has 96% identity to one or more of the antibody polypeptides listed in Table 3, or variants or fragments thereof.

In some embodiments, the payload antibody has 97% identity to one or more of the antibody polypeptides listed in Table 3, or variants or fragments thereof.

In some embodiments, the payload antibody has 98% identity to one or more of the antibody polypeptides listed in Table 3, or variants or fragments thereof.

In some embodiments, the payload antibody has 99% identity to one or more of the antibody polypeptides listed in Table 3, or variants or fragments thereof.

In some embodiments, the payload antibody has 100% identity to one or more of the antibody polypeptides listed in Table 3, or variants or fragments thereof.

In some embodiments, the payload region of the AAV particle comprises a nucleic acid sequence with at least 50% identity to one or more nucleic acid sequences listed in Table 3, or variants or fragments thereof. The payload nucleic acid sequence may have 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to one or more nucleic acid sequences listed in Table 3, or variants or fragments thereof.

In some embodiments, the payload nucleic acid sequence has 90% identity to one or more of the nucleic acid sequences listed in Table 3, or variants or fragments thereof.

In some embodiments, the payload nucleic acid sequence has 91% identity to one or more of the nucleic acid sequences listed in Table 3, or variants or fragments thereof.

In some embodiments, the payload nucleic acid sequence has 92% identity to one or more of the nucleic acid sequences listed in Table 3, or variants or fragments thereof.

In some embodiments, the payload nucleic acid sequence has 93% identity to one or more of the nucleic acid sequences listed in Table 3, or variants or fragments thereof.

In some embodiments, the payload nucleic acid sequence has 94% identity to one or more of the nucleic acid sequences listed in Table 3, or variants or fragments thereof.

In some embodiments, the payload nucleic acid sequence has 95% identity to one or more of the nucleic acid sequences listed in Table 3, or variants or fragments thereof.

In some embodiments, the payload nucleic acid sequence has 96% identity to one or more of the nucleic acid sequences listed in Table 3, or variants or fragments thereof.

In some embodiments, the payload nucleic acid sequence has 97% identity to one or more of the nucleic acid sequences listed in Table 3, or variants or fragments thereof.

In some embodiments, the payload nucleic acid sequence has 98% identity to one or more of the nucleic acid sequences listed in Table 3, or variants or fragments thereof.

In some embodiments, the payload nucleic acid sequence has 99% identity to one or more of the nucleic acid sequences listed in Table 3, or variants or fragments thereof.

In some embodiments, the payload nucleic acid sequence has 100% identity to one or more of the nucleic acid sequences listed in Table 3, or variants or fragments thereof.

TABLE 3
Tau Associated Disease Antibodies

| | | Type | | | | SEQ |
|---|---|---|---|---|---|---|
| | | (PRT/ | | | | ID |
| Ab ID | Component | DNA) | Description | Reference | Len. | NO |
| Ab1 | VH Signal | DNA | IPN002 (VH Signal) | | 57 | 1740 |
| Ab2 | VH Signal | DNA | PHF1 (VH Signal) | | 57 | 1741 |
| Ab3 | VH Constant | DNA | VH (constant - 969) | | 969 | 1742 |
| Ab4 | VH Constant | DNA | VH (constant - 972) | | 972 | 1743 |
| Ab5 | VH CDR | PRT | 7295-M6 HCDR | WO2015122922; SEQ ID | 10 | 1744 |
| | | | | NO: 12 and 20 | | |
| Ab6 | VH CDR | PRT | 7295-M6 HCDR | WO2015122922; SEQ ID | 16 | 1745 |
| | | | | NO: 13 and 21 | | |
| Ab7 | VH CDR | PRT | 7295-M6 HCDR | WO2015122922; SEQ ID | 4 | 1746 |
| | | | | NO: 14 and 22 | | |
| Ab8 | VH CDR | PRT | 7297-2M1 HCDR | WO2015122922; SEQ ID | 16 | 1747 |
| | | | | NO: 69 | | |
| Ab9 | VH CDR | PRT | 7298-M1 HCDR | WO2015122922; SEQ ID | 11 | 1748 |
| | | | | NO: 28 | | |
| Ab10 | VH CDR | PRT | 7298-M1 HCDR | WO2015122922; SEQ ID | 16 | 1749 |
| | | | | NO: 29 | | |
| Ab11 | VH CDR | PRT | 7298-M1 HCDR | WO2015122922; SEQ ID | 4 | 1750 |
| | | | | NO: 30 | | |
| Ab12 | VH CDR | PRT | 7298-M2 HCDR | WO2015122922; SEQ ID | 10 | 1751 |
| | | | | NO: 36 | | |
| Ab13 | VH CDR | PRT | 7298-M2 HCDR | WO2015122922; SEQ ID | 14 | 1752 |
| | | | | NO: 37 | | |
| Ab14 | VH CDR | PRT | 7298-M2 HCDR | WO2015122922; SEQ ID | 8 | 1753 |
| | | | | NO: 38 | | |
| Ab15 | VH CDR | PRT | 7299-M2 HCDR | WO2015122922; SEQ ID | 10 | 1754 |
| | | | | NO: 44 and 60 | | |
| Ab16 | VH CDR | PRT | 7299-M2 HCDR | WO2015122922; SEQ ID | 16 | 1755 |
| | | | | NO: 45 | | |
| Ab17 | VH CDR | PRT | 7299-M2 HCDR | WO2015122922; SEQ ID | 4 | 1756 |
| | | | | NO: 46 and 62 | | |
| Ab18 | VH CDR | PRT | 7299-M5 HCDR | WO2015122922; SEQ ID | 10 | 1757 |
| | | | | NO: 52 and 68 | | |
| Ab19 | VH CDR | PRT | 7299-M5 HCDR | WO2015122922; SEQ ID | 16 | 1758 |
| | | | | NO: 53 | | |
| Ab20 | VH CDR | PRT | 7299-M5 HCDR | WO2015122922; SEQ ID | 10 | 1759 |
| | | | | NO: 54 and 70 | | |
| Ab21 | VH CDR | PRT | 7299-M9 HCDR | WO2015122922; SEQ ID | 15 | 1760 |
| | | | | NO: 61 | | |
| Ab22 | VH CDR | PRT | C10.2 HCDR1 | WO2017009308; SEQ ID | 13 | 1761 |
| | | | | NO: 12, 20 and 28 | | |
| Ab23 | VH CDR | PRT | C10.2 HCDR2 | WO2017009308; SEQ ID | 16 | 1762 |
| | | | | NO: 13 and 29 | | |
| Ab24 | VH CDR | PRT | C10.2 HCDR3 | WO2017009308; SEQ ID | 6 | 1763 |
| | | | | NO: 14 and 30 | | |
| Ab25 | VH CDR | PRT | C5.2 HCDR2 | WO2017009308; SEQ ID | 16 | 1764 |
| | | | | NO: 21 | | |
| Ab26 | VH CDR | PRT | C5.2 HCDR3 | WO2017009308; SEQ ID | 6 | 1765 |
| | | | | NO: 22 | | |
| Ab27 | VH CDR | PRT | D1.2 HCDR1 | WO2017009308; SEQ ID | 13 | 1766 |
| | | | | NO: 4 | | |
| Ab28 | VH CDR | PRT | D1.2 HCDR2 | WO2017009308; SEQ ID | 16 | 1767 |
| | | | | NO: 5 | | |
| Ab29 | VH CDR | PRT | D1.2 HCDR3 | WO2017009308; SEQ ID | 6 | 1768 |
| | | | | NO: 6 | | |
| Ab30 | VH CDR | PRT | HCDR1 | US8980270; SEQ ID NO: 4 | 5 | 1769 |
| Ab31 | VH CDR | PRT | HCDR1 | US8980270; SEQ ID NO: 10 | 5 | 1770 |
| Ab32 | VH CDR | PRT | HCDR2 | US8980270; SEQ ID NO: 5 | 17 | 1771 |
| Ab33 | VH CDR | PRT | HCDR2 | US8980270; SEQ ID NO: 11 | 17 | 1772 |
| Ab34 | VH CDR | PRT | HCDR2 | WO2015081085; SEQ ID | 32 | 1773 |
| | | | | NO: 27 | | |
| Ab35 | VH CDR | PRT | HCDR3 | US8980270; SEQ ID NO: 6 | 8 | 1774 |
| Ab36 | VH CDR | PRT | HCDR3 | US8980270; SEQ ID NO: 12 | 8 | 1775 |
| Ab37 | VH CDR | PRT | HCDR3 | WO2015081085; SEQ ID | 11 | 1776 |
| | | | | NO: 44 | | |
| Ab38 | VH CDR | PRT | IPN002 HCDR1 | WO2014028777; SEQ ID | 14 | 1777 |
| | | | | NO: 84 | | |
| Ab39 | VH CDR | PRT | IPN002 HCDR2 | WO2014028777; SEQ ID | 32 | 1778 |
| | | | | NO: 85 | | |
| Ab40 | VH CDR | PRT | IPN002 HCDR3 | WO2014028777; SEQ ID | 11 | 1779 |
| | | | | NO: 86 | | |
| Ab41 | VH CDR | PRT | MC1 LCDR1 | WO2016137811; SEQ ID | 13 | 1780 |
| | | | | NO: 6 | | |
| Ab42 | VH CDR | PRT | MC1 LCDR2 | WO2016137811; SEQ ID | 18 | 1781 |
| | | | | NO: 7 | | |
| Ab43 | VH CDR | PRT | MC1 LCDR3 | WO2016137811; SEQ ID | 9 | 1782 |
| | | | | NO: 8 | | |
| Ab44 | VH CDR | PRT | PT1 HCDR1 (Chothia) | US9371376; SEQ ID NO: 19 | 7 | 1783 |
| Ab45 | VH CDR | PRT | PT1 HCDR1 (Kabat) | US9371376; SEQ ID NO: 7 | 5 | 1784 |
| Ab46 | VH CDR | PRT | PT1 HCDR2 (Chothia) | US9371376; SEQ ID NO: 20 | 6 | 1785 |
| Ab47 | VH CDR | PRT | PT1 HCDR2 (Kabat) | US9371376; SEQ ID NO: 8 | 17 | 1786 |
| Ab48 | VH CDR | PRT | PT1 HCDR3 (Chothia) | US9371376; SEQ ID NO: 21 | 9 | 1787 |
| Ab49 | VH CDR | PRT | PT1 HCDR3 (Kabat) | US9371376; SEQ ID NO: 9 | 10 | 1788 |
| Ab50 | VH CDR | PRT | PT3 HCDR1 (Chothia) | US9371376; SEQ ID NO: 25 | 7 | 1789 |
| Ab51 | VH CDR | PRT | PT3 HCDR1 (Kabat) | US9371376; SEQ ID NO: 13 | 5 | 1790 |
| Ab52 | VH CDR | PRT | PT3 HCDR2 (Chothia) | US9371376; SEQ ID NO: 26 | 5 | 1791 |
| Ab53 | VH CDR | PRT | PT3 HCDR2 (Kabat) | US9371376; SEQ ID NO: 14 | 16 | 1792 |
| Ab54 | VH CDR | PRT | PT3 HCDR3 (Chothia) | US9371376; SEQ ID NO: 27 | 9 | 1793 |
| Ab55 | VH CDR | PRT | PT3 HCDR3 (Kabat) | US9371376; SEQ ID NO: 15 | 10 | 1794 |
| Ab56 | VH | PRT | 7295-M6 VH | WO2015122922; SEQ ID | 125 | 1795 |
| | | | | NO: 16 and 24 | | |
| Ab57 | VH | PRT | 7297-2M1 VH | WO2015122922; SEQ ID | 131 | 1796 |
| | | | | NO: 72 | | |
| Ab58 | VH | PRT | 7298-M1 VH | WO2015122922; SEQ ID | 127 | 1797 |
| | | | | NO: 32 | | |
| Ab59 | VH | PRT | 7298-M2 VH | WO2015122922; SEQ ID | 128 | 1798 |
| | | | | NO: 40 | | |
| Ab60 | VH | PRT | 7299-M2 VH | WO2015122922; SEQ ID | 125 | 1799 |
| | | | | NO: 48 | | |
| Ab61 | VH | PRT | 7299-M5 VH | WO2015122922; SEQ ID | 131 | 1800 |
| | | | | NO: 56 | | |
| Ab62 | VH | PRT | 7299-M9 VH | WO2015122922; SEQ ID | 125 | 1801 |
| | | | | NO: 64 | | |
| Ab63 | VH | PRT | C10.2 VH | WO2017009308; SEQ ID | 439 | 1802 |
| | | | | NO: 16 | | |
| Ab64 | VH | PRT | C10.2 VH - 115 | | 115 | 1803 |
| Ab65 | VH | DNA | C10.2 VH | | 345 | 1804 |
| | | | Bioinformatics | | | |
| Ab66 | VH | DNA | C10.2 VH EMBOSS | | 345 | 1805 |
| Ab67 | VH | DNA | C10.2 VH GeneInfinity | | 345 | 1806 |
| Ab68 | VH | DNA | C10.2 VH | | 345 | 1807 |
| | | | GregThatcher | | | |
| Ab69 | VH | DNA | C10.2 VH IDT | | 345 | 1808 |
| Ab70 | VH | DNA | C10.2 VH InSilico | | 345 | 1809 |
| Ab71 | VH | DNA | C10.2 VH MolBio | | 345 | 1810 |
| Ab72 | VH | DNA | C10.2 VH N2P | | 345 | 1811 |
| Ab73 | VH | DNA | C10.2 VH Snapgene | | 345 | 1812 |
| Ab74 | VH | DNA | C10.2 VH Vector NTI | | 345 | 1813 |
| Ab75 | VH | PRT | C5.2 VH | WO2017009308; SEQ ID | 439 | 1814 |
| | | | | NO: 24 | | |
| Ab76 | VH | PRT | C8.3 VH | WO2017009308; SEQ ID | 439 | 1815 |
| | | | | NO: 32 | | |
| Ab77 | VH | PRT | D1.2 VH | WO2017009308; SEQ ID | 451 | 1816 |
| | | | | NO: 8 | | |
| Ab78 | VH | PRT | hC10.2 VH | WO2017009308; SEQ ID | 444 | 1817 |
| | | | | NO: 35 | | |
| Ab79 | VH | DNA | IPN001 VH - 351 | US8980270; SEQ ID NO: 18 | 351 | 1818 |
| Ab80 | VH | PRT | IPN001 VH - 117 | US8980270; SEQ ID NO: 14 | 117 | 1819 |
| Ab81 | VH | DNA | IPN002 VH - 1377 | | 1377 | 1820 |
| Ab82 | VH | DNA | IPN002 VH - 351 | US8980270; SEQ ID NO: 20 | 351 | 1821 |
| Ab83 | VH | PRT | IPN002 VH - 117 | US8980270; SEQ ID NO: 16 | 117 | 1822 |
| Ab84 | VH | PRT | IPN002 VH - 30 | WO2014028777; SEQ ID | 30 | 1823 |
| | | | | NO: 83 | | |
| Ab85 | VH | DNA | IPN002 VH variant 1 | US8980270; SEQ ID NO: 28 | 351 | 1824 |
| Ab86 | VH | PRT | IPN002 VH variant 1 | US8980270; SEQ ID NO: 36 | 117 | 1825 |
| Ab87 | VH | DNA | IPN002 VH variant 2 | US8980270; SEQ ID NO: 29 | 351 | 1826 |
| Ab88 | VH | PRT | IPN002 VH variant 2 | US8980270; SEQ ID NO: 37 | 117 | 1827 |
| Ab89 | VH | DNA | IPN002 VH variant 3 | US8980270; SEQ ID NO: 30 | 351 | 1828 |
| Ab90 | VH | PRT | IPN002 VH variant 3 | US8980270; SEQ ID NO: 38 | 117 | 1829 |
| Ab91 | VH | DNA | IPN002 VH variant 4 | US8980270; SEQ ID NO: 31 | 351 | 1830 |
| Ab92 | VH | PRT | IPN002 VH variant 4 | US8980270; SEQ ID NO: 39 | 117 | 1831 |
| Ab93 | VH | DNA | MC1 VH - 1374 | | 1374 | 1832 |
| Ab94 | VH | DNA | MC1 VH - 1326 | WO2016137811; SEQ ID | 1326 | 1833 |
| | | | | NO: 12 | | |
| Ab95 | VH | PRT | MC1 VH - 458 | | 458 | 1834 |
| Ab96 | VH | PRT | MC1 VH - 442 | WO2016137811; SEQ ID | 442 | 1835 |
| | | | | NO: 2 | | |
| Ab97 | VH | PRT | MC1 VH - 116 | WO2016137811; SEQ ID | 116 | 1836 |
| | | | | NO: 10 | | |
| Ab98 | VH | DNA | PHF1 VH - 1320 | | 1320 | 1837 |
| Ab99 | VH | DNA | PHF1 VH - 1377 | | 1377 | 1838 |
| Ab100 | VH | DNA | PHF1 VH - 351 | | 351 | 1839 |
| Ab101 | VH | DNA | PHF1 VH - 699 | | 699 | 1840 |
| Ab102 | VH | DNA | PHF1 VH - 717 | | 717 | 1841 |
| Ab103 | VH | DNA | PHF1 VH - 744 | | 744 | 1842 |
| Ab104 | VH | DNA | PHF1 VH - 753 | | 753 | 1843 |
| Ab105 | VH | PRT | PHF1 VH - 459 | | 459 | 1844 |
| Ab106 | VH | DNA | PHF1 VH + | | 1383 | 1845 |
| Ab107 | VH | DNA | PT1 VH - 357 | US9371376; SEQ ID NO: 31 | 357 | 1846 |
| Ab108 | VH | PRT | PT1 VH - 119 | US9371376; SEQ ID NO: 35 | 119 | 1847 |
| Ab109 | VH | DNA | PT3 VH - 354 | US9371376; SEQ ID NO: 33 | 354 | 1848 |
| Ab110 | VH | PRT | PT3 VH - 118 | US9371376; SEQ ID NO: 37 | 118 | 1849 |
| Ab111 | VH | DNA | PT3 VH Bioinformatics | | 354 | 1850 |
| Ab112 | VH | DNA | PT3 VH Bioinformatics | | 354 | 1851 |
| | | | 2 | | | |
| Ab113 | VH | DNA | PT3 VH EMBOSS | | 354 | 1852 |
| Ab114 | VH | DNA | PT3 VH Geneinfinity | | 354 | 1853 |
| Ab115 | VH | DNA | PT3 VH Genescript | | 354 | 1854 |
| Ab116 | VH | DNA | PT3 VH IDT | | 354 | 1855 |
| Ab117 | VH | DNA | PT3 VH NUS | | 354 | 1856 |
| Ab118 | VH | DNA | PT3 VH NUS2 | | 354 | 1857 |
| Ab119 | VH | DNA | PT3 VH NUS3 | | 354 | 1858 |
| Ab120 | VH | DNA | PT3 VH Snapgene | | 354 | 1859 |
| Ab121 | VH | PRT | VH | WO2015081085; SEQ ID | 30 | 1860 |
| | | | | NO: 25 | | |
| Ab122 | VL Signal | DNA | IPN002 (VL Signal) | | 57 | 1861 |
| Ab123 | VL Signal | DNA | PHF1 (VL Signal) | | 57 | 1862 |
| Ab124 | VL Signal | DNA | PHF1 (VL Signal 72) | | 72 | 1863 |
| Ab125 | VL Constant | DNA | VL (constant) | | 321 | 1864 |
| Ab126 | VL CDR | PRT | 7295-M6 LCDR | WO2015122922; SEQ ID | 16 | 1865 |
| | | | | NO: 9 and 17 | | |
| Ab127 | VL CDR | PRT | 7295-M6 LCDR | WO2015122922; SEQ ID | 9 | 1866 |
| | | | | NO: 11 and 19 | | |
| Ab128 | VL CDR | PRT | 7297-2M1 LCDR | WO2015122922; SEQ ID | 11 | 1867 |
| | | | | NO: 65 | | |
| Ab129 | VL CDR | PRT | 7297-2M1 LCDR | WO2015122922; SEQ ID | 7 | 1868 |
| | | | | NO: 66 | | |
| Ab130 | VL CDR | PRT | 7297-2M1 LCDR | WO2015122922; SEQ ID | 8 | 1869 |
| | | | | NO: 67 | | |
| Ab131 | VL CDR | PRT | 7298-M1 LCDR | WO2015122922; SEQ ID | 15 | 1870 |
| | | | | NO: 25 and 33 | | |
| Ab132 | VL CDR | PRT | 7298-M1 LCDR | WO2015122922; SEQ ID | 7 | 1871 |
| | | | | NO: 26 and 34 | | |
| Ab133 | VL CDR | PRT | 7298-M1 LCDR | WO2015122922; SEQ ID | 9 | 1872 |
| | | | | NO: 27 and 35 | | |
| Ab134 | VL CDR | PRT | 7299-M2 LCDR | WO2015122922; SEQ ID | 16 | 1873 |
| | | | | NO: 41 | | |
| Ab135 | VL CDR | PRT | 7299-M2 LCDR | WO2015122922; SEQ ID | 7 | 1874 |
| | | | | NO: 42 | | |
| Ab136 | VL CDR | PRT | 7299-M2 LCDR | WO2015122922; SEQ ID | 9 | 1875 |
| | | | | NO: 43 | | |
| Ab137 | VL CDR | PRT | 7299-M5 LCDR | WO2015122922; SEQ ID | 16 | 1876 |
| | | | | NO: 49 and 57 | | |
| Ab138 | VL CDR | PRT | 7299-M5 LCDR | WO2015122922; SEQ ID | 7 | 1877 |
| | | | | NO: 50 and 58 | | |
| Ab139 | VL CDR | PRT | 7299-M5 LCDR | WO2015122922; SEQ ID | 9 | 1878 |
| | | | | NO: 51 | | |
| Ab140 | VL CDR | PRT | 7299-M9 LCDR | WO2015122922; SEQ ID | 8 | 1879 |
| | | | | NO: 59 | | |
| Ab141 | VL CDR | PRT | C10.2 LCDR1 | WO2017009308; SEQ ID | 11 | 1880 |
| | | | | NO: 9 and 25 | | |
| Ab142 | VL CDR | PRT | C10.2 LCDR2 | WO2017009308; SEQ ID | 7 | 1881 |
| | | | | NO: 10 and 18 | | |
| Ab143 | VL CDR | PRT | C10.2 LCDR3 | WO2017009308; SEQ ID | 7 | 1882 |
| | | | | NO: 11 and 19 | | |
| Ab144 | VL CDR | PRT | C5.2 LCDR1 | WO2017009308; SEQ ID | 11 | 1883 |
| | | | | NO: 17 | | |
| Ab145 | VL CDR | PRT | C8.3 LCDR2 | WO2017009308; SEQ ID | 7 | 1884 |
| | | | | NO: 26 | | |
| Ab146 | VL CDR | PRT | C8.3 LCDR3 | WO2017009308; SEQ ID | 7 | 1885 |
| | | | | NO: 27 | | |
| Ab147 | VL CDR | PRT | D1.2 LCDR1 | WO2017009308; SEQ ID | 16 | 1886 |
| | | | | NO: 1 | | |
| Ab148 | VL CDR | PRT | D1.2 LCDR2 | WO2017009308; SEQ ID | 7 | 1887 |
| | | | | NO: 2 | | |
| Ab149 | VL CDR | PRT | D1.2 LCDR3 | WO2017009308; SEQ ID | 7 | 1888 |
| | | | | NO: 3 | | |
| Ab150 | VL CDR | PRT | IPN002 LCDR1 | WO2014028777; SEQ ID | 15 | 1889 |
| | | | | NO: 55 | | |
| Ab151 | VL CDR | PRT | IPN002 LCDR2 | WO2014028777; SEQ ID | 32 | 1890 |
| | | | | NO: 56 | | |
| Ab152 | VL CDR | PRT | IPN002 LCDR3 | WO2014028777; SEQ ID | 10 | 1891 |
| | | | | NO: 57 | | |
| Ab153 | VL CDR | PRT | LCDR1 | US8980270; SEQ ID NO: 1 | 16 | 1892 |
| Ab154 | VL CDR | PRT | LCDR1 | US8980270; SEQ ID NO: 7 | 16 | 1893 |
| Ab155 | VL CDR | PRT | LCDR1 | WO2015081085; SEQ ID | 15 | 1894 |
| | | | | NO: 46 | | |
| Ab156 | VL CDR | PRT | LCDR2 | US8980270; SEQ ID NO: 2 | 7 | 1895 |
| Ab157 | VL CDR | PRT | LCDR2 | WO2015081085; SEQ ID | 32 | 1896 |
| | | | | NO: 47 | | |
| Ab158 | VL CDR | PRT | LCDR3 | US8980270; SEQ ID NO: 3 | 9 | 1897 |
| | | | | and 9 | | |
| Ab159 | VL CDR | PRT | MC1 LCDR1 | WO2016137811; SEQ ID | 16 | 1898 |
| | | | | NO: 3 | | |
| Ab160 | VL CDR | PRT | MC1 LCDR2 | WO2016137811; SEQ ID | 8 | 1899 |
| | | | | NO: 4 | | |
| Ab161 | VL CDR | PRT | MC1 LCDR3 | WO2016137811; SEQ ID | 9 | 1900 |
| | | | | NO: 5 | | |
| Ab162 | VL CDR | PRT | PT1 LCDR1 (Chothia) | US9371376; SEQ ID NO: 22 | 12 | 1901 |
| Ab163 | VL CDR | PRT | PT1 LCDR1 (Kabat) | US9371376; SEQ ID NO: 10 | 16 | 1902 |
| Ab164 | VL CDR | PRT | PT1 LCDR2 (Chothia) | US9371376; SEQ ID NO: 23 | 3 | 1903 |
| Ab165 | VL CDR | PRT | PT1 LCDR2 (Kabat) | US9371376; SEQ ID NO: 11 | 7 | 1904 |
| Ab166 | VL CDR | PRT | PT1 LCDR3 (Chothia) | US9371376; SEQ ID NO: 24 | 7 | 1905 |
| Ab167 | VL CDR | PRT | PT1 LCDR3 (Kabat) | US9371376; SEQ ID NO: 12 | 9 | 1906 |
| Ab168 | VL CDR | PRT | PT3 LCDR1 (Chothia) | US9371376; SEQ ID NO: 28 | 7 | 1907 |
| Ab169 | VL CDR | PRT | PT3 LCDR1 (Kabat) | US9371376; SEQ ID NO: 16 | 11 | 1908 |
| Ab170 | VL CDR | PRT | PT3 LCDR2 (Chothia) | US9371376; SEQ ID NO: 29 | 3 | 1909 |
| Ab171 | VL CDR | PRT | PT3 LCDR2 (Kabat) | US9371376; SEQ ID NO: 17 | 7 | 1910 |
| Ab172 | VL CDR | PRT | PT3 LCDR3 (Chothia) | US9371376; SEQ ID NO: 30 | 6 | 1911 |
| Ab173 | VL CDR | PRT | PT3 LCDR3 (Kabat) | US9371376; SEQ ID NO: 18 | 10 | 1912 |
| Ab174 | VL | PRT | 7295-M6 VL | WO2015122922; SEQ ID | 122 | 1913 |
| | | | | NO: 15 and 23 | | |
| Ab175 | VL | PRT | 7297-2M1 VL | WO2015122922; SEQ ID | 116 | 1914 |
| | | | | NO: 71 | | |
| Ab176 | VL | PRT | 7298-M1 VL | WO2015122922; SEQ ID | 121 | 1915 |
| | | | | NO: 31 and 39 | | |
| Ab177 | VL | PRT | 7299-M2 VL | WO2015122922; SEQ ID | 122 | 1916 |
| | | | | NO: 47 | | |
| Ab178 | VL | PRT | 7299-M5 VL | WO2015122922; SEQ ID | 122 | 1917 |
| | | | | NO: 55 | | |
| Ab179 | VL | PRT | 7299-M9 VL | WO2015122922; SEQ ID | 122 | 1918 |
| | | | | NO: 63 | | |
| Ab180 | VL | PRT | C10.2 VL - 107 | | 107 | 1919 |
| Ab181 | VL | PRT | C10.2 VL - 214 | WO2017009308; SEQ ID | 214 | 1920 |
| | | | | NO: 15 | | |
| Ab182 | VL | DNA | C10.2 VL | | 321 | 1921 |
| | | | Bioinformatics | | | |
| Ab183 | VL | DNA | C10.2 VL EMBOSS | | 321 | 1922 |
| Ab184 | VL | DNA | C10.2 VL GeneInfinity | | 321 | 1923 |
| Ab185 | VL | DNA | C10.2 VL | | 321 | 1924 |
| | | | GregThatcher | | | |
| Ab186 | VL | DNA | C10.2 VL IDT | | 321 | 1925 |
| Ab187 | VL | DNA | C10.2 VL InSilico | | 321 | 1926 |
| Ab188 | VL | DNA | C10.2 VL MolBio | | 321 | 1927 |
| Ab189 | VL | DNA | C10.2 VL N2P | | 321 | 1928 |
| Ab190 | VL | DNA | C10.2 VL Snapgene | | 321 | 1929 |
| Ab191 | VL | DNA | C10.2 VL Vector NTI | | 321 | 1930 |
| Ab192 | VL | PRT | C5.2 VL | WO2017009308; SEQ ID | 214 | 1931 |
| | | | | NO: 23 | | |
| Ab193 | VL | PRT | C8.3 VL | WO2017009308; SEQ ID | 214 | 1932 |
| | | | | NO: 31 | | |
| Ab194 | VL | PRT | D1.2 VL | WO2017009308; SEQ ID | 219 | 1933 |
| | | | | NO: 7 | | |
| Ab195 | VL | PRT | D1.2* VL | WO2017009308; SEQ ID | 219 | 1934 |
| | | | | NO: 34 | | |
| Ab196 | VL | PRT | hC10.2 VL | WO2017009308; SEQ ID | 214 | 1935 |
| | | | | NO: 36 | | |
| Ab197 | VL | DNA | IPN001 VL - 336 | US8980270; SEQ ID NO: 17 | 336 | 1936 |
| Ab198 | VL | PRT | IPN001 VL | US8980270; SEQ ID NO: 13 | 112 | 1937 |
| Ab199 | VL | PRT | IPN002 VL | WO2014028777; SEQ ID | 23 | 1938 |
| | | | | NO: 54 | | |
| Ab200 | VL | DNA | IPN002 VL - 336 | US8980270; SEQ ID NO: 19 | 336 | 1939 |
| Ab201 | VL | DNA | IPN002 VL - 714 | | 714 | 1940 |
| Ab202 | VL | PRT | IPN002 VL - 112 | US8980270; SEQ ID NO: 15 | 112 | 1941 |
| Ab203 | VL | DNA | IPN002 VL variant 1 | US8980270; SEQ ID NO: 32 | 336 | 1942 |
| Ab204 | VL | PRT | IPN002 VL variant 1 | US8980270; SEQ ID NO: 40 | 112 | 1943 |
| Ab205 | VL | DNA | IPN002 VL variant 2 | US8980270; SEQ ID NO: 33 | 336 | 1944 |
| Ab206 | VL | PRT | IPN002 VL variant 2 | US8980270; SEQ ID NO: 41 | 112 | 1945 |
| Ab207 | VL | DNA | IPN002 VL variant 3 | US8980270; SEQ ID NO: 34 | 336 | 1946 |
| Ab208 | VL | PRT | IPN002 VL variant 3 | US8980270; SEQ ID NO: 42 | 112 | 1947 |
| Ab209 | VL | DNA | IPN002 VL variant 4 | US8980270; SEQ ID NO: 35 | 336 | 1948 |
| Ab210 | VL | PRT | IPN002 VL variant 4 | US8980270; SEQ ID NO: 43 | 112 | 1949 |
| Ab211 | VL | DNA | MC1 VL - 714 | | 714 | 1950 |
| Ab212 | VL | DNA | MC1 VL - 717 | | 717 | 1951 |
| Ab213 | VL | DNA | MC1 VL - 657 | WO2016137811; SEQ ID | 657 | 1952 |
| | | | | NO: 11 | | |
| Ab214 | VL | PRT | MC1 VL - 238 | | 238 | 1953 |
| Ab215 | VL | PRT | MC1 VL - 219 | WO2016137811; SEQ ID | 219 | 1954 |
| | | | | NO: 1 | | |
| Ab216 | VL | PRT | MC1 VL - 112 | WO2016137811; SEQ ID | 112 | 1955 |
| | | | | NO: 9 | | |
| Ab217 | VL | DNA | PHF1 VL - 339v1 | | 339 | 1956 |
| Ab218 | VL | DNA | PHF1 VL - 339v2 | | 339 | 1957 |
| Ab219 | VL | DNA | PHF1 VL - 396 | | 396 | 1958 |
| Ab220 | VL | DNA | PHF1 VL - 657 | | 657 | 1959 |
| Ab221 | VL | DNA | PHF1 VL - 660 | | 660 | 1960 |
| Ab222 | VL | DNA | PHF1 VL - 678 | | 678 | 1961 |
| Ab223 | VL | DNA | PHF1 VL - 714 | | 714 | 1962 |
| Ab224 | VL | DNA | PHF1 VL - 720 | | 720 | 1963 |
| Ab225 | VL | PRT | PHF1 VL - 239 | | 239 | 1964 |
| Ab226 | VL | DNA | PHF1 VL + (732) | | 732 | 1965 |
| Ab227 | VL | DNA | PHF1 VL + (747) | | 747 | 1966 |
| Ab228 | VL | DNA | PT1 VL - 336 | US9371376; SEQ ID NO: 32 | 336 | 1967 |
| Ab229 | VL | PRT | PT1 VL - 112 | US9371376; SEQ ID NO: 36 | 112 | 1968 |
| Ab230 | VL | DNA | PT3 VL - 321 | US9371376; SEQ ID NO: 34 | 321 | 1969 |
| Ab231 | VL | PRT | PT3 VL - 107 | US9371376; SEQ ID NO: 38 | 107 | 1970 |
| Ab232 | VL | DNA | PT3 VL Bioinformatics | | 321 | 1971 |
| Ab233 | VL | DNA | PT3 VL Bioinformatics | | 321 | 1972 |
| | | | 2 | | | |
| Ab234 | VL | DNA | PT3 VL EMBOSS | | 321 | 1973 |
| Ab235 | VL | DNA | PT3 VL Geneinfinity | | 321 | 1974 |
| Ab236 | VL | DNA | PT3 VL Genescript | | 321 | 1975 |
| Ab237 | VL | DNA | PT3 VL IDT | | 321 | 1976 |
| Ab238 | VL | DNA | PT3 VL NUS | | 321 | 1977 |
| Ab239 | VL | DNA | PT3 VL NUS2 | | 321 | 1978 |
| Ab240 | VL | DNA | PT3 VL NUS3 | | 321 | 1979 |
| Ab241 | VL | DNA | PT3 VL Snapgene | | 321 | 1980 |
| Ab242 | VL | PRT | VL | WO2015081085; SEQ ID | 23 | 1981 |
| | | | | NO: 45 | | |
| Ab243 | Fab | PRT | AT8 Fab1 | Malia, T.J. et al, “Epitope | 18 | 1982 |
| | | | | mapping and structural basis | | |
| | | | | for the recognition of | | |
| | | | | phosphorylated tau by the | | |
| | | | | anti-tau antibody AT8”, | | |
| | | | | Proteins 84 (4), 427-434 | | |
| | | | | (2016), Accession number | | |
| | | | | 5E2V_P | | |
| Ab244 | Fab | PRT | AT8 Fab2 | Malia, T.J. et al, “Epitope | 18 | 1983 |
| | | | | mapping and structural basis | | |
| | | | | for the recognition of | | |
| | | | | phosphorylated tau by the | | |
| | | | | anti-tau antibody AT8”, | | |
| | | | | Proteins 84 (4), 427-434 | | |
| | | | | (2016), Accession number | | |
| | | | | 5E2W_P | | |
| Ab245 | Full | DNA | MC1 Furin 2A - 2168 | WO2015035190; SEQ ID | 2168 | 1984 |
| | antibody | | | NO: 2 | | |
| Ab246 | Full | PRT | MC1 Furin 2A - 718 | WO2015035190; SEQ ID | 718 | 1985 |
| | antibody | | | NO: 4 | | |
| Ab247 | Full | DNA | MC1 optimized seq | WO2015035190; SEQ ID | 2168 | 1986 |
| | antibody | | | NO: 6 | | |
| Ab248 | Full | DNA | PHF1 Furin 2A - 2174 | WO2015035190; SEQ ID | 2174 | 1987 |
| | antibody | | | NO: 1 | | |
| Ab249 | Full | PRT | PHF1 Furin 2A - 720 | WO2015035190; SEQ ID | 720 | 1988 |
| | antibody | | | NO: 3 | | |
| Ab250 | Full | DNA | PHF1 optimized seq | WO2015035190; SEQ ID | 2174 | 1989 |
| | antibody | | | NO: 5 | | |
| Ab251 | VH | DNA | PT3 | | 348 | 2241 |
| Ab252 | VH | DNA | VH (constant - 309) | | 309 | 2242 |
| | Constant | | | | | |
| Ab253 | VH | DNA | VH (constant - 291) | | 291 | 2243 |
| | Constant | | | | | |
| Ab254 | VH | DNA | PHF1 VH-435 | | 435 | 2169 |
| Ab255 | VH | DNA | PHF1 VH-408 | | 408 | 2170 |
| Ab256 | VL Signal | DNA | PHF1 (VL Signal 60) | | 60 | 4564 |
| Ab257 | VL | DNA | PHF1 VL - 449 | | 449 | 4565 |

In some embodiments, the payload region of the AAV particle comprises a nucleic acid sequence encoding a polypeptide which is an antibody, an antibody-based composition, or a fragment thereof. As a non-limiting example, the antibody may be one or more of the polypeptides listed in Table 3, or variants or fragments thereof. As another non-limiting example, the antibody may be one or more of the heavy chain sequences listed in Table 3. As a non-limiting example, the antibody may be one or more of the light chain sequences listed in Table 3, or variants or fragments thereof.

In some embodiments, the payload region of the AAV particle comprises a nucleic acid sequence encoding a polypeptide comprising a heavy chain and a light chain sequence listed in Table 3, or variants or fragments thereof. The payload region may also comprise a linker between the heavy and light chain sequences. The linker may be a sequence known in the art or described in Table 2.

In some embodiments, the payload region of the AAV particle comprises a nucleic acid sequence encoding a polypeptide comprising a heavy chain and a light chain sequence listed in Table 3, or variants or fragments thereof, where the heavy chain sequence is from a different antibody than the light chain sequence. The payload region may also comprise a linker between the heavy and light chain sequences. The linker may be a sequence known in the art or described in Table 2.

In some embodiments, the payload region comprises, in the 5′ to 3′ direction, an antibody light chain sequence, a linker and a heavy chain sequence. In another embodiment, the linker is not used.

In some embodiments, the payload region comprises a nucleic acid sequence encoding, in the 5′ to 3′ direction, an antibody light chain sequence from Table 3, a linker from Table 2 and a heavy chain sequence from Table 3. Non-limiting examples are included in Tables 4-6 and Tables 15-95.

In some embodiments, the payload region comprises, in the 5′ to 3′ direction, an antibody heavy chain sequence, a linker region (may comprise one or more linkers) and a light chain sequence. In another embodiment, the linker is not used.

In some embodiments, the payload region comprises a nucleic acid sequence encoding, in the 5′ to 3′ direction, an antibody heavy chain sequence from Table 3, one or more linkers from Table 2, and a light chain sequence from Table 3. Non-limiting examples are included in Tables 4-6 and Tables 15-95.

In some embodiments, the payload region comprises a nucleic acid sequence encoding a single heavy chain. As a non-limiting example, the heavy chain is an amino acid sequence or fragment thereof described in Table 3.

Shown in Table 3 are a listing of antibodies and their polynucleotides and/or polypeptides sequences. These sequences may be encoded by or included in the AAV particles of the present disclosure. Variants or fragments of the antibody sequences described in Table 3 may be utilized in the AAV particles of the present disclosure.

In some embodiments, the AAV particles may comprise a viral genome, wherein one or more components may be codon-optimized. Codon-optimization may be achieved by any method known to one with skill in the art such as, but not limited to, by a method according to Genescript, EMBOSS, Bioinformatics, NUS, NUS2, Geneinfinity, IDT, NUS3, GregThatcher, Insilico, Molbio, N2P, Snapgene, and/or VectorNTI. Antibody heavy and/or light chain sequences within the same viral genome may be codon-optimized according to the same or according to different methods.

In some cases, the payload region of the AAV particles may encode one or more isoforms or variants of heavy and light chain antibody domains. Such variants may be humanized or optimized antibody domains comprising one or more complementarity determining regions (CDRs) from the heavy and light chains listed in Table 3. CDRs of the antibodies encoded by the viral genomes of the present disclosure may be 50%, 60%, 70%, 80%, 90%, 95% identical to CDRs listed in or incorporated in the sequences of Table 3. Methods of determining CDRs are well known in the art and are described herein. Payload regions may encode antibody variants with one or more heavy chain variable domain (V_{H}) or light chain variable domain (V_{L}) derived from the antibody sequences in Table 3. In some cases, such variants may include bispecific antibodies. Bispecific antibodies encoded by payload regions may comprise variable domain pairs from two different antibodies.

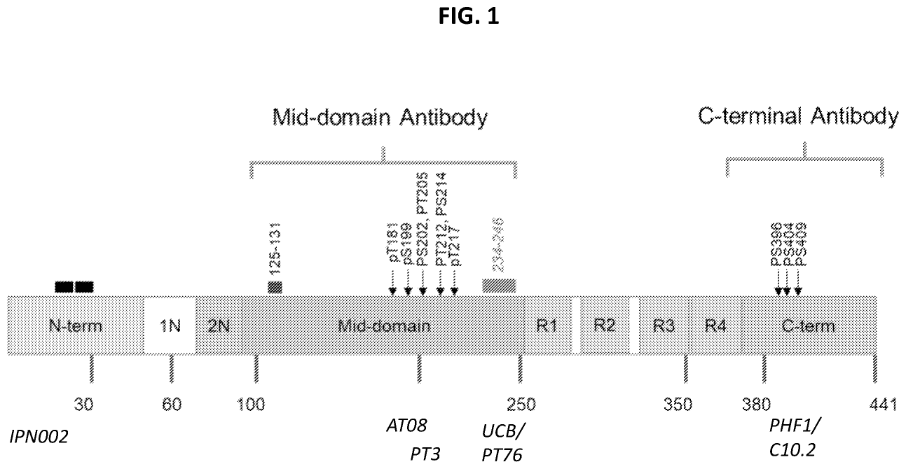

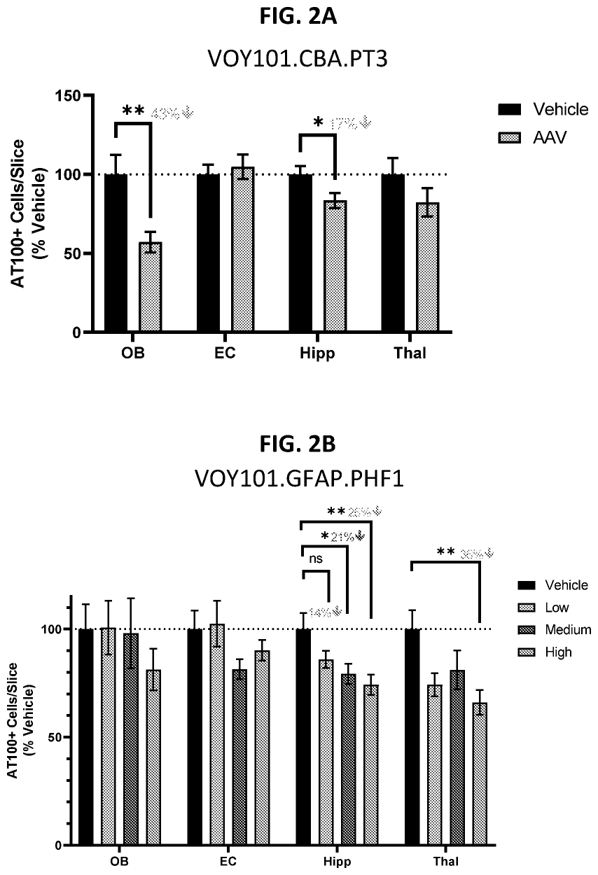

In some embodiments, the AAV particles may comprise a heavy and a light chain of an antibody described herein and two promoters. As a non-limiting example, the AAV particles may comprise a nucleic acid sequence of a genome as described in FIG. 1 or FIG. 2 of US Patent Publication No. US20030219733, the contents of which are herein incorporated by reference in their entirety. As another non-limiting example, the AAV particles may be a dual-promoter AAV for antibody expression as described by Lewis et al. (J. of. Virology, September 2002, Vol. 76(17), p 8769-8775; the contents of which are herein incorporated by reference in their entirety).

Payload regions of the viral genomes may encode any anti-tau antibodies, or tau-associated antibodies, not limited to those described in Table 3, including antibodies that are known in the art and/or antibodies that are commercially available. This may include fragments of such antibodies or antibodies that have been developed to comprise one or more of such fragments [e.g., variable domains or complementarity determining regions (CDRs)]. Anti-tau antibodies that may be encoded by payloads include, but are not limited to, AT8 (pSer²⁰²/pThr²⁰⁵; ThermoFisher, Waltham, MA; described in International Publication No. WO1995017429, the contents of which are herein incorporated in their entirety), AT100 (pThr²¹²/pSer²¹⁴; ThermoFisher, Waltham, MA; described in U.S. Pat. No. 6,121,003, the contents of which are herein incorporated in their entirety), AT180 (pThr²³¹; ThermoFisher, Waltham, MA; described in International Publication No. WO1995017429, the contents of which are herein incorporated by reference in their entirety), MC-1 (or MC1) (Tau^{2-18/312-342} conformational antibody; as described in International Publication WO199620218, the contents of which are herein incorporated by reference in their entirety), MC-6 (pSer²³⁵; described in U.S. Pat. No. 5,811,310, the contents of which are herein incorporated in their entirety), TG-3 (pThr²³¹; described in Jicha, G A et al., 1997 J Neurochem 69(5):2087-95, the contents of which are herein incorporated by reference in their entirety), CP13 (pSer²⁰²), CP27 (human Tau¹³⁰⁻¹⁵⁰), Tau12 (human Tau⁹⁻¹⁸; Abcam, Cambridge, MA), TG5 (Tau²²⁰⁻²⁴²; described in U.S. Pat. No. 5,811,310), DA9 (Tau¹⁰²⁻¹⁴⁰; described in U.S. Pat. No. 5,811,310), PHF1 (or PHF-1) (pSer³⁹⁶/pSer⁴⁰⁴; described in International Publication WO199620218), Alz50 (Tau⁷⁻⁹ and Tau³¹²⁻³⁴² conformational epitope; described in U.S. Pat. No. 5,811,310 and Carmel, G et al 1996 J Biol Chem 271(51):32780-32795 and Jicha, G A et al, 1997 J Neurosci Res 48(2):128-132, the contents of each of which are herein incorporated by reference in their entirety), Tau-1 (de-phosphorylated Ser¹⁹⁵/Ser¹⁹⁸/Ser¹⁹⁹/Ser²⁰²; ThermoFisher, Waltham, MA), Tau46 (Tau⁴⁰⁴⁻⁴⁴¹; Abcam, Cambridge, MA), pS199 (ThermoFisher, Waltham, MA), pT205, pS396 (ThermoFisher, Waltham, MA; described in U.S. Pat. No. 8,647,631, the contents of which are herein incorporated by reference in their entirety), pS404 (ThermoFisher, Waltham, MA; described in U.S. Pat. No. 8,647,631, the contents of which are herein incorporated by reference in their entirety), pS422 (ThermoFisher, Waltham, MA), A0024 (hTau²⁴³⁻⁴⁴¹; Dako, Glostrup, Denmark), HT7 (hTau¹⁵⁹⁻¹⁶³; ThermoFisher, Waltham, MA), Tau2 (hTau⁵²⁻⁶⁸; Abcam, Cambridge, MA), AD2 (pSer³⁹⁶/pSer⁴⁰⁴; Bio-Rad Laboratories, Hercules, CA), AT120 (hTau²¹⁶⁻²²⁴; described in U.S. Pat. No. 5,843,779, the contents of which are herein incorporated by reference in their entirety), AT270 (pThr¹⁸¹; ThermoFisher, Waltham, MA), 12E8 (pSer²⁶² and/or Ser³⁵⁶), K9JA (hTau²⁴³⁻⁴⁴¹; Dako, Caprinteria, CA), TauC3 (hTau Asp441; Santa Cruz Biotechnology, Dallas, TX; described in United States Patent Publication US20120244174 and Gamblin, T C et al 2003 PNAS 100(17):10032-7, the contents of each of which are herein incorporated by reference in their entirety), 4E6G7 (pSer³⁹⁶/pSer⁴⁰⁴; described in United States Patent Publication No. US2010316564 and Congdon, E. E. et al., 2016. Molecular Neurodegeneration August 30; 11(1):62, the contents of which are herein incorporated by reference in their entirety), 6B2 and variants thereof, described in International Patent Publication WO2016007414, the contents of which are herein incorporated by reference in their entirety, RZ3 (pThr²³¹), PG5 (pSer⁴⁰⁹), BT2 (pS^{199/202}), DA31 (Tau¹⁵⁰⁻¹⁹⁰), CP9 (pThr²³¹) Ta1505 (phospho site between Tau⁴¹⁰⁻⁴²¹, particularly pSer⁴¹³ as described in United States Patent Publication US20150183854 and Umeda, T. et al., 2015. Ann Clin Trans Neurol 2(3): 241-255, the contents of each of which are herein incorporated by reference in their entirety), PHF-6 (pThr²³¹, as described in Hoffman R et al., 1997. Biochemistry 36; 8114-8124, the contents of which are herein incorporated by reference in their entirety), PHF-13 (pSer³⁹⁶, as described in Hoffman R et al., 1997. Biochemistry 36; 8114-8124), 16B5 (Tau²⁵⁻⁴⁶, as described in United States Publication US20160031976, the contents of which are herein incorporated by reference in their entirety), DC8E8 (as described in United States Patent Publication US20150050215, the contents of which are herein incorporated by reference in their entirety), PT1 or PT3 (as described in U.S. Pat. No. 9,371,376, the contents of which are herein incorporated by reference in their entirety), 4G11 (Tau⁵⁷⁻⁶⁴, as described in International Publication WO2016137950, the contents of which are herein incorporated by reference in their entirety), 1A6 (Tau⁷-1⁷ and Tau²¹⁵-2²⁰, as described in International Publication WO2016137950), Tau15 or Tau81 (as described in International Publication WO2016055941, the contents of which are herein incorporated by reference in their entirety), TOC-1 (dimerized or aggregated tau, as described in International Publication WO2012149365, the contents of which are herein incorporated by reference in their entirety), pS404IgG2a/k (Neotope Biosciences, South San Francisco, CA; as described in Ittner et al., 2015. Neurochemistry 132:135-145, the contents of which are herein incorporated by reference in their entirety), TOMA (tau oligomer monoclonal antibody; as described in U.S. Pat. Nos. 8,778,343 and 9,125,846, International Publications WO2012051498 and WO2011026031, or United States Publication Nos. US20150004169 and US20150322143, and Castillo-Carranza, D L et al., 2014 J Neurosci 34(12)4260-72, the contents of each of which are herein incorporated by reference in their entirety), TTC-99 (oligomeric tau), BMS-986168 (as described in United States Patent Publication US2014294831, International Publication WO2015081085 and U.S. Pat. No. 8,980,271, the contents of which are herein incorporated by reference in their entirety), 3H3 (pan-amyloid epitope; described in Levites, Y et al 2015 J Neurosci 35(16)6265-76, the contents of which are herein incorporated by reference in their entirety), cis-pT231 (described in International Publications WO2012149334 and WO2011056561, the contents of which are herein incorporated by reference in their entirety), CP-3 (pSer²¹⁴; described in Jicha et al 1999 J Neurosci 19(17):7486-94, the contents of which are herein incorporated by reference in their entirety), TNT1 (Tau²⁻¹⁸; as described in United States Patent Publication 20160031978, the contents of which are herein incorporated by reference in their entirety), Tau-nY29 (nTyr²⁹; described in Reynolds M R, et al., 2006 J Neurosci 26(42):10636-45, the contents of which are herein incorporated by reference in their entirety), Tau-nY197 (nTyr¹⁹⁷; described in Reyes, J F et al., 2012 Acta Neuropathol 123(1):119-32, the contents of which are herein incorporated by reference in their entirety), Tau-nY394 (nTyr³⁹⁴; described in Reyes, J F et al 2012), 4E4 (Tau³³⁷⁻³⁴³ Tau³⁸⁷⁻³⁹⁷; described in International Publication WO2012049570 and United States Patent Publication US20150252102, the contents of each of which are herein incorporated by reference in their entirety), ADx210 (described in United States Patent Publication US20140161875, the contents of which are herein incorporated by reference in their entirety), ADx215 (described in United States Patent Publication US20140161875), ADx202 (as described in International Publication WO2015004163, the contents of which are herein incorporated by reference in their entirety), AP422 (pSer⁴²²; described in Hasegawa, M et al 1996 FEBS Lett 384:25-30, the contents of which are herein incorporated by reference in their entirety), Tau5 (Tau²¹⁰⁻²⁴¹) RTA2 (Tau²⁷³⁻²⁸³), RTAC (Tau⁴²⁶⁻⁴⁴¹), RTA1 (Tau²⁵⁷⁻²⁷⁴), T46 (Tau³⁹⁵⁻⁴³²), T49, MIGT4, O.BG.15, 525, 3-39, 4F1, MapTau (Tau⁹⁵⁻¹⁰⁸; SMI Covance), T1, HYB33801 (Tau⁵⁻¹²), Tau13 (Tau²⁻¹⁸), B11E8, 5J20 (14-3-3 tau), DC25 (Tau³⁴⁷⁻³⁵³), DC39N1 (Tau⁴⁵⁻⁷³), DC-11 (Tau³²¹⁻³⁹¹; described in U.S. Pat. No. 7,746,180, the contents of which are herein incorporated by reference in their entirety), DC39 (Tau⁴⁰¹⁻⁴¹¹), DC4R, n847 (nitrated tau), SPM452, TI4, 1E1/A6 (Tau²⁷⁵⁻²⁹¹), 5E2, 8E6/C11 (Tau²⁰⁹⁻²²⁴), 2E12 (pT231), NFT200, 248E5 (Tau³⁻²¹⁴), IG2 (Thr¹⁷⁵, Thr¹⁸¹, Thr²³¹; as described in International Publication WO2016041553, the contents of which are herein incorporated by reference in their entirety), YP3 (as described in WO2007019273, the contents of which are herein incorporated by reference in their entirety), YP4 (as described in WO2007019273) and 14-3-3 Tau (pSer 14-3-3 binding motif; Abcam, Cambridge, MA). Further, anti-tau antibodies may be any of those listed in the antibody section of Alzforum.org or at the Antibody Resource Page.com, the contents of each of which are herein incorporated by reference in their entirety. Further, anti-tau antibodies may be any commercially available anti-tau antibody. Additional antibodies may include any of those taught in Petry, F. R. et al., 2014. PLoS One 9(5): e94251, the contents of which are herein incorporated by reference in their entirety. In one example, such antibodies may include any of those described in Jicha, G. A. et al., 1997. Journal of Neuroscience Research 48:128-132, the contents of which are herein incorporated by reference in their entirety. One such antibody, MC-1 (or MC1), recognizes distinct conformations of tau that are associated with neurological disease.

In some embodiments, the AAV particles may have a payload region comprising any of the anti-tau antibodies as described in International Publication WO2017189963, the contents of which are herein incorporated by reference in their entirety. As a non-limiting example, the payload region may comprise one or more of the anti-tau antibodies as described in Table 3 of International Publication WO2017189963. In some embodiments, the payload region encodes one or more anti-tau antibodies selected from SEQ ID NO: 2948-4269 as described in WO2017189963.

In some embodiments, payloads may encode anti-tau antibodies (or fragments thereof) taught in United States Publication No. US2014294831, the contents of which are herein incorporated by reference in their entirety. Such antibodies may include IPN001 and/or IPN002 antibodies or fragments of such antibodies. In some cases, variable domains of IPN002 as presented in FIGS. 2A and 2B of US2014294831 may be used (e.g., incorporated into another antibody). In some cases, CDR regions of IPN002 as underlined in FIGS. 2A and 2B may be used (e.g., incorporated into another antibody or used to prepare humanized versions of IPN002). In some cases, anti-tau antibodies may include any of the IPN001 or IPN002 antibody variants taught in US2014294831 (e.g., in FIGS. 9-16 of that publication). In some embodiments, this antibody is also referred to as BMS-986168.

In some cases, payloads may encode anti-tau antibodies (or fragments thereof) taught in Otvos, L. et al., 1994. J Neurosci. Res 39(6):669-73, the contents of which are herein incorporated by reference in their entirety. Such antibodies may include monoclonal antibody PHF-1 or fragments thereof. The PHF-1 antibody binds to tau paired helical filaments, a pathological conformation of tau, found in certain neurological disorders, including Alzheimer's disease. Further, antibody affinity is increased when either serine 396 or serine 404 of tau is phosphorylated and even further increased when both are phosphorylated.

In some embodiments, payloads may encode anti-tau antibodies (or fragments thereof) taught in U.S. Pat. No. 5,811,310, the contents of which are herein incorporated by reference in their entirety. Such embodiments may include monoclonal antibodies PHF-1 or MC1 or fragments thereof. MC1 is a conformational antibody binding to the epitopes presented in Jicha, G. A., et al., 1997. J Neurosci Res 48(128-132).

In some embodiments, payloads may encode anti-tau antibodies (or fragments thereof) taught in International Publication Number WO2015035190, the contents of which are herein incorporated by reference in their entirety. Such embodiments may include, but are not limited to, antibodies PHF-1 or MC1 or fragments thereof. Viral genomes of the AAV particles of the present disclosure may comprise or encode any of SEQ ID NO: 1-6 of WO2015035190.

In some embodiments, viral genomes may encode anti-tau antibody MC1 scFv as described in Vitale et al 2018, (Acta Neuropath Commun. 6:82) the contents of which are herein incorporated by reference in their entirety.

In some embodiments, viral genomes may encode anti-tau antibody MC1 as described in International Publication WO2016137811, the contents of which are herein incorporated by reference in their entirety.

Anti-tau antibodies (or fragments thereof) encoded by viral genomes may include antibodies that bind to one or more of the epitopes presented in Otvos, L. et al., 1994. J Neurosci. Res 39(6):669-73 (e.g., any of those presented in Table 1 of that publication).

In some embodiments, payloads may encode anti-tau antibodies (or fragments thereof) taught in U.S. Pat. No. 7,746,180, the contents of which are herein incorporated by reference in their entirety. Such embodiments may include antibody DC-11 or fragments thereof.

In some embodiments, the antibodies encoded by the viral genomes of the present disclosure may target any of the antigenic regions or epitopes described in United States Patent Publication No US2008050383 or US20100316564, the contents of which are herein incorporated by reference in their entirety. In some embodiments, the antibody targets pS396/pS404. Such embodiments may include antibody 4E6 and/or variants or fragments thereof. The affinity of antibody 4E6 for soluble PHF and its ability to reduce soluble phospho tau has been described in Congdon, E. E. et al., 2016. Molecular Neurodegeneration August 30; 11(1):62, the contents of which are herein incorporated by reference in their entirety.

In some embodiments, the antibodies encoded by the viral genomes of the present disclosure may target any of the antigenic regions or epitopes described in International Patent Publication WO1998022120, the contents of which are herein incorporated by reference in their entirety. In some embodiments, the antibody may be PHF-6 (pT231), or fragments or variants thereof. In another embodiment, the antibody may be PHF-13 (pS396), or a fragment of variant thereof. These antibodies are further described in Hoffman et al., 1997. Biochemistry 36: 8114-8124, the contents of which are herein incorporated by reference in their entirety.

In some embodiments, the antibodies encoded by the viral genomes of the present disclosure may target any of the antigenic regions or epitopes described in International Publication WO2016126993, the contents of which are herein incorporated by reference in their entirety. The antibodies may be derived from any of the tau epitopes described in Table A of WO2016126993. In some embodiments, the antibody of the present disclosure may comprise any of the sequences listed in Table B or Table 1 of WO2016126993.

In some embodiments, the antibodies encoded by the viral genomes of the present disclosure may target any of the antigenic regions or epitopes described in United States Patent Publication US20120244174, the contents of which are herein incorporated by reference in their entirety. In some embodiments, the antibody may bind to caspase-cleaved tau. In some embodiments, the epitope for antibodies targeting caspase cleaved tau is aspartic acid 421. In another embodiment, the epitope for antibodies targeting caspase cleaved tau may be the C-terminus after glutamic residue Glu391. In yet another embodiment, the epitope for antibodies targeting caspase cleaved tau may be at the N-terminus at aspartic acid residue 13. In another embodiment, the antibody may be TauC3.

In some embodiments, the antibodies encoded by the viral genomes of the present disclosure may target any of the antigenic regions or epitopes described in United States Patent Publication US20160031978, the contents of which are herein incorporated by reference in their entirety. In some embodiments, the antibody may bind to tau N-terminal residues associated with the PP1/GSK3 signaling cascade. In some embodiments, the antibody may be TNT1.

In some embodiments, the antibodies encoded by the viral genomes of the present disclosure may be any of those described in d'Abramo, C et al., 2015. PLOS One 10(8):e0135774, the contents of which are herein incorporated by reference in their entirety. In some embodiments, the antibody may be CP13 (pS202), or a fragment or variant thereof. In another embodiment, the antibody may be RZ3 (pT231), or a fragment or variant thereof. In another embodiment, the antibody may be PG5 (pS409), or a fragment or variant thereof.

Anti-tau antibodies or fragments thereof encoded by the viral genomes of the present disclosure may target tau in any antigenic form. As non-limiting examples, antigenic tau may be an unphosphorylated or unmodified tau protein, a phosphorylated or otherwise post-translationally modified tau protein (O-GlnAcylated, or nitrosylated), an oligomeric species of tau protein, a soluble species of tau protein, an insoluble species of tau protein, a conformationally abnormal species of tau protein, a neuropathological form of tau protein and/or a neurofibrillary tangle or a precursor thereof.

Anti-tau antibodies or fragments thereof encoded by the viral genomes, may target any antigenic region or epitope along the full length of any of the six human tau protein isoforms, such as, but not limited to, tau441 (SEQ ID NO: 2127). As non-limiting examples, the targeted antigenic peptides of the tau protein may be any of the following phosphorylated sites pT50, pS396, pS396-pS404, pS404, pS396-pS404-pS422, pS409, pS413, pS422, pS198, pS1⁹⁹, pS199-pS202, pS202, pT205, pT212, pS214, pT212-pS214, pT181, pT231, cis-pT231, pS235, pS238, pT245, pS262, pY310, pY394, pS324, pS356, pTau¹⁷⁷⁻¹⁸⁷, pY18, pS610, pS622, nitrosylated tau (nY18, nY29), methylated tau (di-meK281, dimeK311), O-GlnAcylated tau at S400, any of the following acetylated sites acK174, acK274, acK280, acK281 and/or any combination thereof. Acetylated tau proteins and associated antigenic peptides are described in Min et al., 2010, Neuron., 67, 953-966, Min et al., 2015, Nature Medicine., 10, 1154-1162, Cohen et al., 2011, Nature Communications., 2, 252, Gorsky et al., 2016, Scientific Report., 6, 22685, Tracy et al., 2016, Neuron., 90, 245-260, the contents of each of which are herein incorporated by reference in their entirety. Phosphorylated tau proteins and associated antigenic peptides are described in Asuni et al., 2007, J Neurosci., 27, 9115-9129, Boutajangout et al., 2010, J Neurosci., 30, 16559-16566, Boutajangout et al., 2011, J Neurochem., 118, 658-667, Chai et al., 2011, J Biol Chem., 286, 34457-34467, Gu et al., 2011, J Biol Chem., 288, 33081-33095, Sankaranarayanan et al., 2015, PLoS One, 10, e0125614, Ittner et al., 2015, J Neurochem., 132, 135-145, D'Abramo et al., 2016, Neurobiol Aging., 37, 58-65, Collin et al., 2014, Brain., 137, 2834-2846, Kondo et al., 2015, Nature., 523, 431-436, the contents of each of which are herein incorporated by reference in their entirety.

In some embodiments, the targeted antigenic peptides of the tau protein may comprise a sequence selected from SEQ ID NO: 2128-2136.

In some embodiments, the antibody encoded by the viral genomes of the present disclosure may be a pS409 targeting antibody as described in Lee et al., 2016, Cell Reports, 16, 1690-1700, or International Patent Publication WO2013151762, the contents of each of which are herein incorporated by reference in their entirety. In some embodiments, this antibody may be RG6100 or R071057 or variants or fragments thereof.

In some embodiments, the antibody encoded by the viral genomes of the present disclosure may be a pS413 targeting antibody as described in Umeda et al., 2015, Ann Clin Trans Neurol., 2(3), 241-255 or International Patent Publication WO2013180238, the contents of each of which are herein incorporated by reference in their entirety. In some embodiments, the antibody is Ta1505 or variants or fragments thereof.

In some embodiments, the antibody encoded by the viral genomes of the present disclosure may target a tau epitope with amino acid residues 210-275, more specifically pS238 and/or pT245, as described in International Publication WO2011053565, the contents of which are herein incorporated by reference in their entirety.

In some embodiments, the CDRs of an antibody encoded by the viral genomes of the present disclosure may be any of those listed in or incorporated in the antibody sequences of Table 3. In some embodiments, the CDRs may be any of those described in International Publication WO2015122922, the contents of which are herein incorporated by reference in their entirety. In some embodiments, a CDR may be any of those chosen from the group of SEQ ID NO: 41, 49, or 57 of WO2015122922. Further a CDR of an antibody encoded by the viral genomes of the present disclosure may have 50%, 60%, 70%, 80%, 90%, or 95% identity to SEQ ID NO: 41, 49, or 57 of WO2015122922.

In some embodiments, the antibodies encoded by the viral genomes of the present disclosure may be any of those described in International Publication WO2016097315, the contents of which are herein incorporated by reference in their entirety. In some embodiments, the antibody may have an amino acid sequence as shown by SEQ ID NO: 2, 11, 20, 29, 38, 47, 56, 65, 74, 83, 92, 101, 110, 119, 128, 137, 146, 155, 164, 173, 182, 191, 209, 218, 226, or 227 of WO2016097315.

In some embodiments, the antibodies encoded by the viral genomes of the present disclosure may be a multispecific blood brain barrier receptor antibody that also targets tau, as described in International Publication WO2016094566, the contents of which are herein incorporated by reference in their entirety. In some embodiments, the antibody may have a sequence as shown by SEQ ID NO: 1, 2, 17, 18, 33, 34, 49, 50, 65, 66, 81, 82, 9-16, 25-32, 41-48, 57-64, 73-80, 89-96 of WO2016094566.

In some embodiments, the antibodies (or fragments thereof) encoded by the viral genomes of the present disclosure may be any of those taught in U.S. Pat. Nos. 8,778,343 and 9,125,846, International Publications WO2012051498 and WO2011026031, or United States Publication Nos. US20150004169 and US20150322143, the contents of each of which are herein incorporated by reference in their entirety. Such antibodies may include those that bind to oligomeric species of tau. Further, such an antibody may be referred to as TOMA (tau oligomer monoclonal antibody), as described in Castillo-Carranza et at (Castillo-Carranza, D L et al., 2014 J Neurosci 34(12)4260-72) the contents of which are herein incorporated by reference in their entirety. In some embodiments, the antibody that binds oligomeric tau may be TTC-99.

In some embodiments, the antibodies (or fragments thereof) encoded by the viral genomes of the present disclosure may be any of those taught in International Publications WO2014059442, the contents of which are herein incorporated by reference in their entirety. Such antibodies may include those that bind to oligomeric species of tau.

In some embodiments, the antibodies (or fragments thereof) encoded by the viral genomes of the present disclosure may be any of those taught in the International Publications WO2014008404 and WO2016126993, United States Patent Publication US20150183855, Yanamandra, K et al., 2013 Neuron 80(2):402-14 and Yanamandra, K et al 2015 Ann Clin Transl Neurol 2(3):278-88, the contents of each of which are herein incorporated by reference in their entirety. Such antibodies may block tau seeding. Non-limiting examples of antibodies described in these publications include HJ8.1.1, HJ8.1.2, HJ8.2, HJ8.3, HJ8.4, HJ8.5, HJ8.7, HJ8.8, HJ9.1, HJ9.2, HJ9.3, HJ9.4, HJ9.5, and variants thereof. Non-limiting examples of targeted epitopes of tau may include amino acids 22-34, 385-391, 405-411, 3-6, 118-122, 386-401, 7-13, and/or 272-281 of human tau.

In some embodiments, the antibodies (or fragments thereof) encoded by the viral genomes of the present disclosure may be any of those taught in the International Publications WO2002062851, the contents of which are herein incorporated by reference in their entirety.

In some embodiments, the antibodies (or fragments thereof) encoded by the viral genomes of the present disclosure may be as described in Bright, J et al., 2015 Neurobiol of Aging 36:693-709; Pedersen, JT and Sigurdsson E M, 2015 Trends Mol Med 21(6):394-402; Levites, Y et al 2015 J Neurosci 35(16)6265-76; Jicha et al 1999 J Neurosci 19(17):7486-94; Reyes μF et al., 2012 Acta Neuropathol 123(1):119-32; Reynolds M R, et al., 2006 J Neurosci 26(42):10636-45; Gamblin, T C et al 2003 PNAS 100(17):10032-7; Castillo-Carranza, D L et al., 2014 J Neurosci 34(12)4260-72; Walls, K C et al., 2014 Neurosci Lett 575:96-100; Yanamandra, K et al., 2013 Neuron 80(2):402-14; Yanamandra, K et al 2015 Ann Clin Transl Neurol 2(3):278-88; Allen B, et al., 2002 J Neurosci 22(21):9340-51; Gotz, J et al., 2010 Biochem Biophys Acta 1802(10):860-71; Hasegawa, M et al 1996 FEBS Lett 384:25-30; Carmel, G et al 1996 J Biol Chem 271(51):32780-32795; Jicha, G A et al, 1997 J Neurosci Res 48(2):128-132; Jicha, G A et al., 1997 J Neurochem 69(5):2087-95; the contents of each of which are herein incorporated by reference in their entirety.

Anti-tau antibodies or fragments thereof encoded by the viral genomes of the present disclosure may be any commercially available anti-tau antibody known in the art or developed by a person with skill in the art. Non-limiting examples of commercially available anti-tau antibodies include EPR2396(2) (pThr⁵⁰; Abcam, Cambridge, MA), 5H911 (pThr¹⁸¹; ThermoFisher, Waltham, MA), M7004D06 (pThr¹⁸¹; BioLegend, San Diego, CA), 1E7 (pThr¹⁸¹. EMD Millipore, Billerica, MA), EPR2400 (pSer¹⁹⁸; Abcam, Cambridge, MA), EPR2401Y (pSer¹⁹⁹; Abcam, Cambridge, MA), 2H23L4 (pSer¹⁹⁹; ThermoFisher, Waltham, MA), EPR2402 (pSer²⁰²; Abcam, Cambridge, MA), 10F8 (pSer²⁰²; Abcam, Cambridge, MA), EPR2403(2) (pThr²⁰⁵; Abcam, Cambridge, MA), EPR1884(2) (pSer²¹⁴; Abcam, Cambridge, MA), EPR2488 (pThr²³¹; Abcam, Cambridge, MA), 1H6L6 (pThr²³¹; ThermoFisher, Waltham, MA), 3G3 (pThr²³¹, pSer²³s; Abcam, Cambridge, MA), EPR2452 (pSer²³⁵; Abcam, Cambridge, MA), 12G10 (pSer²³⁸; Abcam, Cambridge, MA), EPR2454 (pSer²⁶²; Abcam, Cambridge, MA), EPR2457(2) (pSer³²⁴; Abcam, Cambridge, MA), EPR2603 (pSer³⁵⁶; Abcam, Cambridge, MA), EPR2731 (pSer³⁹⁶; Abcam, Cambridge, MA), EPR2605 (pSer⁴⁰⁴; Abcam, Cambridge, MA), EPR2866 (pSer⁴²²; Abcam, Cambridge, MA), 1A4 (pTau¹⁷⁷⁻¹⁸⁷; Origene, Rockville, MD), 7G9 (pTau¹⁷⁷⁻¹⁸⁷; Origene, Rockville, MD), 9B4 (pTau¹⁷⁷⁻¹⁸⁷; Origene, Rockville, MD), 2A4 (pTau¹⁷⁷⁻¹⁸⁷; Origene, Rockville, MD), 9G3 (pTyr¹⁸; NovusBio, Littleton, CO), EPR2455(2) (pSer⁶¹⁰; Abcam, Cambridge, MA), EP2456Y (pSer⁶²²; Abcam, Cambridge, MA; EMD Millipore, Billerica, MA), SMI 51 (PHF Tau⁹⁵⁻¹⁰⁸; BioLegend, San Diego, CA), TOMA-1 (Oligomeric Tau; EMD Millipore, Billerica, MA), Tau-nY18 (nTyr¹⁸; Origene, Rockville, MD; BioLegend, San Diego, CA; EMD Millipore, Billerica, MA), Tau-nY29 (nTyr²⁹; BioLegend, San Diego, CA; EMD Millipore, Billerica, MA; Abcam, Cambridge, MA), 1C9.G6 (di-methyl-Lys²⁸¹; BioLegend, San Diego, CA), 7G5.F4 (di-methyl-Lys³¹¹; BioLegend, San Diego, CA), TNT-1 (Tau²⁻¹⁸; EMD Millipore, Billerica, MA), TNT-2 (Tau²⁻¹⁸; EMD Millipore, Billerica, MA), 7B8 (Tau⁵⁻¹²; Abcam, Cambridge, MA), Tau-13 (Tau²⁰⁻³⁵; BioLegend, San Diego, CA), 1-100 (Tau¹⁻¹⁰⁰; BioLegend, San Diego, CA), 2G9.F10 (Tau¹⁵⁷-1⁶⁸; BioLegend, San Diego, CA; Origene, Rockville, MD), 39E10 (Tau¹⁸⁹-1⁹⁵; BioLegend, San Diego, CA; Origene, Rockville, MD), 77E9 (Tau¹⁸⁵⁻¹⁹⁵; BioLegend, San Diego, CA; Origene, Rockville, MD), AT8 (pSer²⁰², pThr²⁰⁵; ThermoFisher, Waltham, MA), AT100 (pThr²¹², pSer²¹⁴; ThermoFisher, Waltham, MA), PHF-6 (pThr²³¹; NovusBio, Littleton, CO; EMD Millipore, Billerica, MA; BioLegend, San Diego, CA; ThermoFisher, Waltham, MA), AT180 (pThr²³¹; ThermoFisher, Waltham, MA), AT270 (pThr¹⁸¹; ThermoFisher, Waltham, MA), PHF-13 (pSer³⁹⁶; ThermoFisher, Waltham, MA; BioLegend, San Diego, CA), TauC3 (Asp⁴²¹; BioLegend, San Diego, CA; EMD Millipore, Billerica, MA; ThermoFisher, Waltham, MA), Tau12 (Tau⁶⁻¹⁸; BioLegend, San Diego, CA; EMD Millipore, Billerica, MA), Tau5 (Tau²¹⁰⁻²⁴¹; BioLegend, San Diego, CA; EMD Millipore, Billerica, MA; Abcam, Cambridge MA; ThermoFisher, Waltham, MA), HT7 (Tau¹⁵⁹⁻¹⁶³; ThermoFisher, Waltham, MA), 77G7 (Tau³¹⁶⁻³⁵⁵; BioLegend, San Diego, CA), Tau46 (Tau⁴⁰⁴⁻⁴⁴¹; BioLegend, San Diego, CA; NovusBio, Littleton, CO; Abeam, Cambridge, MA), UMAB239 (Tau⁶²³⁻⁷⁵⁸; Origene, Rockville, MD), OTI6G3 (Tau⁶²³⁻⁷⁵⁸; Origene, Rockville, MD), OTI13E11 (Tau⁶²³⁻⁷⁵⁸; Origene, Rockville, MD), OTI13B5 (Tau⁶²³⁻⁷⁵⁸; Origene, Rockville, MD), E178 (Tau⁷⁰⁰⁻⁸⁰⁰; Abeam, Cambridge, MA), SP70 (N-terminal Tau; Origene, Rockville, MD; NovusBio, Littleton, CO; ThermoFisher, Waltham, MA; Abeam, Cambridge, MA), C45 (N-terminal Tau; Origene, Rockville, MD), Tau7 (C-terminal Tau; EMD Millipore, Billerica, MA), S.125.0 (C-terminal Tau; ThermoFisher, Waltham, MA), 8E6/C11 (Three-repeat Tau²⁰⁹⁻²²⁴; EMD Millipore, Billerica, MA), 1E1/A6 (Four-repeat Tau²⁷⁵⁻²⁹¹; EMD Millipore, Billerica, MA), 7D12.1 (Four-repeat Tau²⁷⁵⁻²⁹¹; EMD Millipore, Billerica, MA), 5C7 (Four-repeat Tau²⁶⁷⁻²⁷⁸; BioLegend, San Diego, CA; Origene, Rockville, MD), 5F9 (Four-repeat Tau²⁷⁵⁻²⁹¹. BioLegend, San Diego, CA; Origene, Rockville, MD), 3H6.H7 (0N Tau³⁹⁻⁵⁰; BioLegend, San Diego, CA; Origene, Rockville, MD), 4H5.B9 (1N Tau⁶⁸⁻⁷⁹; BioLegend, San Diego, CA; Origene, Rockville, MD), 71C11 (2N Tau; BioLegend, San Diego, CA), PC1C6 (unphosphorylated tau; EMD Millipore, Billerica, MA), Tau2 (BioLegend, San Diego, CA; Origene, Rockville, MD; EMD Millipore, Billerica, MA), 2E9 (Origene, Rockville, MD; NovusBio, Littleton, CO), 4F1 (Origene, Rockville, MD; NovusBio, Littleton, CO), 5B10 (NovusBio, Littleton, CO); 5E2 (EMD Millipore, Billerica, MA), Tau-93 (Origene, Rockville, MD), T14 (ThermoFisher, Waltham, MA), T46 (ThermoFisher, Waltham, MA), BT2 (ThermoFisher, Waltham, MA) and/or variants or derivates thereof.

In some embodiments, the antibodies encoded by the viral genomes of the present disclosure may be multispecific antibodies for transferrin receptor and a brain antigen, wherein the brain antigen may be tau, as described in International Publication WO2016081643, the contents of which are herein incorporated by reference in their entirety. In some embodiments, the antibody may have a sequence as given by SEQ ID NO: 160 or 161 of WO2016081643.

In some embodiments, the antibodies encoded by the viral genomes of the present disclosure are any of those described in U.S. Pat. Nos. 8,871,447, 8,420,613, International Publication No. WO2014193935, WO2010011999, or in United States Publication Nos. US20110250217, US20110020237, US20100316590, or US20120225864, the contents of each of which are herein incorporated by reference in their entirety. In some embodiments, the antibody recognizes a misfolded, amyloidogenic or aggregating protein.

In some embodiments, the viral genome of the AAV particle of the present disclosure encodes anti-tau antibody PT3, or a fragment or variant thereof.

In some embodiments, the viral genome of the AAV particle of the present disclosure encodes anti-tau antibody AT8, or a fragment or variant thereof.

In some embodiments, the viral genome of the AAV particle of the present disclosure encodes anti-tau antibody IPN002, or a fragment or variant thereof.

In some embodiments, the viral genome of the AAV particle of the present disclosure encodes anti-tau antibody MC1, or a fragment or variant thereof.

In some embodiments, the viral genome of the AAV particle of the present disclosure encodes anti-tau antibody PHF1, or a fragment or variant thereof.

In some embodiments, the viral genome of the AAV particle of the present disclosure encodes anti-tau antibody CP13, or a fragment or variant thereof.

In some embodiments, the viral genome of the AAV particle of the present disclosure encodes anti-tau antibody C10.2, or a fragment or variant thereof.

In some embodiments, the viral genome of the AAV particle of the present disclosure encodes anti-tau antibody PHF-13, or a fragment or variant thereof.

In some embodiments, the viral genome of the AAV particle of the present disclosure encodes anti-tau antibody PHF-6, or a fragment or variant thereof.

### The Nature of the Polypeptides and Variants

Antibodies encoded by payload regions of the viral genomes may be translated as a whole polypeptide, a plurality of polypeptides or fragments of polypeptides, which independently may be encoded by one or more nucleic acids, fragments of nucleic acids or variants of any of the aforementioned. As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not be confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least about 70% homology to a native sequence, and preferably, they will be at least about 80%, more preferably at least about 90% homologous to a native sequence. “Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to include polypeptide variants which differ by one or more amino acid alterations, e.g., substitutions, additions, or deletions of amino acid residues that still maintain the properties of the parent polypeptide.

Sequence tags or amino acids, such as one or more lysines, can be added to the peptide sequences (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to proteins are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to proteins, are those with one or more amino acids in the native or starting amino acid sequence removed. Ordinarily, deletional variants will have one or more amino acids deleted in a particular region of the molecule.

As used herein, the term “derivative” is used synonymously with the term “variant” and refers to a molecule that has been modified or changed in any way relative to a reference molecule or starting molecule. In some embodiments, derivatives include native or starting proteins that have been modified with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present disclosure.

Other post-translational modifications include hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

“Features” when referring to proteins are defined as distinct amino acid sequence-based components of a molecule. Features of the proteins of the present disclosure include surface manifestations, local conformational shape, folds, loops, half-loops, domains, half-domains, sites, termini or any combination thereof.

As used herein when referring to proteins the term “surface manifestation” refers to a polypeptide-based component of a protein appearing on an outermost surface.

As used herein when referring to proteins the term “local conformational shape” means a polypeptide based structural manifestation of a protein which is located within a definable space of the protein.

As used herein when referring to proteins the term “fold” means the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to proteins the term “loop” refers to a structural feature of a peptide or polypeptide which reverses the direction of the backbone of a peptide or polypeptide and comprises four or more amino acid residues. Oliva et al. have identified at least 5 classes of protein loops (J. Mol Biol 266 (4): 814-830; 1997).

As used herein when referring to proteins the term “half-loop” refers to a portion of an identified loop having at least half the number of amino acid residues as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7-amino acid loop could produce half-loops of 3 amino acids or 4 amino acids (7/2=3.5+/−0.5 being 3 or 4).

As used herein when referring to proteins the term “domain” refers to a motif of a polypeptide having one or more identifiable structural or functional characteristics or properties (e.g., binding capacity, serving as a site for protein-protein interactions).

As used herein when referring to proteins the term “half-domain” means portion of an identified domain having at least half the number of amino acid residues as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7-amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to proteins the terms “site” as it pertains to amino acid-based embodiments is used synonymous with “amino acid residue” and “amino acid side chain”. A site represents a position within a peptide or polypeptide that may be modified, manipulated, altered, derivatized or varied within the polypeptide-based molecules of the present disclosure.

As used herein the terms “termini or terminus” when referring to proteins refers to an extremity of a peptide or polypeptide. Such extremity is not limited only to the first or final site of the peptide or polypeptide but may include additional amino acids in the terminal regions. The polypeptide-based molecules of the present disclosure may be characterized as having both an N-terminus (terminated by an amino acid with a free amino group (NH2)) and a C-terminus (terminated by an amino acid with a free carboxyl group (COOH)). Proteins are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide-based moiety such as an organic conjugate.

Once any of the features have been identified or defined as a component of a molecule, any of several manipulations and/or modifications of these features may be performed by moving, swapping, inverting, deleting, randomizing, or duplicating. Furthermore, it is understood that manipulation of features may result in the same outcome as a modification to the molecules. For example, a manipulation which involves deleting a domain would result in the alteration of the length of a molecule just as modification of a nucleic acid to encode less than a full-length molecule would.

Modifications and manipulations can be accomplished by methods known in the art such as site directed mutagenesis. The resulting modified molecules may then be tested for activity using in vitro or in vivo assays such as those described herein or any other suitable screening assay known in the art.

### AAV Production

The present disclosure provides methods for the generation of parvoviral particles, e.g. AAV particles, by viral genome replication in a viral replication cell.

In accordance with the disclosure, the viral genome comprising a payload region encoding an antibody, an antibody-based composition or fragment thereof, will be incorporated into the AAV particle produced in the viral replication cell. Methods of making AAV particles are well known in the art and are described in e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353, and WO2001023597; Methods In Molecular Biology, ed. Richard, Humana Press, N J (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir., 219:37-44 (1996); Zhao et al., Vir. 272:382-93 (2000); the contents of each of which are herein incorporated by reference in their entirety. In some embodiments, the AAV particles are made using the methods described in WO2015191508, the contents of which are herein incorporated by reference in their entirety.

Viral replication cells commonly used for production of recombinant AAV viral vectors include but are not limited to 293 cells, COS cells, HeLa cells, KB cells, and other mammalian cell lines as described in U.S. Pat. Nos. 6,156,303, 5,387,484, 5,741,683, 5,691,176, and 5,688,676; U.S. patent publication No. 2002/0081721, and International Patent Publication Nos. WO 00/47757, WO 00/24916, and WO 96/17947, the contents of each of which are herein incorporated by reference in their entireties.

In some embodiments, the AAV particles of the present disclosure may be produced in insect cells (e.g., Sf9 cells).

In some embodiments, the AAV particles of the present disclosure may be produced using triple transfection.

In some embodiments, the AAV particles of the present disclosure may be produced in mammalian cells.

In some embodiments, the AAV particles of the present disclosure may be produced by triple transfection in mammalian cells.

In some embodiments, the AAV particles of the present disclosure may be produced by triple transfection in HEK293 cells.

The present disclosure provides a method for producing an AAV particle comprising the steps of: 1) co-transfecting competent bacterial cells with a bacmid vector and either a viral construct vector and/or AAV payload construct vector, 2) isolating the resultant viral construct expression vector and AAV payload construct expression vector and separately transfecting viral replication cells, 3) isolating and purifying resultant payload and viral construct particles comprising viral construct expression vector or AAV payload construct expression vector, 4) co-infecting a viral replication cell with both the AAV payload and viral construct particles comprising viral construct expression vector or AAV payload construct expression vector, 5) harvesting and purifying the viral particle comprising a parvoviral genome.

In some embodiments, the present disclosure provides a method for producing an AAV particle comprising the steps of 1) simultaneously co-transfecting mammalian cells, such as, but not limited to HEK293 cells, with a payload region, a construct expressing rep and cap genes and a helper construct, 2) harvesting and purifying the AAV particle comprising a viral genome.

In some embodiments, the viral construct vector(s) used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the contents of which are herein incorporated by reference in its entirety.

In some embodiments, the viral construct vector(s) used for AAV production may contain a nucleotide sequence encoding the AAV rep proteins where the initiation codon of the AAV rep protein or proteins is a non-ATG. In some embodiments, a single coding sequence is used for the Rep78 and Rep52 proteins, wherein initiation codon for translation of the Rep78 protein is a suboptimal initiation codon, selected from the group consisting of ACG, TTG, CTG and GTG, that effects partial exon skipping upon expression in insect cells, as described in U.S. Pat. No. 8,512,981, the contents of which is herein incorporated by reference in its entirety, for example to promote less abundant expression of Rep78 as compared to Rep52, which may be advantageous in that it promotes high vector yields.

In some embodiments, the viral genome of the AAV particle optionally encodes a selectable marker. The selectable marker may comprise a cell-surface marker, such as any protein expressed on the surface of the cell including, but not limited to receptors, CD markers, lectins, integrins, or truncated versions thereof.

In some embodiments, selectable marker reporter genes are selected from those described in International Application No. WO 96/23810; Heim et al., Current Biology 2:178-182 (1996); Heim et al., Proc. Natl. Acad. Sci. USA (1995); or Heim et al., Science 373:663-664 (1995); WO 96/30540, the contents of each of which are incorporated herein by reference in their entireties).

The AAV viral genomes encoding an anti-tau antibody payload described herein may be useful in the fields of human disease, veterinary applications and a variety of in vivo and in vitro settings. The AAV particles of the present disclosure may be useful in the field of medicine for the treatment, prophylaxis, palliation or amelioration of neurological diseases and/or disorders. In some embodiments, the AAV particles are used for the prevention and/or treatment of a tauopathy.

Various embodiments herein provide a pharmaceutical composition comprising the AAV particles described herein and a pharmaceutically acceptable excipient.

Various embodiments herein provide a method of treating a subject in need thereof comprising administering to the subject a therapeutically effective amount of the pharmaceutical composition described herein.

Certain embodiments of the method provide that the subject is treated by a route of administration of the pharmaceutical composition selected from the group consisting of intravenous, intracerebroventricular, intraparenchymal, intrathecal, subpial and intramuscular, or a combination thereof. Certain embodiments of the method provide that the subject is treated for a tauopathy and/or other neurological disorder. In one aspect of the method, a pathological feature of the tauopathy or other neurological disorder is alleviated and/or the progression of the tauopathy or other neurological disorder is halted, slowed, ameliorated or reversed.

Various embodiments herein describe a method of decreasing the level of soluble tau in the central nervous system of a subject in need thereof comprising administering to said subject an effective amount of the pharmaceutical composition described herein.

Also described herein are compositions, methods, processes, kits and devices for the design, preparation, manufacture and/or formulation of AAV particles. In some embodiments, payloads, such as but not limited to anti-tau antibodies, may be encoded by payload constructs or contained within plasmids or vectors or recombinant adeno-associated viruses (AAVs).

The present disclosure also provides administration and/or delivery methods for vectors and viral particles, e.g., AAV particles, for the treatment or amelioration of neurological disease, such as, but not limited to tauopathy.

### AAV Particles Comprising Anti-Tau Antibody Payloads

In some embodiments, the AAV particle comprises a viral genome with a payload region comprising one or more anti-tau antibody polynucleotide sequences.

In some embodiments, a viral genome encoding more than one polypeptide may be replicated and packaged into a viral particle. A target cell transduced with a viral particle comprising one or more anti-tau antibody polynucleotides may express the encoded antibody or antibodies in a single cell.

In some embodiments, the AAV particles are useful in the field of medicine for the treatment, prophylaxis, palliation or amelioration of neurological diseases and/or disorders.

Non-limiting examples of ITR to ITR sequences of AAV particles comprising a viral genome with a payload region comprising an anti-tau antibody polynucleotide sequence are described in Table 4-6.

TABLE 4
ITR to ITR Sequences of AAV particles
comprising anti-tau antibody
polynucleotide sequences

| ITR to ITR | Length | SEQ ID |
|---|---|---|
| Construct Name | (nt) | NO: |
| TAU_ITR1 | 4450 | 1990 |
| TAU_ITR2 | 4471 | 1991 |
| TAU_ITR3 | 3983 | 1992 |
| TAU_ITR4 | 3962 | 1993 |
| TAU_ITR5 | 4041 | 1994 |
| TAU_ITR6 | 4020 | 1995 |
| TAU_ITR7 | 3899 | 1996 |
| TAU_ITR8 | 3878 | 1997 |
| TAU_ITR17 | 3932 | 2006 |
| TAU_ITR18 | 3932 | 2007 |
| TAU_ITR19 | 3932 | 2008 |
| TAU_ITR20 | 3932 | 2009 |
| TAU_ITR21 | 3932 | 2010 |
| TAU_ITR22 | 3932 | 2011 |
| TAU_ITR23 | 3932 | 2012 |
| TAU_ITR24 | 3932 | 2013 |
| TAU_ITR25 | 3932 | 2014 |
| TAU_ITR26 | 4309 | 2015 |
| TAU_ITR27 | 4309 | 2016 |
| TAU_ITR28 | 4309 | 2017 |
| TAU_ITR29 | 4309 | 2018 |
| TAU_ITR30 | 4309 | 2019 |
| TAU_ITR31 | 4309 | 2020 |
| TAU_ITR32 | 4309 | 2021 |
| TAU_ITR33 | 4309 | 2022 |
| TAU_ITR34 | 4309 | 2023 |
| TAU_ITR35 | 4309 | 2024 |
| TAU_ITR87 | 3950 | 2137 |
| TAU_ITR88 | 3962 | 2138 |
| TAU_ITR89 | 4450 | 2139 |
| TAU_ITR90 | 4020 | 2140 |
| TAU_ITR91 | 3878 | 2141 |
| TAU_ITR92 | 3950 | 2142 |
| TAU_ITR93 | 4438 | 2143 |
| TAU_ITR94 | 4008 | 2144 |
| TAU_ITR95 | 3866 | 2145 |
| TAU_ITR96 | 3941 | 2146 |
| TAU_ITR97 | 4429 | 2147 |
| TAU_ITR98 | 3999 | 2148 |
| TAU_ITR99 | 3857 | 2149 |
| TAU_ITR100 | 4420 | 2150 |
| TAU_ITR101 | 3990 | 2151 |
| TAU_ITR102 | 3848 | 2152 |
| TAU_ITR103 | 3971 | 2153 |
| TAU_ITR104 | 4459 | 2154 |
| TAU_ITR105 | 4029 | 2155 |
| TAU_ITR106 | 3887 | 2156 |
| TAU_ITR107 | 3938 | 2157 |
| TAU_ITR108 | 4447 | 2158 |
| TAU_ITR109 | 4017 | 2159 |
| TAU_ITR110 | 3875 | 2160 |
| TAU_ITR111 | 3950 | 2161 |
| TAU_ITR112 | 4438 | 2162 |
| TAU_ITR113 | 4008 | 2163 |
| TAU_ITR160 | 3097 | 2210 |
| TAU_ITR161 | 3082 | 2211 |
| TAU_ITR162 | 3097 | 2212 |
| TAU_ITR163 | 3112 | 2213 |
| TAU_ITR164 | 2652 | 2214 |
| TAU_ITR165 | 2667 | 2215 |
| TAU_ITR166 | 2730 | 2216 |
| TAU_ITR167 | 2510 | 2217 |
| TAU_ITR168 | 2525 | 2218 |
| TAU_ITR169 | 2540 | 2219 |
| TAU_ITR170 | 3091 | 2220 |
| TAU_ITR171 | 3082 | 2221 |
| TAU_ITR172 | 3103 | 2222 |
| TAU_ITR173 | 3109 | 2223 |
| TAU_ITR174 | 3115 | 2224 |
| TAU_ITR175 | 3121 | 2225 |
| TAU_ITR176 | 3097 | 2226 |
| TAU_ITR177 | 3124 | 2227 |
| TAU_ITR178 | 3109 | 2228 |
| TAU_ITR179 | 3157 | 2229 |
| TAU_ITR180 | 3760 | 2230 |
| TAU_ITR181 | 3781 | 2231 |
| TAU_ITR182 | 3778 | 2232 |
| TAU_ITR183 | 3748 | 2233 |
| TAU_ITR184 | 3769 | 2234 |
| TAU_ITR185 | 3766 | 2235 |
| TAU_ITR186 | 3760 | 2236 |
| TAU_ITR187 | 3778 | 2237 |
| TAU_ITR188 | 3188 | 2260 |
| TAU_ITR189 | 3330 | 2261 |
| TAU_ITR190 | 3976 | 2262 |
| TAU_ITR191 | 3404 | 2263 |
| TAU_ITR192 | 3546 | 2264 |
| TAU_ITR193 | 3997 | 2265 |
| TAU_ITR194 | 3425 | 2266 |
| TAU_ITR195 | 3567 | 2267 |
| TAU_ITR196 | 3088 | 2268 |
| TAU_ITR197 | 3558 | 2269 |
| TAU_ITR198 | 3360 | 2270 |
| TAU_ITR199 | 3384 | 2271 |
| TAU_ITR200 | 2625 | 2272 |
| TAU_ITR9 | 4459 | 1998 |
| TAU_ITR10 | 4438 | 1999 |
| TAU_ITR11 | 3971 | 2000 |
| TAU_ITR12 | 4029 | 2001 |
| TAU_ITR13 | 4008 | 2002 |
| TAU_ITR14 | 3887 | 2003 |
| TAU_ITR15 | 3866 | 2004 |
| TAU_ITR16 | 3932 | 2005 |
| TAU_ITR114 | 3866 | 2164 |
| TAU_ITR115 | 3938 | 2165 |
| TAU_ITR116 | 4426 | 2166 |
| TAU_ITR117 | 3996 | 2167 |
| TAU_ITR118 | 3854 | 2168 |
| TAU_ITR119 | 3950 | 2161 |
| TAU_ITR120 | 4438 | 2162 |
| TAU_ITR121 | 3455 | 2171 |
| TAU_ITR122 | 3498 | 2172 |
| TAU_ITR123 | 3282 | 2173 |
| TAU_ITR124 | 3300 | 2174 |
| TAU_ITR125 | 3943 | 2175 |
| TAU_ITR126 | 3988 | 2176 |
| TAU_ITR127 | 3772 | 2177 |
| TAU_ITR128 | 3790 | 2178 |
| TAU_ITR129 | 3324 | 2179 |
| TAU_ITR130 | 3814 | 2180 |
| TAU_ITR131 | 2567 | 2181 |
| TAU_ITR132 | 2582 | 2182 |
| TAU_ITR133 | 2597 | 2183 |
| TAU_ITR134 | 2612 | 2184 |
| TAU_ITR135 | 2627 | 2185 |
| TAU_ITR136 | 2642 | 2186 |
| TAU_ITR137 | 3055 | 2187 |
| TAU_ITR138 | 3070 | 2188 |
| TAU_ITR139 | 3085 | 2189 |
| TAU_ITR140 | 3100 | 2190 |
| TAU_ITR141 | 3115 | 2191 |
| TAU_ITR142 | 3130 | 2192 |
| TAU_ITR143 | 3160 | 2193 |
| TAU_ITR144 | 2569 | 2194 |
| TAU_ITR145 | 2584 | 2195 |
| TAU_ITR146 | 2599 | 2196 |
| TAU_ITR147 | 2614 | 2197 |
| TAU_ITR148 | 2627 | 2198 |
| TAU_ITR149 | 2642 | 2199 |
| TAU_ITR150 | 3055 | 2200 |
| TAU_ITR151 | 3070 | 2201 |
| TAU_ITR152 | 3084 | 2202 |
| TAU_ITR153 | 3100 | 2203 |
| TAU_ITR154 | 3115 | 2204 |
| TAU_ITR155 | 3130 | 2205 |
| TAU_ITR156 | 3097 | 2206 |
| TAU_ITR157 | 3082 | 2207 |
| TAU_ITR158 | 3097 | 2208 |
| TAU_ITR159 | 3112 | 2209 |
| TAU_ITR201 | 2655 | 2273 |
| TAU_ITR202 | 2685 | 2274 |
| TAU_ITR203 | 2730 | 2275 |
| TAU_ITR204 | 2625 | 2276 |
| TAU_ITR205 | 2655 | 2277 |
| TAU_ITR206 | 2685 | 2278 |
| TAU_ITR207 | 3416 | 2279 |
| TAU_ITR208 | 3218 | 2280 |
| TAU_ITR209 | 3242 | 2281 |
| TAU_ITR210 | 2483 | 2282 |
| TAU_ITR211 | 2513 | 2283 |
| TAU_ITR212 | 2543 | 2284 |
| TAU_ITR213 | 2588 | 2285 |
| TAU_ITR214 | 2483 | 2286 |
| TAU_ITR215 | 2513 | 2287 |
| TAU_ITR216 | 2543 | 2288 |
| TAU_ITR217 | 3967 | 2289 |
| TAU_ITR218 | 3769 | 2290 |
| TAU_ITR219 | 3792 | 2291 |
| TAU_ITR220 | 3751 | 2292 |
| TAU_ITR221 | 3058 | 2293 |
| TAU_ITR222 | 3088 | 2294 |
| TAU_ITR223 | 3118 | 2295 |
| TAU_ITR224 | 3163 | 2296 |
| TAU_ITR225 | 3537 | 2297 |
| TAU_ITR226 | 3339 | 2298 |
| TAU_ITR227 | 3362 | 2299 |
| TAU_ITR228 | 3321 | 2300 |
| TAU_ITR229 | 2628 | 2301 |
| TAU_ITR230 | 2658 | 2302 |
| TAU_ITR231 | 2688 | 2303 |
| TAU_ITR232 | 2733 | 2304 |
| TAU_ITR233 | 3395 | 2305 |
| TAU_ITR234 | 3197 | 2306 |
| TAU_ITR235 | 3220 | 2307 |
| TAU_ITR236 | 3179 | 2308 |
| TAU_ITR237 | 2486 | 2309 |
| TAU_ITR238 | 2516 | 2310 |
| TAU_ITR239 | 2546 | 2311 |
| TAU_ITR240 | 2591 | 2312 |

TABLE 5A
ITR to ITR Sequences of AAV particles comprising
anti-tau antibody polynucleotide sequences

| ITR to ITR | Sequence | ITR to ITR |
|---|---|---|
| Construct Name | Length (nt) | SEQ ID NO |
| TAU_ITR36 | 4253 | 2025 |
| TAU_ITR37 | 4716 | 2026 |
| TAU_ITR38 | 4716 | 2027 |
| TAU_ITR39 | 4698 | 2028 |
| TAU_ITR40 | 4689 | 2029 |
| TAU_ITR41 | 4716 | 2030 |
| TAU_ITR42 | 4716 | 2031 |
| TAU_ITR43 | 4698 | 2032 |
| TAU_ITR44 | 4689 | 2033 |
| TAU_ITR45 | 4253 | 2034 |
| TAU_ITR46 | 2350 | 2035 |
| TAU_ITR47 | 2370 | 2036 |
| TAU_ITR48 | 2282 | 2037 |
| TAU_ITR49 | 2314 | 2038 |
| TAU_ITR50 | 2350 | 2039 |
| TAU_ITR51 | 2370 | 2040 |
| TAU_ITR52 | 2624 | 2041 |
| TAU_ITR53 | 3778 | 2042 |
| TAU_ITR54 | 2029 | 2043 |
| TAU_ITR55 | 2218 | 2044 |
| TAU_ITR56 | 3778 | 2045 |
| TAU_ITR57 | 2624 | 2046 |
| TAU_ITR58 | 2350 | 2047 |
| TAU_ITR59 | 2029 | 2048 |
| TAU_ITR60 | 2218 | 2049 |
| TAU_ITR61 | 4188 | 2050 |
| TAU_ITR62 | 3435 | 2051 |
| TAU_ITR63 | 4395 | 2052 |
| TAU_ITR64 | 3510 | 2053 |
| TAU_ITR65 | 4131 | 2054 |
| TAU_ITR66 | 3378 | 2055 |
| TAU_ITR67 | 4395 | 2056 |
| TAU_ITR68 | 3510 | 2057 |
| TAU_ITR69 | 4016 | 2058 |
| TAU_ITR70 | 4016 | 2059 |
| TAU_ITR71 | 3608 | 2060 |
| TAU_ITR241 | 3900 | 2313 |
| TAU_ITR242 | 4042 | 2314 |
| TAU_ITR243 | 3984 | 2315 |
| TAU_ITR244 | 4472 | 2316 |
| TAU_ITR245 | 2549 | 2317 |
| TAU_ITR246 | 2445 | 2318 |
| TAU_ITR247 | 2350 | 2319 |
| TAU_ITR248 | 4698 | 2320 |
| TAU_ITR249 | 4223 | 2321 |

TABLE 5B
ITR to ITR Sequences of AAV particles comprising
anti-tau antibody polynucleotide sequences

| ITR to ITR | Sequence | ITR to ITR |
|---|---|---|
| Construct Name | Length (nt) | SEQ ID NO |
| TAU_ITR250 | 4010 | 4547 |
| TAU_ITR251 | 3812 | 4548 |
| TAU_ITR252 | 3109 | 4549 |
| TAU_ITR253 | 3115 | 4550 |
| TAU_ITR254 | 3522 | 4551 |
| TAU_ITR255 | 3324 | 4552 |
| TAU_ITR256 | 2627 | 4553 |
| TAU_ITR257 | 2621 | 4554 |
| TAU_ITR258 | 3580 | 4555 |
| TAU_ITR259 | 3382 | 4556 |
| TAU_ITR260 | 2685 | 4557 |
| TAU_ITR261 | 2705 | 4558 |
| TAU_ITR262 | 3438 | 4559 |
| TAU_ITR263 | 3240 | 4560 |
| TAU_ITR264 | 2543 | 4561 |
| TAU_ITR265 | 2537 | 4562 |

TABLE 6
ITR to ITR Sequences of AAV particles comprising
anti-tau antibody polynucleotide sequences

| ITR to ITR | Sequence | ITR to ITR |
|---|---|---|
| Construct Name | Length (nt) | SEQ ID NO |
| TAU_ITR72 | 3619 | 2061 |
| TAU_ITR73 | 3610 | 2062 |
| TAU_ITR74 | 3607 | 2063 |
| TAU_ITR75 | 4158 | 2064 |
| TAU_ITR76 | 3598 | 2065 |
| TAU_ITR77 | 3622 | 2066 |
| TAU_ITR78 | 3619 | 2067 |
| TAU_ITR79 | 3607 | 2068 |
| TAU_ITR80 | 4158 | 2069 |
| TAU_ITR81 | 3607 | 2070 |
| TAU_ITR82 | 3598 | 2071 |
| TAU_ITR83 | 3610 | 2072 |
| TAU_ITR84 | 3610 | 2073 |
| TAU_ITR85 | 3622 | 2074 |
| TAU_ITR86 | 3610 | 2075 |

In some embodiments, the AAV particle comprises a viral genome which comprises a sequence which has a percent identity to any of SEQ ID NOs: 1990-2075, 2137-2168, 2171-2237, 2260-2321, and 4547-4562. The viral genome may have 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99% or 100% identity to any of SEQ ID NOs: 1990-2075, 2137-2168, 2171-2237, 2260-2321, and 4547-4562. The viral genome may have 1-10%, 10-20%, 30-40%, 50-60%, 50-70%, 50-80%, 50-90%, 50-99%, 50-100%, 60-70%, 60-80%, 60-90%, 60-99%, 60-100%, 70-80%, 70-90%, 70-99%, 70-100%, 80-85%, 80-90%, 80-95%, 80-99%, 80-100%, 90-95%, 90-99%, or 90-100% to any of SEQ ID NOs: 1990-2075, 2137-2168, 2171-2237, 2260-2321, and 4547-4562. As a non-limiting example, the viral genome comprises a sequence which has 80% identity to any of SEQ ID NO: 1990-2075, 2137-2168, 2171-2237, 2260-2321, and 4547-4562. As another non-limiting example, the viral genome comprises a sequence which has 85% identity to any of SEQ ID NO: 1990-2075, 2137-2168, 2171-2237, 2260-2321, and 4547-4562. As another non-limiting example, the viral genome comprises a sequence which has 90% identity to any of SEQ ID NO: 1990-2075, 2137-2168, 2171-2237, 2260-2321, and 4547-4562. As another non-limiting example, the viral genome comprises a sequence which has 95% identity to any of SEQ ID NO: 1990-2075, 2137-2168, 2171-2237, 2260-2321, and 4547-4562. As another non-limiting example, the viral genome comprises a sequence which has 99% identity to any of SEQ ID NO: 1990-2075, 2137-2168, 2171-2237, 2260-2321, and 4547-4562.

In some embodiments, the AAV particles comprising anti-tau antibody polynucleotide sequences which comprise a nucleic acid sequence encoding at least one antibody heavy and/or light chain may be introduced into mammalian cells.

The AAV viral genomes encoding anti-tau antibody polynucleotides described herein may be useful in the fields of human disease, viruses, infections veterinary applications and a variety of in vivo and in vitro settings. In some embodiments, the AAV viral genomes encoding anti-tau antibody polynucleotides are used for the prevention and/or treatment of a tauopathy.

The viral genome of the AAV particles of the present disclosure may comprise any combination of the sequence regions described in Tables 7-14 encapsulated in any of the capsids listed in Table 1 or described herein.

In some embodiments, the AAV particle viral genome may comprise at least one sequence region as described herein, such as the elements in Tables 7-14, including ITR sequences, promoters, introns and exons, UTR regions, miR binding sites, tags, fillers, polyA sequence. The regions may be located before or after any of the other sequence regions described herein. Viral genomes may further comprise more than one copy of one or more sequence regions as described in Tables 7-14.

Additional sequence regions of the AAV viral genomes within the payload region, such as the signal sequence regions for the encoded polypeptide payload (e.g., antibody payload, such as anti-tau antibody payload), and any linkers (such as linkers between the antibody VH and VL coding sequences, are described below.

### Viral Genome (Payload) Component: Signal Sequence Region

In some embodiments, the nucleic acid sequence comprising the transgene encoding the payload, e.g., an antibody (such as an anti-tau antibody), comprises a nucleic acid sequence encoding a signal sequence (e.g., a signal sequence region herein). In some embodiments, the nucleic acid sequence comprising the transgene encoding the payload comprises two signal sequence regions. In some embodiments, the nucleic acid sequence comprising the transgene encoding the payload comprises three or more signal sequence regions. In certain embodiments, the signal sequence region is not derived from an antibody. In another embodiment, the signal sequence region is derived from an antibody sequence.

In some embodiments, the nucleic acid sequence encoding the signal sequence is located 5′ relative to the nucleic acid sequence encoding the VH and/or the heavy chain. In some embodiments, the nucleotide sequence encoding the signal sequence is located 5′ relative to the nucleic acid sequence encoding the VL and/or the light chain. In some embodiments, the encoded VH, VL, heavy chain, and/or light chain of the encoded antibody (e.g., full length, or antigen binding fragment thereof such as Fab, F(ab′)₂, scFv, etc) comprises a signal sequence at the N-terminus, wherein the signal sequence is optionally cleaved during cellular processing and/or localization of the antibody molecule.

In some embodiments, the signal sequence comprises any one of the signal sequences provided in Table 11 or a functional variant thereof. In some embodiments, the encoded signal sequence comprises an amino acid sequence encoded by any one of the nucleotide sequences provided in Table 11, or an amino acid sequence with at least 70%, 75%, 80%, 85%, 90%, 95%, or 99% sequence identity thereto. In some embodiments, the nucleic acid sequence encoding the signal sequence comprises any one of the nucleotide sequences provided in Table 11, or a nucleotide sequence with at least 70%, 75%, 80%, 85%, 90%, 95%, or 99% sequence identity thereto.

TABLE 11
Signal Sequence Regions

| Sequence | | |
|---|---|---|
| Region Name | Sequence Length | SEQ ID NO |
| Signal1 | 84 | 2106 |
| Signal2 | 93 | 2107 |
| Signal3 | 96 | 2108 |
| Signal4 | 66 | 2109 |
| Signal5 | 72 | 2110 |
| Signal6 | 93 | 2111 |
| Signal7 | 69 | 2112 |
| Signal8 | 81 | 2113 |
| Signal9 | 12 | 2114 |
| Signal10 | 81 | 2115 |
| Signal11 | 66 | 2116 |
| Signal12 | 78 | 2117 |
| Signal13 | 57 | 1740 |
| Signal14 | 57 | 1741 |
| Signal15 | 57 | 1861 |
| Signal16 | 411 | 2241 |
| Signal17 | 60 | 4564 |
| Signal18 | 57 | 1862 |

In some embodiments, the nucleic acid sequence encoding the signal sequence comprises the nucleotide sequence of SEQ ID NO: 4564, or nucleotide sequence with at least 70%, 75%, 80%, 85%, 90%, 95%, or 99% sequence identity thereto. In some embodiments, the nucleic acid sequence encoding the signal sequence comprises the nucleotide sequence of SEQ ID NO: 1862, or nucleotide sequence with at least 70%, 75%, 80%, 85%, 90%, 95%, or 99% sequence identity thereto. In some embodiments, the nucleotide sequence encoding the signal sequence comprises the nucleotide sequence of SEQ ID NO: 1741, or nucleotide sequence with at least 70%, 75%, 80%, 85%, 90%, 95%, or 99% sequence identity thereto. In some embodiments, the nucleic acid sequence encoding the signal sequence comprises the nucleotide sequence of SEQ ID NO: 1741 and is located 5′ relative to the nucleic acid sequence encoding the VH (including the N-terminal VH in scFv) and/or the heavy chain of the antibody molecule. In some embodiments, the nucleic acid sequence encoding the signal sequence comprises the nucleotide sequence of SEQ ID NO: 4564 or 1862, and is located 5′ relative to the nucleic acid sequence encoding the VL (including the N-terminal VL in scFv) and/or the light chain of the antibody molecule.

The signal sequence region(s) may, independently, have a length such as, but not limited to 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 nucleotides. The length of the signal region in the viral genome may be 45-75, or 60-66 nucleotides. As a non-limiting example, the viral genome comprises a signal sequence region that is about 45 nucleotides in length, 57 nucleotides in length, 66 nucleotides in length, about 69 nucleotides in length, about 72 nucleotides in length, about 78 nucleotides in length, about 81 nucleotides in length, about 84 nucleotides in length, about 93 nucleotides in length, about 96 nucleotides in length.

In some embodiments, the AAV particle viral genome comprises one signal sequence region. In some embodiments, the AAV particle viral genome comprises two signal sequence regions. In some embodiments, the AAV particle viral genome comprises three signal sequence regions. In some embodiments, the AAV particle viral genome comprises more than three signal sequence regions. In some embodiments, the signal sequences of a viral genome comprising more than one signal sequence, are the same. In another embodiment, the signal sequences of a viral genome comprising more than one signal sequence, are not the same.

In some embodiments, the AAV particle viral genome comprises one signal sequence region. In some embodiments, the signal sequence region is the Signal14 sequence region. In some embodiments, the signal sequence region is the Signal17 sequence region. In some embodiments, the signal sequence region is the Signal18 sequence region.

In some embodiments, the AAV particle viral genome comprises two signal sequence regions. In some embodiments, the signal sequence regions are Signal14 sequence region and Signal17 sequence region.

In some embodiments, the signal sequence is derived from an antibody sequence. As a non-limiting example, a signal sequence may be derived from the heavy chain or the light chain of an anti-tau antibody, such as, but not limited to, IPN002, PHF1 and/or MC1. While not wishing to be bound by theory, the first approximately 57-60 nucleotides of an antibody heavy chain or light chain sequence may be considered a signal sequence. Non-limiting examples of antibody derived signal sequences include Ab1 (SEQ ID NO: 1740), Ab2 (SEQ ID NO: 1741), Ab122-124 (SEQ ID NO: 1861-1863), and Ab256 (SEQ ID NO: 4564) herein.

### Viral Genome (Payload) Component: Linkers

In certain embodiments, the viral genome includes one or more spacer or linker regions to separate different coding regions, and/or coding and non-coding regions.

In some embodiments, the nucleic acid encoding the payload, e.g., antibody, comprises a nucleic acid sequence encoding a linker. In some embodiments, the nucleic acid encoding the payload encodes two or more linkers. In some embodiments, the encoded linker comprises a linker provided in Table 2. In some embodiments, the encoded linker comprises an amino acid sequence encoded by any one of the nucleotide sequences provided in Table 2, or an amino acid sequence with at least 70%, 75%, 80%, 85%, 90%, 95%, or 99% sequence identity thereto. In some embodiments, the nucleic acid sequence encoding the linker comprises any one of the nucleotide sequences provided in Table 2, or a nucleotide sequence with at least 70%, 75%, 80%, 85%, 90%, 95%, or 99% sequence identity thereto.

In some embodiments, the linker may be a peptide linker that connects the polypeptides encoded by the payload region (e.g., light and heavy antibody chains or VH and VL during expression). Some peptide linkers may be cleaved after expression to separate heavy and light chain domains, allowing assembly of mature antibodies or antibody fragments. Linker cleavage may be enzymatic. In some cases, linkers comprise an enzymatic cleavage site to facilitate intracellular or extracellular cleavage. Some payload regions encode linkers that interrupt polypeptide synthesis during translation of the linker sequence from an mRNA transcript. Such linkers may facilitate the translation of separate protein domains (e.g., heavy and light chain antibody domains) from a single transcript. In some cases, two or more linkers are encoded by a payload region of the viral genome. Non-limiting examples of linkers that may be encoded by the payload region of an AAV particle viral genome are given in Table 2.

TABLE 2
Linkers

| | | | SEQ |
|---|---|---|---|
| Linker ID | Description | Length | ID NO |
| Linker1 | Furin | 12 | 1724 |
| Linker2 | Furin | 12 | 1725 |
| Linker3 | T2A | 54 | 1726 |
| Linker4 | F2A | 75 | 1727 |
| Linker5 | P2A | 66 | 1728 |
| Linker6 | SG4S (SEQ ID NO: 4536) | 18 | 1729 |
| Linker7 | (G4S)3 (SEQ ID NO: 4537) | 45 | 1730 |
| Linker8 | (G4S)5 (SEQ ID NO: 4538) | 75 | 1731 |
| Linker9 | IRES | 609 | 1732 |
| Linker10 | IRES-2 | 623 | 1733 |
| Linker11 | hIgG2 hinge | 54 | 1734 |
| Linker12 | hIgG3 hinge | 108 | 1735 |
| Linker13 | hIgG3-2 hinge | 153 | 1736 |
| Linker14 | hIgG3-3 hinge | 198 | 1737 |
| Linker15 | msiGG-1 hinge | 45 | 1738 |
| Linker16 | msiGG1 hinge | 18 | 1739 |
| Linker17 | HigG3 hinge | 198 | 2244 |
| Linker18 | G4S (SEQ ID NO: 4535) | 15 | 2245 |
| Linker19 | (G4S)2 (SEQ ID NO: 4539) | 30 | 2246 |
| Linker20 | (G4S)3 (SEQ ID NO: 4537) | 45 | 2247 |
| Linker21 | (G4S)4 (SEQ ID NO: 4540) | 60 | 2248 |
| Linker22 | (G4S)5 (SEQ ID NO: 4538) | 75 | 2249 |
| Linker23 | (G4S)5 (SEQ ID NO: 4538) | 75 | 2250 |
| Linker24 | (G4S)6 (SEQ ID NO: 4541) | 90 | 2251 |
| Linker25 | (G4S)8 (SEQ ID NO: 4542) | 120 | 2252 |
| Linker26 | (G4S)8 (SEQ ID NO: 4542) | 120 | 2253 |
| Linker27 | (G4S)4 (SEQ ID NO: 4540) | 60 | 2254 |
| Linker28 | (G4S)6 (SEQ ID NO: 4541) | 90 | 2259 |

In some embodiments, any of the antibodies described herein can have a linker, e.g., a flexible polypeptide linker, of varying lengths, connecting the variable domains (e.g., the VH and the VL) of the antigen binding domain of the antibody molecule. For example, a (Gly4-Ser)n linker, wherein n is 0, 1, 2, 3, 4, 5, 6, 7, or 8 can be used (e.g., any one of SEQ ID NOs: 1730-1731, 2245-2254, or 2259). In some embodiments, the antibody binds to tau (e.g., N-terminal, mid-region, repeat region, or C-terminal region of tau), such as phosphorylated tau.

In some embodiments, the encoded linker comprises an enzymatic cleavage site, e.g., for intracellular and/or extracellular cleavage. In some embodiments, the linker is cleaved to separate the VH and the VL of the antigen binding domain and/or the heavy chain and light chain of the encoded antibodies (e.g., an anti-tau antibody molecule). In some embodiments, the encoded linker comprises a furin linker or a functional variant. In some embodiments, the nucleotide sequence encoding the furin linker comprises the nucleotide sequence of SEQ ID NO: 1724, or a nucleotide sequence with at least 70%, 75%, 80%, 85%, 90%, 95%, or 99% sequence identity thereto. In some embodiments, furin cleaves proteins downstream of a basic amino acid target sequence (e.g., Arg-X-(Arg/Lys)-Arg) (e.g., as described in Thomas, G., 2002. Nature Reviews Molecular Cell Biology 3(10): 753-66; the contents of which are herein incorporated by reference in its entirety). In some embodiments, the encoded linker comprises a 2A self-cleaving peptide (e.g., a 2A peptide derived from foot-and-mouth disease virus (F2A), porcine teschovirus-1 (P2A), Thoseaasigna virus (T2A), or equine rhinitis A virus (E2A)). In some embodiments, the encoded linker comprises a T2A self-cleaving peptide linker. In some embodiments, the nucleotide sequence encoding the T2A linker comprises the nucleotide sequence of SEQ ID NO: 1726, or a nucleotide sequence with at least 70%, 75%, 80%, 85%, 90%, 95%, or 99% sequence identity thereto. In some embodiments, the nucleic acid encoding the payload encodes a furin linker (such as SEQ ID NO: 1724) and a T2A linker (such as SEQ ID NO: 1726).

In certain embodiments, the nucleic acid encoding the payload encodes a furin linker (such as SEQ ID NO: 1724) or a nucleotide sequence with at least 70%, 75%, 80%, 85%, 90%, 95%, or 99% sequence identity thereto; followed by a T2A linker (such as SEQ ID NO: 1726) or a nucleotide sequence with at least 70%, 75%, 80%, 85%, 90%, 95%, or 99% sequence identity thereto. In certain embodiments, the nucleic acid encoding the payload further comprises a msiGG1 hinge sequence (such as SEQ ID NO: 1739) or a nucleotide sequence with at least 70%, 75%, 80%, 85%, 90%, 95%, or 99% sequence identity thereto, followed by a HigG3 hinge sequence (such as SEQ ID NO: 2244) or a nucleotide sequence with at least 70%, 75%, 80%, 85%, 90%, 95%, or 99% sequence identity thereto, both of which are 5′ to the polynucleotide encoding the furin cleavage site and T2A sequence.

In some embodiments, the encoded linkers comprises a cathepsin, a matrix metalloproteinases or a legumain cleavage sites, such as those described e.g. by Cizeau and Macdonald in International Publication No. WO2008052322, the contents of which are herein incorporated in their entirety.

In some embodiments, the encoded linker comprises an internal ribosomal entry site (IRES) is a nucleotide sequence (>500 nucleotides) for initiation of translation in the middle of a nucleotide sequence, e.g., an mRNA sequence (Kim et al., 2011. PLoS One 6(4): e18556; the contents of which are herein incorporated by reference in its entirety), which can be used, for example, to modulate expression of one or more transgenes. In some embodiments, the encode linker comprises a small and unbranched serine-rich peptide linker, such as those described by Huston et al. in U.S. Pat. No. 5,525,491, the contents of which are herein incorporated in their entirety. In some embodiments, polypeptides comprising a serine-rich linker has increased solubility. In some embodiments, the encoded linker comprises an artificial linker, such as those described by Whitlow and Filpula in U.S. Pat. No. 5,856,456 and Ladner et al. in U.S. Pat. No. 4,946,778, the contents of each of which are herein incorporated by their entirety.

In some embodiments, payload regions may encode linkers that are not cleaved. Such linkers may include a simple amino acid sequence, such as a glycine rich sequence. In some cases, linkers may comprise flexible peptide linkers comprising glycine and serine residues. The linker may comprise flexible peptide linkers of different lengths, e.g. nxG4S, where n=1-10 (SEQ ID NO: 4544), and the length of the encoded linker varies between 5 and 50 amino acids. In a non-limiting example, the linker may be 5xG4S (SEQ ID NO: 4544). These flexible linkers are small and without side chains so they tend not to influence secondary protein structure while providing a flexible linker between antibody segments (George et al., 2002. Protein Engineering 15(11): 871-9; Huston et al., 1988. PNAS 85:5879-83; and Shan et al., 1999. Journal of Immunology. 162(11):6589-95; the contents of each of which are herein incorporated by reference in their entirety). Furthermore, the polarity of the serine residues improves solubility and prevents aggregation problems. In some embodiments, the payload region encodes at least one G4S3 linker (“G4S3” disclosed as SEQ ID NO: 4537). In some embodiments, the payload region encodes at least one G4S linker (“G4S” disclosed as SEQ ID NO: 4535). In some embodiments, the payload region encodes at least one G4S5 linker (“G455” disclosed as SEQ ID NO: 4538).

In some embodiments, payload regions may encode small and unbranched serine-rich peptide linkers, such as those described by Huston et al. in U.S. Pat. No. 5,525,491, the contents of which are herein incorporated in their entirety. Polypeptides encoded by the payload region, linked by serine-rich linkers, have increased solubility.

In some embodiments, payload regions may encode artificial linkers, such as those described by Whitlow and Filpula in U.S. Pat. No. 5,856,456 and Ladner et al. in U.S. Pat. No. 4,946,778, the contents of each of which are herein incorporated by their entirety.

In some embodiments, the payload region encodes at least one hinge region. As a non-limiting example, the hinge is an IgG hinge.

In some embodiments, the nucleic acid sequence encoding the linker comprises about 10 to about 700 nucleotides in length, e.g., about 10 to about 700 nucleotides, e.g. about 10 to about 100, e.g., about 50-200 nucleotides, about 150-300 nucleotides, about 250-400 nucleotides, about 350-500 nucleotides, about 450-600 nucleotides, about 550-700 nucleotides, about 650-700 nucleotides. In some embodiments, the nucleic acid sequence encoding the linker comprises about 5 to about 20 nucleotides in length, e.g., about 12 nucleotides in length. In some embodiments, the nucleic acid sequence encoding the linker comprises about 40 to about 60 nucleotides in length, e.g., about 54 nucleotides in length.

Any, or all components of a viral genome may be modified or optimized to improve expression or targeting of the payload. Such components include, but are not limited to, intron, signal peptide sequences, antibody heavy chain and/or light chain 5′ to 3′ order, antibody heavy chain and/or light chain codons, linkers, cleavage sites, polyadenylation sequences, stuffer sequences, other regulatory sequences, and/or the backbone of the ITR to ITR sequence.

This disclosure also provides in some embodiments, nucleic acids, cells, AAV vectors, and AAV particles comprising the above elements.

Exemplary Viral Genome Sequences

In some embodiments, the AAV particle viral genome may comprise any of the sequences shown in Tables 15-91 of WO/2020/223276, incorporated herein by reference. Representative tables are replicated below.

TABLE 67
Sequence Regions in ITR to ITR Sequences

| | TAU_ITR243 | | TAU_ITR242 | | TAU_ITR241 | |
|---|---|---|---|---|---|---|
| | (SEQ ID NO: | | (SEQ ID NO: | | (SEQ ID NO: | |
| | 2315) | | 2314) | | 2313) | |
| | Region | | Region | | Region | |
| Sequence | SEQ | Region | SEQ | Region | SEQ | Region |
| Regions | ID NO | length | ID NO | length | ID NO | length |
| 5′ ITR | 2076 | 130 | 2076 | 130 | 2076 | 130 |
| Promoter | 2084 | 654 | 2085 | 699 | 5086 | 557 |
| Intron | 2240 | 566 | 2240 | 566 | 2240 | 566 |
| VH | 1838 | 1377 | 1838 | 1377 | 1838 | 1377 |
| Linker | | 12 | 1724 | 12 | 1724 | 12 |
| Linker | 1726 | 54 | 1726 | 54 | 1726 | 54 |
| VL | 1936 | 720 | 1936 | 720 | 1936 | 720 |
| PolyA | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2315 (TAU_ITR243) which comprises a 5′ inverted terminal repeat (ITR) sequence region and a 3′ ITR sequence region, a CBA promoter, a human beta-globin intron region, a PHF1 antibody heavy chain variable region, a furin cleavage site, a T2A linker, an PHF1 antibody light chain variable region, and a rabbit globin polyadenylation sequence.

In some embodiments, the AAV particle genome comprises SEQ ID NO: 2314 (TAU_ITR242) which comprises a 5′ inverted terminal repeat (ITR) sequence region and a 3′ ITR sequence region, a GFAP promoter, a human beta-globin intron region, a PHF1 antibody heavy chain variable region, a furin cleavage site, a T2A linker, an PHF1 antibody light chain variable region, and a rabbit globin polyadenylation sequence.

TABLE 82
Sequence Regions in ITR to ITR Sequences

| | TAU_ITR105 | | TAU_ITR113 | | TAU_ITR189 | |
|---|---|---|---|---|---|---|
| | (SEQ ID NO: | | (SEQ ID NO: | | (SEQ ID NO: | |
| | 2155) | | 2163) | | 2261) | |
| | Region | | Region | | Region | |
| Sequence | SEQ | Region | SEQ | Region | SEQ | Region |
| Regions | ID NO | length | ID NO | length | ID NO | length |
| 5′ ITR | 2076 | 130 | 2076 | 130 | 2076 | 130 |
| GFAP | 2085 | 699 | 2085 | 699 | 2085 | 699 |
| Promoter | | | | | | |
| Intron | 2240 | 566 | 2240 | 566 | 2240 | 566 |
| Signal | 1740 | 57 | 1740 | 57 | 1740 | 57 |
| VH | 1859 | 354 | 1859 | 354 | 1859 | 354 |
| Constant | 1742 | 969 | 1742 | 969 | 2243 | 291 |
| Linker | 1725 | 12 | 1724 | 12 | 1724 | 12 |
| Linker | 1727 | 75 | 1726 | 54 | 1726 | 54 |
| Signal | 1980 | 321 | 1980 | 321 | 1980 | 321 |
| VL | 1864 | 321 | 1864 | 321 | 1864 | 321 |
| PolyA | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2155 (TAU_ITR105) which comprises a 5′ inverted terminal repeat (ITR) sequence region and a 3′ ITR sequence region, a GFAP promoter, a human beta-globin intron region, a heavy chain signal, a PT3 antibody heavy chain variable and constant region, a furin cleavage site, a F2A linker, a light chain signal, a PT3 antibody light chain variable and constant region and a rabbit beta globin polyadenylation signal sequence.

In some embodiments, the AAV particle genome comprises SEQ ID NO: 2163 (TAU_ITR513) which comprises a 5′ inverted terminal repeat (ITR) sequence region and a 3′ ITR sequence region, a GFAP promoter, a human beta-globin intron region, a heavy chain signal, a PT3 antibody heavy chain variable and constant region, a furin cleavage site, a T2A linker, a light chain signal, a PT3 antibody light chain variable and constant region and a rabbit beta globin polyadenylation signal sequence.

TABLE 86
Sequence Regions in ITR to ITR Sequences

| | TAU_ITR106 | | TAU_ITR114 | | TAU_ITR188 | |
|---|---|---|---|---|---|---|
| | (SEQ ID NO: 2156) | | (SEQ ID NO: 2164) | | (SEQ ID NO: 2260) | |
| Sequence | Region SEQ | Region | Region SEQ | Region | Region SEQ | Region |
| Regions | ID NO | length | ID NO | length | ID NO | length |
| 5′ ITR | 2076 | 130 | 2076 | 130 | 2076 | 130 |
| SYN Promoter | 2086 | 557 | 2086 | 557 | 2086 | 557 |
| Intron | 2240 | 566 | 2240 | 566 | 2240 | 566 |
| Signal | 1740 | 57 | 1740 | 57 | 1740 | 57 |
| VH | 1859 | 354 | 1859 | 354 | 1859 | 354 |
| Constant | 1742 | 969 | 1742 | 969 | 2243 | 291 |
| Linker | 1725 | 12 | 1724 | 12 | 1724 | 12 |
| Linker | 1727 | 75 | 1726 | 54 | 1726 | 54 |
| Signal | 1861 | 57 | 1861 | 57 | 1861 | 57 |
| VL | 1980 | 321 | 1980 | 231 | 1980 | 231 |
| Constant | 1864 | 321 | 1864 | 321 | 1864 | 321 |
| PolyA | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2156 (TAU_ITR106) which comprises a 5′ inverted terminal repeat (ITR) sequence region and a 3′ ITR sequence region, a synapsin promoter, a human beta-globin intron region, a heavy chain signal, a PT3 antibody heavy chain variable and constant region, a furin cleavage site, a F2A linker, a light chain signal, a PT3 antibody light chain variable and constant region and a rabbit beta globin polyadenylation signal sequence.

In some embodiments, the AAV particle genome comprises SEQ ID NO: 2164 (TAU_ITR114) which comprises a 5′ inverted terminal repeat (ITR) sequence region and a 3′ ITR sequence region, a synapsin promoter, a human beta-globin intron region, a heavy chain signal, a PT3 antibody heavy chain variable and constant region, a furin cleavage site, a F2A linker, a light chain signal, a PT3 antibody light chain variable and constant region and a rabbit beta globin polyadenylation signal sequence.

TABLE 90
Sequence Regions in ITR to ITR Sequences

| | TAU_ITR103 | | TAU_ITR111 | | TAU_ITR119 | |
|---|---|---|---|---|---|---|
| | (SEQ ID NO: | | (SEQ ID NO: | | (SEQ ID NO: | |
| | 2153) | | 2161) | | 2161) | |
| | Region | | Region | | Region | |
| Sequence | SEQ | Region | SEQ | Region | SEQ | Region |
| Regions | ID NO | length | ID NO | length | ID NO | length |
| 5′ ITR | 2076 | 130 | 2076 | 130 | 2076 | 130 |
| CMVie | 2239 | 380 | 2239 | 380 | 2239 | 380 |
| CB Promoter | 2083 | 260 | 2083 | 260 | 2083 | 260 |
| Intron | 2240 | 566 | 2240 | 566 | 2240 | 566 |
| Signal | 1740 | 57 | 1740 | 57 | 1740 | 57 |
| VH | 1859 | 354 | 1859 | 354 | 1859 | 354 |
| Constant | 1742 | 969 | 1742 | 969 | 1742 | 969 |
| Linker | 1725 | 12 | 1724 | 12 | 1724 | 12 |
| Linker | 1727 | 75 | 1726 | 54 | 1726 | 54 |
| Signal | 1861 | 57 | 1861 | 57 | 1861 | 57 |
| VL | 1980 | 321 | 1980 | 321 | 1980 | 321 |
| Constant | 1864 | 321 | 1864 | 321 | 1864 | 321 |
| PolyA | 2122 | 127 | 2122 | 127 | 2122 | 127 |
| 3′ ITR | 2078 | 130 | 2078 | 130 | 2078 | 130 |

In some embodiments, the AAV particle genome comprises SEQ ID NO: 2161 (TAU_ITR111) which comprises a 5′ inverted terminal repeat (ITR) sequence region and a 3′ ITR sequence region, a CMVie region and a CB promoter, a human beta-globin intron region, a heavy chain signal, a PT3 antibody heavy chain variable and constant region, a furin cleavage site, a T2A linker, a light chain signal, a PT3 antibody light chain variable and constant region and a rabbit beta globin polyadenylation signal sequence.

In some embodiments, the AAV particle viral genome may comprise any of the sequences shown in Tables 92-95.

TABLE 92
Sequence Regions in ITR to ITR Sequences

| | TAU_ITR250 | | TAU_ITR254 | | TAU_ITR258 | | TAU_ITR262 | |
|---|---|---|---|---|---|---|---|---|
| | (SEQ ID NO: | | (SEQ ID NO: | | (SEQ ID NO: | | (SEQ ID NO: | |
| | 4547) | | 4551) | | 4555) | | 4559) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| Promoter | 2080 | 1715 | — | — | 2085 | 699 | 2086 | 557 |
| CMVie | 2081 | 299 | 2087 | 382 | — | — | — | — |
| CBA min. | 2082 | 283 | 2083 | 260 | — | — | — | — |
| Exon | — | — | 2090 | 134 | 2090 | 134 | 2090 | 134 |
| Intron | — | — | 2095 | 32 | 2095 | 32 | 2095 | 32 |
| Intron | — | — | 2097 | 347 | 2097 | 347 | 2097 | 347 |
| Exon | — | — | 2093 | 53 | 2093 | 53 | 2093 | 53 |
| BSA | 4563 | 26 | 4563 | 26 | 4563 | 26 | 4563 | 26 |
| Signal | 1741 | 57 | 1741 | 57 | 1741 | 57 | 1741 | 57 |
| VH | 1841 | 717 | 1841 | 717 | 1841 | 717 | 1841 | 717 |
| Linker | 1739 | 18 | 1739 | 18 | 1739 | 18 | 1739 | 18 |
| Linker | 2244 | 198 | 2244 | 198 | 2244 | 198 | 2244 | 198 |
| Linker | 1724 | 12 | 1724 | 12 | 1724 | 12 | 1724 | 12 |
| Linker | 1726 | 54 | 1726 | 54 | 1726 | 54 | 1726 | 54 |
| Signal | 4564 | 60 | 4564 | 60 | 4564 | 60 | 4564 | 60 |
| VL | 4565 | 449 | 4565 | 449 | 4565 | 449 | 4565 | 449 |
| PolyA | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID NO: 4547 (TAU_ITR250) which comprises a 5′ inverted terminal repeat (ITR) sequence region and a 3′ ITR sequence region, a CAG promoter comprising a CMVie region and a minimal CBA promoter region, a BSA sequence, a PHF1 antibody heavy chain signal sequence, a PHF1 antibody heavy chain variable region, two antibody hinge sequences (herein listed as linker sequences), a furin cleavage site, a T2A linker, a PHF1 antibody light chain signal sequence, a PHF1 antibody light chain variable region and a rabbit globin polyadenylation sequence.

In some embodiments, the AAV particle genome comprises SEQ ID NO: 4551 (TAU_ITR254) which comprises a 5′ inverted terminal repeat (ITR) sequence region and a 3′ ITR sequence region, a CMVie region, a CB promoter, an ie1 exon 1 region, an ie1 intron 1 region, a human beta-globin intron region, a human beta-globin exon region, a BSA sequence, a PHF1 antibody heavy chain signal sequence, a PHF1 antibody heavy chain variable region, two antibody hinge sequences (herein listed as linker sequences), a furin cleavage site, a T2A linker, a PHF1 antibody light chain signal sequence, a PHF1 antibody light chain variable region and a rabbit globin polyadenylation sequence.

In some embodiments, the AAV particle genome comprises SEQ ID NO: 4555 (TAU_ITR258) which comprises a 5′ inverted terminal repeat (ITR) sequence region and a 3′ ITR sequence region, a GFAP promoter, an ie1 exon 1 region, an ie1 intron 1 region, a human beta-globin intron region, a human beta-globin exon region, a BSA sequence, a PHF1 antibody heavy chain signal sequence, a PHF1 antibody heavy chain variable region, two antibody hinge sequences (herein listed as linker sequences), a furin cleavage site, a T2A linker, a PHF1 antibody light chain signal sequence, a PHF1 antibody light chain variable region and a rabbit globin polyadenylation sequence.

In some embodiments, the AAV particle genome comprises SEQ ID NO: 4559 (TAU_ITR262) which comprises a 5′ inverted terminal repeat (ITR) sequence region and a 3′ ITR sequence region, a synapsin promoter, an ie1 exon 1 region, an ie1 intron 1 region, a human beta-globin intron region, a human beta-globin exon region, a BSA sequence, a PHF1 antibody heavy chain signal sequence, a PHF1 antibody heavy chain variable region, two antibody hinge sequences (herein listed as linker sequences), a furin cleavage site, a T2A linker, a PHF1 antibody light chain signal sequence, a PHF1 antibody light chain variable region and a rabbit globin polyadenylation sequence.

TABLE 93
Sequence Regions in ITR to ITR Sequences

| | TAU_ITR251 | | TAU_ITR255 | | TAU_ITR259 | | TAU_ITR263 | |
|---|---|---|---|---|---|---|---|---|
| | (SEQ ID NO: | | (SEQ ID NO: | | (SEQ ID NO: | | (SEQ ID NO: | |
| | 4548) | | 4552) | | 4556) | | 4560) | |
| | Region | | Region | | Region | | Region | |
| Sequence | SEQ | Region | SEQ | Region | SEQ | Region | SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| Promoter | 2080 | 1715 | — | — | 2085 | 699 | 2086 | 557 |
| CMVie | 2081 | 299 | 2087 | 382 | — | — | — | — |
| CBA min. | 2082 | 283 | 2083 | 260 | — | — | — | — |
| Exon | — | — | 2090 | 134 | 2090 | 134 | 2090 | 134 |
| Intron | — | — | 2095 | 32 | 2095 | 32 | 2095 | 32 |
| Intron | — | — | 2097 | 347 | 2097 | 347 | 2097 | 347 |
| Exon | — | — | 2093 | 53 | 2093 | 53 | 2093 | 53 |
| BSA | 4563 | 26 | 4563 | 26 | 4563 | 26 | 4563 | 26 |
| Signal | 1741 | 57 | 1741 | 57 | 1741 | 57 | 1741 | 57 |
| VH | 1841 | 717 | 1841 | 717 | 1841 | 717 | 1841 | 717 |
| Linker | 1724 | 12 | 1724 | 12 | 1724 | 12 | 1724 | 12 |
| Linker | 1726 | 54 | 1726 | 54 | 1726 | 54 | 1726 | 54 |
| Signal | 4564 | 60 | 4564 | 60 | 4564 | 60 | 4564 | 60 |
| VL | 4565 | 449 | 4565 | 449 | 4565 | 449 | 4565 | 449 |
| PolyA | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID: 4548 (TAU_ITR251) which comprises a 5′ inverted terminal repeat (ITR) sequence region and a 3′ ITR sequence region, a CAG promoter comprising a CMVie region and a minimal CBA promoter region, a BSA sequence, a PHF1 antibody heavy chain signal sequence, a PHF1 antibody heavy chain variable region, a furin cleavage site, a T2A linker, a PHF1 antibody light chain signal sequence, a PHF1 antibody light chain variable region and a rabbit globin polyadenylation sequence.

In some embodiments, the AAV particle genome comprises SEQ ID NO: 4552 (TAU_ITR255) which comprises a 5′ inverted terminal repeat (ITR) sequence region and a 3′ ITR sequence region, a CMVie region, a CB promoter, an ie1 exon 1 region, an ie1 intron 1 region, a human beta-globin intron region, a human beta-globin exon region, a BSA sequence, a PHF1 antibody heavy chain signal sequence, a PHF1 antibody heavy chain variable region, a furin cleavage site, a T2A linker, a PHF1 antibody light chain signal sequence, a PHF1 antibody light chain variable region and a rabbit globin polyadenylation sequence.

In some embodiments, the AAV particle genome comprises SEQ ID NO: 4556 (TAU_ITR259) which comprises a 5′ inverted terminal repeat (ITR) sequence region and a 3′ ITR sequence region, a GFAP promoter, an ie1 exon 1 region, an ie1 intron 1 region, a human beta-globin intron region, a human beta-globin exon region, a BSA sequence, a PHF1 antibody heavy chain signal sequence, a PHF1 antibody heavy chain variable region, a furin cleavage site, a T2A linker, a PHF1 antibody light chain signal sequence, a PHF1 antibody light chain variable region and a rabbit globin polyadenylation sequence.

In some embodiments, the AAV particle genome comprises SEQ ID NO: 4560 (TAU_ITR263) which comprises a 5′ inverted terminal repeat (ITR) sequence region and a 3′ ITR sequence region, a synapsin promoter, an ie1 exon 1 region, an ie1 intron 1 region, a human beta-globin intron region, a human beta-globin exon region, a BSA sequence, a PHF1 antibody heavy chain signal sequence, a PHF1 antibody heavy chain variable region, a furin cleavage site, a T2A linker, a PHF1 antibody light chain signal sequence, a PHF1 antibody light chain variable region and a rabbit globin polyadenylation sequence.

TABLE 94
Sequence Regions in ITR to ITR Sequences

| | TAU_ITR252 (SEQ | | TAU_ITR257 (SEQ | | TAU_ITR261 (SEQ | | TAU_ITR265 (SEQ | |
|---|---|---|---|---|---|---|---|---|
| | ID NO: 4549) | | ID NO: 4554) | | ID NO: 4558) | | ID NO: 4562) | |
| Sequence | Region SEQ | Region | Region SEQ | Region | Region SEQ | Region | Region SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| Promoter | 2080 | 1715 | — | — | 2085 | 699 | 2086 | 557 |
| CMVie | 2081 | 299 | 2087 | 382 | — | — | — | — |
| CBA min. | 2082 | 283 | 2083 | 260 | — | — | — | — |
| Exon | — | — | 2090 | 134 | 2090 | 134 | 2090 | 134 |
| Intron | — | — | 2095 | 32 | 2095 | 32 | 2095 | 32 |
| Intron | — | — | 2097 | 347 | 2097 | 347 | 2097 | 347 |
| Exon | — | — | 2093 | 53 | 2093 | 53 | 2093 | 53 |
| BSA | 4563 | 26 | 4563 | 26 | 4563 | 26 | 4563 | 26 |
| Signal | 1741 | 57 | 1741 | 57 | 1741 | 57 | 1741 | 57 |
| VH | 2170 | 408 | 2170 | 408 | 2170 | 408 | 2170 | 408 |
| Linker | 1730 | 45 | 1730 | 45 | 1730 | 45 | 1730 | 45 |
| VL | 1957 | 339 | 1957 | 339 | 1957 | 339 | 1957 | 339 |
| Tag | 2118 | 27 | 2118 | 27 | 2118 | 27 | 2118 | 27 |
| PolyA | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID NO: 4549 (TAU_ITR252) which comprises a 5′ inverted terminal repeat (ITR) sequence region and a 3′ ITR sequence region, a CAG promoter comprising a CMVie region and a minimal CBA promoter region, a BSA sequence, a PHF1 antibody heavy chain signal sequence, a PHF1 antibody heavy chain variable region, a G4S3 linker, a PHF1 antibody light chain variable region, an HA tag, and a rabbit globin polyadenylation sequence.

In some embodiments, the AAV particle genome comprises SEQ ID NO: 4554 (TAU_ITR257) which comprises a 5′ inverted terminal repeat (ITR) sequence region and a 3′ ITR sequence region, a CMVie region, a CB promoter, an ie1 exon 1 region, an ie1 intron 1 region, a human beta-globin intron region, a human beta-globin exon region, a BSA sequence, a PHF1 antibody heavy chain signal sequence, a PHF1 antibody heavy chain variable region, a G4S3 linker, a PHF1 antibody light chain variable region, an HA tag, and a rabbit globin polyadenylation sequence.

In some embodiments, the AAV particle genome comprises SEQ ID NO: 4558 (TAU_ITR261) which comprises a 5′ inverted terminal repeat (ITR) sequence region and a 3′ ITR sequence region, a GFAP promoter, an Tel exon 1 region, an el intron 1 region, a human beta-globin intron region, a human beta-globin exon region, a BSA sequence, a PHF1 antibody heavy chain signal sequence, a PHF1 antibody heavy chain variable region, a G4S3 linker, a PHF1 antibody light chain variable region, an HA tag, and a rabbit globin polyadenylation sequence.

In some embodiments, the AAV particle genome comprises SEQ ID NO: 4562 (TAU_ITR265) which comprises a 5′ inverted terminal repeat (ITR) sequence region and a 3′ ITR sequence region, a synapsin promoter, an ie1 exon 1 region, an ie1 intron 1 region, a human beta-globin intron region, a human beta-globin exon region, a BSA sequence, a PHF1 antibody heavy chain signal sequence, a PHF1 antibody heavy chain variable region, a G4S3 linker, a PHF1 antibody light chain variable region, an HA tag, and a rabbit globin polyadenylation sequence.

TABLE 95
Sequence Regions in ITR to ITR Sequences

| | TAU_ITR253 (SEQ | | TAU_ITR256 (SEQ | | TAU_ITR260 (SEQ | | TAU_ITR264 (SEQ | |
|---|---|---|---|---|---|---|---|---|
| | ID NO: 4550) | | ID NO: 4553) | | ID NO: 4557) | | ID NO: 4561) | |
| Sequence | Region SEQ | Region | Region SEQ | Region | Region SEQ | Region | Region SEQ | Region |
| Regions | ID NO | length | ID NO | length | ID NO | length | ID NO | length |
| 5′ ITR | 2076 | 130 | 2076 | 130 | 2076 | 130 | 2076 | 130 |
| Promoter | 2080 | 1715 | — | — | 2085 | 699 | 2086 | 557 |
| CMVie | 2081 | 299 | 2087 | 382 | — | — | — | — |
| CBA min. | 2082 | 283 | 2083 | 260 | — | — | — | — |
| Exon | — | — | 2090 | 134 | 2090 | 134 | 2090 | 134 |
| Intron | — | — | 2095 | 32 | 2095 | 32 | 2095 | 32 |
| Intron | — | — | 2097 | 347 | 2097 | 347 | 2097 | 347 |
| Exon | — | — | 2093 | 53 | 2093 | 53 | 2093 | 53 |
| BSA | 4563 | 26 | 4563 | 26 | 4563 | 26 | 4563 | 26 |
| Signal | 1862 | 57 | 1862 | 57 | 1862 | 57 | 1862 | 57 |
| VL | 1957 | 339 | 1957 | 339 | 1957 | 339 | 1957 | 339 |
| Linker | 1730 | 45 | 1730 | 45 | 1730 | 45 | 1730 | 45 |
| VH | 1839 | 351 | 1839 | 351 | 1839 | 351 | 1839 | 351 |
| Tag | 2118 | 27 | 2118 | 27 | 2118 | 27 | 2118 | 27 |
| PolyA | 2122 | 127 | 2122 | 127 | 2122 | 127 | 2122 | 127 |
| 3′ ITR | 2078 | 130 | 2078 | 130 | 2078 | 130 | 2078 | 130 |

In some embodiments, the AAV particle genome comprises SEQ ID NO: 4550 (TAU_ITR253) which comprises a 5′ inverted terminal repeat (ITR) sequence region and a 3′ ITR sequence region, a CAG promoter comprising a CMVie region and a minimal CBA promoter region, a BSA sequence, a PHF1 antibody signal sequence, a PHF1 antibody light chain variable region, a G4S3 linker, a PHF1 antibody heavy chain variable region, an HA tag, and a rabbit globin polyadenylation sequence.

In some embodiments, the AAV particle genome comprises SEQ ID NO: 4553 (TAU_ITR256) which comprises a 5′ inverted terminal repeat (ITR) sequence region and a 3′ ITR sequence region, a CMVie region, a CB promoter, an ie1 exon 1 region, an ie1 intron 1 region, a human beta-globin intron region, a human beta-globin exon region, a BSA sequence, a PHF1 antibody signal sequence, a PHF1 antibody light chain variable region, a G4S3 linker, a PHF1 antibody heavy chain variable region, an HA tag, and a rabbit globin polyadenylation sequence.

In some embodiments, the AAV particle genome comprises SEQ ID NO: 4557 (TAU_ITR260) which comprises a 5′ inverted terminal repeat (ITR) sequence region and a 3′ ITR sequence region, a GFAP promoter, an ie1 exon 1 region, an ie1 intron 1 region, a human beta-globin intron region, a human beta-globin exon region, a BSA sequence, a PHF1 antibody signal sequence, a PHF1 antibody light chain variable region, a G4S3 linker, a PHF1 antibody heavy chain variable region, an HA tag, and a rabbit globin polyadenylation sequence.

In some embodiments, the AAV particle genome comprises SEQ ID NO: 4561 (TAU_ITR264) which comprises a 5′ inverted terminal repeat (ITR) sequence region and a 3′ ITR sequence region, a synapsin promoter, an ie1 exon 1 region, an ie1 intron 1 region, a human beta-globin intron region, a human beta-globin exon region, a BSA sequence, a PHF1 antibody signal sequence, a PHF1 antibody light chain variable region, a G4S3 linker, a PHF1 antibody heavy chain variable region, an HA tag, and a rabbit globin polyadenylation sequence.

In some embodiments, the viral genome may comprise any combination of the following components, including, but not limited to, a 5′ ITR, a promoter region (may comprise one or more component pieces), an intronic region, a Kozak sequence, one or more signal sequences (antibody signal sequences or signal sequence derived from another protein), one or more furin cleavage sites, one or more linker sequences, one or more antibody light chain variable regions, one or more antibody light chain constant regions, one or more antibody heavy chain variable regions, one or more antibody heavy chain constant regions, a polyadenylation sequence, a stuffer sequence, and/or a filler sequence.

In some embodiments, the AAV viral genome comprises, when read in the 5′ to 3′ direction, a 5′ ITR, a promoter region, an optional intronic region, a signal sequence, an antibody light chain region, a linker region, a signal sequence, an antibody heavy chain region, a polyadenylation sequence, an optional filler sequence, and a 3′ ITR.

The viral genome may encode an antibody fragment, such as, but not limited to Fab, F(ab′)₂ or scFv fragments. In some embodiments, the viral genome encodes a Fab antibody fragment. In another embodiment, the viral genome encodes an F(ab′)₂ antibody fragment. In some embodiments, the viral genome encodes an scFv (either VH-linker-VL or VL-linker-VH).

In some embodiments, the viral genome comprising the ITR to ITR sequence, or a fragment thereof, described in Tables 4-6 and 15-91 of WO/2020/223276 (incorporated herein by reference), and Tables 92-95 herein, is packaged in a capsid having a serotype selected from Table 1 to generate an AAV particle. For example, each and every of the ITR to ITR sequences in Tables 4-6 and 15-91 of WO/2020/223276 (incorporated herein by reference), and in Tables 92-95 herein, is explicitly contemplated to be used with each and every one of the capsids in Table 1, such as the capsid serotype selected form VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), AAVPHP.N (PHP.N), PHP.S, AAV1, AAV2, AAV2 variant, AAV2/3 variant, AAV4, AAV5, AAV6, AAV7, AAV8, AAV9.47, AAV9(hu14), AAV9, AAV9 K449R, AAV10, AAV11, AAV12, AAVrh8, AAVrh10, AAVDJ, AAVDJ8, AAV2G9, and a functional variant thereof with amino acid sequence identity of at least 70%, 75%, 80%, 85%, 90%, 91%, 92%, 93%, 94%, 95%, 96%, 97%, 98%, 99%, or greater than 99%; optionally, the AAV capsid is VOY101, VOY201, AAVPHP.B, AAVPHP.N, AAV1, AAV2, AAV2 variant, AAV3, AAV2/3 variant, AAV4, AAV5, AAV6, AAV7, AAV8, AAV9, AAV9.47, AAV9(hu14), AAV9 K449R, AAV10, AAV11, AAV12, AAVrh8, AAVrh10, AAVDJ, or AAVDJ8, or any variant thereof.

In some embodiments, each and every of the ITR to ITR sequences in Tables 4-6 and 15-91 of WO/2020/223276 (incorporated herein by reference), and in Tables 92-95 herein, as well as variant sequences thereof sharing at least 60, 65%, 70%, 75%, 80%, 85%, 90%, 91%, 92%, 93%, 94%, 95%, 96%, 97%, 98%, 99%, or greater than 99% nucleotide sequence identity to any one of the ITR to ITR sequences in Tables 4-6 and 15-95, is explicitly contemplated to be used with each and every one of the capsids selected from VOY101, VOY201, AAVPHP.B, AAVPHP.N, AAV2, AAV2/3 variant, AAV9, AAV9 K449R, AAV6, AAVrh10, and/or AAVDJ, and a functional variant thereof with amino acid sequence identity of at least 70%, 75%, 80%, 85%, 90%, 91%, 92%, 93%, 94%, 95%, 96%, 97%, 98%, 99%, or greater than 99%; optionally, the capsid is encoded by a nucleic acid sequence comprising any one of SEQ ID NOs: 1-6, 11, 137, 138, 2679, 2809, 2871 and 4534, and a functional variant thereof with amino acid sequence identity of at least 70%, 75%, 80%, 85%, 90%, 91%, 92%, 93%, 94%, 95%, 96%, 97%, 98%, 99%, or greater than 99%.

In some embodiments, the AAV particle comprises a viral genome comprising SEQ ID NO: 4547 and a VOY101 capsid. In some embodiments, the AAV particle comprises a viral genome comprising SEQ ID NO: 4547 and a VOY201 capsid. In some embodiments, the AAV particle comprises a viral genome comprising SEQ ID NO: 4547 and an AAV9 capsid. In some embodiments, the AAV particle comprises a viral genome comprising SEQ ID NO: 4547 and an AAV9 K449R capsid. In some embodiments, the AAV particle comprises a viral genome comprising SEQ ID NO: 4547 and an AAVPHP.B capsid. In some embodiments, the AAV particle comprises a viral genome comprising SEQ ID NO: 4547 and an AAVPHP.N capsid. In some embodiments, the AAV particle comprises a viral genome comprising SEQ ID NO: 4547 and an AAV2 variant capsid. In some embodiments, the AAV particle comprises a viral genome comprising SEQ ID NO: 4547 and an AAV2/3 variant capsid. In some embodiments, the AAV particle comprises a viral genome comprising SEQ ID NO: 4547 and a capsid comprising SEQ ID NO: 1. In some embodiments, the AAV particle comprises a viral genome comprising SEQ ID NO: 4547 and a capsid encoded by a nucleic acid sequence comprising SEQ ID NO: 2. In some embodiments, the AAV particle comprises a viral genome comprising SEQ ID NO: 4547 and a capsid comprising SEQ ID NO: 4534. In some embodiments, the AAV particle comprises a viral genome comprising SEQ ID NO: 4547 and a capsid encoded by a nucleic acid sequence comprising SEQ ID NO: 3. In some embodiments, the AAV particle comprises a viral genome comprising SEQ ID NO: 4547 and a capsid comprising SEQ ID NO: 138. In some embodiments, the AAV particle comprises a viral genome comprising SEQ ID NO: 4547 and a capsid encoded by a nucleic acid sequence comprising SEQ ID NO: 137. In some embodiments, the AAV particle comprises a viral genome comprising SEQ ID NO: 4547 and a capsid comprising SEQ ID NO: 5. In some embodiments, the AAV particle comprises a viral genome comprising SEQ ID NO: 4547 and a capsid encoded by a nucleic acid sequence comprising SEQ ID NO: 6. In some embodiments, the AAV particle comprises a viral genome comprising SEQ ID NO: 4547 and a capsid comprising SEQ ID NO: 4. In some embodiments, the AAV particle comprises a viral genome comprising SEQ ID NO: 4547 and a capsid comprising SEQ ID NO: 11. In some embodiments, the AAV particle comprises a viral genome comprising SEQ ID NO: 4547 and a capsid comprising SEQ ID NO: 2679. In some embodiments, the AAV particle comprises a viral genome comprising SEQ ID NO: 4547 and a capsid comprising SEQ ID NO: 2809. In some embodiments, the AAV particle comprises a viral genome comprising SEQ ID NO: 4547 and a capsid comprising SEQ ID NO: 2871.

In some embodiments, the AAV particle comprises a viral genome comprising SEQ ID NO: 4548 and a VOY101 capsid. In some embodiments, the AAV particle comprises a viral genome comprising SEQ ID NO: 4548 and a VOY201 capsid. In some embodiments, the AAV particle comprises a viral genome comprising SEQ ID NO: 4548 and an AAV9 capsid. In some embodiments, the AAV particle comprises a viral genome comprising SEQ ID NO: 4548 and an AAV9 K449R capsid. In some embodiments, the AAV particle comprises a viral genome comprising SEQ ID NO: 4548 and an AAVPHP.B capsid. In some embodiments, the AAV particle comprises a viral genome comprising SEQ ID NO: 4548 and an AAVPHP.N capsid. In some embodiments, the AAV particle comprises a viral genome comprising SEQ ID NO: 4548 and an AAV2 variant capsid. In some embodiments, the AAV particle comprises a viral genome comprising SEQ ID NO: 4548 and an AAV2/3 variant capsid. In some embodiments, the AAV particle comprises a viral genome comprising SEQ ID NO: 4548 and a capsid comprising SEQ ID NO: 1. In some embodiments, the AAV particle comprises a viral genome comprising SEQ ID NO: 4548 and a capsid encoded by a nucleic acid sequence comprising SEQ ID NO: 2. In some embodiments, the AAV particle comprises a viral genome comprising SEQ ID NO: 4548 and a capsid comprising SEQ ID NO: 4534. In some embodiments, the AAV particle comprises a viral genome comprising SEQ ID NO: 4548 and a capsid encoded by a nucleic acid sequence comprising SEQ ID NO: 3. In some embodiments, the AAV particle comprises a viral genome comprising SEQ ID NO: 4548 and a capsid comprising SEQ ID NO: 138. In some embodiments, the AAV particle comprises a viral genome comprising SEQ ID NO: 4548 and a capsid encoded by a nucleic acid sequence comprising SEQ ID NO: 137. In some embodiments, the AAV particle comprises a viral genome comprising SEQ ID NO: 4548 and a capsid comprising SEQ ID NO: 5. In some embodiments, the AAV particle comprises a viral genome comprising SEQ ID NO: 4548 and a capsid encoded by a nucleic acid sequence comprising SEQ ID NO: 6. In some embodiments, the AAV particle comprises a viral genome comprising SEQ ID NO: 4548 and a capsid comprising SEQ ID NO: 4. In some embodiments, the AAV particle comprises a viral genome comprising SEQ ID NO: 4548 and a capsid comprising SEQ ID NO: 11. In some embodiments, the AAV particle comprises a viral genome comprising SEQ ID NO: 4548 and a capsid comprising SEQ ID NO: 2679. In some embodiments, the AAV particle comprises a viral genome comprising SEQ ID NO: 4548 and a capsid comprising SEQ ID NO: 2809. In some embodiments, the AAV particle comprises a viral genome comprising SEQ ID NO: 4548 and a capsid comprising SEQ ID NO: 2871.

In some embodiments, the AAV particle comprises a viral genome comprising SEQ ID NO: 4549 and a VOY101 capsid. In some embodiments, the AAV particle comprises a viral genome comprising SEQ ID NO: 4549 and a VOY201 capsid. In some embodiments, the AAV particle comprises a viral genome comprising SEQ ID NO: 4549 and an AAV9 capsid. In some embodiments, the AAV particle comprises a viral genome comprising SEQ ID NO: 4549 and an AAV9 K449R capsid. In some embodiments, the AAV particle comprises a viral genome comprising SEQ ID NO: 4549 and an AAVPHP.B capsid. In some embodiments, the AAV particle comprises a viral genome comprising SEQ ID NO: 4549 and an AAVPHP.N capsid. In some embodiments, the AAV particle comprises a viral genome comprising SEQ ID NO: 4549 and an AAV2 variant capsid. In some embodiments, the AAV particle comprises a viral genome comprising SEQ ID NO: 4549 and an AAV2/3 variant capsid. In some embodiments, the AAV particle comprises a viral genome comprising SEQ ID NO: 4549 and a capsid comprising SEQ ID NO: 1. In some embodiments, the AAV particle comprises a viral genome comprising SEQ ID NO: 4549 and a capsid encoded by a nucleic acid sequence comprising SEQ ID NO: 2. In some embodiments, the AAV particle comprises a viral genome comprising SEQ ID NO: 4549 and a capsid comprising SEQ ID NO: 4534. In some embodiments, the AAV particle comprises a viral genome comprising SEQ ID NO: 4549 and a capsid encoded by a nucleic acid sequence comprising SEQ ID NO: 3. In some embodiments, the AAV particle comprises a viral genome comprising SEQ ID NO: 4549 and a capsid comprising SEQ ID NO: 138. In some embodiments, the AAV particle comprises a viral genome comprising SEQ ID NO: 4549 and a capsid encoded by a nucleic acid sequence comprising SEQ ID NO: 137. In some embodiments, the AAV particle comprises a viral genome comprising SEQ ID NO: 4549 and a capsid comprising SEQ ID NO: 5. In some embodiments, the AAV particle comprises a viral genome comprising SEQ ID NO: 4549 and a capsid encoded by a nucleic acid sequence comprising SEQ ID NO: 6. In some embodiments, the AAV particle comprises a viral genome comprising SEQ ID NO: 4549 and a capsid comprising SEQ ID NO: 4. In some embodiments, the AAV particle comprises a viral genome comprising SEQ ID NO: 4549 and a capsid comprising SEQ ID NO: 11. In some embodiments, the AAV particle comprises a viral genome comprising SEQ ID NO: 4549 and a capsid comprising SEQ ID NO: 2679. In some embodiments, the AAV particle comprises a viral genome comprising SEQ ID NO: 4549 and a capsid comprising SEQ ID NO: 2809. In some embodiments, the AAV particle comprises a viral genome comprising SEQ ID NO: 4549 and a capsid comprising SEQ ID NO: 2871.

In some embodiments, the AAV particle comprises a viral genome comprising SEQ ID NO: 4550 and a VOY101 capsid. In some embodiments, the AAV particle comprises a viral genome comprising SEQ ID NO: 4550 and a VOY201 capsid. In some embodiments, the AAV particle comprises a viral genome comprising SEQ ID NO: 4550 and an AAV9 capsid. In some embodiments, the AAV particle comprises a viral genome comprising SEQ ID NO: 4550 and an AAV9 K449R capsid. In some embodiments, the AAV particle comprises a viral genome comprising SEQ ID NO: 4550 and an AAVPHP.B capsid. In some embodiments, the AAV particle comprises a viral genome comprising SEQ ID NO: 4550 and an AAVPHP.N capsid. In some embodiments, the AAV particle comprises a viral genome comprising SEQ ID NO: 4550 and an AAV2 variant capsid. In some embodiments, the AAV particle comprises a viral genome comprising SEQ ID NO: 4550 and an AAV2/3 variant capsid. In some embodiments, the AAV particle comprises a viral genome comprising SEQ ID NO: 4550 and a capsid comprising SEQ ID NO: 1. In some embodiments, the AAV particle comprises a viral genome comprising SEQ ID NO: 4550 and a capsid encoded by a nucleic acid sequence comprising SEQ ID NO: 2. In some embodiments, the AAV particle comprises a viral genome comprising SEQ ID NO: 4550 and a capsid comprising SEQ ID NO: 4534. In some embodiments, the AAV particle comprises a viral genome comprising SEQ ID NO: 4550 and a capsid encoded by a nucleic acid sequence comprising SEQ ID NO: 3. In some embodiments, the AAV particle comprises a viral genome comprising SEQ ID NO: 4550 and a capsid comprising SEQ ID NO: 138. In some embodiments, the AAV particle comprises a viral genome comprising SEQ ID NO: 4550 and a capsid encoded by a nucleic acid sequence comprising SEQ ID NO: 137. In some embodiments, the AAV particle comprises a viral genome comprising SEQ ID NO: 4550 and a capsid comprising SEQ ID NO: 5. In some embodiments, the AAV particle comprises a viral genome comprising SEQ ID NO: 4550 and a capsid encoded by a nucleic acid sequence comprising SEQ ID NO: 6. In some embodiments, the AAV particle comprises a viral genome comprising SEQ ID NO: 4550 and a capsid comprising SEQ ID NO: 4. In some embodiments, the AAV particle comprises a viral genome comprising SEQ ID NO: 4550 and a capsid comprising SEQ ID NO: 11. In some embodiments, the AAV particle comprises a viral genome comprising SEQ ID NO: 4550 and a capsid comprising SEQ ID NO: 2679. In some embodiments, the AAV particle comprises a viral genome comprising SEQ ID NO: 4550 and a capsid comprising SEQ ID NO: 2809. In some embodiments, the AAV particle comprises a viral genome comprising SEQ ID NO: 4550 and a capsid comprising SEQ ID NO: 2871.

In some embodiments, the AAV particle comprises a viral genome comprising SEQ ID NO: 4551 and a VOY101 capsid. In some embodiments, the AAV particle comprises a viral genome comprising SEQ ID NO: 4551 and a VOY201 capsid. In some embodiments, the AAV particle comprises a viral genome comprising SEQ ID NO: 4551 and an AAV9 capsid. In some embodiments, the AAV particle comprises a viral genome comprising SEQ ID NO: 4551 and an AAV9 K449R capsid. In some embodiments, the AAV particle comprises a viral genome comprising SEQ ID NO: 4551 and an AAVPHP.B capsid. In some embodiments, the AAV particle comprises a viral genome comprising SEQ ID NO: 4551 and an AAVPHP.N capsid. In some embodiments, the AAV particle comprises a viral genome comprising SEQ ID NO: 4551 and an AAV2 variant capsid. In some embodiments, the AAV particle comprises a viral genome comprising SEQ ID NO: 4551 and an AAV2/3 variant capsid. In some embodiments, the AAV particle comprises a viral genome comprising SEQ ID NO: 4551 and a capsid comprising SEQ ID NO: 1. In some embodiments, the AAV particle comprises a viral genome comprising SEQ ID NO: 4551 and a capsid encoded by a nucleic acid sequence comprising SEQ ID NO: 2. In some embodiments, the AAV particle comprises a viral genome comprising SEQ ID NO: 4551 and a capsid comprising SEQ ID NO: 4534. In some embodiments, the AAV particle comprises a viral genome comprising SEQ ID NO: 4551 and a capsid encoded by a nucleic acid sequence comprising SEQ ID NO: 3. In some embodiments, the AAV particle comprises a viral genome comprising SEQ ID NO: 4551 and a capsid comprising SEQ ID NO: 138. In some embodiments, the AAV particle comprises a viral genome comprising SEQ ID NO: 4551 and a capsid encoded by a nucleic acid sequence comprising SEQ ID NO: 137. In some embodiments, the AAV particle comprises a viral genome comprising SEQ ID NO: 4551 and a capsid comprising SEQ ID NO: 5. In some embodiments, the AAV particle comprises a viral genome comprising SEQ ID NO: 4551 and a capsid encoded by a nucleic acid sequence comprising SEQ ID NO: 6. In some embodiments, the AAV particle comprises a viral genome comprising SEQ ID NO: 4551 and a capsid comprising SEQ ID NO: 4. In some embodiments, the AAV particle comprises a viral genome comprising SEQ ID NO: 4551 and a capsid comprising SEQ ID NO: 11. In some embodiments, the AAV particle comprises a viral genome comprising SEQ ID NO: 4551 and a capsid comprising SEQ ID NO: 2679. In some embodiments, the AAV particle comprises a viral genome comprising SEQ ID NO: 4551 and a capsid comprising SEQ ID NO: 2809. In some embodiments, the AAV particle comprises a viral genome comprising SEQ ID NO: 4551 and a capsid comprising SEQ ID NO: 2871.

In some embodiments, the AAV particle comprises a viral genome comprising SEQ ID NO: 4552 and a VOY101 capsid. In some embodiments, the AAV particle comprises a viral genome comprising SEQ ID NO: 4552 and a VOY201 capsid. In some embodiments, the AAV particle comprises a viral genome comprising SEQ ID NO: 4552 and an AAV9 capsid. In some embodiments, the AAV particle comprises a viral genome comprising SEQ ID NO: 4552 and an AAV9 K449R capsid. In some embodiments, the AAV particle comprises a viral genome comprising SEQ ID NO: 4552 and an AAVPHP.B capsid. In some embodiments, the AAV particle comprises a viral genome comprising SEQ ID NO: 4552 and an AAVPHP.N capsid. In some embodiments, the AAV particle comprises a viral genome comprising SEQ ID NO: 4552 and an AAV2 variant capsid. In some embodiments, the AAV particle comprises a viral genome comprising SEQ ID NO: 4552 and an AAV2/3 variant capsid. In some embodiments, the AAV particle comprises a viral genome comprising SEQ ID NO: 4552 and a capsid comprising SEQ ID NO: 1. In some embodiments, the AAV particle comprises a viral genome comprising SEQ ID NO: 4552 and a capsid encoded by a nucleic acid sequence comprising SEQ ID NO: 2. In some embodiments, the AAV particle comprises a viral genome comprising SEQ ID NO: 4552 and a capsid comprising SEQ ID NO: 4534. In some embodiments, the AAV particle comprises a viral genome comprising SEQ ID NO: 4552 and a capsid encoded by a nucleic acid sequence comprising SEQ ID NO: 3. In some embodiments, the AAV particle comprises a viral genome comprising SEQ ID NO: 4552 and a capsid comprising SEQ ID NO: 138. In some embodiments, the AAV particle comprises a viral genome comprising SEQ ID NO: 4552 and a capsid encoded by a nucleic acid sequence comprising SEQ ID NO: 137. In some embodiments, the AAV particle comprises a viral genome comprising SEQ ID NO: 4552 and a capsid comprising SEQ ID NO: 5. In some embodiments, the AAV particle comprises a viral genome comprising SEQ ID NO: 4552 and a capsid encoded by a nucleic acid sequence comprising SEQ ID NO: 6. In some embodiments, the AAV particle comprises a viral genome comprising SEQ ID NO: 4552 and a capsid comprising SEQ ID NO: 4. In some embodiments, the AAV particle comprises a viral genome comprising SEQ ID NO: 4552 and a capsid comprising SEQ ID NO: 11. In some embodiments, the AAV particle comprises a viral genome comprising SEQ ID NO: 4552 and a capsid comprising SEQ ID NO: 2679. In some embodiments, the AAV particle comprises a viral genome comprising SEQ ID NO: 4552 and a capsid comprising SEQ ID NO: 2809. In some embodiments, the AAV particle comprises a viral genome comprising SEQ ID NO: 4552 and a capsid comprising SEQ ID NO: 2871.

In some embodiments, the AAV particle comprises a viral genome comprising SEQ ID NO: 4553 and a VOY101 capsid. In some embodiments, the AAV particle comprises a viral genome comprising SEQ ID NO: 4553 and a VOY201 capsid. In some embodiments, the AAV particle comprises a viral genome comprising SEQ ID NO: 4553 and an AAV9 capsid. In some embodiments, the AAV particle comprises a viral genome comprising SEQ ID NO: 4553 and an AAV9 K449R capsid. In some embodiments, the AAV particle comprises a viral genome comprising SEQ ID NO: 4553 and an AAVPHP.B capsid. In some embodiments, the AAV particle comprises a viral genome comprising SEQ ID NO: 4553 and an AAVPHP.N capsid. In some embodiments, the AAV particle comprises a viral genome comprising SEQ ID NO: 4553 and an AAV2 variant capsid. In some embodiments, the AAV particle comprises a viral genome comprising SEQ ID NO: 4553 and an AAV2/3 variant capsid. In some embodiments, the AAV particle comprises a viral genome comprising SEQ ID NO: 4553 and a capsid comprising SEQ ID NO: 1. In some embodiments, the AAV particle comprises a viral genome comprising SEQ ID NO: 4553 and a capsid encoded by a nucleic acid sequence comprising SEQ ID NO: 2. In some embodiments, the AAV particle comprises a viral genome comprising SEQ ID NO: 4553 and a capsid comprising SEQ ID NO: 4534. In some embodiments, the AAV particle comprises a viral genome comprising SEQ ID NO: 4553 and a capsid encoded by a nucleic acid sequence comprising SEQ ID NO: 3. In some embodiments, the AAV particle comprises a viral genome comprising SEQ ID NO: 4553 and a capsid comprising SEQ ID NO: 138. In some embodiments, the AAV particle comprises a viral genome comprising SEQ ID NO: 4553 and a capsid encoded by a nucleic acid sequence comprising SEQ ID NO: 137. In some embodiments, the AAV particle comprises a viral genome comprising SEQ ID NO: 4553 and a capsid comprising SEQ ID NO: 5. In some embodiments, the AAV particle comprises a viral genome comprising SEQ ID NO: 4553 and a capsid encoded by a nucleic acid sequence comprising SEQ ID NO: 6. In some embodiments, the AAV particle comprises a viral genome comprising SEQ ID NO: 4553 and a capsid comprising SEQ ID NO: 4. In some embodiments, the AAV particle comprises a viral genome comprising SEQ ID NO: 4553 and a capsid comprising SEQ ID NO: 11. In some embodiments, the AAV particle comprises a viral genome comprising SEQ ID NO: 4553 and a capsid comprising SEQ ID NO: 2679. In some embodiments, the AAV particle comprises a viral genome comprising SEQ ID NO: 4553 and a capsid comprising SEQ ID NO: 2809. In some embodiments, the AAV particle comprises a viral genome comprising SEQ ID NO: 4553 and a capsid comprising SEQ ID NO: 2871.

In some embodiments, the AAV particle comprises a viral genome comprising SEQ ID NO: 4554 and a VOY101 capsid. In some embodiments, the AAV particle comprises a viral genome comprising SEQ ID NO: 4554 and a VOY201 capsid. In some embodiments, the AAV particle comprises a viral genome comprising SEQ ID NO: 4554 and an AAV9 capsid. In some embodiments, the AAV particle comprises a viral genome comprising SEQ ID NO: 4554 and an AAV9 K449R capsid. In some embodiments, the AAV particle comprises a viral genome comprising SEQ ID NO: 4554 and an AAVPHP.B capsid. In some embodiments, the AAV particle comprises a viral genome comprising SEQ ID NO: 4554 and an AAVPHP.N capsid. In some embodiments, the AAV particle comprises a viral genome comprising SEQ ID NO: 4554 and an AAV2 variant capsid. In some embodiments, the AAV particle comprises a viral genome comprising SEQ ID NO: 4554 and an AAV2/3 variant capsid. In some embodiments, the AAV particle comprises a viral genome comprising SEQ ID NO: 4554 and a capsid comprising SEQ ID NO: 1. In some embodiments, the AAV particle comprises a viral genome comprising SEQ ID NO: 4554 and a capsid encoded by a nucleic acid sequence comprising SEQ ID NO: 2. In some embodiments, the AAV particle comprises a viral genome comprising SEQ ID NO: 4554 and a capsid comprising SEQ ID NO: 4534. In some embodiments, the AAV particle comprises a viral genome comprising SEQ ID NO: 4554 and a capsid encoded by a nucleic acid sequence comprising SEQ ID NO: 3. In some embodiments, the AAV particle comprises a viral genome comprising SEQ ID NO: 4554 and a capsid comprising SEQ ID NO: 138. In some embodiments, the AAV particle comprises a viral genome comprising SEQ ID NO: 4554 and a capsid encoded by a nucleic acid sequence comprising SEQ ID NO: 137. In some embodiments, the AAV particle comprises a viral genome comprising SEQ ID NO: 4554 and a capsid comprising SEQ ID NO: 5. In some embodiments, the AAV particle comprises a viral genome comprising SEQ ID NO: 4554 and a capsid encoded by a nucleic acid sequence comprising SEQ ID NO: 6. In some embodiments, the AAV particle comprises a viral genome comprising SEQ ID NO: 4554 and a capsid comprising SEQ ID NO: 4. In some embodiments, the AAV particle comprises a viral genome comprising SEQ ID NO: 4554 and a capsid comprising SEQ ID NO: 11. In some embodiments, the AAV particle comprises a viral genome comprising SEQ ID NO: 4554 and a capsid comprising SEQ ID NO: 2679. In some embodiments, the AAV particle comprises a viral genome comprising SEQ ID NO: 4554 and a capsid comprising SEQ ID NO: 2809. In some embodiments, the AAV particle comprises a viral genome comprising SEQ ID NO: 4554 and a capsid comprising SEQ ID NO: 2871.

In some embodiments, the AAV particle comprises a viral genome comprising SEQ ID NO: 4555 and a VOY101 capsid. In some embodiments, the AAV particle comprises a viral genome comprising SEQ ID NO: 4555 and a VOY201 capsid. In some embodiments, the AAV particle comprises a viral genome comprising SEQ ID NO: 4555 and an AAV9 capsid. In some embodiments, the AAV particle comprises a viral genome comprising SEQ ID NO: 4555 and an AAV9 K449R capsid. In some embodiments, the AAV particle comprises a viral genome comprising SEQ ID NO: 4555 and an AAVPHP.B capsid. In some embodiments, the AAV particle comprises a viral genome comprising SEQ ID NO: 4555 and an AAVPHP.N capsid. In some embodiments, the AAV particle comprises a viral genome comprising SEQ ID NO: 4555 and an AAV2 variant capsid. In some embodiments, the AAV particle comprises a viral genome comprising SEQ ID NO: 4555 and an AAV2/3 variant capsid. In some embodiments, the AAV particle comprises a viral genome comprising SEQ ID NO: 4555 and a capsid comprising SEQ ID NO: 1. In some embodiments, the AAV particle comprises a viral genome comprising SEQ ID NO: 4555 and a capsid encoded by a nucleic acid sequence comprising SEQ ID NO: 2. In some embodiments, the AAV particle comprises a viral genome comprising SEQ ID NO: 4555 and a capsid comprising SEQ ID NO: 4534. In some embodiments, the AAV particle comprises a viral genome comprising SEQ ID NO: 4555 and a capsid encoded by a nucleic acid sequence comprising SEQ ID NO: 3. In some embodiments, the AAV particle comprises a viral genome comprising SEQ ID NO: 4555 and a capsid comprising SEQ ID NO: 138. In some embodiments, the AAV particle comprises a viral genome comprising SEQ ID NO: 4555 and a capsid encoded by a nucleic acid sequence comprising SEQ ID NO: 137. In some embodiments, the AAV particle comprises a viral genome comprising SEQ ID NO: 4555 and a capsid comprising SEQ ID NO: 5. In some embodiments, the AAV particle comprises a viral genome comprising SEQ ID NO: 4555 and a capsid encoded by a nucleic acid sequence comprising SEQ ID NO: 6. In some embodiments, the AAV particle comprises a viral genome comprising SEQ ID NO: 4555 and a capsid comprising SEQ ID NO: 4. In some embodiments, the AAV particle comprises a viral genome comprising SEQ ID NO: 4555 and a capsid comprising SEQ ID NO: 11. In some embodiments, the AAV particle comprises a viral genome comprising SEQ ID NO: 4555 and a capsid comprising SEQ ID NO: 2679. In some embodiments, the AAV particle comprises a viral genome comprising SEQ ID NO: 4555 and a capsid comprising SEQ ID NO: 2809. In some embodiments, the AAV particle comprises a viral genome comprising SEQ ID NO: 4555 and a capsid comprising SEQ ID NO: 2871.

In some embodiments, the AAV particle comprises a viral genome comprising SEQ ID NO: 4556 and a VOY101 capsid. In some embodiments, the AAV particle comprises a viral genome comprising SEQ ID NO: 4556 and a VOY201 capsid. In some embodiments, the AAV particle comprises a viral genome comprising SEQ ID NO: 4556 and an AAV9 capsid. In some embodiments, the AAV particle comprises a viral genome comprising SEQ ID NO: 4556 and an AAV9 K449R capsid. In some embodiments, the AAV particle comprises a viral genome comprising SEQ ID NO: 4556 and an AAVPHP.B capsid. In some embodiments, the AAV particle comprises a viral genome comprising SEQ ID NO: 4556 and an AAVPHP.N capsid. In some embodiments, the AAV particle comprises a viral genome comprising SEQ ID NO: 4556 and an AAV2 variant capsid. In some embodiments, the AAV particle comprises a viral genome comprising SEQ ID NO: 4556 and an AAV2/3 variant capsid. In some embodiments, the AAV particle comprises a viral genome comprising SEQ ID NO: 4556 and a capsid comprising SEQ ID NO: 1. In some embodiments, the AAV particle comprises a viral genome comprising SEQ ID NO: 4556 and a capsid encoded by a nucleic acid sequence comprising SEQ ID NO: 2. In some embodiments, the AAV particle comprises a viral genome comprising SEQ ID NO: 4556 and a capsid comprising SEQ ID NO: 4534. In some embodiments, the AAV particle comprises a viral genome comprising SEQ ID NO: 4556 and a capsid encoded by a nucleic acid sequence comprising SEQ ID NO: 3. In some embodiments, the AAV particle comprises a viral genome comprising SEQ ID NO: 4556 and a capsid comprising SEQ ID NO: 138. In some embodiments, the AAV particle comprises a viral genome comprising SEQ ID NO: 4556 and a capsid encoded by a nucleic acid sequence comprising SEQ ID NO: 137. In some embodiments, the AAV particle comprises a viral genome comprising SEQ ID NO: 4556 and a capsid comprising SEQ ID NO: 5. In some embodiments, the AAV particle comprises a viral genome comprising SEQ ID NO: 4556 and a capsid encoded by a nucleic acid sequence comprising SEQ ID NO: 6. In some embodiments, the AAV particle comprises a viral genome comprising SEQ ID NO: 4556 and a capsid comprising SEQ ID NO: 4. In some embodiments, the AAV particle comprises a viral genome comprising SEQ ID NO: 4556 and a capsid comprising SEQ ID NO: 11. In some embodiments, the AAV particle comprises a viral genome comprising SEQ ID NO: 4556 and a capsid comprising SEQ ID NO: 2679. In some embodiments, the AAV particle comprises a viral genome comprising SEQ ID NO: 4556 and a capsid comprising SEQ ID NO: 2809. In some embodiments, the AAV particle comprises a viral genome comprising SEQ ID NO: 4556 and a capsid comprising SEQ ID NO: 2871.

In some embodiments, the AAV particle comprises a viral genome comprising SEQ ID NO: 4557 and a VOY101 capsid. In some embodiments, the AAV particle comprises a viral genome comprising SEQ ID NO: 4557 and a VOY201 capsid. In some embodiments, the AAV particle comprises a viral genome comprising SEQ ID NO: 4557 and an AAV9 capsid. In some embodiments, the AAV particle comprises a viral genome comprising SEQ ID NO: 4557 and an AAV9 K449R capsid. In some embodiments, the AAV particle comprises a viral genome comprising SEQ ID NO: 4557 and an AAVPHP.B capsid. In some embodiments, the AAV particle comprises a viral genome comprising SEQ ID NO: 4557 and an AAVPHP.N capsid. In some embodiments, the AAV particle comprises a viral genome comprising SEQ ID NO: 4557 and an AAV2 variant capsid. In some embodiments, the AAV particle comprises a viral genome comprising SEQ ID NO: 4557 and an AAV2/3 variant capsid. In some embodiments, the AAV particle comprises a viral genome comprising SEQ ID NO: 4557 and a capsid comprising SEQ ID NO: 1. In some embodiments, the AAV particle comprises a viral genome comprising SEQ ID NO: 4557 and a capsid encoded by a nucleic acid sequence comprising SEQ ID NO: 2. In some embodiments, the AAV particle comprises a viral genome comprising SEQ ID NO: 4557 and a capsid comprising SEQ ID NO: 4534. In some embodiments, the AAV particle comprises a viral genome comprising SEQ ID NO: 4557 and a capsid encoded by a nucleic acid sequence comprising SEQ ID NO: 3. In some embodiments, the AAV particle comprises a viral genome comprising SEQ ID NO: 4557 and a capsid comprising SEQ ID NO: 138. In some embodiments, the AAV particle comprises a viral genome comprising SEQ ID NO: 4557 and a capsid encoded by a nucleic acid sequence comprising SEQ ID NO: 137. In some embodiments, the AAV particle comprises a viral genome comprising SEQ ID NO: 4557 and a capsid comprising SEQ ID NO: 5. In some embodiments, the AAV particle comprises a viral genome comprising SEQ ID NO: 4557 and a capsid encoded by a nucleic acid sequence comprising SEQ ID NO: 6. In some embodiments, the AAV particle comprises a viral genome comprising SEQ ID NO: 4557 and a capsid comprising SEQ ID NO: 4. In some embodiments, the AAV particle comprises a viral genome comprising SEQ ID NO: 4557 and a capsid comprising SEQ ID NO: 11. In some embodiments, the AAV particle comprises a viral genome comprising SEQ ID NO: 4557 and a capsid comprising SEQ ID NO: 2679. In some embodiments, the AAV particle comprises a viral genome comprising SEQ ID NO: 4557 and a capsid comprising SEQ ID NO: 2809. In some embodiments, the AAV particle comprises a viral genome comprising SEQ ID NO: 4557 and a capsid comprising SEQ ID NO: 2871.

In some embodiments, the AAV particle comprises a viral genome comprising SEQ ID NO: 4558 and a VOY101 capsid. In some embodiments, the AAV particle comprises a viral genome comprising SEQ ID NO: 4558 and a VOY201 capsid. In some embodiments, the AAV particle comprises a viral genome comprising SEQ ID NO: 4558 and an AAV9 capsid. In some embodiments, the AAV particle comprises a viral genome comprising SEQ ID NO: 4558 and an AAV9 K449R capsid. In some embodiments, the AAV particle comprises a viral genome comprising SEQ ID NO: 4558 and an AAVPHP.B capsid. In some embodiments, the AAV particle comprises a viral genome comprising SEQ ID NO: 4558 and an AAVPHP.N capsid. In some embodiments, the AAV particle comprises a viral genome comprising SEQ ID NO: 4558 and an AAV2 variant capsid. In some embodiments, the AAV particle comprises a viral genome comprising SEQ ID NO: 4558 and an AAV2/3 variant capsid. In some embodiments, the AAV particle comprises a viral genome comprising SEQ ID NO: 4558 and a capsid comprising SEQ ID NO: 1. In some embodiments, the AAV particle comprises a viral genome comprising SEQ ID NO: 4558 and a capsid encoded by a nucleic acid sequence comprising SEQ ID NO: 2. In some embodiments, the AAV particle comprises a viral genome comprising SEQ ID NO: 4558 and a capsid comprising SEQ ID NO: 4534. In some embodiments, the AAV particle comprises a viral genome comprising SEQ ID NO: 4558 and a capsid encoded by a nucleic acid sequence comprising SEQ ID NO: 3. In some embodiments, the AAV particle comprises a viral genome comprising SEQ ID NO: 4558 and a capsid comprising SEQ ID NO: 138. In some embodiments, the AAV particle comprises a viral genome comprising SEQ ID NO: 4558 and a capsid encoded by a nucleic acid sequence comprising SEQ ID NO: 137. In some embodiments, the AAV particle comprises a viral genome comprising SEQ ID NO: 4558 and a capsid comprising SEQ ID NO: 5. In some embodiments, the AAV particle comprises a viral genome comprising SEQ ID NO: 4558 and a capsid encoded by a nucleic acid sequence comprising SEQ ID NO: 6. In some embodiments, the AAV particle comprises a viral genome comprising SEQ ID NO: 4558 and a capsid comprising SEQ ID NO: 4. In some embodiments, the AAV particle comprises a viral genome comprising SEQ ID NO: 4558 and a capsid comprising SEQ ID NO: 11. In some embodiments, the AAV particle comprises a viral genome comprising SEQ ID NO: 4558 and a capsid comprising SEQ ID NO: 2679. In some embodiments, the AAV particle comprises a viral genome comprising SEQ ID NO: 4558 and a capsid comprising SEQ ID NO: 2809. In some embodiments, the AAV particle comprises a viral genome comprising SEQ ID NO: 4558 and a capsid comprising SEQ ID NO: 2871.

In some embodiments, the AAV particle comprises a viral genome comprising SEQ ID NO: 4559 and a VOY101 capsid. In some embodiments, the AAV particle comprises a viral genome comprising SEQ ID NO: 4559 and a VOY201 capsid. In some embodiments, the AAV particle comprises a viral genome comprising SEQ ID NO: 4559 and an AAV9 capsid. In some embodiments, the AAV particle comprises a viral genome comprising SEQ ID NO: 4559 and an AAV9 K449R capsid. In some embodiments, the AAV particle comprises a viral genome comprising SEQ ID NO: 4559 and an AAVPHP.B capsid. In some embodiments, the AAV particle comprises a viral genome comprising SEQ ID NO: 4559 and an AAVPHP.N capsid. In some embodiments, the AAV particle comprises a viral genome comprising SEQ ID NO: 4559 and an AAV2 variant capsid. In some embodiments, the AAV particle comprises a viral genome comprising SEQ ID NO: 4559 and an AAV2/3 variant capsid. In some embodiments, the AAV particle comprises a viral genome comprising SEQ ID NO: 4559 and a capsid comprising SEQ ID NO: 1. In some embodiments, the AAV particle comprises a viral genome comprising SEQ ID NO: 4559 and a capsid encoded by a nucleic acid sequence comprising SEQ ID NO: 2. In some embodiments, the AAV particle comprises a viral genome comprising SEQ ID NO: 4559 and a capsid comprising SEQ ID NO: 4534. In some embodiments, the AAV particle comprises a viral genome comprising SEQ ID NO: 4559 and a capsid encoded by a nucleic acid sequence comprising SEQ ID NO: 3. In some embodiments, the AAV particle comprises a viral genome comprising SEQ ID NO: 4559 and a capsid comprising SEQ ID NO: 138. In some embodiments, the AAV particle comprises a viral genome comprising SEQ ID NO: 4559 and a capsid encoded by a nucleic acid sequence comprising SEQ ID NO: 137. In some embodiments, the AAV particle comprises a viral genome comprising SEQ ID NO: 4559 and a capsid comprising SEQ ID NO: 5. In some embodiments, the AAV particle comprises a viral genome comprising SEQ ID NO: 4559 and a capsid encoded by a nucleic acid sequence comprising SEQ ID NO: 6. In some embodiments, the AAV particle comprises a viral genome comprising SEQ ID NO: 4559 and a capsid comprising SEQ ID NO: 4. In some embodiments, the AAV particle comprises a viral genome comprising SEQ ID NO: 4559 and a capsid comprising SEQ ID NO: 11. In some embodiments, the AAV particle comprises a viral genome comprising SEQ ID NO: 4559 and a capsid comprising SEQ ID NO: 2679. In some embodiments, the AAV particle comprises a viral genome comprising SEQ ID NO: 4559 and a capsid comprising SEQ ID NO: 2809. In some embodiments, the AAV particle comprises a viral genome comprising SEQ ID NO: 4559 and a capsid comprising SEQ ID NO: 2871.

In some embodiments, the AAV particle comprises a viral genome comprising SEQ ID NO: 4560 and a VOY101 capsid. In some embodiments, the AAV particle comprises a viral genome comprising SEQ ID NO: 4560 and a VOY201 capsid. In some embodiments, the AAV particle comprises a viral genome comprising SEQ ID NO: 4560 and an AAV9 capsid. In some embodiments, the AAV particle comprises a viral genome comprising SEQ ID NO: 4560 and an AAV9 K449R capsid. In some embodiments, the AAV particle comprises a viral genome comprising SEQ ID NO: 4560 and an AAVPHP.B capsid. In some embodiments, the AAV particle comprises a viral genome comprising SEQ ID NO: 4560 and an AAVPHP.N capsid. In some embodiments, the AAV particle comprises a viral genome comprising SEQ ID NO: 4560 and an AAV2 variant capsid. In some embodiments, the AAV particle comprises a viral genome comprising SEQ ID NO: 4560 and an AAV2/3 variant capsid. In some embodiments, the AAV particle comprises a viral genome comprising SEQ ID NO: 4560 and a capsid comprising SEQ ID NO: 1. In some embodiments, the AAV particle comprises a viral genome comprising SEQ ID NO: 4560 and a capsid encoded by a nucleic acid sequence comprising SEQ ID NO: 2. In some embodiments, the AAV particle comprises a viral genome comprising SEQ ID NO: 4560 and a capsid comprising SEQ ID NO: 4534. In some embodiments, the AAV particle comprises a viral genome comprising SEQ ID NO: 4560 and a capsid encoded by a nucleic acid sequence comprising SEQ ID NO: 3. In some embodiments, the AAV particle comprises a viral genome comprising SEQ ID NO: 4560 and a capsid comprising SEQ ID NO: 138. In some embodiments, the AAV particle comprises a viral genome comprising SEQ ID NO: 4560 and a capsid encoded by a nucleic acid sequence comprising SEQ ID NO: 137. In some embodiments, the AAV particle comprises a viral genome comprising SEQ ID NO: 4560 and a capsid comprising SEQ ID NO: 5. In some embodiments, the AAV particle comprises a viral genome comprising SEQ ID NO: 4560 and a capsid encoded by a nucleic acid sequence comprising SEQ ID NO: 6. In some embodiments, the AAV particle comprises a viral genome comprising SEQ ID NO: 4560 and a capsid comprising SEQ ID NO: 4. In some embodiments, the AAV particle comprises a viral genome comprising SEQ ID NO: 4560 and a capsid comprising SEQ ID NO: 11. In some embodiments, the AAV particle comprises a viral genome comprising SEQ ID NO: 4560 and a capsid comprising SEQ ID NO: 2679. In some embodiments, the AAV particle comprises a viral genome comprising SEQ ID NO: 4560 and a capsid comprising SEQ ID NO: 2809. In some embodiments, the AAV particle comprises a viral genome comprising SEQ ID NO: 4560 and a capsid comprising SEQ ID NO: 2871.

In some embodiments, the AAV particle comprises a viral genome comprising SEQ ID NO: 4561 and a VOY101 capsid. In some embodiments, the AAV particle comprises a viral genome comprising SEQ ID NO: 4561 and a VOY201 capsid. In some embodiments, the AAV particle comprises a viral genome comprising SEQ ID NO: 4561 and an AAV9 capsid. In some embodiments, the AAV particle comprises a viral genome comprising SEQ ID NO: 4561 and an AAV9 K449R capsid. In some embodiments, the AAV particle comprises a viral genome comprising SEQ ID NO: 4561 and an AAVPHP.B capsid. In some embodiments, the AAV particle comprises a viral genome comprising SEQ ID NO: 4561 and an AAVPHP.N capsid. In some embodiments, the AAV particle comprises a viral genome comprising SEQ ID NO: 4561 and an AAV2 variant capsid. In some embodiments, the AAV particle comprises a viral genome comprising SEQ ID NO: 4561 and an AAV2/3 variant capsid. In some embodiments, the AAV particle comprises a viral genome comprising SEQ ID NO: 4561 and a capsid comprising SEQ ID NO: 1. In some embodiments, the AAV particle comprises a viral genome comprising SEQ ID NO: 4561 and a capsid encoded by a nucleic acid sequence comprising SEQ ID NO: 2. In some embodiments, the AAV particle comprises a viral genome comprising SEQ ID NO: 4561 and a capsid comprising SEQ ID NO: 4534. In some embodiments, the AAV particle comprises a viral genome comprising SEQ ID NO: 4561 and a capsid encoded by a nucleic acid sequence comprising SEQ ID NO: 3. In some embodiments, the AAV particle comprises a viral genome comprising SEQ ID NO: 4561 and a capsid comprising SEQ ID NO: 138. In some embodiments, the AAV particle comprises a viral genome comprising SEQ ID NO: 4561 and a capsid encoded by a nucleic acid sequence comprising SEQ ID NO: 137. In some embodiments, the AAV particle comprises a viral genome comprising SEQ ID NO: 4561 and a capsid comprising SEQ ID NO: 5. In some embodiments, the AAV particle comprises a viral genome comprising SEQ ID NO: 4561 and a capsid encoded by a nucleic acid sequence comprising SEQ ID NO: 6. In some embodiments, the AAV particle comprises a viral genome comprising SEQ ID NO: 4561 and a capsid comprising SEQ ID NO: 4. In some embodiments, the AAV particle comprises a viral genome comprising SEQ ID NO: 4561 and a capsid comprising SEQ ID NO: 11. In some embodiments, the AAV particle comprises a viral genome comprising SEQ ID NO: 4561 and a capsid comprising SEQ ID NO: 2679. In some embodiments, the AAV particle comprises a viral genome comprising SEQ ID NO: 4561 and a capsid comprising SEQ ID NO: 2809. In some embodiments, the AAV particle comprises a viral genome comprising SEQ ID NO: 4561 and a capsid comprising SEQ ID NO: 2871.

In some embodiments, the AAV particle comprises a viral genome comprising SEQ ID NO: 4562 and a VOY101 capsid. In some embodiments, the AAV particle comprises a viral genome comprising SEQ ID NO: 4562 and a VOY201 capsid. In some embodiments, the AAV particle comprises a viral genome comprising SEQ ID NO: 4562 and an AAV9 capsid. In some embodiments, the AAV particle comprises a viral genome comprising SEQ ID NO: 4562 and an AAV9 K449R capsid. In some embodiments, the AAV particle comprises a viral genome comprising SEQ ID NO: 4562 and an AAVPHP.B capsid. In some embodiments, the AAV particle comprises a viral genome comprising SEQ ID NO: 4562 and an AAVPHP.N capsid. In some embodiments, the AAV particle comprises a viral genome comprising SEQ ID NO: 4562 and an AAV2 variant capsid. In some embodiments, the AAV particle comprises a viral genome comprising SEQ ID NO: 4562 and an AAV2/3 variant capsid. In some embodiments, the AAV particle comprises a viral genome comprising SEQ ID NO: 4562 and a capsid comprising SEQ ID NO: 1. In some embodiments, the AAV particle comprises a viral genome comprising SEQ ID NO: 4562 and a capsid encoded by a nucleic acid sequence comprising SEQ ID NO: 2. In some embodiments, the AAV particle comprises a viral genome comprising SEQ ID NO: 4562 and a capsid comprising SEQ ID NO: 4534. In some embodiments, the AAV particle comprises a viral genome comprising SEQ ID NO: 4562 and a capsid encoded by a nucleic acid sequence comprising SEQ ID NO: 3. In some embodiments, the AAV particle comprises a viral genome comprising SEQ ID NO: 4562 and a capsid comprising SEQ ID NO: 138. In some embodiments, the AAV particle comprises a viral genome comprising SEQ ID NO: 4562 and a capsid encoded by a nucleic acid sequence comprising SEQ ID NO: 137. In some embodiments, the AAV particle comprises a viral genome comprising SEQ ID NO: 4562 and a capsid comprising SEQ ID NO: 5. In some embodiments, the AAV particle comprises a viral genome comprising SEQ ID NO: 4562 and a capsid encoded by a nucleic acid sequence comprising SEQ ID NO: 6. In some embodiments, the AAV particle comprises a viral genome comprising SEQ ID NO: 4562 and a capsid comprising SEQ ID NO: 4. In some embodiments, the AAV particle comprises a viral genome comprising SEQ ID NO: 4562 and a capsid comprising SEQ ID NO: 11. In some embodiments, the AAV particle comprises a viral genome comprising SEQ ID NO: 4562 and a capsid comprising SEQ ID NO: 2679. In some embodiments, the AAV particle comprises a viral genome comprising SEQ ID NO: 4562 and a capsid comprising SEQ ID NO: 2809. In some embodiments, the AAV particle comprises a viral genome comprising SEQ ID NO: 4562 and a capsid comprising SEQ ID NO: 2871.

### II. Formulation and Delivery

### Pharmaceutical Compositions

According to the present disclosure the AAV particles may be prepared as pharmaceutical compositions. It will be understood that such compositions necessarily comprise one or more active ingredients and, most often, a pharmaceutically acceptable excipient.

Relative amounts of the active ingredient (e.g. AAV particle), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the AAV particle pharmaceutical compositions described herein may comprise at least one payload. As a non-limiting example, the pharmaceutical compositions may contain an AAV particle with 1, 2, 3, 4 or 5 payloads. In some embodiments, the pharmaceutical composition may contain a nucleic acid encoding a payload construct encoding proteins selected from antibodies and/or antibody-based compositions.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients, or subjects.

### Formulations

The AAV particles can be formulated using one or more excipients to: (1) increase stability; (2) increase cell transfection or transduction; (3) permit the sustained or delayed expression of the payload; (4) alter the biodistribution (e.g., target the viral particle to specific tissues or cell types); (5) increase the translation of encoded protein; (6) alter the release profile of encoded protein; and/or (7) allow for regulatable expression of the payload.

Formulations of the present disclosure can include, without limitation, saline, liposomes, lipid nanoparticles, polymers, peptides, proteins, cells transfected with viral vectors (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients. As used herein, the phrase “active ingredient” generally refers either to an AAV particle carrying a payload region encoding the polypeptides or to the antibody or antibody-based composition encoded by a viral genome of by an AAV particle as described herein.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In some embodiments, the AAV particles may be formulated in phosphate buffered saline (PBS), in combination with an ethylene oxide/propylene oxide copolymer (also known as Pluronic or poloxamer).

In some embodiments, the AAV particles may be formulated in PBS with 0.001% Pluronic acid (F-68) (poloxamer 188) at a pH of about 7.0.

In some embodiments, the AAV particles may be formulated in PBS with 0.001% Pluronic acid (F-68) (poloxamer 188) at a pH of about 7.3.

In some embodiments, the AAV particles may be formulated in PBS with 0.001% Pluronic acid (F-68) (poloxamer 188) at a pH of about 7.4.

In some embodiments, the AAV particles may be formulated in a solution comprising sodium chloride, sodium phosphate and an ethylene oxide/propylene oxide copolymer.

In some embodiments, the AAV particles may be formulated in a solution comprising sodium chloride, sodium phosphate dibasic, sodium phosphate monobasic and poloxamer 188/Pluronic acid (F-68).

In some embodiments, the AAV particles may be formulated in a solution comprising 10 mM disodium phosphate, 2 mM monopotassium phosphate, 2.7 mM potassium chloride, 192 mM sodium chloride and 0.001% Pluronic Acid (F-68).

In some embodiments, the AAV particles may be formulated in a solution comprising about 180 mM sodium chloride, about 10 mM sodium phosphate and about 0.001% poloxamer 188. In some embodiments, this formulation may be at a pH of about 7.3. The concentration of sodium chloride in the final solution may be 150 mM-200 mM. As non-limiting examples, the concentration of sodium chloride in the final solution may be 150 mM, 160 mM, 170 mM, 180 mM, 190 mM or 200 mM. The concentration of sodium phosphate in the final solution may be 1 mM-50 mM. As non-limiting examples, the concentration of sodium phosphate in the final solution may be 1 mM, 2 mM, 3 mM, 4 mM, 5 mM, 6 mM, 7 mM, 8 mM, 9 mM, 10 mM, 15 mM, 20 mM, 25 mM, 30 mM, 40 mM, or 50 mM. The concentration of poloxamer 188 (Pluronic acid (F-68)) may be 0.0001%-1%. As non-limiting examples, the concentration of poloxamer 188 (Pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

In some embodiments, the AAV particles of the disclosure may be formulated in a solution comprising about 1.05% sodium chloride, about 0.212% sodium phosphate dibasic, heptahydrate, about 0.025% sodium phosphate monobasic, monohydrate, and 0.001% poloxamer 188, at a pH of about 7.4. As a non-limiting example, the concentration of AAV particle in this formulated solution may be about 0.001%. The concentration of sodium chloride in the final solution may be 0.1-2.0%, with non-limiting examples of 0.1%, 0.25%, 0.5%, 0.75%, 0.95%, 0.96%, 0.97%, 0.98%, 0.99%, 1.00%, 1.01%, 1.02%, 1.03%, 1.04%, 1.05%, 1.06%, 1.07%, 1.08%, 1.09%, 1.10%, 1.25%, 1.5%, 1.75%, or 2%. The concentration of sodium phosphate dibasic in the final solution may be 0.100-0.300% with non-limiting examples including 0.100%, 0.125%, 0.150%, 0.175%, 0.200%, 0.210%, 0.211%, 0.212%, 0.213%, 0.214%, 0.215%, 0.225%, 0.250%, 0.275%, 0.300%. The concentration of sodium phosphate monobasic in the final solution may be 0.010-0.050%, with non-limiting examples of 0.010%, 0.015%, 0.020%, 0.021%, 0.022%, 0.023%, 0.024%, 0.025%, 0.026%, 0.027%, 0.028%, 0.029%, 0.030%, 0.035%, 0.040%, 0.045%, or 0.050%. The concentration of poloxamer 188 (Pluronic acid (F-68)) may be 0.0001%-1%. As non-limiting examples, the concentration of poloxamer 188 (Pluronic acid (F-68)) may be 0.0001%, 0.0005%, 0.001%, 0.005%, 0.01%, 0.05%, 0.1%, 0.5%, or 1%. The final solution may have a pH of 6.8-7.7. Non-limiting examples for the pH of the final solution include a pH of 6.8, 6.9, 7.0, 7.1, 7.2, 7.3, 7.4, 7.5, 7.6, or 7.7.

Relative amounts of the active ingredient (e.g. AAV particle), the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, or at least 80% (w/w) active ingredient.

In some embodiments, the AAV formulations described herein may contain sufficient AAV particles for expression of at least one expressed functional antibody or antibody-based composition. As a non-limiting example, the AAV particles may contain viral genomes encoding 1, 2, 3, 4, or 5 functional antibodies.

According to the present disclosure AAV particles may be formulated for CNS delivery. Agents that cross the brain blood barrier may be used. For example, some cell penetrating peptides that can target molecules to the brain blood barrier endothelium may be used for formulation (e.g., Mathupala, Expert Opin Ther Pat., 2009, 19, 137-140; the content of which is incorporated herein by reference in its entirety).

### Excipients and Diluents

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, M D, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, AAV particle formulations may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present disclosure may be approved by the US Food and Drug Administration (FDA).

Pharmaceutical composition formulations of AAV particles disclosed herein may include cations or anions. In some embodiments, the formulations include metal cations such as, but not limited to, Zn2+, Ca2+, Cu2+, Mn2+, Mg+ and combinations thereof. As a non-limiting example, formulations may include polymers and complexes with a metal cation (See e.g., U.S. Pat. Nos. 6,265,389 and 6,555,525, each of which is herein incorporated by reference in its entirety).

### III. Administration and Dosing

### Administration

The AAV particles of the present disclosure may be administered by any delivery route which results in a therapeutically effective outcome. These include, but are not limited to, enteral (into the intestine), gastroenteral, epidural (into the dura mater), oral (by way of the mouth), transdermal, intracerebral (into the cerebrum), intracerebroventricular (into the cerebral ventricles), epicutaneous (application onto the skin), intradermal (into the skin itself), subcutaneous (under the skin), nasal administration (through the nose), intravenous (into a vein), intravenous bolus, intravenous drip, intra-arterial (into an artery), intramuscular (into a muscle), intracardiac (into the heart), intraosseous infusion (into the bone marrow), intrathecal (into the spinal canal), intraparenchymal (into brain tissue), intraperitoneal (infusion or injection into the peritoneum), intravesical infusion, intravitreal (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracoronal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis, and spinal. In some embodiments, an AAV particle comprising a viral genome encoding an antibody, e.g., an antibody fragment, described herein is administered intravenously, intracerebrally, intrathecally, intracerebroventricularly, intramuscularly, via intraparenchymal administration, via focused ultrasound (FUS), e.g., coupled with the intravenous administration of microbubbles (FUS-MB), or MRI-guided FUS coupled with intravenous administration, or via intra-cisterna magna injection (ICM).

In some embodiments, compositions may be administered in a way which allows them to cross the blood-brain barrier, vascular barrier, or other epithelial barrier. The AAV particles of the present disclosure may be administered in any suitable form, either as a liquid solution or suspension, as a solid form suitable for liquid solution or suspension in a liquid solution. The AAV particles may be formulated with any appropriate and pharmaceutically acceptable excipient.

In some embodiments, the AAV particles of the present disclosure may be delivered to a subject via a single route administration.

In some embodiments, the AAV particles of the present disclosure may be delivered to a subject via a multi-site route of administration. A subject may be administered at 2, 3, 4, 5, or more than 5 sites.

In some embodiments, a subject may be administered the AAV particles of the present disclosure using a bolus infusion.

In some embodiments, a subject may be administered the AAV particles of the present disclosure using sustained delivery over a period of minutes, hours, or days. The infusion rate may be changed depending on the subject, distribution, formulation or another delivery parameter.

In some embodiments, the AAV particles may be delivered by more than one route of administration. As non-limiting examples of combination administrations, AAV particles may be delivered by intrathecal and intracerebroventricular, or by intravenous and intraparenchymal administration.

### Intravenous Administration

In some embodiments, the AAV particles may be administered to a subject by systemic administration.

In some embodiments, the systemic administration is intravenous administration.

In another embodiment, the systemic administration is intraarterial administration.

In some embodiments, the AAV particles of the present disclosure may be administered to a subject by intravenous administration.

In some embodiments, the intravenous administration may be achieved by subcutaneous delivery.

In some embodiments, the intravenous administration may be achieved by a tail vein injection (e.g., in a mouse model).

In some embodiments, the intravenous administration may be achieved by retro-orbital injection.

### Administration to the CNS

In some embodiments, the AAV particles may be delivered by direct injection into the brain. As a non-limiting example, the brain delivery may be by intrahippocampal administration.

In some embodiments, the AAV particles of the present disclosure may be administered to a subject by intraparenchymal administration. In some embodiments, the intraparenchymal administration is to tissue of the central nervous system.

In some embodiments, the AAV particles of the present disclosure may be administered to a subject by intracranial delivery (See, e.g., U.S. Pat. No. 8,119,611; the content of which is incorporated herein by reference in its entirety).

In some embodiments, the AAV particles may be delivered by injection into the CSF pathway. Non-limiting examples of delivery to the CSF pathway include intrathecal and intracerebroventricular administration.

In some embodiments, the AAV particles may be delivered to the brain by systemic delivery. As a non-limiting example, the systemic delivery may be by intravascular administration. As a non-limiting example, the systemic or intravascular administration may be intravenous.

In some embodiments, the AAV particles of the present disclosure may be delivered by intraocular delivery route. A non-limiting example of intraocular administration include an intravitreal injection.

### Intramuscular Administration

In some embodiments, the AAV particles may be delivered by intramuscular administration. Whilst not wishing to be bound by theory, the multi-nucleated nature of muscle cells provides an advantage to gene transduction subsequent to AAV delivery. Cells of the muscle are capable of expressing recombinant proteins with the appropriate post-translational modifications. The enrichment of muscle tissue with vascular structures allows for transfer to the blood stream and whole-body delivery. Examples of intramuscular administration include systemic (e.g., intravenous), subcutaneous or directly into the muscle. In some embodiments, more than one injection is administered.

In some embodiments, the AAV particles of the present disclosure may be delivered by intramuscular delivery route. (See, e.g., U.S. Pat. No. 6,506,379; the content of which is incorporated herein by reference in its entirety). Non-limiting examples of intramuscular administration include an intravenous injection or a subcutaneous injection.

In some embodiments, the AAV particles of the present disclosure are administered to a subject and transduce muscle of a subject. As a non-limiting example, the AAV particles are administered by intramuscular administration.

In some embodiments, the AAV particles of the present disclosure may be administered to a subject by subcutaneous administration.

In some embodiments, the intramuscular administration is via systemic delivery.

In some embodiments, the intramuscular administration is via intravenous delivery.

In some embodiments, the intramuscular administration is via direct injection to the muscle.

In some embodiments, the muscle is transduced by administration, and this is referred to as intramuscular administration.

In some embodiments, the intramuscular delivery comprises administration at one site.

In some embodiments, the intramuscular delivery comprises administration at more than one site. In some embodiments, the intramuscular delivery comprises administration at two sites. In some embodiments, the intramuscular delivery comprises administration at three sites. In some embodiments, the intramuscular delivery comprises administration at four sites. In some embodiments, the intramuscular delivery comprises administration at more than four sites.

In some embodiments, intramuscular delivery is combined with at least one other method of administration.

In some embodiments, the AAV particles that may be administered to a subject by peripheral injections. Non-limiting examples of peripheral injections include intraperitoneal, intramuscular, intravenous, conjunctival, or joint injection. It was disclosed in the art that the peripheral administration of AAV vectors can be transported to the central nervous system, for example, to the motor neurons (e.g., U.S. Patent Publication Nos. US20100240739 and US20100130594; the content of each of which is incorporated herein by reference in their entirety).

In some embodiments, the AAV particles of the present disclosure may be administered to a subject by intraparenchymal administration. In some embodiments, the intraparenchymal administration is to muscle tissue.

In some embodiments, the AAV particles of the present disclosure are delivered as described in Bright et al 2015 (Neurobiol Aging. 36(2):693-709), the contents of which are herein incorporated by reference in their entirety.

In some embodiments, the AAV particles of the present disclosure are administered to the gastrocnemius muscle of a subject.

In some embodiments, the AAV particles of the present disclosure are administered to the bicep femorii of the subject.

In some embodiments, the AAV particles of the present disclosure are administered to the tibialis anterior muscles.

In some embodiments, the AAV particles of the present disclosure are administered to the soleus muscle.

### Depot Administration

As described herein, in some embodiments, pharmaceutical compositions, AAV particles of the present disclosure are formulated in depots for extended release. Generally, specific organs or tissues (“target tissues”) are targeted for administration.

In some aspects, pharmaceutical compositions, AAV particles of the present disclosure are spatially retained within or proximal to target tissues. Provided are methods of providing pharmaceutical compositions, AAV particles, to target tissues of mammalian subjects by contacting target tissues (which comprise one or more target cells) with pharmaceutical compositions, AAV particles, under conditions such that they are substantially retained in target tissues, meaning that at least 10, 20, 30, 40, 50, 60, 70, 80, 85, 90, 95, 96, 97, 98, 99, 99.9, 99.99 or greater than 99.99% of the composition is retained in the target tissues. Advantageously, retention is determined by measuring the amount of pharmaceutical compositions, AAV particles, that enter one or more target cells. For example, at least 1%, 5%, 10%, 20%, 30%, 40%, 50%, 60%, 70%, 80%, 85%, 90%, 95%, 96%, 97%, 98%, 99%, 99.9%, 99.99%, or greater than 99.99% of pharmaceutical compositions, AAV particles, administered to subjects are present intracellularly at a period of time following administration. For example, intramuscular injection to mammalian subjects may be performed using aqueous compositions comprising pharmaceutical compositions, AAV particles of the present disclosure and one or more transfection reagents, and retention is determined by measuring the amount of pharmaceutical compositions, AAV particles, present in muscle cells.

Certain aspects are directed to methods of providing pharmaceutical compositions, AAV particles of the present disclosure to a target tissues of mammalian subjects, by contacting target tissues (comprising one or more target cells) with pharmaceutical compositions, AAV particles under conditions such that they are substantially retained in such target tissues. Pharmaceutical compositions, AAV particles comprise enough active ingredient such that the effect of interest is produced in at least one target cell. In some embodiments, pharmaceutical compositions, AAV particles generally comprise one or more cell penetration agents, although “naked” formulations (such as without cell penetration agents or other agents) are also contemplated, with or without pharmaceutically acceptable carriers.

### Delivery

In some embodiments, the AAV particles or pharmaceutical compositions of the present disclosure may be administered or delivered using the methods for treatment of disease described in U.S. Pat. No. 8,999,948, or International Publication No. WO2014178863, the contents of which are herein incorporated by reference in their entirety.

In some embodiments, the AAV particles or pharmaceutical compositions of the present disclosure may be administered or delivered using the methods for delivering gene therapy in Alzheimer's Disease or other neurodegenerative conditions as described in US Application No. 20150126590, the contents of which are herein incorporated by reference in their entirety.

In some embodiments, the AAV particles or pharmaceutical compositions of the present disclosure may be administered or delivered using the methods for delivery of a CNS gene therapy as described in U.S. Pat. Nos. 6,436,708, and 8,946,152, and International Publication No. WO2015168666, the contents of which are herein incorporated by reference in their entirety.

In some embodiments, the AAV particle or pharmaceutical compositions of the present disclosure may be administered or delivered using the methods for delivering proteins using AAV vectors described in European Patent Application No. EP2678433, the contents of which are herein incorporated by reference in their entirety.

In some embodiments, the AAV particle or pharmaceutical compositions of the present disclosure may be administered or delivered using the methods for delivering DNA to the bloodstream described in U.S. Pat. No. 6,211,163, the contents of which are herein incorporated by reference in their entirety.

In some embodiments, the AAV particle or pharmaceutical compositions of the present disclosure may be administered or delivered using the methods for delivering a payload to the central nervous system described in U.S. Pat. No. 7,588,757, the contents of which are herein incorporated by reference in their entirety.

In some embodiments, the AAV particle or pharmaceutical compositions of the present disclosure may be administered or delivered using the methods for delivering a payload described in U.S. Pat. No. 8,283,151, the contents of which are herein incorporated by reference in their entirety.

In some embodiments, the AAV particle or pharmaceutical compositions of the present disclosure may be administered or delivered using the methods for delivering a payload using a glutamic acid decarboxylase (GAD) delivery vector described in International Patent Publication No. WO2001089583, the contents of which are herein incorporated by reference in their entirety.

In some embodiments, the AAV particle or pharmaceutical compositions of the present disclosure may be administered or delivered using the methods for delivering a payload to neural cells described in International Patent Publication No. WO2012057363, the contents of which are herein incorporated by reference in their entirety.

### Delivery to Cells

The present disclosure provides a method of delivering to a cell or tissue any of the above-described AAV particles, comprising contacting the cell or tissue with said AAV particle or contacting the cell or tissue with a formulation comprising said AAV particle, or contacting the cell or tissue with any of the described compositions, including pharmaceutical compositions. The method of delivering the AAV particle to a cell or tissue can be accomplished in vitro, ex vivo, or in vivo.

### Delivery to Subjects

The present disclosure additionally provides a method of delivering to a subject, including a mammalian subject, any of the above-described AAV particles comprising administering to the subject said AAV particle, or administering to the subject a formulation comprising said AAV particle, or administering to the subject any of the described compositions, including pharmaceutical compositions.

### Dose and Regimen

The present disclosure provides methods of administering AAV particles in accordance with the disclosure to a subject in need thereof. The pharmaceutical, diagnostic, or prophylactic AAV particles and compositions of the present disclosure may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal. Compositions in accordance with the disclosure are typically formulated in unit dosage form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, route of administration, and rate of excretion of the specific AAV particle employed; the duration of the treatment; drugs used in combination or coincidental with the specific AAV particle employed; and like factors well known in the medical arts.

In certain embodiments, AAV particle pharmaceutical compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect. It will be understood that the above dosing concentrations may be converted to vg or viral genomes per kg or into total viral genomes administered by one of skill in the art.

In certain embodiments, AAV particle pharmaceutical compositions in accordance with the present disclosure may be administered at about 10 to about 600 μl/site, 50 to about 500 μl/site, 100 to about 400 μl/site, 120 to about 300 μl/site, 140 to about 200 μl/site, about 160 μl/site. As non-limiting examples, AAV particles may be administered at 50 μl/site and/or 150 μl/site.

In certain embodiments, the desired dosage may be delivered using multiple administrations (e.g., two, three, four, or more than four administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event.

The desired dosage of the AAV particles of the present disclosure may be administered as a “pulse dose” or as a “continuous flow”. As used herein, a “pulse dose” is a series of single unit doses of any therapeutic administered with a set frequency over a period of time. As used herein, a “continuous flow” is a dose of therapeutic administered continuously for a period of time in a single route/single point of contact, i.e., continuous administration event. A total daily dose, an amount given or prescribed in 24-hour period, may be administered by any of these methods, or as a combination of these methods, or by any other methods suitable for a pharmaceutical administration.

In some embodiments, delivery of the AAV particles of the present disclosure to a subject provides neutralizing activity to a subject. The neutralizing activity can be for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years.

In some embodiments, delivery of the AAV particles of the present disclosure results in minimal serious adverse events (SAEs) as a result of the delivery of the AAV particles.

In some embodiments, delivery of AAV particles may comprise a total dose between about 1×10⁶ VG and about 1×10¹⁶ VG. In some embodiments, delivery may comprise a total dose of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.9×10¹⁰, 2×10¹⁰, 3×10¹⁰, 3.73×10¹⁰, 4×10¹⁰, 5×10¹⁰, 6×10¹⁰, 7×10¹⁰, 8×10¹⁰, 9×10¹⁰, 1×10¹¹, 2×10¹¹, 2.5×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG. As a non-limiting example, the total dose is 1×10¹³ VG. As another non-limiting example, the total dose is 2.1×10¹² VG.

In some embodiments, delivery of AAV particles may comprise a composition concentration between about 1×10⁶ VG/mL and about 1×10¹⁶ VG/mL. In some embodiments, delivery may comprise a composition concentration of about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, or 1×10¹⁶ VG/mL. In some embodiments, the delivery comprises a composition concentration of 1×10¹³ VG/mL. In some embodiments, the delivery comprises a composition concentration of 2.1×10¹² VG/mL.

### Combinations

The AAV particles may be used in combination with one or more other therapeutic, prophylactic, research or diagnostic agents. By “in combination with,” it is not intended to imply that the agents must be administered at the same time and/or formulated for delivery together, although these methods of delivery are within the scope of the present disclosure. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, research, or diagnostic compositions in combination with agents that may improve their bioavailability, reduce and/or modify their metabolism, inhibit their excretion, and/or modify their distribution within the body.

### Measurement of Expression

Expression of payloads from viral genomes may be determined using various methods known in the art such as, but not limited to immunochemistry (e.g., IHC), in situ hybridization (ISH), enzyme-linked immunosorbent assay (ELISA), affinity ELISA, ELISPOT, flow cytometry, immunocytology, immunohistochemistry, surface plasmon resonance analysis, kinetic exclusion assay, liquid chromatography-mass spectrometry (LCMS), high-performance liquid chromatography (HPLC), BCA assay, immunoelectrophoresis, Western blot, SDS-PAGE, protein immunoprecipitation, and/or PCR.

In some embodiments, the ELISA assays used are those described in Liu et al 2016, the contents of which are herein incorporated by reference in their entirety (Liu, W et al., 2016 J Neurosci 36(49):12425-12435).

### IV. Methods and Uses of the Compositions

The present disclosure provides a method for treating a disease, disorder and/or condition in a mammalian subject, including a human subject, comprising administering to the subject any of the AAV particles described herein or administering to the subject any of the described compositions, including pharmaceutical compositions, described herein.

In some embodiments, the AAV particles of the present disclosure are administered to a subject prophylactically.

In some embodiments, the AAV particles of the present disclosure are administered to a subject having at least one of the diseases described herein.

In some embodiments, the AAV particles of the present disclosure are administered to a subject to treat a disease or disorder described herein. The subject may have the disease or disorder or may be at-risk to developing the disease or disorder.

In some embodiments, the AAV particles of the present disclosure are part of an active immunization strategy to protect against diseases and disorders. In an active immunization strategy, a vaccine or AAV particles are administered to a subject to prevent an infectious disease by activating the subject's production of antibodies that can fight off invading bacteria or viruses.

In some embodiments, the AAV particles of the present disclosure are part of a passive immunization strategy. In a passive immunization strategy, antibodies against a particular infectious agent are given directly to the subject.

In some embodiments, the AAV particles of the present disclosure may be used for passive immunotherapy of tauopathy, (e.g. Alzheimer Disease or Frontotemporal Dementia), as described in Liu et al, the contents of which are herein incorporated by reference in their entirety (Liu, W et al., 2016 J Neurosci 36(49):12425-12435). As a non-limiting example, the AAV particles of the present disclosure may encode a PHF1 antibody. Heavy and light chains of the PHF1 antibody may be linked by a Tav2A and/or Furin 2A linker sequence. Antibody expression may be under the control of a CAG promoter. The AAV particle may comprise, as a non-limiting example, an AAVrh.10 serotype capsid. Further, these PHF1 encoding AAV particles may be administered by bilateral intraparenchymal delivery directly to the hippocampus. Such treatment with AAV-PHF1 may result in a 50-fold increase in antibody levels in the hippocampus as compared to antibody levels subsequent to systemic administration. Neuropathological tau species in the hippocampus may be reduced as much as 80-90% and hippocampal atrophy may be fully rescued after treatment with AAV particles of the present disclosure.

In some embodiments, the AAV particles of the present disclosure may be used to treat tauopathy as described in Ising et al, the contents of which are herein incorporated by reference in their entirety (Ising, C et al., 2017 J Exp Med April 17, Epub ahead of print). As a non-limiting example, the AAV particles of the present disclosure may encode an HJ8.5, HJ8.7, or Tau5 antibody or a single chain variable fragment (scFv) derived therefrom. Heavy and light chains of the HJ8.5 antibody or scFv may be linked by variable length linker sequences and may be flexible glycine and/or serine linkers. The AAV particle may comprise, as a non-limiting example, an AAV2/8 serotype. Further, these HJ8.5, HJ8.7 or Tau5 encoding AAV particles may be administered by bilateral intracerebroventricular delivery. Such treatment with HJ8.5, HJ8.7 or Tau5 encoding AAV particles may result in a significant reduction in neuropathological tau species in the hippocampus.

Passive immunization by intravenous (or intraperitoneal in mice) delivery of antibody has been shown to result in substantial serum levels of antibody (Chai et al., 2011, J Biol Chem., 286, 34457-34467, the contents of which are herein incorporated by reference in their entirety) and reduced tau pathology in mouse models of tauopathy (e.g., P301L or P301S mice). However, these reductions in tau pathology are considered modest (e.g., about 34%) and require high and repeated dosing to achieve. Passive immunization strategies are thought to be limited in their ability to deliver large quantity of antibody to the brain, which may limit efficacy, and are also challenged in addressing intracellular aggregates. In some embodiments, delivery of an AAV particle comprising a viral genome encoding an anti-tau antibody can be used to overcome the limitations seen with passive immunization strategies.

In some embodiments, the administration of AAV particles of the present disclosure may result in substantially higher antibody levels in the target tissue (e.g., CNS) of the subject than if anti-tau antibodies were administered by passive immunization. In some embodiments, AAV mediated delivery can result in 1.5-16 fold higher antibody levels in the brain than if delivered by passive immunization. Whilst not wishing to be bound by theory, passive immunization is thought to generate 20-40 ng of antibody per mg of protein in the brain of the subject. In some embodiments, AAV-mediated delivery results in antibody levels 2-5× above the levels seen with passive immunization. In some embodiments, AAV-mediated delivery results in antibody levels 1.5-3× above the levels seen with passive immunization. In some embodiments, AAV-mediated delivery results in antibody levels 5-10× above the levels seen with passive immunization. In some embodiments, AAV-mediated delivery results in antibody levels 8-16× above the levels seen with passive immunization.

In some embodiments, AAV mediated delivery of anti-tau antibody may be used to reduce tau seeding, prevent tau seeding and/or prevent the propagation of tau seeds in a subject. Tau may exist in both a monomeric form and in different aggregated forms. As used herein, the term “tau aggregate” refers to a molecular complex that comprises two or more tau monomers. A tau aggregate may include a nearly unlimited number of monomers bound together. For example, a tau aggregate may comprise 2, 3, 4, 5, 6, 7, 8, 9, 10 or more tau monomers. Alternatively, a tau aggregate may include 20, 30, 40, 50, 60, 70, 80, 90, 100 or more tau monomers. A tau aggregate may also include 500, 1000, 1500, 2000, 2500, 3000, 3500, 4000, 4500, 5000 or more tau monomers. The terms “fibrillar tau aggregate” and “tau fibril” may refer to forms of tau aggregates, and these terms are used interchangeably herein. A fibrillar tau aggregate is a polymeric, ordered fiber comprising tau. Tau fibrils are generally not soluble, but shorter assemblies, or oligomers, can be soluble. Tau aggregate also refers to soluble tau oligomers and protofibrils, which may act as intermediates during tau aggregation. Also included in the definition of tau aggregate is the term “tau seed”, which refers to a tau aggregate that is capable of nucleating or “seeding” intracellular tau aggregation when internalized by a cell, or when exposed to monomeric tau in vitro.

Tau seeding activity may be assessed in vitro in cellular tau models or in vivo in mouse models. Tau seeds for efficacy studies may be variant Tau fibrils such as, but not limited to, htau40, P301S, P301L, and K18. Tau seeds may also be prepared from brain lysates of subjects with Alzheimer's disease and/or with a P301S mutation. Tau seeds may also include enriched PHFs (ePHF) or immuno purified PHF (iPHF).

In vivo, the accumulation of intracellular tau amyloids defines tauopathies. In early disease stages, the disease pathology is generally localized to discrete regions of the brain, but with disease progression, pathology invariably spreads along distinct neural networks. Accumulating evidence suggests transcellular propagation of tau seeds underlies this pathology. In this model, tau are released from donor cells and enter neighboring cells, transforming native tau protein into the misfolded form via templated conformational change. AAV particles or pharmaceutical formulations containing the AAV particles of the disclosure may be administered to a subject in order to prevent, delay or reduce the progression of tau seeds through neural networks.

In some embodiments, antibodies may be generated and/or screened using methods such as but not limited to, hybridoma technology, recombinant antibody productions, reverse translation for antibody generation, as described in Example 1 of copending commonly owned U.S. Ser. No. 62,839/891, filed on Apr. 29, 2019, the contents of which are incorporated herein by reference in their entirety.

In some embodiments, codon-optimization methods utilized in antibody preparation may include any of the methods described in Example 1 of copending commonly owned U.S. Ser. No. 62,839/891, filed on Apr. 29, 2019, the contents of which are incorporated herein by reference in their entirety.

In some embodiments, the present disclosure provides methods for engineering viral genomes for the expression of anti-tau antibodies such as, but not limited to IPN002, C10.2, PT3, MC1 including codon optimized variants and promoter and configuration variants of the same, as described in Example 2 of copending commonly owned U.S. Ser. No. 62,839/891, filed on Apr. 29, 2019, the contents of which are incorporated herein by reference in their entirety.

In some embodiments, the present disclosure provides methods for production and purification of AAV particles as described in Example 3 of copending commonly owned U.S. Ser. No. 62,839/891, filed on Apr. 29, 2019, the contents of which are incorporated herein by reference in their entirety.

In some embodiments, any of the assays including ELISA assay developed to determine the affinity of anti-tau antibodies to ePHF tau may be useful in the present disclosure, including assays described in Example 4 of copending commonly owned US Ser. No. 62,839/891, filed on Apr. 29, 2019, the contents of which are incorporated herein by reference in their entirety.

In some embodiments, antibodies described herein may be detected using ELISA assay as described in Example 5 of copending commonly owned U.S. Ser. No. 62,839/891, filed on Apr. 29, 2019, the contents of which are incorporated herein by reference in their entirety.

In some embodiments, antibodies described herein may be detected using western blotting as described in Example 6 of copending commonly owned US Ser. No. 62,839/891, filed on Apr. 29, 2019, the contents of which are incorporated herein by reference in their entirety.

In some embodiments, the present disclosure may include any of the methods for purification of anti-tau antibody constructs e.g. MC1, or PT3 described in Example 7 of copending commonly owned U.S. Ser. No. 62,839/891, filed on Apr. 29, 2019, the contents of which are incorporated herein by reference in their entirety.

In some embodiments, viral genomes may be optimized for antibody expression as described in Example 8 of copending commonly owned U.S. Ser. No. 62,839/891, filed on Apr. 29, 2019, the contents of which are incorporated herein by reference in their entirety.

In some embodiments, optimization may involve promoter selection, incorporation of linkers, cleavage sites and/or optimizing the configuration of the viral genome for expression as described in Example 8 of copending commonly owned U.S. Ser. No. 62,839/891, filed on Apr. 29, 2019, the contents of which are incorporated herein by reference in their entirety.

In some embodiments, rAAV production of anti-tau antibodies using HEK293T cells may be performed as described in Example 9 of copending commonly owned US Ser. No. 62,839/891, filed on Apr. 29, 2019, the contents of which are incorporated herein by reference in their entirety.

In some embodiments, viral genomes may be constructed for the expression of antibody fragments as described in Example 10 of copending commonly owned US Ser. No. 62,839/891, filed on Apr. 29, 2019, the contents of which are incorporated herein by reference in their entirety.

In some embodiments, AAV particles comprising viral genomes encoding anti-tau antibodies are modified for enhanced transduction to muscular tissue as described in Example 13 of copending commonly owned U.S. Ser. No. 62,839/891, filed on Apr. 29, 2019, the contents of which are incorporated herein by reference in their entirety.

In some embodiments, doses suitable for intramuscular dosing of anti-tau antibody AAV particles may be identified as described in Example 14 of copending commonly owned U.S. Ser. No. 62,839/891, filed on Apr. 29, 2019, the contents of which are incorporated herein by reference in their entirety.

In some embodiments, methods of treatment of Tau-Associated Disease using AAV particles of the disclosure are described in Example 16 of copending commonly owned US Ser. No. 62,839/891, filed on Apr. 29, 2019, the contents of which are incorporated herein by reference in their entirety.

In some embodiments, vectorized PT3 and IPN002 antibodies or fragments thereof may be expressed in primary hippocampal neurons as described in Example 18 of copending commonly owned U.S. Ser. No. 63,002/011, filed on Mar. 30, 2020, the contents of which are incorporated herein by reference in their entirety.

In some embodiments, viral genomes comprising a T2A cleavage site may contribute to greater antibody expression than when an F2A cleavage site is used as described in Example 18 of copending commonly owned U.S. Ser. No. 63,002/011, filed on Mar. 30, 2020, the contents of which are incorporated herein by reference in their entirety.

In some embodiments, the expression and distribution of scFv based anti-tau antibody constructs are tested and optimized as described in Example 19 of copending commonly owned U.S. Ser. No. 63,002/011, filed on Mar. 30, 2020, the contents of which are incorporated herein by reference in their entirety.

In some embodiments, the effects of promoters and viral genome configurations on brain distribution, cellular tropism and expression levels of anti-tau antibody PT3, after intravenous delivery, viral genomes may be undertaken as described in Example 20 of copending commonly owned U.S. Ser. No. 63,002/011, filed on Mar. 30, 2020, the contents of which are incorporated herein by reference in their entirety.

In some embodiments, the biodistribution, tissue tropism and expression patterns of anti-tau antibody IPN002 or PT3 fragments (Fab vs (Fab′)2) driven by CAG promoter in serum, CSF and/or CNS tissue of mice after intravenous delivery of AAV particles, viral genomes are analyzed as described in Example 21 of copending commonly owned US Ser. No. 63,002/011, filed on Mar. 30, 2020, the contents of which are incorporated herein by reference in their entirety.

### Diagnostic Applications

The AAV particles of the present disclosure may be used for diagnostic purposes or as diagnostic tools for any disease or disorder. As non-limiting examples, the AAV particles of the present disclosure or the antibodies encoded within the viral genome therein may be used as a biomarker for disease diagnosis. As a second non-limiting example, the AAV particles of the present disclosure or the antibodies encoded within the viral genome therein may be used for diagnostic imaging purposes, e.g., MRI, PET, CT or ultrasound.

### Preventative Applications

The AAV particles of the present disclosure or the antibodies encoded by the viral genome therein may be used to prevent disease or stabilize the progression of disease. In some embodiments, the AAV particles of the present disclosure are used to as a prophylactic to prevent a disease or disorder in the future. In some embodiments, the AAV particles of the present disclosure are used to halt further progression of a disease or disorder. As a non-limiting example, the AAV particles may be used in a manner similar to that of a vaccine.

### Research Applications

The AAV particles of the present disclosure or the antibodies encoded by the viral genome therein may also be used as research tools. The AAV particles may be used as in any research experiment, e.g., in vivo or in vitro experiments. In a non-limiting example, the AAV particles may be used in cultured cells. The cultured cells may be derived from any origin known to one with skill in the art, and may be as non-limiting examples, derived from a stable cell line, an animal model or a human patient or control subject. In a non-limiting example, the AAV particles may be used in in vivo experiments in animal models (i.e., mouse, rat, rabbit, dog, cat, non-human primate, guinea pig, ferret, C-elegans, Drosophila, zebrafish, or any other animal used for research purposes, known in the art). In another non-limiting example, the AAV particles may be used in human research experiments or human clinical trials.

### Combination Applications

The AAV particles may be used as a combination therapy with any other therapeutic molecule known in the art. The therapeutic molecule may be approved by the US Food and Drug Administration or may be in clinical trial or at the preclinical research stage. The therapeutic molecule may utilize any therapeutic modality known in the art, with non-limiting examples including gene silencing or interference (i.e., miRNA, siRNA, RNAi, shRNA), gene editing (i.e., TALEN, CRISPR/Cas9 systems, zinc finger nucleases), and gene, protein or enzyme replacement.

### Therapeutic Applications

The present disclosure additionally provides a method for treating neurological diseases and/or disorders in a mammalian subject, including a human subject, comprising administering to the subject any of the AAV particles. In some cases, neurological diseases and/or disorders treated according to methods described herein include indications involving irregular expression or aggregation of tau. Such indications may include, but are not limited to Alzheimer's disease (AD), frontotemporal dementia and parkinsonism linked to chromosome 17 (FTDP-17), Frontotemporal lobar degeneration (FTLD), Frontotemporal dementia (FTD), chronic traumatic encephalopathy (CTE), Progressive Supranuclear Palsy (PSP), Down's syndrome, Pick's disease, Corticobasal degeneration (CBD), Corticobasal syndrome, Amyotrophic lateral sclerosis (ALS), Prion diseases, Creutzfeldt-Jakob disease (CJD), Multiple system atrophy, Tangle-only dementia, and Progressive subcortical gliosis.

In some embodiments, methods of treating neurological diseases and/or disorders in a subject in need thereof may comprise the steps of: (1) deriving, generating and/or selecting an anti-tau antibody, antibody-based composition or fragment thereof; (2) producing an AAV particle with a viral genome that includes a payload region encoding the selected antibody of (1); and (3) administering the AAV particle (or pharmaceutical composition thereof) to the subject.

The present disclosure provides a method for administering to a subject in need thereof, including a human subject, a therapeutically effective amount of the AAV particles to slow, stop or reverse disease progression. As a non-limiting example, disease progression may be measured by cognitive tests such as, but not limited to, the Mini-Mental State Exam (MMSE) or other similar diagnostic tool(s), known to those skilled in the art. As another non-limiting example, disease progression may be measured by change in the pathological features of the brain, CSF or other tissues of the subject, such as, but not limited to a decrease in levels of tau (either soluble or insoluble). In some embodiments, the levels of insoluble hyperphosphorylated tau are decreased. In some embodiments levels of soluble tau are decreased. In some embodiments both soluble and insoluble tau are decreased. In some embodiments, levels of insoluble hyperphosphorylated tau are increased. In some embodiments levels of soluble tau are increased. In some embodiments both insoluble and soluble tau levels are increased. In some embodiments, neurofibrillary tangles are decreased in size, number, density, or combination thereof. In another embodiment, neurofibrillary tangles are increased in size, number, density or combination thereof.

### Alzheimer's Disease

Alzheimer Disease (AD) is a debilitating neurodegenerative disease currently afflicting more than 35 million people worldwide, with that number expected to double in coming decades. Symptomatic treatments have been available for many years but these treatments do not address the underlying pathophysiology. Recent clinical trials using these and other treatments have largely failed and, to date, no known cure has been identified.

The AD brain is characterized by the presence of two forms of pathological aggregates, the extracellular plaques composed of β-amyloid (Aβ) and the intracellular neurofibrillary tangles (NFT) comprised of hyperphosphorylated microtubule associated protein tau. Based on early genetic findings, β-amyloid alterations were thought to initiate disease, with changes in tau considered downstream. Thus, most clinical trials have been Aβ-centric. Although no mutations of the tau gene have been linked to AD, such alterations have been shown to result in a family of dementias known as tauopathies, demonstrating that changes in tau can contribute to neurodegenerative processes. Tau is normally a very soluble protein known to associate with microtubules based on the extent of its phosphorylation. Hyperphosphorylation of tau depresses its binding to microtubules and microtubule assembly activity. In tauopathies, the tau becomes hyperphosphorylated, misfolds and aggregates as NFT of paired helical filaments (PHF), twisted ribbons or straight filaments. In AD, NFT pathology, rather than plaque pathology, correlates more closely with neuropathological markers such as neuronal loss, synaptic deficits, severity of disease and cognitive decline. NFT pathology marches through the brain in a stereotyped manner and animal studies suggest a trans-cellular propagation mechanism along neuronal connections.

Several approaches have been proposed for therapeutically interfering with progression of tau pathology and preventing the subsequent molecular and cellular consequences. Given that NFT are composed of a hyperphosphorylated, misfolded and aggregated form of tau, interference at each of these stages has yielded the most avidly pursued set of targets. Introducing agents that limit phosphorylation, block misfolding or prevent aggregation have all generated promising results. Passive and active immunization with late stage anti-phospho-tau antibodies in mouse models has led to dramatic decreases in tau aggregation and improvements in cognitive parameters. It has also been suggested that introduction of anti-tau antibodies can prevent the trans-neuronal spread of tau pathology.

The vectored antibody delivery (VAD) of tau disease associated antibodies of the present disclosure may be used to treat subjects suffering from AD and other tauopathies. In some cases, methods of the present disclosure may be used to treat subjects suspected of developing AD or other tauopathies.

### Frontotemporal Dementia and Parkinsonism Linked to Chromosome 17 (FTDP-17)

Although Alzheimer's disease is, in part, characterized by the presence of tau pathology, no known mutations in the tau gene have been causally linked to the disease. Mutations in the tau gene have been shown to lead to an autosomal dominantly inherited tauopathy known as frontotemporal dementia and parkinsonism linked to chromosome 17 (FTDP-17) and demonstrate that alterations in tau can lead to neurodegenerative changes in the brain. Mutations in the tau gene that lead to FTDP-17 are thought to influence splicing patterns, thereby leading to an elevated proportion of tau with four microtubule binding domains (rather than three). These molecules are considered to be more amyloidogenic, meaning they are more likely to become hyperphosphorylated and more likely to aggregate into NFT (Hutton, M. et al., 1998, Nature 393(6686):702-5). Although physically and behaviorally, FTDP-17 patients can appear quite similar to Alzheimer's disease patients, at autopsy FTDP-17 brains lack the prominent Aβ plaque pathology of an AD brain (Gotz, J. et al., 2012, British Journal of Pharmacology 165(5):1246-59). Therapeutically targeting the aggregates of tau protein may ameliorate and prevent degenerative changes in the brain and potentially lead to improved cognitive ability.

As of today, there is no treatment to prevent, slow the progression, or cure FTDP-17. Medication may be prescribed to reduce aggressive, agitated or dangerous behavior. There remains a need for therapy affecting the underlying pathophysiology, such as antibody therapies targeting tau protein.

In some embodiments, the vectored antibody delivery of the present disclosure may be used to treat subjects suffering from FTDP-17. In some cases, methods of the present disclosure may be used to treat subjects suspected of developing FTDP-17.

### Chronic Traumatic Encephalopathy

Unlike the genetically linked tauopathies, chronic traumatic encephalopathy is a degenerative tauopathy linked to repeated head injuries. The disease was first described in boxers who behaved “punch drunk” and has since been identified primarily in athletes that play American football, ice hockey, wrestling and other contact sports. The brains of those suffering from CTE are characterized by distinctive patterns of brain atrophy accompanied by accumulation of hyperphosphorylated species of aggregated tau in NFT. In CTE, pathological changes in tau are accompanied by a number of other pathobiological processes, such as inflammation (Daneshvar, D. H. et al., 2015 Mol Cell Neurosci 66(Pt B): 81-90). Targeting the tau aggregates may provide reprieve from the progression of the disease and may allow cognitive improvement.

As of today, there is no medical therapy to treat or cure CTE. The condition is only diagnosed after death, due to lack of in vivo techniques to identify CTE specific biomarkers. There remains a need for therapy affecting the underlying pathophysiology, such as antibody therapies targeting tau protein.

In some embodiments, the vectored antibody delivery methods of the present disclosure may be used to treat subjects suffering from CTE. In some cases, methods of the present disclosure may be used to treat subjects suspected of developing CTE.

### Prion Diseases

Prion diseases, also known as transmissible spongiform encephalopathies (TSEs), are a group of rare progressive conditions affecting the nervous system. The related conditions are rare and are typically caused by mutations in the PRNP gene which enables production of the prion protein. Gene mutations lead to an abnormally structured prion protein. Alternatively, the abnormal prion may be acquired by exposure from an outside source, e.g. by consumption of beef products containing the abnormal prion protein. Abnormal prions are misfolded, causing the brain tissue to degenerate rapidly. Prion diseases include, but are not limited to, Creutzfeldt-Jakob disease (CJD), Gerstmann-Straussler-Scheinker syndrome (GSS), fatal insomnia (FFI), variably protease-sensitive prionopathy (VPSPr), and kuru. Prion diseases are rare. Approximately 350 cases of prion diseases are diagnosed in the US annually.

CJD is a degenerative brain disorder characterized by problems with muscular coordination, personality changes including mental impairment, impaired vision, involuntary muscle jerks, weakness and eventually coma. The most common categories of CJD are sporadic, hereditary due to a genetic mutation, and acquired. Sporadic CJD is the most common form affecting people with no known risk factors for the disease. The acquired form of CJD is transmitted by exposure of the brain and nervous system tissue to the prion. As an example, variant CJD (vCDJ) is linked to a bovine spongiform encephalopathy (BSE), also known as a ‘mad cow’ disease. CJD is fatal and patients typically die within one year of diagnosis.

Prion diseases are associated with an infectious agent consisting of an alternative conformational isoform of the prion protein, PrPSc. PrPSc replication is considered to occur through an induction of the infectious prion in the normal prion protein (PrPC). The replication occurs without a nucleic acid.

As of today, there is no therapy to manage or cure CJD, or other prion diseases. Typically, treatment is aimed at alleviating symptoms and increasing comfortability of the patient, e.g. with pain relievers. There remains a need for therapy affecting the underlying pathophysiology, such as antibody therapies targeting the prion protein.

In some embodiments, vectored antibody delivery methods of the present disclosure may be used to treat subjects suffering from a prion disease. In some cases, methods of the present disclosure may be used to treat subjects suspected of developing a prion disease.

### Neurodegeneration and Stroke

Neurodegenerative diseases and other diseases of the nervous system share many common features. Neurodegenerative diseases, in particular, are a group of conditions characterized by progressive loss of neuronal structure and function, ultimately leading to neuronal cell death. Neurons are the building blocks of the nervous system(s) and are generally not able to reproduce and/or be replaced, and therefore neuron damage and/or death is especially devastating. Other, non-degenerating diseases that lead to neuronal cell loss, such as stroke, have similarly debilitating outcomes. Targeting molecules that contribute to the deteriorating cell structure or function may prove beneficial generally for treatment of nervous system diseases, neurodegenerative disease and/or stroke.

Certain molecules are believed to have inhibitory effects on neurite outgrowth, contributing to the limited ability of the central nervous system to repair. Such molecules include, but are not limited to, myelin associated proteins, such as, but not limited to, RGM (Repulsive guidance molecule), NOGO (Neurite outgrowth inhibitor), NOGO receptor, MAG (myelin associated glycoprotein), and MAI (myelin associated inhibitor). In some embodiments, the vectored antibody delivery of the present disclosure is utilized to target the aforementioned antigens (e.g., neurite outgrowth inhibitors).

Many neurodegenerative diseases are associated with aggregation of misfolded proteins, including, but not limited to, alpha synuclein, tau, amyloid β, prion proteins, TDP-43, and huntingtin (see, e.g. De Genst et al., 2014, Biochim Biophys Acta; 1844(11):1907-1919, and Yu et al., 2013, Neurotherapeutics.; 10(3): 459-472, references therein). The aggregation results from disease-specific conversion of soluble proteins to an insoluble, highly ordered fibrillary deposit. This conversion is thought to prevent the proper disposal or degradation of the misfolded protein, thereby leading to further aggregation. Conditions associated with alpha synuclein and tau may be referred to as “synucleinopathies” and “tauopathies”, respectively. In some embodiments, the vectored antibody delivery of the present disclosure is utilized to target the aforementioned antigens (e.g., misfolded or aggregated proteins).

AAV Particles and methods of using the AAV particles described in the present disclosure may be used to prevent, manage and/or treat tauopathies or tau associated disease. As a non-limiting example, the AAV particles of the present disclosure comprise a nucleic acid sequence encoding at least one of the sequences described in Table 3.

### V. Kits and Devices

### Kits

In some embodiments, the disclosure provides a variety of kits for conveniently and/or effectively carrying out methods of the present disclosure. Typically, kits will comprise sufficient amounts and/or numbers of components to allow a user to perform multiple treatments of a subject(s) and/or to perform multiple experiments.

Any of the AAV particles of the present disclosure may be comprised in a kit. In some embodiments, kits may further include reagents and/or instructions for creating and/or synthesizing compounds and/or compositions of the present disclosure. In some embodiments, kits may also include one or more buffers. In some embodiments, kits may include components for making protein or nucleic acid arrays or libraries and thus, may include, for example, solid supports.

In some embodiments, kit components may be packaged either in aqueous media or in lyophilized form. The container means of the kits will generally include at least one vial, test tube, flask, bottle, syringe or other container means, into which a component may be placed, and preferably, suitably aliquoted. Where there is more than one kit component, (labeling reagent and label may be packaged together), kits may also generally contain second, third or other additional containers into which additional components may be separately placed. In some embodiments, kits may also comprise second container means for containing sterile, pharmaceutically acceptable buffers and/or other diluents. In some embodiments, various combinations of components may be comprised in one or more vial. Kits of the present disclosure may also typically include means for containing compounds and/or compositions of the present disclosure, e.g., proteins, nucleic acids, and any other reagent containers in close confinement for commercial sale. Such containers may include injection or blow-molded plastic containers into which desired vials are retained.

In some embodiments, kit components are provided in one and/or more liquid solutions. In some embodiments, liquid solutions are aqueous solutions, with sterile aqueous solutions being particularly preferred. In some embodiments, kit components may be provided as dried powder(s). When reagents and/or components are provided as dry powders, such powders may be reconstituted by the addition of suitable volumes of solvent. In some embodiments, it is envisioned that solvents may also be provided in another container means. In some embodiments, labeling dyes are provided as dried powders. In some embodiments, it is contemplated that 10, 20, 30, 40, 50, 60, 70, 80, 90, 100, 120, 120, 130, 140, 150, 160, 170, 180, 190, 200, 300, 400, 500, 600, 700, 800, 900, 1000 micrograms or at least or at most those amounts of dried dye are provided in kits. In such embodiments, dye may then be resuspended in any suitable solvent, such as DMSO.

In some embodiments, kits may include instructions for employing kit components as well the use of any other reagent not included in the kit. Instructions may include variations that may be implemented.

### Devices

In some embodiments, the AAV particles may delivered to a subject using a device to deliver the AAV particles and a head fixation assembly. The head fixation assembly may be, but is not limited to, any of the head fixation assemblies sold by MRI interventions. As a non-limiting example, the head fixation assembly may be any of the assemblies described in U.S. Pat. Nos. 8,099,150, 8,548,569, and 9,031,636 and International Patent Publication Nos. WO201108495 and WO2014014585, the contents of each of which are incorporated by reference in their entireties. A head fixation assembly may be used in combination with an MRI compatible drill such as, but not limited to, the MRI compatible drills described in International Patent Publication No. WO2013181008 and US Patent Publication No. US20130325012, the contents of which are herein incorporated by reference in its entirety.

In some embodiments, the AAV particles may be delivered using a method, system and/or computer program for positioning apparatus to a target point on a subject to deliver the AAV particles. As a non-limiting example, the method, system and/or computer program may be the methods, systems and/or computer programs described in U.S. Pat. No. 8,340,743, the contents of which are herein incorporated by reference in its entirety. The method may include: determining a target point in the body and a reference point, wherein the target point and the reference point define a planned trajectory line (PTL) extending through each; determining a visualization plane, wherein the PTL intersects the visualization plane at a sighting point; mounting the guide device relative to the body to move with respect to the PTL, wherein the guide device does not intersect the visualization plane; determining a point of intersection (GPP) between the guide axis and the visualization plane; and aligning the GPP with the sighting point in the visualization plane.

In some embodiments, the AAV particles may be delivered to a subject using a convention-enhanced delivery device. Non-limiting examples of targeted delivery of drugs using convection are described in US Patent Publication Nos. US20100217228, US20130035574, and US 20130035660 and International Patent Publication No. WO2013019830 and WO2008144585, the contents of each of which are herein incorporated by reference in their entireties.

In some embodiments, a subject may be imaged prior to, during and/or after delivery of the AAV particles. The imaging method may be a method known in the art and/or described herein, such as but not limited to, magnetic resonance imaging (MRI). As a non-limiting example, imaging may be used to assess therapeutic effect. As another non-limiting example, imaging may be used for assisted delivery of AAV particles.

In some embodiments, the AAV particles may be delivered using an MRI-guided device. Non-limiting examples of MRI-guided devices are described in U.S. Pat. Nos. 9,055,884, 9,042,958, 8,886,288, 8,768,433, 8,396,532, 8,369,930, 8,374,677, and 8,175,677 and US Patent Application No. US20140024927 the contents of each of which are herein incorporated by reference in their entireties. As a non-limiting example, the MRI-guided device may be able to provide data in real time such as those described in U.S. Pat. Nos. 8,886,288 and 8,768,433, the contents of each of which is herein incorporated by reference in its entirety. As another non-limiting example, the MRI-guided device or system may be used with a targeting cannula such as the systems described in U.S. Pat. Nos. 8,175,677 and 8,374,677, the contents of each of which are herein incorporated by reference in their entireties. As yet another non-limiting example, the MRI-guided device includes a trajectory guide frame for guiding an interventional device as described, for example, in U.S. Pat. No. 9,055,884 and US Patent Application No. US20140024927, the contents of each of which are herein incorporated by reference in their entireties.

In some embodiments, the AAV particles may be delivered using an MRI-compatible tip assembly. Non-limiting examples of MRI-compatible tip assemblies are described in US Patent Publication No. US20140275980, the contents of which is herein incorporated by reference in its entirety.

In some embodiments, the AAV particles may be delivered using a cannula which is MRI-compatible. Non-limiting examples of MRI-compatible cannulas include those taught in International Patent Publication No. WO2011130107, the contents of which are herein incorporated by reference in its entirety.

In some embodiments, the AAV particles may be delivered using a catheter which is MRI-compatible. Non-limiting examples of MRI-compatible catheters include those taught in International Patent Publication No. WO2012116265, U.S. Pat. No. 8,825,133 and US Patent Publication No. US20140024909, the contents of each of which are herein incorporated by reference in their entireties.

In some embodiments, the AAV particles may be delivered using a device with an elongated tubular body and a diaphragm as described in US Patent Publication Nos. US20140276582 and US20140276614, the contents of each of which are herein incorporated by reference in their entireties.

In some embodiments, the AAV particles may be delivered using an MRI compatible localization and/or guidance system such as, but not limited to, those described in US Patent Publication Nos. US20150223905 and US20150230871, the contents of each of which are herein incorporated by reference in their entireties. As a non-limiting example, the MRI compatible localization and/or guidance systems may comprise a mount adapted for fixation to a patient, a targeting cannula with a lumen configured to attach to the mount so as to be able to controllably translate in at least three dimensions, and an elongate probe configured to snugly advance via slide and retract in the targeting cannula lumen, the elongate probe comprising at least one of a stimulation or recording electrode.

In some embodiments, the AAV particles may be delivered to a subject using a trajectory frame as described in US Patent Publication Nos. US20150031982 and US20140066750 and International Patent Publication Nos. WO2015057807 and WO2014039481, the contents of each of which are herein incorporated by reference in their entireties.

In some embodiments, the AAV particles may be delivered to a subject using a gene gun.

### VI. Definitions

At various places in the present specification, substituents of compounds of the present disclosure are disclosed in groups or in ranges. It is specifically intended that the present disclosure include each and every individual sub combination of the members of such groups and ranges.

About: As used herein, the term “about” means +/−10% of the recited value.

Adeno-associated virus: The term “adeno-associated virus” or “AAV” as used herein refers to members of the dependovirus genus comprising any particle, sequence, gene, protein, or component derived therefrom.

AAV Particle: As used herein, an “AAV particle” is a virus which comprises a viral genome with at least one payload region and at least one ITR region. AAV vectors of the present disclosure may be produced recombinantly and may be based on adeno-associated virus (AAV) parent or reference sequences. AAV particle may be derived from any serotype, described herein or known in the art, including combinations of serotypes (i.e., “pseudotyped” AAV) or from various genomes (e.g., single stranded or self-complementary). In addition, the AAV particle may be replication defective and/or targeted.

Activity: As used herein, the term “activity” refers to the condition in which things are happening or being done. Compositions may have activity and this activity may involve one or more biological events.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently closely together such that a combinatorial (e.g., a synergistic) effect is achieved.

Amelioration: As used herein, the term “amelioration” or “ameliorating” refers to a lessening of severity of at least one indicator of a condition or disease. For example, in the context of neurodegeneration disorder, amelioration includes the reduction of neuron loss.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Antibody: As used herein, the term “antibody” is referred to in the broadest sense and specifically covers various embodiments including, but not limited to monoclonal antibodies, polyclonal antibodies, multispecific antibodies (e.g. bispecific antibodies formed from at least two intact antibodies), and antibody fragments (e.g., diabodies) so long as they exhibit a desired biological activity (e.g., “functional”). Antibodies are primarily amino-acid based molecules but may also comprise one or more modifications (including, but not limited to the addition of sugar moieties, fluorescent moieties, chemical tags, etc.). Non-limiting examples of antibodies or fragments thereof include V_{H} and V_{L} domains, scFvs, Fab, Fab′, F(ab′)2, Fv fragment, diabodies, linear antibodies, single chain antibody molecules, multispecific antibodies, bispecific antibodies, intrabodies, monoclonal antibodies, polyclonal antibodies, humanized antibodies, codon-optimized antibodies, tandem scFv antibodies, bispecific T-cell engagers, mAb2 antibodies, chimeric antigen receptors (CAR), tetravalent bispecific antibodies, biosynthetic antibodies, native antibodies, miniaturized antibodies, unibodies, maxibodies, antibodies to senescent cells, antibodies to conformers, antibodies to disease specific epitopes, or antibodies to innate defense molecules.

Antibody-based composition: As used herein, “antibody-based” or “antibody-derived” compositions are monomeric or multi-meric polypeptides which comprise at least one amino-acid region derived from a known or parental antibody sequence and at least one amino acid region derived from a non-antibody sequence, e.g., mammalian protein.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunctional: As used herein, the term “bifunctional” refers to any substance, molecule or moiety which is capable of or maintains at least two functions. The functions may affect the same outcome or a different outcome. The structure that produces the function may be the same or different.

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, an AAV particle of the present disclosure may be considered biologically active if even a portion of the encoded payload is biologically active or mimics an activity considered biologically relevant.

Capsid: As used herein, the term “capsid” refers to the protein shell of a virus particle. In some embodiments, the term capsid may refer to the nucleic acid encoding the protein shell of the virus particle.

Chimeric antigen receptor (CAR): As used herein, the term “chimeric antigen receptor” or “CAR” refers to an artificial chimeric protein comprising at least one antigen specific targeting region (ASTR), a transmembrane domain and an intracellular signaling domain, wherein the antigen specific targeting region comprises a full-length antibody or a fragment thereof. As a non-limiting example, the ASTR of a CAR may be any of the antibodies listed in Table 3, antibody-based compositions or fragments thereof. Any molecule that is capable of binding a target antigen with high affinity can be used in the ASTR of a CAR. The CAR may optionally have an extracellular spacer domain and/or a co-stimulatory domain. A CAR may also be used to generate a cytotoxic cell carrying the CAR.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity. As used herein, the term “substantially complementary” means that the siRNA has a sequence (e.g., in the antisense strand) which is sufficient to bind the desired target mRNA, and to trigger the RNA silencing of the target mRNA.

Compound: Compounds of the present disclosure include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

Comprehensive Positional Evolution (CPE™): As used herein, the term “comprehensive positional evolution” refers to an antibody evolution technology that allows for mapping of the effects of amino acid changes at every position along an antibody variable domain's sequence. This comprehensive mutagenesis technology can be used to enhance one or more antibody properties or characteristics.

Comprehensive Protein Synthesis (CPS™): As used herein, the term “comprehensive protein synthesis” refers to a combinatorial protein synthesis technology that can be used to optimize antibody properties or characteristics by combining the best properties into a new, high-performance antibody.

Conditionally active: As used herein, the term “conditionally active” refers to a mutant or variant of a wild-type polypeptide, wherein the mutant or variant is more or less active at physiological conditions than the parent polypeptide. Further, the conditionally active polypeptide may have increased or decreased activity at aberrant conditions as compared to the parent polypeptide. A conditionally active polypeptide may be reversibly or irreversibly inactivated at normal physiological conditions or aberrant conditions.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

Control Elements: As used herein, “control elements”, “regulatory control elements”, or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription and translation of a coding sequence in a recipient cell. Not all of these control elements need always be present as long as the selected coding sequence is capable of being replicated, transcribed and/or translated in an appropriate host cell.

Controlled Release: As used herein, the term “controlled release” refers to a pharmaceutical composition or compound release profile that conforms to a particular pattern of release to affect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering an AAV particle, a compound, substance, entity, moiety, cargo or payload.

Delivery Agent: As used herein, “delivery agent” refers to any substance which facilitates, at least in part, the in vivo delivery of an AAV particle to targeted cells.

Destabilized: As used herein, the term “destable”, “destabilize”, or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the peptides or proteins disclosed herein. They may be within the amino acids, the peptides, or proteins, or located at the N- or C-termini.

Digest: As used herein, the term “digest” means to break apart into smaller pieces or components. When referring to polypeptides or proteins, digestion results in the production of peptides.

Distal: As used herein, the term “distal” means situated away from the center or away from a point or region of interest.

Dosing regimen: As used herein, a “dosing regimen” is a schedule of administration or physician determined regimen of treatment, prophylaxis, or palliative care.

Encapsulate: As used herein, the term “encapsulate” means to enclose, surround or encase.

Engineered: As used herein, embodiments are “engineered” when they are designed to have a feature or property, whether structural or chemical, that varies from a starting point, wild type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Epitope: As used herein, an “epitope” refers to a surface or region on a molecule that is capable of interacting with a biomolecule. For example, a protein may contain one or more amino acids, e.g., an epitope, which interacts with an antibody, e.g., a biomolecule. In some embodiments, when referring to a protein or protein module, an epitope may comprise a linear stretch of amino acids or a three-dimensional structure formed by folded amino acid chains.

EvoMap™: As used herein, an EvoMap™ refers to a map of a polypeptide, wherein detailed informatics are presented about the effects of single amino acid mutations within the length of the polypeptide and their influence on the properties and characteristics of that polypeptide.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one AAV particle and a delivery agent.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

Heterologous Region: As used herein the term “heterologous region” refers to a region which would not be considered a homologous region.

Homologous Region: As used herein the term “homologous region” refers to a region which is similar in position, structure, evolution origin, character, form or function.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Substantially isolated: By “substantially isolated” is meant that a substance is substantially separated from the environment in which it was formed or detected. Partial separation can include, for example, a composition enriched in the substance or AAV particles of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Linker: As used herein “linker” refers to a molecule or group of molecules which connects two molecules, such as a V_{H} chain and V_{L} chain or an antibody. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule. Molecules may be modified in many ways including chemically, structurally, and functionally.

Naturally Occurring: As used herein, “naturally occurring” or “wild-type” means existing in nature without artificial aid, or involvement of the hand of man.

Non-human vertebrate: As used herein, a “non-human vertebrate” includes all vertebrates except Homo sapiens, including wild and domesticated species. Examples of non-human vertebrates include, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Off-target: As used herein, “off target” refers to any unintended effect on any one or more target, gene, or cellular transcript.

Open reading frame: As used herein, “open reading frame” or “ORF” refers to a sequence which does not contain a stop codon in a given reading frame.

Operably linked: As used herein, the phrase “operably linked” refers to a functional connection between two or more molecules, constructs, transcripts, entities, moieties or the like.

Particle: As used herein, a “particle” is a virus comprised of at least two components, a protein capsid and a polynucleotide sequence enclosed within the capsid (e.g., viral genome).

Patient: As used herein, “patient” refers to a subject who may seek or be in need of treatment, requires treatment, is receiving treatment, will receive treatment, or a subject who is under care by a trained professional for a particular disease or condition.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide or a modulatory nucleic acid or regulatory nucleic acid.

Payload construct: As used herein, “payload construct” or “construct” is one or more polynucleotide regions encoding or comprising a payload that is flanked on one or both sides by an inverted terminal repeat (ITR) sequence. The payload construct is a template that is replicated in a viral production cell to produce a viral genome.

Payload construct vector: As used herein, “payload construct vector” is a vector encoding or comprising a payload construct, and regulatory regions for replication and expression in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacokinetic: As used herein, “pharmacokinetic” refers to any one or more properties of a molecule or compound as it relates to the determination of the fate of substances administered to a living organism. Pharmacokinetics is divided into several areas including the extent and rate of absorption, distribution, metabolism and excretion. This is commonly referred to as ADME where: (A) Absorption is the process of a substance entering the blood circulation; (D) Distribution is the dispersion or dissemination of substances throughout the fluids and tissues of the body; (M) Metabolism (or Biotransformation) is the irreversible transformation of parent compounds into daughter metabolites; and (E) Excretion (or Elimination) refers to the elimination of the substances from the body. In rare cases, some drugs irreversibly accumulate in body tissue.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Protein of interest: As used herein, the terms “proteins of interest” or “desired proteins” include those provided herein and fragments, mutants, variants, and alterations thereof.

Proximal: As used herein, the term “proximal” means situated nearer to the center or to a point or region of interest.

Purified: As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini. N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In some embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In some embodiments, N-terminal regions may comprise any length of amino acids that includes the N-terminus, but does not include the C-terminus. In some embodiments, C-terminal regions may comprise any length of amino acids, which include the C-terminus, but do not comprise the N-terminus.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and 3′ termini. 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In some embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In some embodiments, 5′ regions may comprise any length of nucleic acids that includes the 5′ terminus, but does not include the 3′ terminus. In some embodiments, 3′ regions may comprise any length of nucleic acids, which include the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Self-complementary viral particle: As used herein, a “self-complementary viral particle” is a particle comprised of at least two components, a protein capsid and a polynucleotide sequence encoding a self-complementary genome enclosed within the capsid.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event. In some embodiments, a single unit dose is provided as a discrete dosage form (e.g., a tablet, capsule, patch, loaded syringe, vial, etc.).

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and preferably capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present disclosure may be chemical or enzymatic.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose. In some embodiments, a therapeutically effective amount is administered in a dosage regimen comprising a plurality of doses. Those skilled in the art will appreciate that in some embodiments, a unit dosage form may be considered to comprise a therapeutically effective amount of a particular agent or entity if it comprises an amount that is effective when administered as part of such a dosage regimen.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hr period. It may be administered as a single unit dose.

Transfection: As used herein, the term “transfection” refers to methods to introduce exogenous nucleic acids into a cell. Methods of transfection include, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Vector: As used herein, a “vector” is any molecule or moiety which transports, transduces or otherwise acts as a carrier of a heterologous molecule. Vectors of the present disclosure may be produced recombinantly and may be based on and/or may comprise adeno-associated virus (AAV) parent or reference sequence. Such parent or reference AAV sequences may serve as an original, second, third or subsequent sequence for engineering vectors. In non-limiting examples, such parent or reference AAV sequences may comprise any one or more of the following sequences: a polynucleotide sequence encoding a polypeptide or multi-polypeptide, which sequence may be wild-type or modified from wild-type and which sequence may encode full-length or partial sequence of a protein, protein domain, or one or more subunits of a protein; a polynucleotide comprising a modulatory or regulatory nucleic acid which sequence may be wild-type or modified from wild-type; and a transgene that may or may not be modified from wild-type sequence. These AAV sequences may serve as either the “donor” sequence of one or more codons (at the nucleic acid level) or amino acids (at the polypeptide level) or “acceptor” sequences of one or more codons (at the nucleic acid level) or amino acids (at the polypeptide level).

Viral genome: As used herein, a “viral genome” or “vector genome” is a polynucleotide comprising at least one inverted terminal repeat (ITR) and at least one encoded payload. A viral genome encodes at least one copy of the payload.

Described herein are compositions, methods, processes, kits and devices for the design, preparation, manufacture and/or formulation of AAV particles. In some embodiments, payloads, such as but not limited to AAV polynucleotides, may be encoded by payload constructs or contained within plasmids or vectors or recombinant adeno-associated viruses (AAVs).

The details of one or more embodiments are set forth in the accompanying description below. Although any materials and methods similar or equivalent to those described herein can be used in the practice or testing of the present disclosure, the preferred materials and methods are now described. Other features, objects and advantages will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

The present disclosure is further illustrated by the following non-limiting examples. In addition, the Examples of U.S. Provisional Patent Application No. 62/839,891, entitled “Compositions and Methods for the Treatment of Tauopathy”, filed Apr. 29, 2019, U.S. Provisional Patent Application No. 63/002,011, entitled “Compositions and Methods for the Treatment of Tauopathy” filed Mar. 30, 2020 and International Patent Application No. PCT/US20/30357, entitled “Compositions and Methods for the Treatment of Tauopathy, filed Apr. 29, 2020, are herein each incorporated by reference in their entirety, particularly as pertains to the making, testing and/or using of AAV particles for delivery of vectorized anti-tau antibodies.

### VII. Examples

### Example 1. Olfactory Bulb Propagation Model-Based Studies

Olfactory deficits are present in numerous neurodegenerative disorders and are accompanied by pathology in related brain regions. In several neurodegenerative disorders, olfactory disturbances appear early and are considered as prodromal symptoms of the disease. In addition, pathological protein aggregates affect olfactory regions prior to other regions, suggesting that the olfactory system might be particularly vulnerable to neurodegenerative diseases.

Distribution and kinetics of AAV particles dosed intravenously were evaluated in htau female mice (B6.Cg-Mapt^{tml(EGFP)Klt} Tg(MAPT)8cPdav/J; The Jackson Laboratory, Bar Harbor, ME). Viral genome TAU_ITR111 (SEQ ID NO: 2161; VOY101.CBA.HL.T2A) was packaged into VOY101 capsids. AAV particles were formulated in formulation buffer (10 mM disodium phosphate, 2 mM monopotassium phosphate, 2.7 mM potassium chloride, 192 mM sodium chloride and 0.001% Pluronic Acid (F-68)) and provided intravenously at a dose of 1.4×10¹³ vg/kg to approximately 18 week-old mice. Mice were euthanized and CNS and/or tissue samples were collected 1, 3, 7, 14, and 28 days post injection. The right half of the brain and lumbar SC tissues were fixed with 4% PFA; whereas the left half of the brain as well as the cervical and thoracic regions of the spinal cord were flash frozen. Peripheral tissues (spleen, liver, skeletal muscle) were also harvested at all time points. Serum and CSF were also harvested at all time points. Harvested tissues were analyzed by ePHF ELISA (specifically, CNS tissue, peripheral tissues, serum) and IgG1immunohistochemistry (IHC).

Samples of hippocampus, cortex, olfactory bulb, and thalamus were subjected to analysis by ePHF ELISA to determine PT3 expression. Data are shown in Table 96 below as ng/mg protein

TABLE 96
PT3 expression from TAU_ITR111 (ng/mg protein)

| Region | Day 1 | Day 3 | Day 7 | Day 14 | Day 28 |
|---|---|---|---|---|---|
| Hippocampus | 0.00 | 0.00 | 7.65 | 124.70 | 271.70 |
| | 0.00 | 0.00 | 9.66 | 88.03 | 128.90 |
| | 0.00 | 0.48 | 15.95 | 196.98 | 148.33 |
| | 0.00 | 0.80 | — | — | — |
| Cortex | 0.00 | 1.57 | 14.02 | 196.60 | 555.51 |
| | 0.00 | 1.25 | 23.15 | 164.22 | 175.41 |
| | 0.00 | 4.69 | 26.68 | 256.15 | 321.62 |
| | 2.91 | 2.08 | — | — | — |
| Olfactory | 0.00 | 15.46 | 56.04 | 398.87 | 559.84 |
| bulb | 0.00 | 14.81 | 99.43 | 328.47 | 433.80 |
| | 0.00 | 19.41 | 76.10 | 442.19 | 589.25 |
| | 0.00 | 13.85 | — | — | — |
| Thalamus | 0.00 | 0.00 | 16.85 | 56.24 | 98.24 |
| | 0.00 | 2.59 | 16.91 | 80.27 | 113.64 |
| | 0.00 | 1.12 | 18.13 | 122.91 | 92.26 |
| | 0.00 | 0.00 | — | — | — |

Analysis of the PT3 expression data by ePHF ELISA showed that PT3 expression could be observed as early as 3 days after intravenous injection of TAU_ITR111. PT3 expression levels were higher in the later measurements made at later time points suggesting that PT3 expression increased over time. These observations were confirmed by immunohistochemistry studies in which PT3 expression could be detected in the olfactory bulb on day 3 and to a lesser extent in the hippocampus and cortex. These studies demonstrate that treating mice 1 day after the injection of tau seeds may be a suitable experimental set up for measuring the therapeutic efficacy of vectored antibody delivery.

### Biodistributions and Expression of Vectorized PT3 Antibody in Olfactory Bulb Propagation Model

To study the effects of intracranial olfactory bulb injection on PT3 antibody distribution, a subsequent study was conducted. Three month-old wild type mice were injected with PBS intracranially into the olfactory bulb. One, two or three days later, viral genome comprising TAU_ITR111 (SEQ ID NO: 2161; VOY101.CBA.HL.T2A) was packaged into VOY101 capsids. AAV particles were formulated in formulation buffer (10 mM disodium phosphate, 2 mM monopotassium phosphate, 2.7 mM potassium chloride, 192 mM sodium chloride and 0.001% Pluronic Acid (F-68)) and provided intravenously at a dose of 1.4×10¹³ vg/kg. 28 days later, mice were euthanized and CNS and/or tissue samples were obtained. The left half of the brain and lumbar SC tissues were fixed with 4% PFA; whereas the right half of the brain and cervical/thoracic SC were flash frozen. Peripheral tissue (specifically the liver), serum and CSF were also harvested. Tissues were analyzed by ePHF ELISA (specifically, CNS tissue, liver, serum); IgG1immunohistochemistry (IHC); and vector genome quantification (specifically, cortex, hippocampus, thalamus, OB, BS, thoracic SC, liver).

Biodistribution, or VG/DC (vector genome/diploid cell) was quantified for a subset of tissues, such as brain using droplet digital PCR. Vector genome biodistribution quantification (VG/DC) data are shown in Table 97, Table 98, Table 99, Table 100, Table 101, Table 102, and Table 103, wherein the “control (no injection)” column refers to VG/DC in animals who have not received an intracranial olfactory bulb injection. Average VG/DC values are shown in Table 104. Significance was analyzed using one-way ANOVA-Tukey's multiple comparison test.

TABLE 97
Vector genome quantification of VOY101.PT3
construct in the olfactory bulb

| | AAV genomes/cell | | | |
|---|---|---|---|---|
| | | 1 day | 2 day | 3 day |
| | Control (no | Post | Post | Post |
| Mouse # | injection) | Injection | Injection | Injection |
| 1 | 137.23 | 19.95 | 56.02 | 84.82 |
| 2 | 47.74 | 59.97 | 42.91 | 60.33 |
| 3 | 33.76 | 73.54 | 136.45 | 37.81 |
| 4 | 82.65 | 83.55 | 55.69 | 15.71 |
| 5 | 51.25 | 56.73 | 32.29 | — |

TABLE 98
Vector genome quantification of VOY101.PT3
construct in the cortex

| | AAV genomes/cell | | | |
|---|---|---|---|---|
| | | 1 day | 2 day | 3 day |
| | Control (no | Post | Post | Post |
| Mouse # | injection) | Injection | Injection | Injection |
| 1 | 83.45 | 19.40 | 31.01 | 47.62 |
| 2 | 36.25 | 76.96 | 43.34 | 59.29 |
| 3 | 32.87 | 47.14 | 46.18 | 29.63 |
| 4 | 55.87 | 68.53 | 52.01 | 24.42 |
| 5 | 28.49 | 53.01 | 41.02 | — |

TABLE 99
Vector genome quantification of VOY101.PT3
construct in the hippocampus

| | AAV genomes/cell | | | |
|---|---|---|---|---|
| | | 1 day | 2 day | 3 day |
| | Control (no | Post | Post | Post |
| Mouse # | injection) | Injection | Injection | Injection |
| 1 | 49.18 | 7.93 | 24.60 | 29.82 |
| 2 | 22.78 | 35.91 | 23.25 | 29.07 |
| 3 | 30.58 | 53.60 | 43.33 | 23.22 |
| 4 | 27.33 | 39.94 | 23.82 | 14.43 |
| 5 | 33.04 | 33.55 | 26.71 | — |

TABLE 100
Vector genome quantification of VOY101.PT3
construct in the thalamus

| | AAV genomes/cell | | | |
|---|---|---|---|---|
| | | 1 day | 2 day | 3 day |
| | Control (no | Post | Post | Post |
| Mouse # | injection) | Injection | Injection | Injection |
| 1 | 82.37 | 28.68 | 47.33 | 69.55 |
| 2 | 61.54 | 60.12 | 41.97 | 67.31 |
| 3 | 67.14 | 68.61 | 65.72 | 48.96 |
| 4 | 77.94 | 93.99 | 68.85 | 94.66 |
| 5 | 65.29 | 68.54 | 48.87 | — |

TABLE 101
Vector genome quantification of VOY101.PT3
construct in the brain stem

| | AAV genomes/cell | | | |
|---|---|---|---|---|
| | | 1 day | 2 day | 3 day |
| | Control (no | Post | Post | Post |
| Mouse # | injection) | Injection | Injection | Injection |
| 1 | 96.75 | 34.00 | 49.02 | 57.36 |
| 2 | 39.55 | 98.51 | 54.63 | 62.27 |
| 3 | 60.14 | 89.47 | 59.32 | 59.91 |
| 4 | 10.67 | 119.91 | 53.75 | 28.97 |
| 5 | 69.99 | 65.56 | 62.49 | — |

TABLE 102
Vector genome quantification of VOY101.PT3
construct in the spinal cord

| | AAV genomes/cell | | | |
|---|---|---|---|---|
| | | 1 day | 2 day | 3 day |
| | Control (no | Post | Post | Post |
| Mouse # | injection) | Injection | Injection | Injection |
| 1 | 60.08 | 16.87 | 37.40 | 40.83 |
| 2 | 32.12 | 61.80 | 15.15 | 57.66 |
| 3 | 41.31 | 61.83 | 48.43 | 44.36 |
| 4 | 46.93 | 99.23 | 34.92 | 92.72 |
| 5 | 43.19 | 67.14 | 65.50 | — |

TABLE 103
Vector genome quantification of
VOY101.PT3 construct in the liver

| | AAV genomes/cell | | | |
|---|---|---|---|---|
| | | 1 day | 2 day | 3 day |
| | Control (no | Post | Post | Post |
| Mouse # | injection) | Injection | Injection | Injection |
| 1 | 14.67 | 0.19 | 9.09 | 13.56 |
| 2 | 4.43 | 6.20 | 8.17 | 15.51 |
| 3 | 6.05 | 5.30 | 7.01 | 8.56 |
| 4 | 10.59 | 7.10 | 9.02 | 9.06 |
| 5 | 4.33 | 5.36 | 17.12 | — |

TABLE 104
Average of vector genome quantification in the tissues

| | AAV genomes/cell | | | |
|---|---|---|---|---|
| | | 1 day | 2 day | 3 day |
| | Control (no | Post | Post | Post |
| Tissue | injection) | Injection | Injection | Injection |
| Olfactory Bulb | 70.53 | 58.75 | 64.67 | 49.67 |
| Cortex | 47.39 | 53.01 | 42.71 | 40.24 |
| Hippocampus | 32.58 | 34.19 | 28.34 | 24.14 |
| Thalamus | 70.86 | 63.99 | 54.55 | 70.12 |
| Brain Stem | 55.42 | 81.49 | 55.84 | 52.13 |
| Spinal cord | 44.73 | 61.37 | 40.28 | 58.89 |
| Liver | 8.01 | 4.83 | 10.08 | 11.67 |

No significant differences were observed in the AAV biodistribution VG/DC values between the different treatment groups. These data show that stereotaxic pre-injections in the olfactory bulb do not affect AAV biodistribution.

Samples of hippocampus, cortex, olfactory bulb, and thalamus were subject to analysis by ePHF ELISA to determine PT3 expression. Data are shown in Table 105 below as ng/mg protein.

TABLE 105
PT3 expression (ng/mg protein)

| | | 1 day | 2 day | 3 day |
|---|---|---|---|---|
| | Control (no | Post | Post | Post |
| Tissue | injection) | Injection | Injection | Injection |
| Olfactory Bulb | 470.77 | 87.35 | 317.08 | 439.18 |
| (OB) | 478.86 | 917.50 | 330.50 | 496.93 |
| | — | 515.00 | 484.76 | 467.54 |
| | 332.60 | 606.31 | 472.74 | 221.15 |
| | 495.24 | 574.86 | 399.38 | — |
| OB Average | 444.37 | 540.21 | 400.89 | 406.20 |
| Cortex | 457.93 | 21.97 | 164.92 | 201.29 |
| | 378.93 | 381.65 | 123.43 | 271.14 |
| | 346.30 | 476.32 | 467.99 | 281.39 |
| | 777.58 | 585.19 | 466.92 | 138.32 |
| | 275.13 | 382.33 | 241.26 | — |
| Cortex Average | 447.17 | 369.49 | 292.90 | 223.03 |
| Hippocampus | 205.95 | 9.05 | 39.01 | 82.04 |
| | 91.37 | 47.39 | 23.81 | 82.61 |
| | 34.06 | 239.05 | 189.39 | 57.04 |
| | 117.98 | 227.08 | 45.77 | 31.25 |
| | 141.39 | 169.86 | 92.90 | — |
| Hippocampus | 118.15 | 138.48 | 78.17 | 63.23 |
| Average | | | | |
| Thalamus | 177.58 | 8.39 | 137.91 | 108.37 |
| | 175.36 | 192.77 | 68.01 | 138.62 |
| | 144.91 | 119.14 | 110.74 | 109.52 |
| | 91.50 | 306.87 | 110.32 | 83.60 |
| | 424.90 | 344.34 | 230.53 | 14.50 |
| Thalamus | 202.85 | 194.30 | 131.50 | 90.92 |
| Average | | | | |

Significance was measured by one-way ANOVA. No significant difference in the PT3 expression was observed among different groups. Similarly, the immunohistochemical analysis of the olfactory bulb, cortex, hippocampus and thalamus showed no obvious differences between the staining pattern observed in the no injection group and the groups that received pre-injections. Thus, stereotaxic pre-injections in the olfactory bulb do not affect vector genome transduction or antibody expression.

### Propagation of Tau Seeds in Olfactory Bulb Propagation Model

Propagation of tau seeds in the olfactory bulb model was evaluated over time. Eighteen to twenty week old htau female mice (B6.Cg-Mapt^{tml(EGFP)Klt} Tg(MAPT)8cPdav/J; The Jackson Laboratory, Bar Harbor, ME) were injected with ePHF purified from AD brain lysates obtained from patients with Alzheimer's disease (herein referred to as AD lysate) via unilateral olfactory bulb injection. Two different ePHF preparations containing different concentrations of ePHF, i.e., 0.4 μg (sample 1) or 0.29 μg (sample 2) were tested in different groups of mice. Control groups were injected with 2 μL PBS. Mice were euthanized at 12 or 16 weeks and whole brain tissues were collected and fixed in formalin. Slides were cut to a thickness of 5 μM, and every fifth tissue section was collected, among which, every seventh section was stained with AT100. The stained sections were 175 μM apart. AT100 antibody detects Tau protein phosphorylated at threonine 212 and serine 214. The number of cells positively stained for AT100 were quantified in the olfactory bulb mitral and tufted cells, piriform cortex, entorhinal cortex, medial thalamus and CA1 hippocampus. Piriform cortex was also analyzed for AT100 (ePHF) staining intensity.

Greater olfactory mitral bulb/tuft cell staining was observed with 0.29 μg ePHF (sample 2) compared to 0.4 μg ePHF (sample 1). Strong AT100 positive cell counts were observed in the olfactory bulb, piriform cortex and CA1 hippocampus while fewer positive cell counts were observed in the medial thalamus.

Analysis of the AT100 staining revealed that Tau propagation to the first synapse (entorhinal and piriform cortex) and second synapse (CA1 hippocampus and medial thalamus) could be observed with both samples of ePHF tested. Tau propagation could be observed both at 12 and 16 weeks following injection and was virtually unchanged between the two groups. Table 106 provides AT100 cell numbers and Table 107 provides AT100 cell number per tissue slice. Table 108 provides data related to AT100 staining in the piriform cortex.

TABLE 106
AT100 staining (cell numbers)

| | | | | | Medial | Greater |
|---|---|---|---|---|---|---|
| | | Entorhinal | | | Thalamus | Medial |
| Dose (end | Olfactory | cortex (1st | CA1 (2nd | CA1/ | (2nd | Thalamus |
| point) | bulb | Synapse) | Synapse) | Sub | Synapse) | area |
| 0.29 μg ePHF | 181 | 25 | 18 | 27 | 9 | 14 |
| (3 month) | | | | | | |
| 0.29 μg ePHF | 63 | 31 | 23 | 43 | 0 | 8 |
| (4 month) | 66 | 23 | 31 | 64 | 0 | 19 |
| Average | 64.5 | 27 | 27 | 53.5 | 0 | 13.5 |
| | 12 | 53 | 66 | 109 | 1 | 11 |
| 0.4 μg ePHF | 64 | 100 | 60 | 140 | 12 | 46 |
| (3 month) | 27 | 60 | 44 | 89 | 2 | 23 |
| | 20 | 50 | 47 | 109 | 6 | 25 |
| | 59 | 57 | 71 | 133 | 7 | 15 |
| Average | 36.4 | 64 | 57.6 | 116 | 5.6 | 24 |
| 0.4 μg ePHF | 20 | 86 | 55 | 99 | 2 | 7 |
| (4 month) | 21 | 39 | 34 | 70 | 0 | 12 |
| | 28 | 76 | 47 | 88 | 4 | 19 |
| | 20 | 40 | 46 | 87 | 5 | 18 |
| | 46 | 45 | 34 | 59 | 1 | 7 |
| Average | 27 | 57.2 | 43.2 | 80.6 | 2.4 | 12.6 |

TABLE 107
AT100 staining (Cell number/slice)

| | | Entorhinal | | | Medial | Greater |
|---|---|---|---|---|---|---|
| | | cortex | | | Thalamus | Medial |
| | Olfactory | (1st | CA1 (2nd | | (2nd | Thalamus |
| | bulb | Synapse) | Synapse) | CA1/Sub | Synapse) | area |
| 0.29 μg ePHF | 16.45 | 2.50 | 2.25 | 3.38 | 2.25 | 3.50 |
| (3 month) | | | | | | |
| 0.29 μg ePHF | 7.00 | 3.44 | 3.83 | 7.17 | 0.00 | 4.00 |
| (4 month) | 7.33 | 2.88 | 4.43 | 9.14 | 0.00 | 4.75 |
| average | 7.17 | 3.16 | 4.13 | 8.15 | 0.00 | 4.38 |
| 0.4 μg ePHF | 1.20 | 4.82 | 8.25 | 13.63 | 0.33 | 2.75 |
| (3 month) | 5.33 | 8.33 | 7.50 | 17.50 | 3.00 | 11.50 |
| | 2.45 | 5.00 | 5.50 | 11.13 | 0.50 | 5.75 |
| | 2.00 | 4.55 | 5.22 | 12.11 | 1.50 | 6.25 |
| | 5.36 | 4.75 | 7.89 | 14.78 | 2.33 | 3.75 |
| average | 3.27 | 5.49 | 6.87 | 13.83 | 1.53 | 6.00 |
| 0.4 μg ePHF | 2.50 | 9.56 | 9.17 | 16.50 | 2.00 | 7.00 |
| (4 month) | 4.20 | 5.57 | 8.50 | 17.50 | 0.00 | 4.00 |
| | 3.11 | 7.60 | 6.71 | 12.57 | 1.33 | 4.75 |
| | 2.22 | 4.44 | 6.57 | 12.43 | 1.67 | 6.00 |
| | 5.75 | 5.63 | 5.67 | 9.83 | 0.50 | 2.33 |
| average | 3.56 | 6.56 | 7.32 | 13.77 | 1.10 | 4.82 |

TABLE 108
AT100 staining in piriform cortex

| | 0.29 μg ePHF | | | | 0.4 μg ePHF | | | |
|---|---|---|---|---|---|---|---|---|
| | | | % area | % area | | | % area | % area |
| | | | covered | covered | | | covered | covered |
| | Cell | Cells/ | (all | (3 | Cell | Cells/ | (all | (3 |
| Duration | number | slice | slices) | slices) | number | slice | slices) | slices) |
| 3 month | 458.00 | 152.67 | 10.21 | 10.21 | 1068.00 | 213.60 | 9.32 | 11.25 |
| | — | — | — | — | 818.00 | 204.50 | 10.49 | 12.46 |
| | — | — | — | — | 885.00 | 177.00 | 11.58 | 16.94 |
| | — | — | — | — | 505.00 | 168.33 | 7.00 | 7.00 |
| | — | — | — | — | 675.00 | 168.75 | 13.23 | 13.23 |
| 4 month | 1007.00 | 167.83 | 7.58 | 11.47 | 1540.00 | 256.67 | 13.60 | 19.79 |
| | 488.00 | 122.00 | 4.77 | 6.03 | 1046.00 | 348.67 | 12.38 | 12.38 |
| | — | — | — | — | 1097.00 | 274.25 | 15.33 | 17.69 |
| | — | — | — | — | 802.00 | 200.50 | 11.68 | 14.42 |
| | — | — | — | — | 1079.00 | 215.80 | 13.53 | 19.13 |

As shown in Table 108, Tau propagation to the piriform cortex could be observed at 3 months and 4 months, with a slight increase at 4 months. These data show that seeding and propagation of tau seeds could be observed using two different samples of ePHF at 3 and 4 months post seeding.

### Efficacy of Vectorized PT3 and PHF1 Antibody in Olfactory Bulb Propagation Model

Using experimental set up as described above, one day after seeding of PHF into the olfactory bulb of hTau mice, AAV particles comprising VOY10.CBA.PT3 construct (TAU ITR_111, SEQ ID NO: 2161) were administered intravenously at a dose of 1.4E13 vg/kg. Vehicle control group was injected with PBS. Mice were terminated after three months, and AT100 staining intensity were analyzed for the olfactory bulb, entorhinal cortex, ipsilateral hippocampus and thalamus. As shown in FIG. 2A, the number of AT100+ cells were significantly reduced in the olfactory bulb (43%) and hippocampus (17%) of AAV-treated mice compared to vehicle control.

Similarly, the efficacy of AAV particles comprising a vectorized PHF1 antibody construct under the GFAP astrocyte-specific promoter were also tested in the hTau olfactory bulb propagation model. Different doses of AAV particles comprising VOY101.GFAP.PHF1 construct (TAU_ITR242; SEQ ID NO: 2314) were injected intravenously one day post-seeding at 1.4E13 (low dose), 4.4E13 (medium dose), and 1.4E14 (high dose) vg/kg doses. Mice treated with the VOY101.GFAP.PHF1 constructs had a significant reduction (21% with the medium dose and 26% with the high dose, with a statistically insignificant 14% reduction in the low dose) in AT100 staining in a dose-dependent manner at least in hippocampus (see FIG. 2B).

### Example 2A. Efficacy of VOY101.PT3 Constructs in a Hippocampal Seeding Model

Exogenous tau seeds that include misfolded tau protein may be used to induce tauopathy that propagates from the site of injection to interconnected regions in the brain. Such models may be used to study the therapeutic efficacy of vectorized antibodies.

To establish a hippocampal tau seeding model in P301S transgenic mice, animals were infused intraparenchymally with AD lysates from patients with Alzheimer's disease. 2 month old female mice were injected at the CA1 of hippocampus unilaterally. The injections were performed at 2 μl per site at the rate of 2 μl per minute. Two doses of AD lysate (28 or 56 ng of ePHF) were administered to the animals. Phosphate buffered saline was used as the vehicle control. The stereotactic coordinates for the hippocampal infusion were as follows: AP: −2.1; ML: −1.5; DV: −1.8 from bregma (−1.6 from dura). Four to six weeks after the injection, mice were euthanized. At necropsy, CSF and serum samples were collected and mice were subjected to transcardiac perfusion with PBS. Brain samples were harvested and immersion-fixed in 10% buffered formalin for immunohistochemistry. For ELISA assays, brain samples were harvested and flash frozen. Tau pathology was measured using AT100 antibody. Additional readouts measured included weekly body weight measurements, and cage side observations for mortality or morbidity.

For IHC, mouse brains fixed in PFA for 48 hours were transferred to PBS. The samples were processed on Leica HistoCore Pearl and embedded in paraffin. Tissue was sectioned through the hippocampus and 3 planes (anterior, mid and posterior) were chosen for analysis. IHC samples were further processed using Ventana discovery ultra-protocol (#12). Briefly, sections were deparaffinized following antigen retrieval, which was performed for 64 minutes at 95 degrees Celsius using cell conditioner #1 containing EDTA. Endogenous peroxidase inhibition was achieved by treatment with inhibitor CM (H₂O₂) for 8 minutes followed by a blocking step using Rodent Block M (Biocare Medical; RBM961, Lot 082819-2) for another 8 minutes. Tissues were incubated with anti-AT-100 (Invitrogen; MN1060, Lot UH2803903) at 1:100 dilution for 60 minutes; followed by a brief washing step to remove excess antibody. This was followed by treatment with the secondary/linking antibody consisting of a combination of IgG1, IgG2a, and IgG3 (Abcam; ab133469, Lot GR3279440-1) at 1:1000 dilution for 20 minutes. Sections were developed using multimer, Omnimap Anti-Rb HRP treatment for 16 minutes followed by chromogen (DAB CM Kit). Slides were then stained with Hematoxylin II (for 4 minutes) and counterstained with Bluing Reagent (for 4 minutes). Following the staining the slides were washed in water, and dehydrated through graded ethanol and xylene treatment. A drop of Cytoseal 60 was added to the tissue section and a cover slip was placed on the slide to seal the tissue.

Analysis of the tissue sections revealed that infusion with AD lysate that contained 56 ng PHF led to robust tau pathology in the ipsilateral hippocampus at 4 weeks and 6 weeks post-seeding. This dose was used in subsequent experiments.

To measure the efficacy of the vectorized antibody delivery in the hippocampal seeding model, mice were transduced intravenously with VOY101.TAU_ITR111 (SEQ ID NO: 2161; VOY101.CBA.HL.T2A) particles. Six-week old female P301S transgenic mice were injected intravenously with AAV particles comprising TAU_ITR111 genome using doses described in Table 109.

TABLE 109
Dose of AAV particles comprising TAU_ITR111 genome

| | | | Age at |
|---|---|---|---|
| Group | Vector | Dose (vg/kg) | treatment (wks) |
| 1 | TAU_ITR111 (SEQ ID NO: | 1.40E+13 | 6 |
| | 2161; VOY101.CBA.HL.T2A) | | |
| 2 | TAU_ITR111 (SEQ ID NO: | 5.00E+12 | 6 |
| | 2161; VOY101.CBA.HL.T2A) | | |
| 3 | PBS | | 6 |

At 8 weeks, AD brain lysate containing 56 ng PHF was injected into the CA1 of the hippocampus unilaterally as described above. Some mice were euthanized at the time of seeding, whereas the rest of the mice were euthanized 6 weeks after the seeding. At week 6, CSF and serum were collected; and mice were subjected to intracardiac perfusion with PBS. Brains were harvested for dissection, and either snap frozen for ELISA analysis or fixed in 4% PFA for IgG IHC. Immunohistochemistry analysis was also performed using AT100 antibody.

Table 110 shows the PT3 expression levels at the time of seeding.

TABLE 110
PT3 levels at the time of seeding (ng/mg)

| | | 5.00E+12 | 1.40E+13 |
|---|---|---|---|
| Tissue type | Vehicle | Vg/Kg | Vg/Kg |
| Cortex | 0.00 | 13.73 | 69.37 |
| | 0.00 | 5.00 | 62.52 |
| | 0.00 | 22.31 | 20.59 |
| | 0.00 | 6.18 | 63.46 |
| | 0.00 | 10.42 | 97.44 |
| Average | 0.00 | 11.53 | 62.68 |
| Hippocampus | 0.00 | 4.26 | 69.04 |
| | 0.00 | 1.17 | 69.71 |
| | 0.00 | 6.99 | 9.36 |
| | 0.00 | 2.58 | 67.02 |
| | 0.00 | 7.00 | 68.28 |
| Average | 0.00 | 4.40 | 56.68 |
| Serum | 0.00 | 8830.00 | 30450.00 |
| | 0.00 | 2665.00 | 35375.00 |
| | 0.00 | 9070.00 | 7075.00 |
| | 0.00 | 5140.00 | 30975.00 |
| | 0.00 | 9050.00 | 46275.00 |
| Average | 0.00 | 6951.00 | 30030.00 |
| CSF | 0.00 | 511.00 | 726.00 |
| | 0.00 | 86.00 | 81.00 |
| | 0.00 | 251.00 | 306.00 |
| | 0.00 | 166.00 | 1941.00 |
| | 0.00 | 275.00 | 965.00 |
| Average | 0.00 | 257.80 | 803.80 |

As shown in Table 110, dose dependent PT3 levels were observed in the CNS at the time of seeding. The CSF PT3 expression levels obtained in these studies were consistent with the CSF PT3 expression observed in previous studies (5.00E+12 AAV particles resulted in average CSF PT3 levels of 337.87 ng/ml and 1.40E+13 resulted in average CSF PT3 levels of 559.98 ng/ml in previous experiments).

Distribution of PT3 expression in CNS was determined by IgG-IHC. AAV particle dose dependent expression of PT3 was observed in the CNS. In addition to cortex, hippocampus and thalamus, PT3 expression was also abundant in the cerebellum and brainstem. The broad expression and distribution of PT3 antibody in the CNS was consistent with previous experiments related to PT3 distribution.

IHC analysis using AT100 antibody was also performed. Anterior and medial levels of the hippocampus were used for quantification. AT-100 IHC analysis showed robust AT100 positive areas in the ipsilateral and contralateral hippocampus of AD-lysate seeded P301S mice. AD-lysate seeding activity was seen from anterior to the posterior hippocampus. Intrinsic AT100 tau pathology was also be observed in the brainstem of P301S female (at 14 weeks).

The efficacy of treatment with vectorized antibody delivery in P301S hippocampal seeding model was measured using AT100 IHC stained tissue sections. Hippocampi of tissue sections were imaged and the individual hippocampi were traced out in the sections. Any imaging or staining artifacts were annotated “out of the analysis”. The tissue sections from each mouse were numbered 1 through 4, with regions 1 and 2 being the animal left (ipsilateral) and 3 and 4 being the animal right (contralateral) tissue sections. Analysis was performed using HALO algorithm “Indica Labs-Area Quantification v2.1.3 settings 1.” The settings were determined empirically using multiple images to assess lower limit of staining intensity that captured stained pixels without picking up unstained pixels. The HALO algorithm settings for AT100 were 0.493, 0.815, 1.064; and the settings for the hematoxylin staining were 0.123, 0.108, 0.076. The minimum tissue optical density (OD) cut off values for classifying staining as weak, moderate or strong were 0.09, 0.5 and 0.9 respectively.

PT3 expression was also quantified as AT100 percentage immunoreactivity (IR) per hippocampal area. The results are shown in Table 111. The final row in the table lists the average value (in bold) for each group. There are two ipsilateral and two contralateral hippocampi per plane for most animals. Each data point in Table 111 is an average of AT100 IR per hippocampal area from anterior/medial hippocampal sections. Significance was calculated using one-way ANOVA-Tukey's multiple comparison test.

TABLE 111
PT3 levels at the time of seeding (ng/mg)

| Ipsilateral | | | Contralateral | | |
|---|---|---|---|---|---|
| | PT3 Low | PT3 high | | PT3 Low | PT3 high |
| | (5.00E+12 | (1.40E+13 | | (5.00E+12 | (1.40E+13 |
| Vehicle | Vg/Kg) | Vg/Kg) | Vehicle | Vg/Kg) | Vg/Kg) |
| 13.96 | 1.11 | 4.71 | 17.30 | 0.36 | 3.88 |
| 13.01 | 10.54 | 3.92 | 18.02 | 12.04 | 6.05 |
| 6.08 | 2.63 | 5.31 | 10.39 | 4.16 | 7.67 |
| 12.88 | 0.85 | 2.98 | 13.53 | 0.34 | 4.64 |
| 8.27 | 7.70 | 5.70 | 10.07 | 10.11 | 2.41 |
| 27.68 | 11.43 | 4.79 | 23.74 | 15.93 | 4.98 |
| 1.47 | 8.10 | 12.51 | 0.69 | 5.27 | 15.46 |
| 14.34 | 4.96 | 19.60 | 6.19 | 1.65 | 20.50 |
| 3.99 | 1.25 | 5.58 | 1.64 | 24.99 | 6.91 |
| 14.06 | 24.13 | 3.42 | 14.17 | 12.98 | 5.00 |
| 5.37 | 15.47 | 1.09 | 9.47 | 3.47 | 0.27 |
| 16.18 | 2.27 | 0.39 | 19.37 | 13.99 | 0.13 |
| 7.69 | 8.70 | 0.26 | 12.59 | 11.92 | 0.07 |
| 11.97 | 9.73 | 9.87 | 12.69 | 7.04 | 12.79 |
| 10.22 | 8.45 | 11.88 | 10.19 | 4.70 | 15.24 |
| — | 5.24 | 3.44 | — | 12.89 | 3.57 |
| — | 9.32 | — | — | 7.94 | — |
| — | 9.24 | — | — | — | — |
| 11.15 | 7.84 | 5.97 | 12.00 | 8.81 | 6.85 |

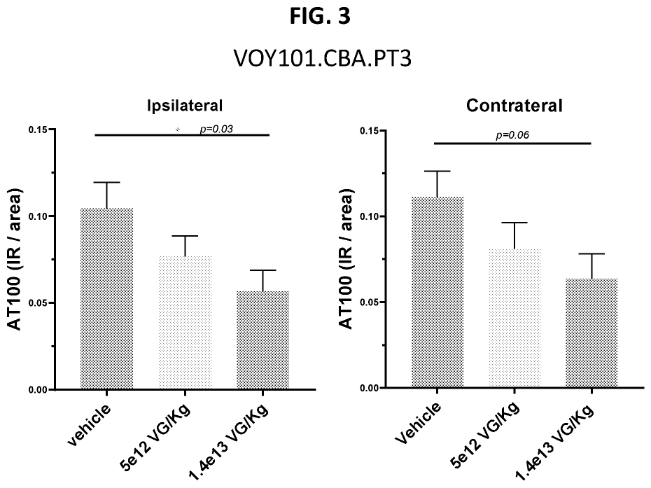

There was a significant reduction in the hippocampal AT100 positive areas in animals treated with 1.40E+13 vg/kg both at the ipsilateral site (46.5% reduction) and at the contralateral sites (43% reduction). A similar trend was observed in mice treated with 5.00E+12 Vg/Kg, but without statistical significance (see FIG. 3). Together, these data demonstrate a dose dependent reduction in AT100 positive staining in mice following IV delivery of VOY101.PT3 particles.

Similar experiments can also be performed using otherwise identical constructs, except for using tissue-specific promoters (as opposed to the ubiquitous CBA promoter), such as the SYN or GFAP promoter.

### Example 2B. Efficacy of VOY101.PHF1 Constructs in a Hippocampal Seeding Model

Similar to the experiment described in Example 2A, efficacy of a VOY101.CBA.PHF1 construct were examined in the hippocampal seeding model. Mice were transduced intravenously AAV particles comprising VOY101.TAU_ITR243 (SEQ ID NO: 2315) particles at 1.4E+13 and 1.4E+14 Vg/Kg doses.

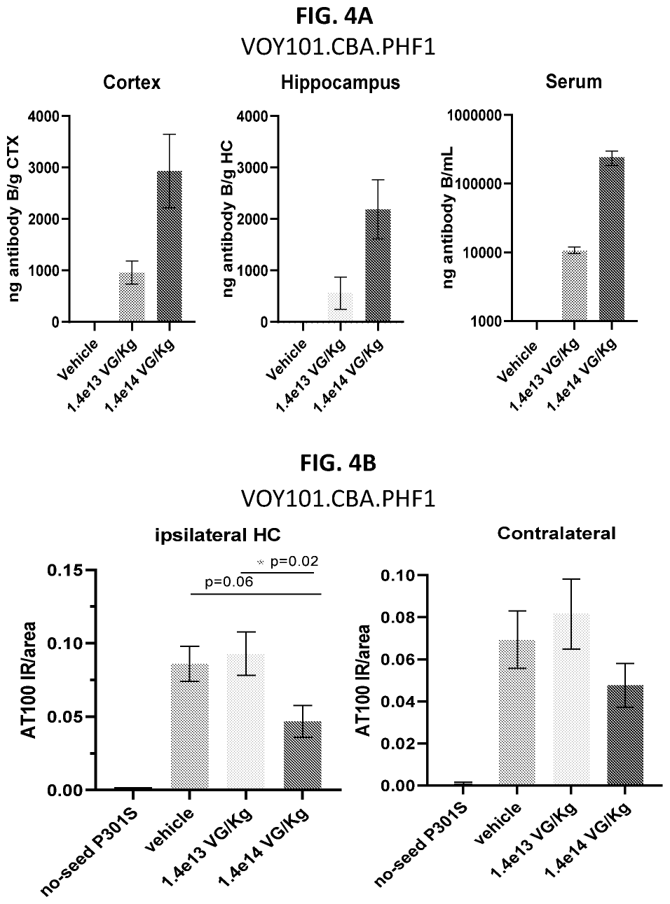

Dose dependent PHF1 levels were observed in the CNS at the time of seeding (see FIG. 4A) and at termination (data not shown). ATR100 staining of the hippocampus showed that injection of vectorized PHF1 construct was effective at the higher dose 1.4E+14 Vg/Kg, achieving about 46% reduction of AT100 positive area (see FIG. 4B). These data show that intravenous delivery of vertorized PHF1, which targets the C-terminal of tau, was effective at reducing tau burden in this hippocampal seeding model.

Similar experiments can also be performed using otherwise identical constructs, except for using tissue-specific promoters (as opposed to the ubiquitous CBA promoter), such as the SYN or GFAP promoter.

### Example 3A. Efficacy of VOY101.PHF1 Constructs in Intrinsic Model

Efficacy of a VOY101.CBA.PHF1 construct in an intrinsic tauopathy P301S mouse model was examined. Briefly, VOY101.TAU_ITR243 (SEQ ID NO: 2315) particles were delivered intravenously into mice at age of 8 weeks at 3 doses: 1.4E+13, 4.4E+13, or 1.4E+14 Vg/Kg. Tauopathy was allowed to progress naturally over 3 months, and mouse tissues were examined after termination.

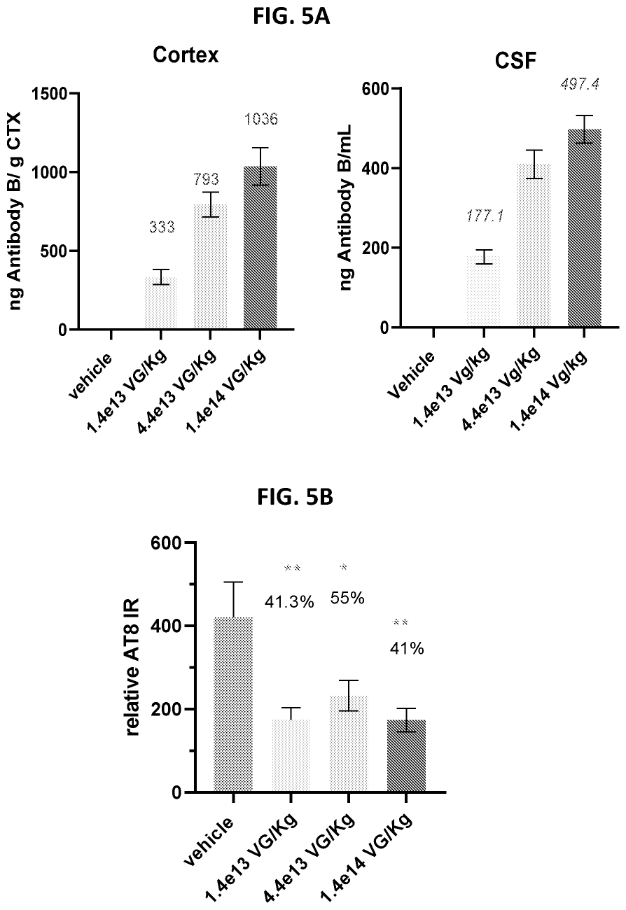

Expression levels of PHF1 antibody increased in an AAV particle dose-dependent manner in the cortex and CSF at termination of the experiment (see FIG. 5A). Efficacy against tau pathology was carried out using AT8 antibody to measure tau levels by ELISA, and at all three doses tested, AT8 immunoreactivity was reduced by at least 45% in average, up to about 59% reduction (all statistically significant). These data demonstrated that intravenous delivery of vertorized PHF1 was effective at reducing tau burden in this intrinsic tauopathy model.

Similar experiments can also be performed using otherwise identical constructs, except for using tissue-specific promoters (as opposed to the ubiquitous CBA promoter), such as the SYN or GFAP promoter.

### Example 3B. Efficacy of VOY101.PT3 Constructs in Intrinsic Model

Efficacy of a VOY101.CBA.PT3 construct in an intrinsic tauopathy P301S mouse model is examined. Briefly, VOY101.TAU_ITR111 (SEQ ID NO: 2161) particles are delivered intravenously into mice at age of 8 weeks at 3 doses: 1.4E+13, 4.4E+13, or 1.4E+14 Vg/Kg. Tauopathy is allowed to progress naturally over 3 months, and mouse tissues are examined after termination.

Expression levels of PT3 antibody increases in an AAV particle dose-dependent manner in the cortex and CSF at termination of the experiment. Efficacy against tau pathology is carried out using AT8 antibody to measure tau levels by ELISA, and at all three doses tested, AT8 immunoreactivity is reduced. These data demonstrates that intravenous delivery of vertorized PT3 is effective at reducing tau burden in the intrinsic tauopathy model.

Similar experiments can also be performed using otherwise identical constructs, except for using tissue-specific promoters (as opposed to the ubiquitous CBA promoter), such as the SYN or GFAP promoter.

### Example 4. Effect of Inflammation at Injection Site on Antibody Staining

The effect of injury/inflammation at the site of injection on the PT3 antibody staining pattern in the glia and the neurons is tested. 19 week-old htau female mice (B6.Cg-Mapt^{tml(EGFP)Klt} Tg(MAPT)8cPdav/J; The Jackson Laboratory, Bar Harbor, ME) are injected with PBS intracranially into the olfactory bulb. One day after the injection, AAV particles comprising viral genome TAU_ITR111 (SEQ ID NO: 2161; VOY101.CBA.HL.T2A) formulated in formulation buffer (10 mM disodium phosphate, 2 mM monopotassium phosphate, 2.7 mM potassium chloride, 192 mM sodium chloride and 0.001% Pluronic Acid (F-68)) are provided intravenously at a dose of 1.4e13/kg. Whole brain samples are collected at 28 days and fixed in formalin. Brains are analyzed by immunohistochemistry for glial and neuronal vector biodistribution.

### Example 5. Efficacy of Vectorized Antibody Delivery in Olfactory Bulb Propagation Model

Nineteen week-old mice are injected with 0.4 μg ePHF in 2 μL or PBS. One day later, AAV particles comprising viral genome CBA.HL.T2A (TAU_ITR111; SEQ ID NO: 2161), GFAP-PT3-HF.T2AL (TAU_ITR113; SEQ ID NO.2163) or SYN-PT3-HF.T2AL (TAU_ITR114; SEQ ID NO. 2164) are provided intravenously at a dose of 1.4e13/kg or 7.0e13/kg. AAV particles are formulated in formulation buffer (10 mM disodium phosphate, 2 mM monopotassium phosphate, 2.7 mM potassium chloride, 192 mM sodium chloride and 0.001% Pluronic Acid (F-68)). Mice injected with formulation buffer alone will serve as the vehicle control group. 16 weeks after the injection, the mice are euthanized and CNS, serum, liver and/or other tissue samples are collected. Additionally, each experiment includes a control group of animals that are not seeded with ePHF, but are treated with the AAV particles described herein or formulation buffer. Animals are euthanized at 16 weeks and brain samples analyzed for PT3 expression by ePHF ELISA and IHC as well as vector biodistribution,

For efficacy studies, the entire brain is fixed in formalin and sectioned horizontally. For expression studies, the right half of the brain and lumbar SC tissues are fixed with 4% PFA; whereas the left half of the brain and cervical/thoracic SC are flash frozen. Liver, serum and CSF are also harvested at the end of the study. Harvested whole brain is analyzed by AT100 IHC analysis of the tissues (specifically, the olfactory bulb, piriform cortex, Entorhinal cortex, thalamus, hippocampus CA1, amygdala); expression samples are analyzed by ePHF ELISA (specifically, CNS tissue, CSF, peripheral tissues, serum); IgG1immunohistochemistry (IHC); and vector genome quantification (specifically, cortex, hippocampus, thalamus, OB, BS, thoracic SC, and liver).

Similar experiments can also be performed using otherwise identical constructs, except for using tissue-specific promoters (as opposed to the ubiquitous CBA promoter), such as the SYN or GFAP promoter.

### Example 6. Kinetics of Antibody Expression after Intravenous Dosing of Vectorized Anti-Tau Antibodies

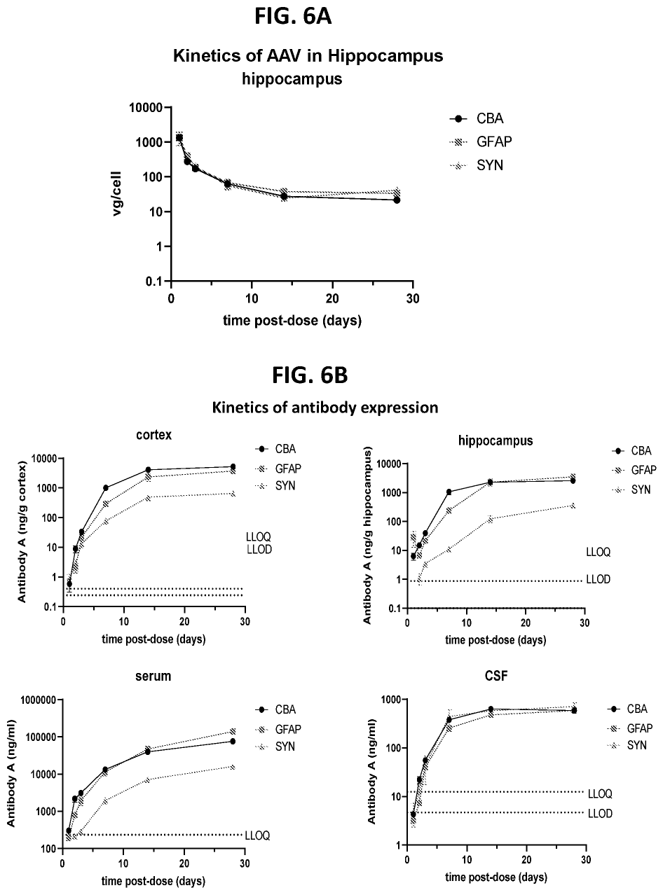

Viral genome comprising vectorized PT3 construct with CBA (TAU_ITR111, SEQ ID NO: 2161), GFAP (TAU_ITR105; SEQ ID NO: 2155) or SYN (TAU_ITR106, SEQ ID NO: 2156) promoters are packaged in VOY101 blood brain barrier-penetrant capsid and injected intravenously into wild-type mice at 1.4E+13 Vg/Kg. Samples were collected on day 1, 2, 3, 7, 14 and 28 after injection. Following i.v. administration, antibody expression could be detected as early as 2 days, approached maximum levels at 7 days, and plateaued 14-28 days post-injection (see FIG. 6B).

Particularly in the hippocampus, AAV biodistribution (FIG. 6A) and antibody expression (FIG. 6B) both approached stable levels by 7 days-post injection and plateaued at 14-28 days post-dose, stabilizing at approximately 30 vg/cell and antibody concentrations of 2750 ng/g tissue for the CBA and GFAP promoters and 450 ng/g tissue for the SYN promoter in the hippocampus. These data demonstrate that the antibody vector constructs have good biodistribution and effective antibody expression.

Additionally, antibody levels in different CNS and fluid compartment 5 weeks after injection of viral particles comprising VOY101.CBA.PT3 or VOY101.CBA.PHF1 were compared to antibody levels after injection of the respective purified PT3 and PHF1 antibodies (passive immunization) (see Table 114). CSF and serum data from passive immunization (PT3 or PHF1, 1× weekly) fall within the range of published data from various antibody by passive immunization with similar dosing paradigm. Under the experimental condition used, PHF1 expression levels were lower in hippocampus (7.5× lower), cortex (10× lower), thalamus (5.4× lower) and brainstem (1.8× lower) compared to PT3 expression levels in the corresponding CNS regions of mice dosed with equal amount of CBA.PT3 vector genome (1.4e3 vg/kg).

Overall, injection of a single dose of vectorized antibody resulted in a much higher level of both PT3 and PHF1 antibodies in the CNS. This data demonstrates superiority of transduction with vectorized antibody over passive immunization.

TABLE 114
Pharmacokinetics of CBA.PT3 and CBA.PHF1 vectors

| | | | | | |
|---|---|---|---|---|---|
| Cortex | Hippocampus | Thalamus | Brainstem | CSF | Serum |
| (ng/g) | (ng/g) | (ng/g ) | (ng/g) | (ng/mL) | (μg/mL) |

Immunization with vectorized antibody

| | | | | | | |
|---|---|---|---|---|---|---|
| CBA.PT3 | 7535 | 2785 | 5000 | 5880 | 596.8 | 75 |
| (1.4e13 Vg/kg) | | | | | | |
| CBA.PHF1 | 699.1 | 367.4 | 922 | 3285 | 86 | 20 |
| (1.4e13 Vg/kg) | | | | | | |
| CBA.PHF1 | 1223 | 899 | 2762 | 9186 | 374 | 130 |
| (4.4e13 Vg/kg) | | | | | | |
| CBA.PHF1 | 2389 | 2567 | 5092 | 15936 | 512 | 534 |
| (1.4e14 Vg/kg) | | | | | | |

Passive immunization

| | | | | | | |
|---|---|---|---|---|---|---|
| PT3 (30 mg/kg, | | | | | 283.2 | 359.2 |
| 1×, weekly) | | | | | | |
| PHF1 (40 mg/kg, | 66.2 | 113 | 375 | 1350 | 292 | 465 |
| 1×, weekly) | | | | | | |
| PHF1 (40 mg/kg, | 92.7 | 142 | 695 | 1200 | 621 | 540 |
| 2× weekly | | | | | | |

Similar experiments can also be performed using otherwise identical constructs, except for using tissue-specific promoters (as opposed to the ubiquitous CBA promoter), such as the SYN or GFAP promoter.

### Example 7. Evaluation of Capsids and PT3 Antibody Constructs in Non-Human Primates

Adult rhesus macaque monkeys pre-screened for low anti-AAV antibody levels will receive intravenous (IV) or intracisternal magna (CM or ICM) administration of capsids and/or PT3 antibody constructs (e.g. TAU_ITR111 (SEQ ID NO: 2161) to assess CNS biodistribution and transduction of capsids and/or PT3 antibody constructs.

AAV capsids and PT3 antibody constructs (e.g. TAU_ITR111, SEQ ID NO: 2161) will be formulated in a solution containing 10 mM Sodium Phosphate Dibasic, 2 mM Potassium Phosphate Monobasic, 2.7 mM Potassium Chloride, 192 mM Sodium Chloride, 0.001% Pluronic F-68. The study design is provided in Table 112.

TABLE 112
Experimental Design Overview

| | | | | | | |
|---|---|---|---|---|---|---|
| Grp | 1 | 2 | 3 | 4 | 5 | 6 |
| Test article | VOY101.CBA. | VOY101.CBA. | VOY1O1. | Vehicle | VOY101.CBA. | Vehicle |
| | PT3.HL.T2A; | PT3.HL.T2A; | Empty | (PBS, | PT3.HL.T2A; | (PBS, |
| | TAU_ITR111 | TAU_ITR111 | capsid | 0.001% | TAU_ITR111 | 0.001% |
| | (SEQ ID NO: | (SEQ ID NO: | | F-68) | (SEQ ID NO: | F-68) |
| | 2161) | 2161) | | | 2161) | |
| Route | IV | IV | IV | IV | ICM | ICM |
| Dose | 1.6e13 vg/kg | 5.0 e13 vg/kg | 7.8 | N/A | 3.0e13 vg/kg | N/A |
| | | | VLP/kg | | | |
| Number (F = | 3 (2F/1M) | 3 (1F/2M) | 3 (2F/1M) | 2 | 3 (1F/2M) | 2 |
| female; M = | | | | (1F/1M) | | (1F/1M) |
| male) | | | | | | |
| Average NHP | 3 | 3 | 3 | 3 | 3 | 3 |
| weight | | | | | | |
| Duration | Day 29 | Day 29 | Day 29 | Day 29 | Day 29 | Day 29 |

The study is performed in stages. For group 1 involving low dose, one animal is dosed intravenously and observed for 10 days; if tolerated, then next two animals are dosed and observed for full period to day 29. If group 1 involving low dose is tolerated, then group 2 is initiated into the study. Similar to group 1, one animal in group 2 is dosed intravenously with the high dose of the test article and observed for 10 days. If tolerated, then the next 2 animals are dosed. A similar strategy is adopted for group three. All intravenous deliveries are administered at an IV infusion rate of 5 ml/kg over 1.5 hours.

For groups related to ICM dosing (i.e. group 5 and group 6), one animal is dosed and observed for 10 days. If the dose is tolerated, then the next two animals are treated. Animals are maintained under isoflurane sedation. A stereotaxic frame and manipulator are used for the procedure. The placement of the needle placement is confirmed by CSF flow. 2 ml of CSF is collected. The TA is infused at 0.5 ml/min (total 3 ml) and the needle is flushed. The needle remains in place for 5 min, and the animal is then placed in the Trendelenburg position (15-30 deg, feet above head) for 10 min. Animals will be monitored for the following clinical parameters (see Table 113).

TABLE 113
Clinical parameters

| Parameters | Frequency |
|---|---|
| Clinical Signs | Daily |
| Body weight/Food Consumption | Weekly |
| FOB cage: fine motor and 10 | Only for ICM arms: Pre (training), Day |
| nerves; open field: body motor | 10, 30 |
| Clinical Pathology (standard | Heme and chem: Pre (week −1, Day 1), |
| serum chem) | Days 3, 5, 7, 14, 21, 29 |
| | Coagulation: Pre (week-1) days 7 & 28 |
| Cytokine and complement panel | Pre (day 1), 6 h, 12 h, 24 h, 48 h, 96 h, |
| | 14 d, 27 d post-dose |
| CSF (lumbar puncture: count | Pre (1x), Day 7, 15, 29 |
| and cell differential), antibody | |
| protein | |
| Gross necropsy including | Day 29 |
| organ weights | |
| Histopathology | Brain, spinal cord, 3 pairs of DRG (C6, |
| | T6, L4), peripheral nerves, liver, |
| | relevant gross observations |

On day 29, animals are euthanized and tissue samples are collected. One hemisphere of the brain is placed in 4% paraformaldehyde (for hematoxylin and eosin studies as well immunohistochemical analysis). Contralateral hemispheres are snap frozen in vapor phase: isopentane. Biodistribution is measured by tissue punches for vector genome level analysis and protein analysis of CNS and PNS tissue. The entire brain is weighed, and volume calculated. The brain is placed in ice-cold PBS for approximately 15 mins before being placed in a refrigerated brain mold and sliced coronally in 3-4 mm slabs. Samples are placed on ice cold board, alphabetized, and photographed. Right hemisphere post-fixed and left hemisphere frozen on 3×5 glass slides and wrapped in foil.

Each pair of dorsal root ganglia are collected i.e. from C1-7, T1-12, L1-L7 and sacral regions. Odd segment DRG pairs are frozen in vapor phase isopentane and dry ice) for vector genome quantification and mRNA analysis. Even segment DRG pairs are placed in 4% paraformaldehyde for H&E and IHC analyses. Antibody expression and biodistribution analyses are carried out in a tiered format i.e. if tier 1 analysis is effective, then tier 2 tissue are analyzed. Tissues analyzed in tier 1 include frontal cortex, motor cortex, hippocampus, putamen, liver, serum, CSF. Tissues analyzed in tier 2 include thalamus, caudate, substantia nigra, hypothalamus, amygdala, basal ganglia, cerebellum, deep cerebellar nuclei, brainstem, DRG, spinal cord, and retina.

For each peripheral tissue sample, one piece is flash-frozen. Approximately 500 mg to 1.5 g of peripheral tissue is collected. Post-collection, all tissues are sliced 3-4 mm thickness and placed in 4% paraformaldehyde for 24-32 hours at room temperature and then transferred to phosphate buffered saline (PBS) after rinse. Peripheral tissues collected include heart, kidney, liver, lung, spleen, lymph node-cervical, lymph node-prescapular, muscle-vastus lateralis, sciatic nerve, rinse eye (prespecified volume collected).

AAV biodistribution is measured by quantifying vector genome levels in the cortex, hippocampus, striatum, and liver. PT3 levels are measured using ePHF ELISA in cortex, hippocampus, striatum, liver, serum, and CSF. Tissues are also immunostained with HRP labeled mouse IgG1 for presence of Tau antibodies. Further, these sample are co-immunostained with NeuN, IbaI or GFAP to identify the cell type. CSF antibody expression level, brain antibody expression level, cell specificity, distribution in the brain, and safety/tolerability parameters are evaluated in this study.

Similar experiments can also be performed using otherwise identical constructs, except for using tissue-specific promoters (as opposed to the ubiquitous CBA promoter), such as the SYN or GFAP promoter.

### Example 8. Construction of Viral Genomes for the Expression of Antibody Fragments

Sixteen viral genomes were designed for the expression of PHF1 F(ab′)2, Fab and scFv fragments (SEQ ID NO: 4547-4562), driven by one of four promoters (CAG, CBA, GFAP or synapsin). scFv fragments constructs were designed in two configurations (heavy-linker-light or light-linker-heavy). These constructs are described in Tables 5B and 92-95.

### VIII. Equivalents and Scope

Those skilled in the art will recognize, or be able to ascertain using no more than routine experimentation, many equivalents to the specific embodiments in accordance with the disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit in its broader aspects.

While the present disclosure has been described at some length and with some particularity with respect to the several described embodiments, it is not intended that it should be limited to any such particulars or embodiments or any particular embodiment, but it is to be construed with reference to the appended claims so as to provide the broadest possible interpretation of such claims in view of the prior art and, therefore, to effectively encompass the intended scope.

1. An isolated, nucleic acid comprising a transgene encoding an antibody or an antibody fragment thereof that binds to tau, comprising a heavy chain variable region (VH) and a light chain variable region (VL), wherein:
(i) the nucleotide sequence encoding the VH, comprises the nucleotide sequence of SEQ ID NO: 1839, 1841, or 2170, or a nucleotide sequence having at least 90%, 92%, 95%, 96%, 97%, 98%, or 99% sequence identity thereto; and/or
(ii) the nucleotide sequence encoding the VL comprises the nucleotide sequence of SEQ ID NO: 1957 or 4565, or a nucleotide sequence having at least 90%, 92%, 95%, 96%, 97%, 98%, or 99% sequence identity thereto.

2. The nucleic acid of claim 1, wherein:
(1) the nucleotide sequence encoding the VH comprises SEQ ID NO: 1841, or a nucleotide sequence having at least 90%, 92%, 95%, 96%, 97%, 98%, or 99% sequence identity thereto, and the nucleotide sequence encoding the VL comprises SEQ ID NO: 4565, or a nucleotide sequence having at least 90%, 92%, 95%, 96%, 97%, 98%, or 99% sequence identity thereto;
(2) the nucleotide sequence encoding the VH comprises SEQ ID NO: 1839, or a nucleotide sequence having at least 90%, 92%, 95%, 96%, 97%, 98%, or 99% sequence identity thereto, and the nucleotide sequence encoding the VL comprises SEQ ID NO: 1957, or a nucleotide sequence having at least 90%, 92%, 95%, 96%, 97%, 98%, or 99% sequence identity thereto; or,
(3) the nucleotide sequence encoding the VH comprises SEQ ID NO: 2170, or a nucleotide sequence having at least 90%, 92%, 95%, 96%, 97%, 98%, or 99% sequence identity thereto, and the nucleotide sequence encoding the VL comprises SEQ ID NO: 1957, or a nucleotide sequence having at least 90%, 92%, 95%, 96%, 97%, 98%, or 99% sequence identity thereto.

3. The nucleic acid of claim 1, which further comprises a nucleotide sequence encoding a signal sequence, optionally wherein the nucleotide sequence encoding the signal sequence:
(i) is located 5′ relative to the nucleotide sequence encoding the VH and/or 5′ relative to nucleotide sequence encoding the VL;
(ii) comprises the nucleotide sequence of signal sequence provided in Table 3 or Table 11, or a nucleotide sequence at least 90%, 92%, 95%, 96%, 97%, 98%, or 99% sequence identity thereto;
(iii) comprises the nucleotide sequence of any one of SEQ ID NOs: 1741, 4564, or 1862, or a nucleotide sequence at least 90%, 92%, 95%, 96%, 97%, 98%, or 99% sequence identity thereto;
(iv) comprises the nucleotide sequence of SEQ ID NO: 1741, or a nucleotide sequence at least 90%, 92%, 95%, 96%, 97%, 98%, or 99% sequence identity thereto and is located 5′ relative to the nucleotide sequence encoding the VH;
(v) comprises the nucleotide sequence of SEQ ID NO: 4564 or 1862, or a nucleotide sequence at least 90%, 92%, 95%, 96%, 97%, 98%, or 99% sequence identity thereto and is located 5′ relative to the nucleotide sequence encoding the VL;
(vi) comprises the nucleotide sequence of SEQ ID NO: 1741, or a nucleotide sequence at least 90%, 92%, 95%, 96%, 97%, 98%, or 99% sequence identity thereto and is located 5′ relative to the nucleotide sequence encoding the VH; and comprises the nucleotide sequence of SEQ ID NO: 4564, or a nucleotide sequence at least 90%, 92%, 95%, 96%, 97%, 98%, or 99% sequence identity thereto and is located 5′ relative to the nucleotide sequence encoding the VL; or
(vii) comprises the nucleotide sequence of SEQ ID NO: 1741, or a nucleotide sequence at least 90%, 92%, 95%, 96%, 97%, 98%, or 99% sequence identity thereto and is located 5′ relative to the nucleotide sequence encoding the VH; and comprises the nucleotide sequence of SEQ ID NO: 1862, or a nucleotide sequence at least 90%, 92%, 95%, 96%, 97%, 98%, or 99% sequence identity thereto and is located 5′ relative to the nucleotide sequence encoding the VL.

4. The nucleic acid of claim 1, wherein:
(i) the nucleotide sequence encoding the VH is located 5′ relative to the nucleotide sequence encoding the VL; or
(ii) the nucleotide sequence encoding the VL is located 5′ to the nucleotide sequence encoding the VH.

5. The nucleic acid of claim 1, comprising:
(i) a nucleotide sequence encoding a VH signal sequence comprising SEQ ID NO: 1741, a nucleotide sequence encoding the VH comprising SEQ ID NO: 1841, a nucleotide sequence encoding a VL signal sequence comprising SEQ ID NO: 4564, and a nucleotide sequence encoding the VL comprising SEQ ID NO: 4565;
(ii) a nucleotide sequence encoding a VH signal sequence comprising SEQ ID NO: 1741, a nucleotide sequence encoding the VH comprising SEQ ID NO: 2170, and a nucleotide sequence encoding the VL comprising SEQ ID NO: 1957; or,
(iii) a nucleotide sequence encoding a VH signal sequence comprising SEQ ID NO: 1862, a nucleotide sequence encoding the VL comprising SEQ ID NO: 1957, and a nucleotide sequence encoding the VH comprising SEQ ID NO: 1839.

6. The nucleic acid of claim 1, wherein: the encoded VH and VL:
(i) are connected directly, e.g., without a linker;
(ii) are connected via a linker;
(iii) are connected via a linker, wherein the linker comprises, or is encoded by a linker polynucleotide comprising:
(a) the sequence of any of the linker polynucleotide sequences provided in Table 2 or a sequence at least 90%, 92%, 95%, 96%, 97%, 98%, or 99% identical thereto;
(b) an encoded furin cleavage site and/or an encoded T2A polypeptide;
(c) an encoded hinge region, e.g., an encoded IgG1 hinge region and/or an encoded IgG3 hinge region;
(d) a (Gly4Ser)n linker, wherein n is 1-10, e.g., n is 3, 4, or 5;
(e) the sequence of any one or more of SEQ ID NOs: 1724, 1726, 1739, 2244, and/or 1730, or a sequence at least 90%, 92%, 95%, 96%, 97%, 98%, or 99% identical thereto;
(f) the sequence of SEQ ID NO: 1739 and SEQ ID NO: 2244, followed by a nucleotide sequence encoding a Furin cleavage site (e.g., SEQ ID NO: 1724), and further followed by a nucleotide sequence encoding a T2A polypeptide (e.g., SEQ ID NO: 1726);
(g) a nucleotide sequence encoding a Furin cleavage site (e.g., SEQ ID NO: 1724), followed by a nucleotide sequence encoding a T2A polypeptide (e.g., SEQ ID NO: 1726); or,
(h) a (Gly4Ser)₃ linker (e.g., SEQ ID NO: 1730).

7. (canceled)

8. The nucleic acid of claim 6, comprising, from 5′ to 3′:
(i) the nucleotide sequences of SEQ ID NOs: 1741, 1841, 1739, 2244, 1724, 1726, 4564, and 4565, or a sequence at least 90%, 92%, 95%, 96%, 97%, 98%, or 99% identical thereto;
(ii) the nucleotide sequences of SEQ ID NOs: 1741, 1841, 1724, 1726, 4564, and 4565, or a sequence at least 90%, 92%, 95%, 96%, 97%, 98%, or 99% identical thereto;
(iii) the nucleotide sequences of SEQ ID NOs: 1741, 2170, 1730, and 1957, or a sequence at least 90%, 92%, 95%, 96%, 97%, 98%, or 99% identical thereto; or,
(iv) the nucleotide sequences of SEQ ID NOs: 1862, 1957, 1730, and 1839, or a sequence at least 90%, 92%, 95%, 96%, 97%, 98%, or 99% identical thereto.

9. An isolated antibody or antibody fragment encoded by the nucleic acid of claim 1.

10. A viral genome, e.g., an adeno-associated (AAV) viral genome, which comprises a first promoter operably linked to the transgene encoded by the nucleic acid of claim 1.

11. The AAV viral genome of claim 10, which further comprises a second promoter, wherein the first promoter is operably linked to the first polynucleotide sequence, and the second promoter is operably linked to the second polynucleotide sequence, optionally wherein, the first promoter is the same as the second promoter, or the first promoter is different from the second promoter.

12. The AAV viral genome of claim 10, wherein the first promoter and/or the second promoter comprises:
(i) a ubiquitous promoter or a CNS-specific promoter, optionally wherein the CNS-specific promoter comprises a neuronal-specific promoter, glial cell-specific promoter, oligodendrocyte-specific promoter, or astrocyte-specific promoter;
(ii) a ubiquitous promoter, where in the ubiquitous promoter is:
(a) CAG (e.g., SEQ ID NO: 2080 or 2238), CBA (e.g., SEQ ID NO: 2084), CB (e.g., SEQ ID NO: 2083), CB6, CMV, EF-1α, PGK, UBC, GUSB (hGBp), UCOE, or a nucleotide sequence having at least 90%, 92%, 95%, 96%, 97%, 98%, or 99% sequence identity thereto;
(b) a CAG promoter, which comprises a minimum CBA promoter (e.g., SEQ ID NO: 2082 or 2083), and a CMVie enhancer (e.g., SEQ ID NO: 2081 or 2087); or
(c) a minimum CBA promoter (e.g., SEQ ID NO: 2082 or 2083) or a nucleotide sequence having at least 90%, 92%, 95%, 96%, 97%, 98%, or 99% sequence identity thereto, and a CMVie enhancer (e.g., SEQ ID NO: 2081 or 2087) or a nucleotide sequence having at least 90%, 92%, 95%, 96%, 97%, 98%, or 99% sequence identity thereto;
(iii) a neuronal-specific promoter, wherein the neuronal-specific promoter is synapsin (Syn) promoter (e.g., SEQ ID NO: 2086), NSE promoter, methyl-CpG binding protein 2 (MeCP2) promoter, Ca2+/calmodulin-dependent protein kinase II (CaMKII) promoter, metabotropic glutamate receptor 2 (mGluR2) promoter, neurofilament light (NFL) or heavy (NFH) promoter, β-globin minigene nβ2 promoter, preproenkephalin (PPE) promoter, enkephalin (Enk) promoter, excitatory amino acid transporter 2 (EAAT2) promoter, or a nucleotide sequence having at least 90%, 92%, 95%, 96%, 97%, 98%, or 99% sequence identity thereto; and/or
(iv) an astrocyte-specific promoter, wherein the astrocyte-specific promoter is glial fibrillary acidic protein (GFAP) promoter (e.g., SEQ ID NO: 2085), or EAAT2 promoter, or a nucleotide sequence having at least 90%, 92%, 95%, 96%, 97%, 98%, or 99% sequence identity thereto; or wherein the oligodendrocyte-specific promoter is myelin basic protein (MBP) promoter, or a nucleotide sequence having at least 90%, 92%, 95%, 96%, 97%, 98%, or 99% sequence identity thereto.

13. (canceled)

14. The AAV viral genome of claim 10, further comprising:
(i) an enhancer, optionally wherein the enhancer comprises a CMVie enhancer (e.g., SEQ ID NO: 2081 or 2087);
(ii) a polyadenylation (polyA) signal region, optionally, wherein the polyA signal region comprises the nucleotide sequence of any of SEQ ID NO: 2122-2124, or a nucleotide sequence with at least 95% identity thereto, optionally wherein the polyA signal region comprises the nucleotide sequence of SEQ ID NO: 2122, or a nucleotide sequence at least 95% identical thereto;
(iii) at least one ITR sequence, optionally wherein:
(a) one ITR sequence is positioned 5′ relative to the encoded transgene and/or one ITR sequence is positioned 3′ relative to the encoded transgene;
(b) said at least one ITR sequence comprises a nucleotide sequence of any one of SEQ ID NOs: 2076-2079, or a nucleotide sequence with at least 80%, 85%, 90%, or 95% sequence identity thereto; and/or
(c) one ITR sequence is positioned 5′ relative to the encoded transgene and comprises a nucleotide sequence of SEQ ID NO: 2076 or a nucleotide sequence with at least 80%, 85%, 90%, or 95% sequence identity thereto; and/or one ITR sequence is positioned 3′ relative to the encoded transgene and comprises a nucleotide sequence of SEQ ID NO: 2078 or a nucleotide sequence with at least 80%, 85%, 90%, or 95% sequence identity thereto;
(iv) at least 1, 2, or 3 intron regions, optionally, wherein each intron region independently comprises a nucleotide sequence of any of the intron regions listed in Table 10, or a nucleotide sequence with at least 95% identity thereto;
(v) at least 1, 2, or 3 exon regions; optionally, wherein each exon region independently comprises a nucleotide sequence of any of the exon sequences in Table 9, or a nucleotide sequence with at least 95% identity thereto;
(vi) a BSA nucleotide sequence comprising SEQ ID NO: 4563 or a nucleotide sequence at least 80% identical thereto; and/or
(vii) a Kozak sequence, optionally wherein the Kozak sequence comprises the nucleotide sequence of SEQ ID NO: 2114 or 4543.

15. The AAV viral genome of claim 14, wherein:
(i) said intron regions comprise an ie intron 1, a beta-globin intron, or a combination thereof;
(ii) said intron regions comprise the nucleotide sequence of SEQ ID NO: 2095, 2097, or a combination thereof, or a nucleotide sequence at least 95% sequence identity thereto;
(iii) said exon regions comprise an ie1 exon 1 or a beta-globin exon; and/or
(iv) said exon regions comprise the nucleotide sequence of SEQ ID NO: 2090, 2093, or a combination thereof, or a nucleotide sequence at least 95% identical thereto.

16. The AAV viral genome of claim 14, wherein the BSA sequence comprising the nucleotide sequence of SEQ ID NO: 4563 is present immediately 5′ to the nucleotide sequence encoding the most 5′ encoded VL or VH.

17. The AAV viral genome of claim 10, further comprising;
(i) a nucleotide sequence encoding a miR binding site, e.g., a miR binding site that modulates, e.g., reduces, expression of the payload encoded by the AAV viral genome in a cell or tissue where the corresponding miRNA is expressed;
(ii) at least 1-5 copies of an encoded miR binding site, e.g., at least 1, 2, 3, 4, or 5 copies;
(iii) at least 3 or 4 copies of an encoded miR binding site, optionally wherein all copies comprise the same miR binding site, or at least one, two, three or all of the copies comprise a different miR binding site, and/or,
(iv) wherein the encoded miR binding site comprises a miR122 binding site, a miR183 binding site, a miR-142-3p, or a combination thereof, optionally wherein:
(a) the encoded miR122 binding site comprises the nucleotide sequence of SEQ ID NO: 4566, or a nucleotide sequence substantially identical (e.g., having at least 90%, 92%, 95%, 97%, 98%, or 99% sequence identity) thereto; or a nucleotide sequence having at least one, two, three, four, five, six, or seven modifications, but no more than ten modifications of SEQ ID NO: 4566;
(b) the encoded miR183 binding site comprises the nucleotide sequence of SEQ ID NO: 4569, or a nucleotide sequence substantially identical (e.g., having at least 90%, 92%, 95%, 97%, 98%, or 99% sequence identity) thereto; or a nucleotide sequence having at least one, two, three, four, five, six, or seven modifications, but no more than ten modifications of SEQ ID NO: 4569; and/or
(c) the encoded miR-142-3p binding site comprises the nucleotide sequence of SEQ ID NO: 4568, or a nucleotide sequence substantially identical (e.g., having at least 90%, 92%, 95%, 97%, 98%, or 99% sequence identity) thereto; or a nucleotide sequence having at least one, two, three, four, five, six, or seven modifications, but no more than ten modifications of SEQ ID NO: 4568.

18.-20. (canceled)

21. An AAV viral genome comprising:
(i) the nucleotide sequence of any one of SEQ ID NOs: 4547, 4548, 4551, 4552, 4555, 4556, 4559, or 4560, or a nucleotide sequence having at least 90%, 92%, 95%, 96%, 97%, 98%, or 99% sequence identity thereto; or
(ii) the nucleotide sequence of any one of SEQ ID NOs: 4549, 4550, 4553, 4554, 4557, 4558, 4561, or 4562, or a nucleotide sequence having at least 90%, 92%, 95%, 96%, 97%, 98%, or 99% sequence identity thereto, optionally wherein the nucleotide sequence comprises or does not comprise a tag, e.g., comprising the nucleotide sequence of SEQ ID NO: 2118.

22. A viral particle, e.g., an AAV viral particle, comprising the AAV viral genome of claim 10, and a capsid protein.

23. The AAV viral particle of claim 22, wherein:
(i) the capsid protein comprises
a VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2Aβ (G2A3), AAVG2B4 (G2B4), AAVG2B5 (G2B5), AAVPHP.N (PHP.N), PHP.S, AAV1, AAV2, AAV2 variant, AAV2/3 variant, AAV4, AAV5, AAV6, AAV7, AAV8, AAV9.47, AAV9(hu14), AAV9, AAV9 K449R, AAV10, AAV11, AAV12, AAVrh8, AAVrh10, AAVDJ, AAVDJ8, AAV2G9, or a functional variant thereof with amino acid sequence identity of at least 70%, 75%, 80%, 85%, 90%, 91%, 92%, 93%, 94%, 95%, 96%, 97%, 98%, 99%, or greater than 99%; optionally, the capsid comprises a VOY101 capsid protein;
(ii) the capsid protein comprises any of the capsid proteins listed in Table 1 or a functional variant thereof having at least 70%, 75%, 80%, 85%, 90%, 91%, 92%, 93%, 94%, 95%, 96%, 97%, 98%, 99%, or greater than 99% sequence identity thereto;
(iii) the capsid comprises the amino acid of SEQ ID NO: 1, or an amino acid sequence at least 95% identical thereto;
(iv) the capsid comprises the amino acid sequence encoded by the nucleotide sequence of SEQ ID NO: 2, or a nucleotide sequence at least 95% identical thereto; or
(v) the nucleotide sequence encoding the capsid comprises the nucleotide sequence of SEQ ID NO: 2, or a nucleotide sequence at least 95% identical thereto.

24. (canceled)

25. A vector comprising the nucleic acid of claim 1.

26. A host cell comprising the nucleic acid of claim 1, optionally wherein the host cell is an insect cell, a bacterial cell or a mammalian cell.

27. A method of making an isolated AAV particle, the method comprising:
(i) providing a cell comprising an AAV viral genome comprising the nucleic acid of claim 1; and
(ii) incubating the cell under conditions suitable to enclose the viral genome in a capsid protein, e.g., a VOY101 capsid protein;
thereby making the isolated AAV particle.

28. A pharmaceutical composition comprising the nucleic acid of claim 1, and a pharmaceutically acceptable carrier or excipient; optionally, wherein the pharmaceutically acceptable carrier or excipient comprises 10 mM disodium phosphate, 2 mM monopotassium phosphate, 2.7 mM potassium chloride, 192 mM sodium chloride, and 0.001% Pluronic Acid (F-68).

29. A method for treating tauopathy in a subject in need thereof, the method comprising administering to said subject a therapeutically effective amount of a composition comprising the pharmaceutical composition of claim 28, thereby treating the tauopathy in the subject.

30. The methods of claim 29, wherein the tauopathy is selected from the group consisting of Alzheimer's disease (AD), frontotemporal dementia and parkinsonism linked to chromosome 17 (FTDP-17), Frontotemporal lobar degeneration (FTLD), Frontotemporal dementia, chronic traumatic encephalopathy (CTE), Progressive Supranuclear Palsy (PSP), Down's syndrome, Pick's disease, Corticobasal degeneration (CBD), Corticobasal syndrome, Amyotrophic lateral sclerosis (ALS), Prion diseases, Creutzfeldt-Jakob disease (CJD), Multiple system atrophy, Tangle-only dementia, and Progressive subcortical gliosis and other tau associated disease.

31. The method of claim 29, comprising administering the composition intravenously, intramuscularly, intravascularly, via intraparenchymal administration, intracerebrally, intracerebroventricularly, via intra-cisterna magna (ICM) injection, intrathecally, via focused ultrasound (FUS), e.g., coupled with the intravenous administration of microbubbles (FUS-MB), or MRI-guided FUS coupled with intravenous administration; optionally, the AAV particle is administered to the subject intravenously; or via intra-cisterna magna injection (ICM).

32.-35. (canceled)
